(19) United States
(12) Patent Application Publication
(10) Pub. No.: US 2025/0339560 A1
(43) Pub. Date: Nov. 6, 2025

(54) GENE THERAPY DELIVERY COMPOSITIONS AND METHODS FOR TREATING HEARING LOSS

(71) Applicant: Akouos, Inc., Boston, MA (US)

(72) Inventors: Emmanuel John SIMONS, Boston, MA (US); Robert NG, Boston, MA (US); Danielle R. LENZ, Boston, MA (US); Hao CHIANG, Boston, MA (US)

(21) Appl. No.: 18/560,064

(22) PCT Filed: May 9, 2022
(86) PCT No.: PCT/US2022/028396

§ 371 Date: Nov. 9, 2023

Related U.S. Application Data
(60) Provisional application No. 63/188,450, filed on May 13, 2021.
(60) Provisional application No. 63/251,025, filed on Sep. 30, 2021.
(60) Provisional application No. 63/277,549, filed on Nov. 9, 2021.

(51) Int. Cl. A61K 48/00; A61K 38/17; A61P 27/16; C12N 15/86

(52) U.S. Cl. CPC A61K 48/0058; A61K 38/1709; A61P 27/16; C12N 15/86; C12N 2750/14122; C12N 2750/14143; C12N 2800/40

(57) ABSTRACT

The present disclosure provides constructs comprising a coding sequence operably linked to a promoter, wherein the coding sequence encodes a polypeptide (e.g., a therapeutic polypeptide). Exemplary constructs include AAV constructs. Also provided are methods of using disclosed constructs for the treatment of hearing loss and/or deafness.

### CROSS-REFERENCE TO RELATED APPLICATIONS

This application claims the benefit of U.S. Provisional Application No. 63/188,450, filed May 13, 2021, U.S. Provisional Application No. 63/251,025, filed Sep. 30, 2021, and U.S. Provisional Application No. 63/277,549, filed Nov. 9, 2021, which are hereby incorporated by reference in their entirety.

### REFERENCE TO SEQUENCE LISTING SUBMITTED ELECTRONICALLY

The content of the electronically submitted sequence listing in ASCII text file (Name: 4833_008CP03_Seglisting_ST25.TXT; Size: 269,049 bytes; and Date of Creation: May 9, 2022) filed with the application is incorporated herein by reference in its entirety.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

Sensorineural hearing loss (SNHL) is the most common congenital sensory impairment, with the most common genetic cause being mutations in the gap junction R 2 gene (GJB2) encoding the connexin 26 (Cx26) protein.

### SUMMARY

Certain aspects of the disclosure are directed to promoters, e.g., cell specific promoters, which are derived from portions of GDF6, PARM1, MMP15, or VIM promoters, and are capable of directing transcription of the coding sequence (e.g., encoding Connexin 26 polypeptide or functional fragment thereof) in an inner ear support cell.

Certain aspects of the disclosure are directed to polynucleotide comprising a sequence having at least 85%, at least 90%, at least 95%, at least 96%, at least 97%, at least 98%, at least 99%, or 100% identity to any one of SEQ ID NOs: 16, 28, 40, 57, or 90-99. In some aspects, the polynucleotide is a promoter.

Certain aspects of the disclosure are directed to a polynucleotide comprising a sequence having at least 85%, at least 90%, at least 95%, at least 96%, at least 97%, at least 98%, at least 99%, or 100% identity to any one of SEQ ID NOs: 40, 90, 96, or 99.

In some aspects, the polynucleotide comprises a nucleic acid sequence having at least 85%, at least 90%, at least 95%, at least 96%, at least 97%, at least 98%, at least 99%, or 100% identity to SEQ ID NO: 90.

In some aspects, the polynucleotide comprises a nucleic acid sequence having at least 85%, at least 90%, at least 95%, at least 96%, at least 97%, at least 98%, at least 99%, or 100% identity to SEQ ID NO: 40.

In some aspects, the polynucleotide comprises a nucleic acid sequence having at least 85%, at least 90%, at least 95%, at least 96%, at least 97%, at least 98%, at least 99%, or 100% identity to SEQ ID NO: 96.

In some aspects, the polynucleotide comprises a nucleic acid sequence having at least 85%, at least 90%, at least 95%, at least 96%, at least 97%, at least 98%, at least 99%, or 100% identity to SEQ ID NO: 99.

In some aspects, the polynucleotide is capable of directing transcription of a coding sequence for a Connexin 26 polypeptide or a functional fragment thereof.

Certain aspects of the disclosure are directed to construct comprising the polynucleotide disclosed herein and a nucleic acid sequence comprising the coding sequence for a/the Connexin 26 polypeptide or functional fragment thereof. In some aspects, the construct is an expression cassette.

In some aspects, the polynucleotide of the construct is a promoter and is operably linked to a/the coding sequence. In some aspects, the polynucleotide is capable of directing transcription of the coding sequence in an inner ear support cell.

In some aspects, polypeptide of the construct is a Connexin 26 polypeptide or functional fragment thereof.

Certain aspects of the disclosure are directed to a construct comprising a construct comprising the polynucleotide. In some aspects, the construct further comprises a nucleic acid sequence encoding a polypeptide. In some aspects, the polynucleotide is operably linked to the nucleic acid sequence encoding the polypeptide. In some aspects, the polynucleotide promotes expression of the nucleic acid in an inner ear support cell.

Certain aspects of the disclosure are directed to a construct comprising a polynucleotide encoding a therapeutic polypeptide operably linked to a promoter which expresses the polynucleotide in an inner ear support cell. In some aspects, the polynucleotide encodes a therapeutic polypeptide or a reporter polypeptide. In some aspects, the promoter selectively expresses the polynucleotide in an inner ear support cell.

Certain aspects of the disclosure are directed to a construct comprising a polynucleotide encoding a polypeptide operably linked to a promoter which expresses the polynucleotide in an inner ear support cell, wherein the promoter is heterologous to the polynucleotide.

Certain aspects of the disclosure are directed to an expression construct comprising a coding sequence for a Connexin 26 polypeptide or a functional fragment thereof operably linked to a promoter, wherein the promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99, wherein the promoter is capable of directing transcription of the coding sequence.

In some aspects, the promoter of the expression construct comprises a nucleic acid sequence having at least 85%, at least 90%, at least 95%, at least 96%, at least 97%, at least 98%, at least 99%, or 100% identity to SEQ ID NO: 90.

In some aspects, the promoter of the expression construct comprises a nucleic acid sequence having at least 85%, at least 90%, at least 95%, at least 96%, at least 97%, at least 98%, at least 99%, or 100% identity to SEQ ID NO: 40.

In some aspects, the promoter of the expression construct comprises a nucleic acid sequence having at least 85%, at least 90%, at least 95%, at least 96%, at least 97%, at least 98%, at least 99%, or 100% identity to SEQ ID NO: 96.

In some aspects, the promoter of the expression construct comprises a nucleic acid sequence having at least 85%, at least 90%, at least 95%, at least 96%, at least 97%, at least 98%, at least 99%, or 100% identity to SEQ ID NO: 99.

In some aspects, the expression construct further comprises a second promoter operably linked to the coding sequence, wherein the second promoter is heterologous or homologous to the coding sequence.

In some aspects, the promoter of the expression construct is capable of directing transcription of the coding sequence in an inner ear support cell.

In some aspects, the inner ear support cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, the polynucleotide, construct, or the expression construct disclosed herein, further comprises a minimal GJB2 promoter which is operably linked to the coding sequence for the Connexin 26 polypeptide or functional fragment thereof.

In some aspects, the construct or the expression construct disclosed herein comprises a GJB2 nucleic acid sequence having at least 85%, at least 90%, at least 95%, at least 96%, at least 97%, at least 98%, at least 99%, or 100% identity to any one of SEQ ID NOs: 117-126.

Certain aspects of the disclosure are directed to an expression construct comprising a coding sequence for a Connexin 26 polypeptide or functional fragment thereof operably linked to an inner ear supporting cell selective promoter and a minimal GJB2 promoter, wherein the polynucleotide is expressed in an inner ear support cell. In some aspects, the inner ear supporting cell selective promoter is heterologous to the coding sequence for the Connexin 26 polypeptide or functional fragment thereof.

In some aspects, the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

In some aspects, the promoter comprises a nucleic acid sequence having at least 85%, at least 90%, at least 95%, at least 96%, at least 97%, at least 98%, at least 99%, or 100% identity to SEQ ID NO: 90.

In some aspects, the promoter comprises a nucleic acid sequence having at least 85%, at least 90%, at least 95%, at least 96%, at least 97%, at least 98%, at least 99%, or 100% identity to SEQ ID NO: 40.

In some aspects, the promoter comprises a nucleic acid sequence having at least 85%, at least 90%, at least 95%, at least 96%, at least 97%, at least 98%, at least 99%, or 100% identity to SEQ ID NO: 96.

In some aspects, the promoter comprises a nucleic acid sequence having at least 85%, at least 90%, at least 95%, at least 96%, at least 97%, at least 98%, at least 99%, or 100% identity to SEQ ID NO: 99.

In some aspects, the inner ear supporting cell selective promoter comprises a nucleic acid sequence having having at least 95% identity to a sequence is selected from one or more of SEQ ID NO: 90, 40, 96, or 99.

In some aspects, the inner ear support cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, the polynucleotide, the construct, or expression construct of the disclosure comprises a minimal GJB2 promoter comprising a nucleic acid sequence with at least 85%, at least 90%, at least 95%, at least 96%, at least 97%, at least 98% at least 99%, or 100% identity to SEQ ID NO: 86.

In some aspects, the expression construct comprises a GJB2 nucleic acid sequence having at least 85%, at least 90%, at least 95%, at least 96%, at least 97%, at least 98%, at least 99%, or 100% identity to any one of SEQ ID NOs: 117-126.

Certain aspects of the disclosure is directed to a viral vector construct comprising: (i) a 5′ inverted terminal repeat (ITR), (ii) a coding sequence for a Connexin 26 polypeptide or functional fragment thereof operably linked to a promoter which is capable of directing transcription of the coding sequence in an inner ear support cell, and (iii) a 3′ ITR, wherein the promoter is heterologous to the coding sequence. In some aspects, the viral construct promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

In some aspects, the viral construct promoter comprises a nucleic acid sequence having at least 85%, at least 90%, at least 95%, at least 96%, at least 97%, at least 98%, at least 99%, or 100% identity to SEQ ID NO: 90.

In some aspects, the viral construct promoter comprises a nucleic acid sequence having at least 85%, at least 90%, at least 95%, at least 96%, at least 97%, at least 98%, at least 99%, or 100% identity to SEQ ID NO: 40.

In some aspects, the viral construct promoter comprises a nucleic acid sequence having at least 85%, at least 90%, at least 95%, at least 96%, at least 97%, at least 98%, at least 99%, or 100% identity to SEQ ID NO: 96.

In some aspects, the viral construct promoter comprises a nucleic acid sequence having at least 85%, at least 90%, at least 95%, at least 96%, at least 97%, at least 98%, at least 99%, or 100% identity to SEQ ID NO: 99.

In some aspects, the viral vector construct further comprises a 5′ untranslated region (UTR.

In some aspects, the viral vector construct further comprises a 3′ untranslated region (UTR).

In some aspects, the viral vector construct comprises: (i) the 5′ inverted terminal repeat (ITR), (ii) a 5′ untranslated region (UTR), (iii) the coding sequence for the Connexin 26 polypeptide or functional fragment thereof operably linked to a promoter which expresses the polynucleotide in an inner ear support cell, (iv) a 3′ UTR, and (v) the 3′ ITR.

In some aspects, the viral vector construct comprises a nucleic acid sequence having at least 85%, at least 90%, at least 95%, at least 96%, at least 97%, at least 98%, at least 99%, or 100% identity to any one of SEQ ID NOs: 117-126.

In some aspects, the viral vector construct comprises: (i) a 5′ inverted terminal repeat (ITR), (ii) a coding sequence for a Connexin 26 polypeptide or functional fragment thereof operably linked to an inner ear supporting cell selective promoter and a minimal GJB2 promoter, and (iii) a 3′ ITR, wherein the inner ear supporting cell selective promoter is heterologous to the coding sequence.

In some aspects, the viral vector construct the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

In some aspects, the viral vector construct comprises: (i) the 5′ inverted terminal repeat (ITR), (ii) a 5′ untranslated region (UTR), (iii) the coding sequence for the Connexin 26 polypeptide or functional fragment thereof operably linked to an inner ear supporting cell selective promoter and a minimal GJB2 promoter, (iv) a 3′ UTR, and (v) the 3′ ITR.

In some aspects, the viral vector construct comprises a GJB2 nucleic acid sequence having at least 85%, at least 90%, at least 95%, at least 96%, at least 97%, at least 98%, at least 99%, or 100% identity to any one of SEQ ID NOs: 117-126.

In some aspects, the viral vector construct comprises a minimal GJB2 promoter comprising a nucleic acid sequence with at least 85%, at least 90%, at least 95%, at least 96%, at least 97%, at least 98% at least 99%, or 100% identity to SEQ ID NO: 86.

In some aspects, the promoter is capable of expressing the coding sequence for the Connexin 26 polypeptide or functional fragment thereof in an inner ear support cell selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, the 5′ UTR comprises a nucleic acid sequence having at least 85%, at least 90%, at least 95%, at least 96%, at least 97%, at least 98%, at least 99%, or at least 100% identity to the sequence of any one of SEQ ID NOs: 20, 21, or 66.

In some aspects, the 3′ UTR comprises a nucleic acid sequence having at least 85%, at least 90%, at least 95%, at least 96%, at least 97%, at least 98%, at least 99%, or at least 100% identity to the sequence of any one of SEQ ID NOs: 22, 67, 68, or 69.

In some aspects, the polynucleotide, the construct, the expression construct, or viral vector construct disclosed herein, further comprises a polyA tail. In some aspects, the polyA tail is a bovine growth hormone, mouse-β-globin, mouse-α-globin, human collagen, polyoma virus, the Herpes simplex virus thymidine kinase gene (HSV TK), IgG heavy-chain gene, human growth hormone, or a SV40 late and early poly(A). In some aspects, the polyA tail is a bovine growth hormone polyA.

In some aspects, the viral vector construct disclosed herein, further comprises a 5′ and a 3′ inverted terminal repeat (ITR). In some aspects, the 5′ ITR and the 3′ ITR flank the promoter and coding sequence. In some aspects, the 5′ ITR and the 3′ ITR are AAV ITRs are derived from a serotype selected from AAV1, AAV2, AAV3, AAV4, AAV5, AAV6, AAV7, AAV8, AAV9, AAV10, AAV 11, and AAV Anc80 ITRs. In some aspects, the AAV ITRs are derived from serotype AAV2.

In some aspects, the 5′ AAV ITR comprises the nucleic acid sequence of SEQ ID NO: 8 or SEQ ID NO: 52.

In some aspects, the 3′ AAV ITR comprises the nucleic acid sequence of SEQ ID NO: 9 or SEQ ID NO: 53.

In some aspects, the viral vector construct disclosed herein comprises: a) the 5′ ITR comprises a nucleic acid sequence according to SEQ ID NO: 8 and the 3′ ITR comprises a nucleic acid sequence according to SEQ ID NO: 9; and/or b) the 5′ ITR comprises a nucleic acid sequence according to SEQ ID NO: 52 and the 3′ ITR comprises a nucleic acid sequence according to SEQ ID NO: 53.

In some aspects, the viral vector comprises (i) the 5′ ITR comprises the nucleic acid sequence of SEQ ID NOs: 8 or 52, (ii) the 5′ UTR comprises the nucleic acid of any one of SEQ ID NOs: 20, 21, or 66, (iii) the promoter comprises the nucleic acid sequence of any one of SEQ ID NOs: 10-16, 28, 40, 57, 90-99, (iv) the 3′ UTR comprises the nucleic acid sequence of SEQ ID NOs: 22, 67, 68, or 69, and (v) the 3′ ITR comprises the nucleic acid sequence of SEQ ID NOs: 9 or 53.

In some aspects, the viral vector comprises (i) the 5′ ITR comprises the nucleic acid sequence of SEQ ID NOs: 8 or 52, (ii) the 5′ UTR comprises the nucleic acid of any one of SEQ ID NOs: 20, 21, or 66, (iii) the inner ear supporting cell selective promoter comprises the nucleic acid sequence of any one of SEQ ID NOs: 10-16, 28, 40, 57, 90-99, the minimal GJB2 promoter comprises the sequence of SEQ ID NO: 86, (v) the 3′ UTR comprises the nucleic acid sequence of SEQ ID NOs: 22, 67, 68, or 69, and (vi) the 3′ ITR comprises the nucleic acid sequence of SEQ ID NOs: 9 or 53.

In some aspects, the construct, the expression construct, or viral vector construct disclosed herein comprises a nucleic acid sequence according to any one of SEQ ID NOs: 7, 17, 38, 45-51, 54, 61, 82-84, 87-88, and 100-107.

In some aspects, the construct, the expression construct, or viral vector construct is selectively expressed in an inner ear supporting cell.

In some aspects, the construct, the expression construct, or viral vector construct comprises nucleotides 12-4557 of SEQ ID NO: 7, nucleotides 12-4338 of SEQ ID NO: 17, nucleotides 12-3976 of SEQ ID NO: 38, nucleotides 12-4754 of SEQ ID NO: 54, nucleotides 12-4429 of SEQ ID NO: 61, nucleotides 12-4645 of SEQ ID NO: 100, nucleotides 12-4708 of SEQ ID NO: 101, nucleotides 12-4993 of SEQ ID NO: 102, nucleotides 12-4496 of SEQ ID NO: 103, nucleotides 12-4253 of SEQ ID NO: 104, nucleotides 12-4320 of SEQ ID NO: 105, nucleotides 12-4464 of SEQ ID NO: 106, or nucleotides 12-4328 of SEQ ID NO: 107.

Certain aspects of the disclosure are directed to a viral vector or AAV particle comprising the polynucleotide, construct, expression construct, or viral vector construct disclosed herein. In some aspects, the viral vector is selected from the group consisting of an adeno-associated viral (AAV), adenovirus, or lentiviral vector. In some aspects, the viral vector is an AAV vector.

In some aspects, the viral vector or AAV particle comprises an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 serotype capsid. In some aspects, the AAV vector or AAV particle comprises an AAV capsid which an AAV Anc80 capsid.

Certain aspects of the disclosure are directed to a composition comprising the polynucleotide, the construct, the expression construct, viral vector construct, or AAV particle disclosed herein. In some aspects, the composition is a pharmaceutical composition further comprising a pharmaceutically acceptable carrier. In some aspects, the pharmaceutical composition is a synthetic perilymph solution.

Certain aspects of the disclosure are directed to ex vivo cell comprising the polynucleotide, the construct, the expression construct, the viral vector construct, the viral vector, or the AAV particle disclosed herein.

In some aspects, the ex vivo cell is an inner ear cell. In some aspects, the ex vivo cell is an inner ear supporting cell. In some aspects, the supporting cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Certain aspects of the disclosure are directed to a method comprising, transducing an ex vivo cell with: a. the polynucleotide, the construct, the expression construct, the viral vector construct, the viral vector, or the AAV particle disclosed herein; and b. one or more helper plasmids collectively comprising an AAV Rep gene, AAV Cap gene, AAV VA gene, AAV E2a gene, and AAV E4 gene.

Certain aspects of the disclosure are directed to a method of expressing the Connexin 26 polypeptide or functional fragment thereof in an inner ear supporting cell, comprising administering the polynucleotide, the construct, the expression construct, the viral vector construct, the viral vector, the AAV particle, or the ex vivo cell disclosed herein.

Certain aspects of the disclosure are directed to a method of increasing expression of the Connexin 26 polypeptide or functional fragment thereof in an inner ear supporting cell, comprising administering the polynucleotide, the construct, the expression construct, the viral vector construct, the viral vector, the AAV particle, or the ex vivo cell disclosed herein to the subject.

In some aspects, the expression of the Connexin 26 polypeptide or functional fragment thereof in the inner ear supporting cell is increased relative to endogenous expression of the polypeptide in the inner ear supporting cell.

Certain aspects of the disclosure are directed to a method of treating hearing loss in a subject suffering from or at risk of hearing loss, comprising administering the polynucleotide, the construct, the expression construct, the viral vector construct, the viral vector, the AAV particle, or the ex vivo cell disclosed herein to the subject.

In some aspects, (i) the Connexin 26 polypeptide or functional fragment thereof is predominately expressed in inner ear supporting cells, (ii) the Connexin 26 polypeptide or functional fragment thereof is selectively expressed at a higher level in inner ear supporting cells than in inner ear hair cells, (iii) the Connexin 26 polypeptide or functional fragment thereof not expressed at levels sufficient to cause toxicity in inner ear hair cells, or (iv) or any combination thereof.

In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), and OC90+ cells (OC90).

In some aspects, the administration is to the inner ear of the subject.

In some aspects, the administration is to the cochlea of the subject.

In some aspects, the administration is via a round window membrane injection.

Certain aspects are directed to the use of the polynucleotide, the construct, the expression construct, the viral vector construct, the viral vector, the AAV particle, or the ex vivo cell disclosed herein, for the treatment of hearing loss in a subject suffering from or at risk of hearing loss.

Certain aspects are directed to the use of polynucleotide, the construct, the expression construct, the viral vector construct, the viral vector, the AAV particle, or the ex vivo cell disclosed herein, in the manufacture of a medicament for the treatment of hearing loss.

In some aspects, the polynucleotide, the construct, the expression construct, the viral vector construct, the viral vector, the AAV particle, or the ex vivo cell disclosed herein, for use as a medicament.

In some aspects, the polynucleotide, the construct, the expression construct, the viral vector construct, the viral vector, the AAV particle, or the ex vivo cell disclosed herein, for use in the treatment of hearing loss.

In some aspects, the construct, vector, AAV particle, composition or ex vivo cell is pre-loaded in a device for administration. In some aspects, the device is a microcatheter. In some aspects, the microcatheter is shaped such that it can enter the middle ear cavity via the external auditory canal and contact the end of the microcatheter with the RWM. In some aspects, a distal end of the microcatheter is comprised of at least one microneedle with diameter of between 10 and 1,000 microns. In some aspects, the kit further comprises a device. In some aspects, the device is a device described in any one of FIGS. 5-8. In some aspects, the device comprises a needle comprising a bent portion and an angled tip.

Certain aspects are directed to a kit comprising the polynucleotide, the construct, the expression construct, the viral vector construct, the viral vector, the AAV particle, or the ex vivo cell disclosed herein. In some aspects, the kit further comprises a device disclosed herein.

Certain aspects of the disclosure are directed to a construct comprising a polynucleotide encoding a polypeptide operably linked to a promoter, wherein the promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99. In some aspects, the promoter is heterologous to the polynucleotide.

Certain aspects of the disclosure are directed to a construct comprising a polynucleotide encoding a polypeptide, an inner ear supporting cell selective promoter and a minimal GJB2 promoter, wherein the polynucleotide is operably linked to the inner ear supporting cell selective promoter and the minimal GJB2 promoter such that the polynucleotide is expressed in an inner ear support cell, wherein the inner ear supporting cell selective promoter is heterologous to the polynucleotide.

Certain aspects of the disclosure are directed to a construct comprising a polynucleotide encoding a polypeptide, an inner ear supporting cell selective promoter and a minimal GJB2 promoter, whererin the polynucleotide is operably linked to the inner ear supporting cell selective promoter and the minimal GJB2 promoter, wherein the inner ear supporting cell selective promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99. In some aspects, the inner ear supporting cell selective promoter is heterologous to the polynucleotide. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

Certain aspects of the disclosure are directed to a construct comprising: (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide.

Certain aspects of the disclosure are directed to a construct comprising: (i) a 5′ inverted terminal repeat (ITR), (ii) a polynucleotide encoding a polypeptide operably linked to a promoter, and (iii) a 3′ ITR, wherein the promoter comprises a nucleic acid sequence havingat least 85%, at least 90%, at least 95%, at least 96%, at least 97%, at least 98%, at least 99%, or 100% identity to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, the promoter is selected from one or more a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, derivatives thereof, or fragments thereof.

In some aspects, the promoter is a GJB2 promoter or a minimal GJB2 promoter.

In some aspects, the construct comprises two or more promoters. In some aspects, the first promoter is selected from a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, or any combination thereof. In some aspects, the second promoter is selected from a GJB2 promoter or a minimal GJB2 promoter.

Certain aspects of the disclosure are directed to a construct comprising: (i) a 5′ inverted terminal repeat (ITR), (ii) a polynucleotide encoding a polypeptide operably linked to an inner ear supporting cell selective promoter and a minimal GJB2 promoter, wherein the polynucleotide is expressed in an inner ear support cell, and (iii) a 3′ ITR, wherein the inner ear supporting cell selective promoter is heterologous to the polynucleotide.

Certain aspects of the disclosure are directed to a construct comprising: (i) a 5′ inverted terminal repeat (ITR), (ii) a polynucleotide encoding a polypeptide operably linked to a inner ear supporting cell selective promoter and a minimal GJB2 promoter, and (iii) a 3′ ITR, wherein the inner ear supporting cell selective promoter comprises a nucleic acid sequence havingat least 85%, at least 90%, at least 95%, at least 96%, at least 97%, at least 98%, at least 99%, or 100% identity to any one of SEQ ID NOs: 16, 28, 40, 57, or 90-99.

In some aspects, inner ear supporting cells include, but are not limited to, inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, the promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99.

In some aspects, the construct comprises a miRNA regulatory target site (miRTS) for a microRNA expressed in an inner ear cell. In some aspects, the microRNA is expressed in an inner ear hair cell. In some aspects, the microRNA is one or more of miR-194, miR-140, miR-18a, miR-99a, miR-30b, miR-15a, miR182, miR-183, or any combination thereof.

Certain aspects of the disclosure are directed to a construct comprising a polynucleotide encoding a polypeptide operably linked to a promoter, wherein the construct comprises a miRNA regulatory target site (miRTS) for a microRNA expressed in an inner ear cell.

In some aspects, the polynucleotide encodes a therapeutic polypeptide (e.g., a Connexin 26 polypeptide) or a reporter polypeptide.

In some aspects, the microRNA is expressed in one or more of inner ear hair cells, spiral ganglion cells, lateral supporting cells, basilar membrane cells, medial supporting cells, spiral limbus cells, or inner sulcus cells.

In some aspects, the microRNA is expressed in inner ear hair cells.

In some aspects, the microRNA is one or more of miR-194, miR-140, miR-18a, miR-99a, miR-30b, miR-15a, miR182, or miR-183.

In some aspects, the construct comprises a 5′ and a 3′ inverted terminal repeat (ITR). In some aspects, the construct comprises a 5′ untranslated region (UTR). In some aspects, the construct comprises a 3′ untranslated region (UTR).

Certain aspects of the disclosure are directed to vectors, viral particles (e.g., AAV), ex vivo cells, and compositions comprising the constructs disclosed herein.

Certain aspects of the disclosure are directed to an adeno-associated virus (AAV) particle comprising a construct disclosed herein.

Certain aspects are directed to an adeno-associated virus (AAV) particle comprising a construct comprising: (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide. In some aspects, the promoter comprises a nucleic acid sequence at least 85%, at least 90%, at least 95%, at least 96%, at least 97%, at least 98%, at least 99%, or 100% identical to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identical to SEQ ID NO: 86.

Certain aspects are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter which expresses the polynucleotide in an inner ear support cell, (iv) a 3′ UTR, and (v) a 3′ ITR, wherein the promoter is heterologous to the polynucleotide.

Certain aspects of the disclosure are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, wherein the polynucleotide is expressed in an inner ear support cell, (iv) a 3′ UTR, and (v) a 3′ ITR, wherein the inner ear supporting cell selective promoter is heterologous to the polynucleotide

Certain aspects of the disclosure are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

Certain adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter, (iv) a miRNA regulatory target site (miRTS) for a microRNA expressed in an inner ear cell, (v) a 3′ UTR, and (vi) a 3′ ITR.

Certain aspects of the disclosure are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and minimal GJB2 promoter, (iv) a 3′ UTR, and (v) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

Certain adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, (iv) a miRNA regulatory target site (miRTS) for a microRNA expressed in an inner ear cell, (v) a 3′ UTR, and (vi) a 3′ ITR.

In some aspects, the inner ear supporting cell selective promoter is selected from one or more a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, derivatives thereof, or fragments thereof.

In some aspects, the minimal GJB2 promoter comprises a nucleic acid sequence with at least 85%, at least 90%, at least 95%, at least 96%, at least 97%, at least 98% at least 99%, or 100% identity to SEQ ID NO: 86.

Certain aspects of the disclosure are directed to methods of using the constructs, vectors, viral particles (e.g., AAV), ex vivo cells, and compositions disclosed herein for expressing a polypeptide in an inner ear cell (e.g., a supporting cells).

Certain aspects of the disclosure are directed to methods of using the constructs, vectors, viral particles (e.g., AAV), ex vivo cells, and compositions disclosed herein for increasing expression of a polypeptide (e.g., a therapeutic polypeptide, a Connexin 26 polypeptide) in an inner ear cell (e.g., a supporting cells). In some aspects, the increasing expression is relative to the corresponding endogenous polypeptide expression in the inner ear cell (e.g., a supporting cells).

Certain aspects of the disclosure are directed to methods of using the constructs, vectors, viral particles (e.g., AAV), ex vivo cells, and compositions disclosed herein for decreasing expression of a polypeptide (e.g., a therapeutic polypeptide) in non-inner ear supporting cells (e.g., inner ear hair cells). In some aspects, the decreasing expression is relative to the corresponding endogenous polypeptide expression in the non-inner ear cell supporting cells (e.g., inner ear hair cells).

Certain aspects of the disclosure are directed to methods of using the constructs, vectors, viral particles (e.g., AAV), ex vivo cells, and compositions disclosed herein for reducing toxicity associated with expression of a polypeptide, (e.g., a therapeutic polypeptide) in an inner ear cell.

Certain aspects of the disclosure are directed to methods of using the constructs, vectors, viral particles (e.g., AAV), ex vivo cells, and compositions disclosed herein for treating hearing loss in a subject suffering from or at risk of hearing loss.

### BRIEF DESCRIPTION OF THE DRAWINGS

FIG. 1A-1B panel (1A) depicts a simplified endogenous AAV genome; panel (1B) depicts a simplified recombinant AAV (rAAV) construct capable of expressing a therapeutic polypeptide (e.g., a GJB2 gene).

FIGS. 2A-2H depict alternative exemplary rAAV constructs comprising a therapeutic polypeptide FIG. 2A depicts an exemplary rAAV construct comprising a 5′ ITR, a CAG promoter, a nucleic acid encoding a therapeutic polypeptide (a hGJB2 gene), a bGH polyA, and a 3′ ITR. FIG. 2B depicts an exemplary rAAV construct comprising a 5′ ITR, a CAG promoter, a nucleic acid encoding a therapeutic polypeptide (a hGJB2 gene), a 3′ UTR, a bGH polyA, and a 3′ ITR. FIG. 2C depicts an exemplary rAAV construct comprising a 5′ ITR, a CAG promoter, a 5′ UTR, a nucleic acid encoding a therapeutic polypeptide (a hGJB2 gene), a FLAG tag, a 3′ UTR, a bGH polyA, and a 3′ ITR. FIG. 2D depicts an exemplary rAAV construct comprising a 5′ ITR, a smCBA promoter, a 5′ UTR, a nucleic acid encoding a therapeutic polypeptide (a hGJB2 gene), a FLAG tag, a 3′ UTR, a bGH polyA, and a 3′ ITR. FIG. 2E depicts an exemplary rAAV construct comprising a 5′ ITR, a promoter comprising a CMV promoter and a hGJB2 promoter, a 5′ UTR, a nucleic acid encoding a hGJB2 gene, a FLAG tag, a 3′ UTR, a bGH polyA, and a 3′ ITR. FIG. 2F depicts an exemplary rAAV construct comprising a 5′ ITR, a CAG promoter, a 5′ UTR, a hGJB2 promoter, a FLAG tag, a microRNA regulatory target site, a 3′ UTR, a bGH polyA, and a 3′ ITR. FIG. 2G depicts an exemplary rAAV construct comprising a 5′ ITR, a promoter comprising an inner ear supporting cell selective promoter and a hGJB2 minimal promoter, a nucleic acid encoding a therapeutic polypeptide (a hGJB2 gene), a FLAG tag, a 5′ UTR, a bGH polyA, and a 3′ ITR. FIG. 2H depicts an exemplary rAAV construct comprising a 5′ ITR, a CAG promoter, a nucleic acid encoding a therapeutic polypeptide (a hGJB2 gene), a FLAG tag, a T2A element, a nucleic acid encoding eGFP, a bGH polyA, and a 3′ ITR.

FIGS. 3A-3Q depict in vitro or ex vivo expression of a transgene in HEK293FT cells transfected or transduced with a contruct with a microRNA targeting site (miRTS) in the presence or absence of a microRNA recognizing that site. FIG. 3A is a schematic that represents construct comprising a gene of interest and a miRTS. FIG. 3B is a Venn diagram representing selection of miRTS based on expression of microRNAs expressed in the different inner ear cell types. FIG. 3C is a graph showing the GFP expression in cells transfected with miRNA-expressing plasmid (pITR.CAG.mScarlet.miRNA) and a plasmid comprising a gene-of-interest and microRNA target site (pITR.CAG.GOI.miRTS). FIG. 3D is a graph showing GFP expression as measured by flow cytometry in HEK293FT cells transduced with an AAVAnc80 vector comprising GFP and a microRNA target site (AAVAncO-CAG.GOI.miRTS) and transfected with a plasmid expressing miRNA targeting the miRTS (pITR.CAG.mScarlet.miRNA). FIG. 3E is a graph showing a gene of interest expression measured by RT-qPCR in cells transduced with an AAVAnc80 expressing the gene of interest with a microRNA targeting site (AAVAnc80-CAG.GOI.miRTS) following transfection with either of two amounts of plasmid expressing a plasmid encoding a miRNA targeting the miRTS (pITR.CAG.mScarlet.miRNA). FIG. 3F is a western blot of protein showing expression of the gene of interest in cells transduced with an AAVAnc80 comprising the gene of interest and a microRNA targeting site (AAVAnc80-CAG.GOI.miRTS) following transfection with either of two amounts of plasmid expressing a miRNA targeting the miRTS (pITR.CAG.mScarlet.miRNA). FIG. 3G is a graph showing the quantification of proteins levels determined from the western blot in FIG. 3F. FIG. 3H is a heat map of gene expression due to in vitro transduction of the gene-of-interest with the microRNA targeting site compared to transduction with the gene-of-interest alone. FIG. 3I is a volcano plot displaying differential gene expression between the samples. FIG. 3J shows expression of a gene of interest in an untreated cochlear explant (left panel) and after transduction with an AAV encoding a FLAG-tagged gene of interest without a microRNA target site (right panel). Immunostaining of the FLAG tag is shown in green. Immunostaining of MYO7A was used to label hair cells in red. White arrowheads indicate hair cells expressing the Connexin 26-FLAG. FIG. 3K shows a cochlear explant transduced with AAVAnc80-CAG-GOI.miRTS1 comprising a FLAG-tagged gene of interest and a microRNA targeting site for a microRNA expressed in hair cells. FIG. 3L shows a cochlear explant transduced with AAVAnc80-CAG-GOI.miRTS1 comprising a FLAG-tagged gene of interest and a microRNA targeting site for a microRNA expressed in hair cells. FIG. 3M shows a cochlear explant transduced with AAVAnc80-CAG-GOI.miRTS2 comprising FLAG-tagged gene of interest and a microRNA targeting site recognized by a microRNA expressed in hair cells. FIG. 3N shows a cochlear explant transduced with AAVAnc80-CAG-GOI.miRTS3 comprising FLAG-tagged gene of interest and a microRNA targeting site recognized by a microRNA expressed in hair cells. FIG. 3O shows a cochlear explant transduced with AAVAnc80-CAG-GOI.miRTS4 comprising a FLAG-tagged gene of interest and a microRNA targeting for a microRNA expressed in hair cells. FIGS. 3P and 3Q depict in vitro expression of GJB2 protein in HEK293FT cells transfected with CAG.5UTR.hGJB2.FLAG.miRTS.3UTR (SEQ ID NO: 87), CAG.5UTR.hGJB2.FLAG.3UTR (SEQ ID NO: 82), or CAG.5UTR.hGJB2.FLAG.GFP constructs. CAG.5UTR.hGJB2.FLAG.miRTS.3UTR comprises miRNA targeting sites (miRTS) for miR-182 and miR-183 in the 3UTR to permit exogenous hGJB2 knockdown in the presence of regulatory miR-182 and/or miR-183. To confirm miRNA regulation of constructs, HEK293FT cells were transfected with hGJB2 comprising plasmids and optionally co-transfected with (+) or without (−) plasmids expressing miR-182 and miR-183. 72 h post transfection the cells were harvested for protein and RNA analysis. FIG. 3P depicts exemplary GJB2 protein levels analyzed using western blot. FIG. 3Q depicts exemplary GJB2 mRNA levels analyzed using qPCR.

FIG. 4A-4C depicts FLAG protein expression in mouse cochlear explants transduced at P2 with exemplary rAAVAnc80 particles comprising constructs driven by CAG, CMVe-GJB2p, or smCBA promoter/enhancer sequences as noted, explants were fixed after 72 h, immunostaining for FLAG is noted in green, immunostaining for hair cell marker Myo7a is noted in red, and nuclear marker DAPI is noted in blue. Panel (4A) depicts exemplary explants transduced with AAVAnc80-CAG.5UTR.hGJB2.3F.3UTR (SEQ ID NO: 82) at 5.8E9 vg/explant. Panel (4B) depicts exemplary explants transduced with AAVAnc80-smCBA.5UTR.hGJB2.3F.3UTR (SEQ ID NO: 83) at 1.4E10 vg/explant. Panel (4C) depicts exemplary explants transduced with AAVAnc80-CMVeGFAPp.5UTR.hGJB2.3F.3UTR (SEQ ID NO: 84) at 1.8E10 vg/explant.

FIG. 5 illustrates a perspective of a device for delivering fluid to an inner ear, according to aspects of the present disclosure.

FIG. 6 illustrates a sideview of a bent needle sub-assembly, according to aspects of the present disclosure.

FIG. 7 illustrates a perspective view of a device for delivering fluid to an inner ear, according to aspects of the present disclosure.

FIG. 8 illustrates a perspective view of a bent needle sub-assembly coupled to the distal end of a device, according to aspects of the present disclosure.

FIGS. 9A-9O depicts in vivo expression of Connexin 26 in wild-type mice. Wild type mice (p20) were administered rAAVAnc80 particles comprising CAG.hGJB2.FLAG.GFP (schematic provided in FIG. 2H) to the cochlea (FIG. 9A). Expression of Connexin 26 in the supporting cells and inner hair cells was detected 10 days after administration. Immunostaining of actin filaments and hair cell stereocilia bundles by phalloidin is noted in blue, GFP is noted in green, FLAG is noted in purple, and endogenous Connexin 26 is noted in red. SC—supporting cells; IHC—inner hair cells; OHC—outer hair cells. Juvenile mice were administered rAAVAnc80 particles comprising AAVAnc80-CMVeGFAPp.mGJB2p.5UTR.hGJB2.FLAG.3UTR (FIG. 9B), AAVAnc80-GDF6p.mGJB2p.5UTR.hGJB2.FLAG.3UTR (FIGS. 9C and 9I) (schematic provided in FIG. 2G), AAVAnc80-IGFBP2p. mGJB2p.5UTR.hGJB2.FLAG.3UTR (FIG. 9D) (schematic provided in FIG. 2G), AAVAnc80-PARM1p.mGJB2p.5UTR.hGJB2.FLAG.3UTR (FIGS. 9E and 9J) (schematic provided in FIG. 2G), AAVAnc80-GFAPp.mGJB2p.hGJB2 (FIG. 9F), AAVAnc80-MMP15p.mGJB2p.hGJB2 (FIGS. 9G and 9L), AAVAnc80-VIMp.mGJB2p-hGJB2 (FIGS. 9H and 9K) to the cochlea. (VIM is also referred to as VIM1 in FIG. 9K.) Expression of Connexin 26 was detected two weeks after administration. Immunostaining of actin filaments and hair cell stereocilia bundles by phalloidin is noted in blue, FLAG is noted in green, and endogenous Connexin 26 or Myo7a is noted in red. FIG. 9M depicts in vivo expression of Connexin 26 in wild-type mice administered AAVAnc80 particles comprising AAVAnc80.CMVe.GFAP.mGJB2p.hGJB2.FLAG. Endogenous Connexin 26 is shown in white, flag-tagged Connexin 26 is shown in green, and hair cells are shown by phalloidin staining in blue. FIGS. 9N-9O) depicts in vivo expression of Connexin 26 in wild-type mice administered AAVAnc80 particles comprising AAVAnc80.CMVe.GDF6.mGJB2p.hGJB2.FLAG or AAVAnc80.CMVe.PARM1.mGJB2p.hGJB2.FLAG. Flag-tagged Connexin 26 is shown in green, phalloidin staining in blue, and Myo7a marking hair cells is shown in red.

FIGS. 10A-10C depicts in vitro expression of GJB2 mRNA and detection of Connexin 26 protein from constructs including supporting cell selective promoters. FIG. 10A shows Connexin 26-FLAG protein levels (“GJB2-FLAG”) in HEK293FT cells transduced with exemplary rAAVAnc80 particles comprising constructs driven by GJB6, IGFBP2, RPB7, PARM1, or GDF6 promoters in combination with a minimal GJB2 promoter. GAPDH is shown as a loading control. FIG. 10B shows GJB2 mRNA levels in HEK293FT cells transduced with rAAVAnc80 particles comprising constructs driven by GFAP and a minimal GJB2 promoter, CMV enhancer/GFAP, GJB2 enhancer/GJB2, CMV enhancer/GJB2, or CAG promoters. FIG. 10C shows Connexin 26-FLAG protein levels (GJB2-FLAG) in HEK293FT cells transfected with plasmids comprising constructs driven by FABP3, KLHL14, DBI2, TSPAN8, MMP15, SPARC, or VIM promoters in combination with a minimal GJB2 promoter. FLAG was used to distinguish protein levels between endogenous and transduced Connexin 26 expression. GAPDH is shown as a loading control.

FIG. 11 shows GJB2 mRNA levels in mouse cochlear explants transduced with rAAVAnc80 particles comprising constructs driven by a CAG promoter, a CMV enhancer/GFAP promoter, or a GFAP and a minimal GJB2 promoter. GJB2 mRNA levels were determined by qPCR.

### DEFINITIONS

The scope of the present disclosure is defined by the claims appended hereto and is not limited by certain aspects described herein. Those skilled in the art, reading the present specification, will be aware of various modifications that may be equivalent to such described aspects, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some aspects, exactly one member of a group is present in, employed in, or otherwise relevant to a given product or process. In some aspects, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where aspects or aspects are referred to as “comprising” particular elements, features, etc., certain aspects or aspects “consist,” or “consist essentially of,” such elements, features, etc. For purposes of simplicity, those aspects have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a construct or composition to a subject or system to achieve delivery of an agent to a subject or system. In some aspects, an agent is, or is included in, a composition; in some aspects, an agent is generated through metabolism of a composition or one or more components thereof. Those of ordinary skill in the art will be aware of a variety of routes that may, in appropriate circumstances, be utilized for administration to a subject, for example a human. For example, in some aspects, administration may be systematic or local. In some aspects, a systematic administration can be intravenous. In some aspects, administration can be local. Local administration can involve delivery to cochlear perilymph via, e.g., injection through a round-window membrane or into scala-tympani, a scala-media injection through endolymph, perilymph and/or endolymph following canalostomy. In some aspects, administration may involve only a single dose. In some aspects, administration may involve application of a fixed number of doses. In some aspects, administration may involve dosing that is intermittent (e.g., a plurality of doses separated in time) and/or periodic (e.g., individual doses separated by a common period of time) dosing. In some aspects, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some aspects, an amino acid has a general structure, e.g., H₂N—C(H)(R)—COOH. In some aspects, an amino acid is a naturally-occurring amino acid. In some aspects, an amino acid is a non-natural amino acid; in some aspects, an amino acid is a D-amino acid; in some aspects, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some aspects, an amino acid, including a carboxy- and/or amino-terminal amino acid in a polypeptide, can contain a structural modification as compared with general structure as shown above. For example, in some aspects, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some aspects, such modification may, for example, alter circulating half-life of a polypeptide containing a modified amino acid as compared with one containing an otherwise identical unmodified amino acid. In some aspects, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some aspects, the term “approximately” or “about” refers to a range of values that fall within ±10% (greater than or less than) of a stated reference value unless otherwise stated or otherwise evident from context (except where such number would exceed 100% of a possible value). For example, in some aspects, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some aspects, two or more entities are physically “associated” with one another if they interact, directly or indirectly, so that they are and/or remain in physical proximity with one another. In some aspects, two or more entities that are physically associated with one another are covalently linked to one another; in some aspects, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some aspects, a specific binding interaction is a biological activity. In some aspects, modulation (e.g., induction, enhancement, or inhibition) of a biological pathway or event is a biological activity. In some aspects, presence or extent of a biological activity is assessed through detection of a direct or indirect product produced by a biological pathway or event of interest.

Cell Selective Promoter: As used herein, the term “cell selective promoter” refers to a promoter that is predominately active in certain cell types (e.g., transcription of a specific gene occurs only within cells expressing transcription regulatory and/or control proteins that bind to the tissue-specific promoter). In some aspects, an inner ear supporting cell selective promoter is a promoter that is predominately active in one or more supporting cells of the inner ear.

Characteristic portion: As used herein, the term “characteristic portion,” in the broadest sense, refers to a portion of a substance whose presence (or absence) correlates with presence (or absence) of a particular feature, attribute, or activity of the substance. In some aspects, a characteristic portion of a substance is a portion that is found in a given substance and in related substances that share a particular feature, attribute or activity, but not in those that do not share the particular feature, attribute or activity. In some aspects, a characteristic portion shares at least one functional characteristic with the intact substance. For example, in some aspects, a “characteristic portion” of a protein or polypeptide is one that contains a continuous stretch of amino acids, or a collection of continuous stretches of amino acids, that together are characteristic of a protein or polypeptide. In some aspects, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some aspects, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some aspects, presence of a characteristic sequence element correlates with presence or level of a particular activity or property of a polymer. In some aspects, presence (or absence) of a characteristic sequence element defines a particular polymer as a member (or not a member) of a particular family or group of such polymers. A characteristic sequence element typically comprises at least two monomers (e.g., amino acids or nucleotides). In some aspects, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some aspects, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some aspects, two or more agents may be administered simultaneously. In some aspects, two or more agents may be administered sequentially. In some aspects, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some aspects, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some aspects, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector, capsid, viral particle and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral vector, capsid, viral particle etc.) that is capable of replicating when associated with proper control elements. Thus, in some aspects, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some aspects, a conservative amino acid substitution can be a substitution of any native residue in a protein with alanine, as used in, for example, alanine scanning mutagenesis. In some aspects, a conservative substitution is made that has a positive value in the PAM250 log-likelihood matrix disclosed in Gonnet et al., 1992, Science 256:1443-1445, which is incorporated herein by reference in its entirety. In some aspects, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. Exemplary conservative amino acid substitutions are shown in Table 1.

TABLE 1
Conservative Amino Acid Substitutions
CONSERVATIVE AMINO ACID SUBSTITUTIONS

| For Amino | | |
|---|---|---|
| Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic | D | D-Asp, D-Asn, Asn, Glu, D-Glu, Gln, D-Gln |
| Acid | | |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic | E | D-Glu, D-Asp, Asp, Asn, D-Asn, Gln, D-Gln |
| Acid | | |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, |
| | | D-Met |
| Leucine | L | D-Leu, Val, D-Val, AdaA, AdaG, Leu, D-Leu, Met, |
| | | D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S—Me-Cys, Ile, D-Ile, Leu, D-Leu, Val, |
| | | D-Val |
| Phenyl- | F | D-Phe, Tyr, D-Thr, L-Dopa, His, D-His, Trp, |
| alanine | | D-Trp, Trans-3,4 or 5-phenylproline, AdaA, AdaG, |
| | | cis-3,4 or 5-phenylproline, Bpa, D-Bpa |
| Proline | P | D-Pro, L-I-thioazolidine-4-carboxylic acid, D-or- |
| | | L-1-oxazolidine-4-carboxylic acid (Kauer, U.S. |
| | | Pat. No. 4,511,390) |
| Serine | S | D-Ser, Thr, D-Thr, allo-Thr, Met, D-Met, Met (O), |
| | | D-Met (O), L-Cys, D-Cys |
| Threonine | T | D-Thr, Ser, D-Ser, allo-Thr, Met, D-Met, Met (O), |
| | | D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, |
| | | AdaG |

Control: As used herein, the term “control” refers to the art-understood meaning of a “control” being a standard against which results are compared. Typically, controls are used to augment integrity in experiments by isolating variables in order to make a conclusion about such variables. In some aspects, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some aspects, a control is a historical control (e.g., of a test or assay performed previously, or an amount or result that is previously known). In some aspects, a control is or comprises a printed or otherwise saved record. In some aspects, a control is a positive control. In some aspects, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some aspects, “Assaying for the presence of” can be determining an amount of something present and/or determining whether or not it is present or absent.

Endogenous: As used herein in reference to a substances or process refers to a naturally occurring substances or processes that originates from within a system such as an organism, tissue, or cell.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some aspects, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some aspects, a gene product can be a transcript. In some aspects, a gene product can be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanked: As used herein, the term “flanked” refers to a position relative to ends of a reference item. More specifically, in referring to reference nucleic acid sequence(s), “flanked” refers to having a sequences upstream and downstream of the reference nucleic acid sequence(s). In some aspects, a flanked referenced nucleic acid sequence has a first sequence or series of nucleotide residues positioned adjacent to the 5′ end of the referenced nucleic acid and a second sequence or series of nucleotide residues positioned adjacent to the 3′ end of the referenced nucleic acid. In some aspects, the upstream and/or downstream flanking sequences are immediately adjacent to the referenced nucleic acid sequence. In some aspects, there are intervening nucleic acids between the upstream and/or downstream flanking sequences and the referenced nucleic acid sequence.

Functional: As used herein, the term “functional” describes something that exists in a form in which it exhibits a property and/or activity by which it is characterized. For example, in some aspects, a “functional” biological molecule is a biological molecule in a form in which it exhibits a property and/or activity by which it is characterized. In some such aspects, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some aspects, a gene includes coding sequence (i.e., sequence that encodes a particular product). In some aspects, a gene includes non-coding sequence. In some particular aspects, a gene may include both coding (e.g., exonic) and non-coding (e.g., intronic) sequence. In some aspects, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some aspects, a gene may encode a polypeptide, but that polypeptide may not be functional, e.g., a gene variant may encode a polypeptide that does not function in the same way, or at all, relative to the wild-type gene. In some aspects, a gene may encode a transcript which, in some aspects, may be toxic beyond a threshold level. In some aspects, a gene may encode a polypeptide, but that polypeptide may not be functional and/or may be toxic beyond a threshold level.

Hearing loss: As used herein, the term “hearing loss” may be used to a partial or total inability of a living organism to hear. In some aspects, hearing loss may be acquired. In some aspects, hearing loss may be hereditary. In some aspects, hearing loss may be genetic. In some aspects, hearing loss may be as a result of disease or trauma (e.g., physical trauma, treatment with one or more agents resulting in hearing loss, etc.). In some aspects, hearing loss may be due to one or more known genetic causes and/or syndromes. In some aspects, hearing loss may be of unknown etiology. In some aspects, hearing loss may or may not be mitigated by use of hearing aids or other treatments.

Heterologous: As used herein, the term “heterologous” the relationship between two or more nucleic acid or protein sequences that are derived from different sources. In some aspects, the promoter operably linked to the nucleic acid encoding the therapeutic protein may be derived from a different gene other than the gene encoding the therapeutic protein.

Identity: As used herein, the term “identity” refers to overall relatedness between polymeric molecules, e.g., between nucleic acid molecules (e.g., DNA molecules and/or RNA molecules) and/or between polypeptide molecules. In some aspects, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some aspects, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some aspects, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some aspects, a value is statistically significantly difference that a baseline or other reference measurement. In some aspects, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some aspects, an appropriate reference measurement may be or comprise a measurement in comparable system known or expected to respond in a particular way, in presence of the relevant agent or treatment. In some aspects, an appropriate reference is a negative reference; in some aspects, an appropriate reference is a positive reference.

Knockdown: As used herein, the term “knockdown” refers to a decrease in expression of one or more gene products. In some aspects, an inhibitory nucleic acid achieve knockdown. In some aspects, a genome editing system described herein achieves knockdown.

Knockout: As used herein, the term “knockout” refers to ablation of expression of one or more gene products. In some aspects, a genome editing system described herein achieve knockout.

Minimal Promoter: As used herein, the term “minimal promoter”, unless indicated otherwise, refers to a promoter that includes less than the full naturally occurring promoter sequence, which is still capable of directing transcription of a coding sequence (e.g., a heterogenous or homogenous coding sequence).

In some aspects, the minimal promoter can comprise one or more regions (including all regions) of the fully naturally occurring promoter that can direct transcription of a coding sequence.

In some aspects, the minimal promoter can comprise a portion or portions of the region(s) of the fully naturally occurring promoter that can direct transcription of a coding sequence.

microRNA: As used herein, the term “microRNA” or “miRNA” refers to a class of biomolecules involved in control of gene expression. A mature miRNA is typically an 18-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

In some aspects, a microRNA is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides).

microRNA regulatory target site: As used herein, the term “microRNA regulatory target site” or “miRTS” refers to a sequence that directly interacts with a miRNA on the mRNA transcript. Often, the miRTS is present in the 3′ untranslated region (UTR) of the mRNA, but it may also be present in the coding sequence, or in the 5′ UTR. miRTS are not necessarily perfect complements to miRNAs, usually having only a few bases of complementarity to the miRNA, and often containing one or more mismatches. The miRTS may be any sequence capable of being bound by a miRNA sufficiently that the translation of a gene to which the miRTS is operably linked is repressed by a miRNA silencing mechanism such as the RISC. In some aspects, inclusion of a miRTS into a nucleic acid construct comprising a polynucleotide (e.g., a therapeutic polynucleotide) can result in degradation of the therapeutic polynucleotide after transcription.

Nucleic acid: As used herein, the term “nucleic acid”, in its broadest sense, refers to any compound and/or substance that is or can be incorporated into an oligonucleotide chain. In some aspects, a nucleic acid is a compound and/or substance that is or can be incorporated into an oligonucleotide chain via a phosphodiester linkage. As will be clear from context, in some aspects, “nucleic acid” refers to an individual nucleic acid residue (e.g., a nucleotide and/or nucleoside); in some aspects, “nucleic acid” refers to an oligonucleotide chain comprising individual nucleic acid residues. In some aspects, a “nucleic acid” is or comprises RNA; in some aspects, a “nucleic acid” is or comprises DNA. In some aspects, a nucleic acid is, comprises, or consists of one or more natural nucleic acid residues. In some aspects, a nucleic acid is, comprises, or consists of one or more nucleic acid analogs. In some aspects, a nucleic acid analog differs from a nucleic acid in that it does not utilize a phosphodiester backbone. Alternatively or additionally, in some aspects, a nucleic acid has one or more phosphorothioate and/or 5′-N-phosphoramidite linkages rather than phosphodiester bonds. In some aspects, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a nucleic acid comprises one or more modified sugars (e.g., 2′-fluororibose, ribose, 2′-deoxyribose, arabinose, and hexose) as compared with those in natural nucleic acids. In some aspects, a nucleic acid has a nucleotide sequence that encodes a functional gene product such as an RNA or protein. In some aspects, a nucleic acid includes one or more introns. In some aspects, nucleic acids are prepared by one or more of isolation from a natural source, enzymatic synthesis by polymerization based on a complementary template (in vivo or in vitro), reproduction in a recombinant cell or system, and chemical synthesis. In some aspects, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a nucleic acid is partly or wholly single stranded; in some aspects, a nucleic acid is partly or wholly double stranded. In some aspects, a nucleic acid has a nucleotide sequence comprising at least one element that encodes, or is complementary to a sequence that encodes, a polypeptide. In some aspects, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some aspects, “operably linked” control elements are contiguous (e.g., covalently linked) with coding elements of interest; in some aspects, control elements act in trans to or otherwise at a from the functional element of interest. In some aspects, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some aspects, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some aspects, an active agent is present in unit dose amount appropriate for administration in a therapeutic regimen that shows a statistically significant probability of achieving a predetermined therapeutic effect when administered to a relevant population. In some aspects, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some aspects, a pharmaceutical composition may be formulated for administration via injection either in a particular organ or compartment, e.g., directly into an ear, or systemic, e.g., intravenously. In some aspects, a formulation may be or comprise drenches (aqueous or non-aqueous solutions or suspensions), tablets, boluses, powders, granules, pastes, capsules, powders, etc. In some aspects, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some aspects, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds. In some aspects, a polypeptide has an amino acid sequence that occurs in nature. In some aspects, a polypeptide has an amino acid sequence that does not occur in nature. In some aspects, a polypeptide has an amino acid sequence that is engineered in that it is designed and/or produced through action of the hand of man. In some aspects, a polypeptide may comprise or consist of natural amino acids, non-natural amino acids, or both. In some aspects, a polypeptide may include one or more pendant groups or other modifications, e.g., modifying or attached to one or more amino acid side chains, at a polypeptide's N-terminus, at a polypeptide's C-terminus, or any combination thereof. In some aspects, such pendant groups or modifications may be acetylation, amidation, lipidation, methylation, pegylation, etc., including combinations thereof. In some aspects, polypeptides may contain L-amino acids, D-amino acids, or both and may contain any of a variety of amino acid modifications or analogs known in the art. In some aspects, useful modifications may be or include, e.g., terminal acetylation, amidation, methylation, etc. In some aspects, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some aspects, a polypeptide can be a therapeutic polypeptide (e.g., a Connexin 26 polypeptide). In some aspects, a polypeptide can be a supporting cell polypeptide (e.g., a Connexin 26 polypeptide). In some aspects, a polypeptide can be a reporter polypeptide.

Polynucleotide: As used herein, the term “polynucleotide” refers to any polymeric chain of nucleic acids. In some aspects, a polynucleotide is or comprises RNA; in some aspects, a polynucleotide is or comprises DNA. In some aspects, a polynucleotide is, comprises, or consists of one or more natural nucleic acid residues. In some aspects, a polynucleotide is, comprises, or consists of one or more nucleic acid analogs. In some aspects, a polynucleotide analog differs from a nucleic acid in that it does not utilize a phosphodiester backbone. Alternatively or additionally, in some aspects, a polynucleotide has one or more phosphorothioate and/or 5′-N-phosphoramidite linkages rather than phosphodiester bonds. In some aspects, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a polynucleotide comprises one or more modified sugars (e.g., 2′-fluororibose, ribose, 2′-deoxyribose, arabinose, and hexose) as compared with those in natural nucleic acids. In some aspects, a polynucleotide has a nucleotide sequence that encodes a functional gene product such as an RNA or protein. In some aspects, a polynucleotide includes one or more introns. In some aspects, a polynucleotide is prepared by one or more of isolation from a natural source, enzymatic synthesis by polymerization based on a complementary template (in vivo or in vitro), reproduction in a recombinant cell or system, and chemical synthesis. In some aspects, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a polynucleotide is partly or wholly single stranded; in some aspects, a polynucleotide is partly or wholly double stranded. In some aspects, a polynucleotide has a nucleotide sequence comprising at least one element that encodes, or is the complement of a sequence that encodes, a polypeptide. In some aspects, a polynucleotide has enzymatic activity.

Promoter: As used herein, the term “promoter” refers to a nucleic acid sequence that functions to control the transcription of one or more coding sequences (e.g., a gene or transgene, e.g., encoding a polypeptide (e.g., a therapeutic polypeptide), located upstream with respect to the direction of transcription of the transcription initiation site of the coding sequence. In some aspects, the promoter is structurally identified by the presence of a binding site for DNA-dependent RNA polymerase, transcription initiation sites or other DNA sequence (e.g., a transcription factor binding site, a repressor and/or activator protein binding site, or other sequences of nucleotides that act directly or indirectly to regulate the amount of transcription from the promoter). In some aspects, the promoter can comprise a naturally occurring promoter sequence, a functional fragment thereof, or a mutant of the naturally occurring promoter sequence or a functional fragment thereof.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some aspects, one or more of such selected sequence elements is found in nature. In some aspects, one or more of such selected sequence elements is designed in silico. In some aspects, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some aspects, an agent, animal, individual, population, sample, sequence or value of interest is compared with a reference or control agent, animal, individual, population, sample, sequence or value. In some aspects, a reference or control is tested and/or determined substantially simultaneously with the testing or determination of interest. In some aspects, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some aspects, a reference is a negative control reference; in some aspects, a reference is a positive control reference. In some aspects, the reference can be a compound, a protein, a polypeptide, or a polynucleotide disclosed in the present disclosure.

Regulatory Element: As used herein, the term “regulatory element” or “regulatory sequence” refers to non-coding regions of DNA that regulate, in some way, expression of one or more particular genes. In some aspects, such genes are apposed or “in the neighborhood” of a given regulatory element. In some aspects, such genes are located quite far from a given regulatory element. In some aspects, a regulatory element impairs or enhances transcription of one or more genes. In some aspects, a regulatory element may be located in cis to a gene being regulated. In some aspects, a regulatory element may be located in trans to a gene being regulated. For example, in some aspects, a regulatory sequence refers to a nucleic acid sequence which is regulates expression of a gene product operably linked to a regulatory sequence. In some such aspects, this sequence may be an enhancer sequence and other regulatory elements which regulate expression of a gene product.

Sample: As used herein, the term “sample” typically refers to an aliquot of material obtained or derived from a source of interest. In some aspects, a source of interest is a biological or environmental source. In some aspects, a source of interest may be or comprise a cell or an organism, such as a microbe (e.g., virus), a plant, or an animal (e.g., a human). In some aspects, a source of interest is or comprises biological tissue or fluid. In some aspects, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some aspects, a biological fluid may be or comprise an intracellular fluid, an extracellular fluid, an intravascular fluid (blood plasma), an interstitial fluid, a lymphatic fluid, and/or a transcellular fluid. In some aspects, a biological fluid may be or comprise a plant exudate. In some aspects, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some aspects, a biological sample is or comprises cells obtained from an individual. In some aspects, a sample is a “primary sample” obtained directly from a source of interest by any appropriate means. In some aspects, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Selective expression: As used herein, the term “selective expression” or “selectively expresses” refers to expression of a gene or polypeptide of interest predominately in certain specific cell types (e.g., inner ear cells, e.g., inner ear supporting cells).

Subject: As used herein, the term “subject” refers to an organism, typically a mammal (e.g., a human, in some aspects including prenatal human forms). In some aspects, a subject is suffering from a relevant disease, disorder or condition. In some aspects, a subject is susceptible to a disease, disorder, or condition. In some aspects, a subject displays one or more symptoms or characteristics of a disease, disorder or condition. In some aspects, a subject does not display any symptom or characteristic of a disease, disorder, or condition. In some aspects, a subject is someone with one or more features characteristic of susceptibility to or risk of a disease, disorder, or condition. In some aspects, a subject is a patient. In some aspects, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Supporting cell: As used herein, the term “support cell,” “supporting cell,” “inner ear support cell,” or “inner ear supporting cell” refers to cells of the inner ear that maintain the structure of the inner ear and maintain the environment of the sensory epithelium of the inner ear. In some aspects, inner ear supporting cells include, but are not limited to, inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Supporting cellpolypeptide: As used herein, the term “supporting cell polypeptide” or “support cell polypeptide” refers to a polypeptide that is endogenously expressed in a supporting cell of the inner ear.

Reporter polypeptide: As used herein, the term “reporter polypeptide” refers to a polypeptide that confers onto an organism or cell, a detectable or selectable phenotype. The detectable phenotype can be colorimetric, fluorescent or luminescent, for example. Reporter polypeptides can include enzymes mediating luminescence reactions (luxA, luxB, luxAB, luc, ruc, nluc), enzymes mediating colorimetric reactions (lacZ, IRP), fluorescent proteins (GFP, eGFP, YFP, RFP, CFP, BFP, mCherry, near-infrared fluorescent proteins), affinity peptides (His-tag, 3×-FLAG), and selectable markers (ampC, tet(M), CAT, erm). The reporter polypeptide can be used as a marker for successful uptake of a nucleic acid molecule or exogenous sequence (plasmid) into a cell. The reporter polypeptide can also be used to indicate the presence of a target gene, target nucleic acid molecule, target polypeptide, target intracellular molecule, or a cell, as described herein.

Therapeutic polypeptide: As used herein, the term “therapeutic polypeptide” refers to a polypeptide possessing biological activity that can be used for the prevention and/or treatment of disease (e.g., hearing loss). Examples of therapeutic polypeptides include those capable of preventing, inhibiting, stabilizing or reversing an inherited or noninherited genetic defect in metabolism, immune regulation, hormonal regulation, enzymatic or membrane associated structural function. For example, therapeutic protein can replace an absent or defective cellular protein or enzyme, or supplement production of a defective or low expressed cellular protein or enzyme

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some aspects, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some aspects, treatment may be of a subject who has been diagnosed as suffering from the relevant disease, disorder, and/or condition. In some aspects, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some aspects, a variant can have the same or a different (e.g., increased or decreased) level of activity or functionality than a wild type sequence. For example, in some aspects, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some aspects, a variant has a reduction or elimination in activity or functionality or a change in activity that results in a negative outcome (e.g., increased electrical activity resulting in chronic depolarization that leads to cell death). Such a variant is referred to herein as a loss-of-function variant. In some aspects, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some aspects, a loss-of-function variant has one or more changes that result in a transcript or polypeptide that is defective in some way (e.g., decreased function, non-functioning) relative to the wild type transcript and/or polypeptide.

### DETAILED DESCRIPTION

In certain aspects, the present disclosure is directed to promoters for selective transgene expression, e.g., preferential expression in inner ear supporting cells.

In some aspects, the present disclosure is directed to constructs comprising a polynucleotide encoding a therapeutic polypeptide (e.g., a Connexin 26 polypeptide) and compositions comprising the same which are designed for selective transgene expression, e.g., preferential expression in inner ear supporting cells and/or reduced expression in other inner ear cells such as hair cells.

In some aspects, the present disclosure is also directed to constructs comprising a polynucleotide encoding a polypeptide and compositions comprising the same which are designed for selective transgene expression, e.g., preferential expression in inner ear supporting cells and/or reduced expression in other inner ear cells such as hair cells.

In some aspects, the present disclosure is directed to constructs comprising a polynucleotide encoding a therapeutic polypeptide (e.g., a Connexin 26 polypeptide) and compositions comprising the same which are designed for transgene expression in inner ear supporting cells, e.g., preferential expression in inner ear supporting cells and/or reduced expression in other inner ear cells such as hair cells. In some aspects, the preferential expression and/or reduced expression is relative to the corresponding endogenous expression.

In some aspects, the present disclosure is directed to AAV particles comprising the promoters or constructs disclosed herein.

In some aspects, the present disclosure is directed to methods of using the promoters, constructs, and AAV particles disclosed herein for treating hearing loss.

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some aspects, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB1 and DFNA3; and Bart-Pumphrey syndrome, hystrix-like ichthyosis with deafness (HID), palmoplantar keratoderma with deafness, keratitis-ichthyosis-deafness (KID) syndrome and Vohwinkel syndrome, respectively). Environmental causes of hearing impairment or loss may include, e.g., certain medications, specific infections before or after birth, and/or exposure to loud noise over an extended period. In some aspects, hearing loss can result from noise, ototoxic agents, presbycusis, disease, infection or cancers that affect specific parts of the ear. In some aspects, ischemic damage can cause hearing loss via pathophysiological mechanisms. In some aspects, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some aspects, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and PCT filing application US2019/060328, each of which is incorporated in its entirety herein by reference).

In some aspects, nonsyndromic hearing loss and/or deafness is not associated with other signs and symptoms. In some aspects, syndromic hearing loss and/or deafness occurs in conjunction with abnormalities in other parts of the body. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness. Between 75 percent and 80 percent of genetically causative hearing loss and/or deafness cases are inherited in an autosomal recessive pattern, which means both copies of the gene in each cell have mutations. Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between 1 to 2 percent of cases of deafness and/or hearing loss show an X-linked pattern of inheritance, which means the mutated gene responsible for the condition is located on the X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons. Mitochondrial nonsyndromic deafness, which results from changes to mitochondrial DNA, occurs in less than one percent of cases in the United States. The altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in the same gene can be associated with different types of deafness and/or hearing loss, and some genes are associated with both syndromic and nonsyndromic deafness and/or hearing loss.

In some aspects, deafness and/or hearing loss can be conductive (arising from the ear canal or middle ear), sensorineural (arising from the inner ear or auditory nerve), or mixed. In some aspects, nonsyndromic deafness and/or hearing loss is associated with permanent hearing loss caused by damage to structures in the inner ear (sensorineural deafness). In some aspects, sensorineural hearing loss can be due to poor hair cell function. In some aspects, sensorineural hearing impairments involve the eighth cranial nerve (the vestibulocochlear nerve) or the auditory portions of the brain. In some such aspects, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some aspects, hair cells may be abnormal at birth, or damaged during the lifetime of an individual. In some aspects, outer hair cells may be able to regenerate. In some aspects, inner hair cells are not capable of regeneration after illness or injury. In some aspects, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfill numerous functions, and include a number of cell types, including but not limited to inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, sensorineural hearing loss is due to abnormalities in supporting cells. In some aspects, supporting cells may be abnormal at birth, or damaged during the lifetime of an individual. In some aspects, supporting cells may be able to regenerate. In some aspects, certain supporting cells may not be capable of regeneration.

### Polypeptides

Certain aspects of the disclosure are directed to polynucleotides encoding a polypeptide. The polynucleotide can encode a polypeptide that is capable of being expressed in a cell (e.g., an inner ear cell). The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding polypeptides include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4).

In some aspects, the polynucleotide can comprise a GJB2 gene. In some aspects, the polynucleotide can comprise a nucleic acid encoding a Connexin 26 polypeptide. In some aspects, the nucleic acid comprises a coding sequence for a Connexin 26 polypeptide.

In some aspects, the polynucleotide or nucleic acid comprises a gap junction beta-2 (GJB2) gene. The GJB2 gene is highly conserved across the mammalian class and encodes connexin 26 (Cx26) (also referred to as gap junction beta-2 (GJB2) protein). Connexin 26 is a member of the gap junction protein family, which is also known as the connexin family. Gap junction proteins are specialized proteins, involved in intracellular communication. Mutations in the human GJB2 gene have been associated with hearing loss and deafness (Amorini et al., Ann. Hum. Genet. 79(5):341-349, 2015; Qing et al., Genet. Test Mol. Biomarkers 19(1):52-58, 2015).

The human GJB2 gene is located on chromosome 13q12. It contains two transcriptional isoforms beginning from alternative transcriptional start sites, both of which contain two exons and a single intron encompassing a total of about 5 kilobases (kb) (approximately 5,469 or 4,675 nucleotides respectively) (NCBI Gene ID 2706, NCBI Reference Sequence: NG 008358.1). Both human GJB2 mRNA isoforms comprise a second exon, which completely encodes a full-length connexin 26 in exon two. This coding sequence is approximately 681 nucleotides, and encodes a connexin 26 that is 226 amino acids in length.

A monomer of connexin 26 includes four transmembrane helices linked by two extracellular loops and one shorter intracellular loop, with N- and C-termini on the cytosolic side of the plasma membrane. Gap junctions between cells can be formed in a homomeric and/or heteromeric manner. Connexin 26 has been shown to form functional homomeric channels, as well as functional heteromeric channels with at least connexin 30, connexin 32, connexin 46, and connexin 50. In some aspects, GJB2 gene associated sensorineural hearing loss (e.g., nonsyndromic or syndromic) may be due to compound heterozygous mutations in GJB2 and in an alternative connexin protein encoding gene. The gap junctions created with connexin 26 transport potassium ions and certain other small molecules across cells. Connexin 26 helps maintain the correct level of intracellular potassium ions, and is required for the maturation of certain cells in the cochlea.

A human GJB2 gene is expressed in a number of tissues, but is known to be involved in important cellular homeostasis functions in the epidermis and inner ear. Within the inner ear, connexin 26 is synthesized by all supporting cell types within the organ of corti, including the inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), root cells, fibrocytes, fibroblasts, basal and intermediate cells from the stria vascularis, and other cells of the lateral wall. In addition, connexin 26 is known to be present in mesenchymal cells in the lateral wall, and type 1 neurons in the spiral ganglion.

The human GJB2 gene has a defined 128 bp long basal/minimal promoter just upstream of the canonical first exon in the most abundant isoform. This sequence includes a TATA box and two GC boxes, which are known to be bound by the SpI and Sp3 TFs.

There are over 200 defined mutations of GJB2, which show some level of pathogenicity, and various mutations in the GJB2 gene have been associated with hearing loss (e.g., non-syndromic sensorineural hearing loss or syndromic sensorineural hearing loss). For example, the c.35delG allele was found on 65.5% of patients from Eastern Sicily (Amorini et al., Ann. Hum. Genet. 79(5):341-349, 2015). Additional exemplary mutations in a GJB2 gene detected in subjects having nonsyndromic sensorineural hearing loss or syndromic sensorineural hearing loss, and methods of sequencing a nucleic acid encoding GJB2 are described in, e.g., Snoeckx et al., Am. J. Hum. Genet 77: 945-957, 2005; Welch et al., Am. J. Med. Genet A 143: 1567-1573, 2007; Zelante et al., Hum. Mol. Genet. 6:1605-1609, 1997; and Tsukada et al., Annals of Otology, Rhinology & Laryngology. 2015, Vol. 124(5S) 61S-76S, each of which is incorporated in its entirety herein by reference. Methods of detecting mutations in a gene are well-known in the art. Non-limiting examples of such techniques include: real-time polymerase chain reaction (RT-PCR), PCR, Sanger sequencing, Next-generation sequencing, Southern blotting, and Northern blotting. Multiple disease states associated with sensorineural hearing loss with either nonsyndromic or syndromic manifestations have been linked with specific mutations of the human GJB2 gene (see Nickel & Forge, Curr Opin Otolaryngol Head Neck Surg. 2008 October; 16(5):452-7, which is incorporated in its entirety herein by reference). Human GBJ2 gene mutations which lead to syndromic or nonsyndromic hearing loss vary from large deletions that remove either the entirety of GJB2 or GJB2 gene regulatory regions, to hundreds of small scale alterations including nonsense, missense, indels (leading to phase shifting), and splice-site point mutations.

In some aspects, GJB2 gene mutations such as Gly59Ser, and Asn52Lys are associated with Bart-Pumphrey syndrome. A syndrome defined by manifestations of thickened skin, wart-like growths, and generally congenital moderate to profound sensorineural hearing loss. In other aspects, GJB2 gene mutations such as Aspn50Asn are associated with Hystrix-like Ichthyosis with deafness & Keratitisichthyosis-deafness syndrome. These syndromes are associated with dry scaly skin, generally congenital profound sensorineural hearing loss, and in Keratitis-ichthyosisdeafness syndrome, additional inflammation of the cornea.

In some aspects, GJB2 gene missense mutations are associated with Palmoplantar keratoderma with deafness. A syndrome associated with thick skin on the palms of the hands and soles of the feet, and mild to profound sensorineural hearing loss which begins in early childhood and gets worse over time, affected individuals may have particular trouble hearing high-pitched sounds. While in other aspects, GJB2 gene missense mutations are associated with Vohwinkel syndrome. A syndrome associated with skin abnormalities (e.g., thick bands of fibrous tissue around their fingers and toes that may cut off the circulation to the digits and result in spontaneous amputation) and sensorineural hearing loss.

In some aspects, GJB2 gene mutations are associated with nonsyndromic hearing loss, which may be inherited in either a dominant (e.g., DFNA3) or recessive manner (DFNB1). In some aspects, loss of function GJB2 gene mutations are associated with nonsyndromic DFNB1 which is inherited in an autosomal recessive manner and presents as mild to profound hearing loss that is generally prelingual and does not become more severe over time. It is estimated that DFNB1 is present in approximately 14 out of every 100,000 live births in the US and EUS. It has been postulated that an early but not always congenital onset of DFNB1 hearing impairment could be followed by a quick progression of the hearing loss. In general, DFNB1 patents treatment options include education, hearing aids, and cochlear implants. Patients generally do not have additional symptoms, and live a normal lifespan. It is estimated that DFNB1 accounts for about 50% of congenital severe-to-profound autosomal recessive non-syndromic hearing loss in many first world countries (e.g., US, France, Britain, and Australia).

In some aspects, sensorineural hearing loss due to GJB2 gene mutations are inherited in an autosomal dominant manner as nonsyndromic DFNA3. These mutations are generally dominant negative missense mutations that prevent the formation of necessary functional gap junctions. This disease state presents with hearing loss that can be either prelingual or postlingual, ranging from mild to profound, which generally becomes more severe over time.

Among other things, the present disclosure provides polynucleotides, e.g., polynucleotides comprising a GJB2 gene or characteristic portion thereof, as well as compositions including such polynucleotides and methods utilizing such polynucleotides and/or compositions.

In some aspects, a polynucleotide comprising a GJB2 gene or characteristic portion thereof can be DNA or RNA. In some aspects, DNA can be genomic DNA or cDNA. In some aspects, RNA can be an mRNA. In some aspects, a polynucleotide comprises exons and/or introns of a GJB2 gene.

In some aspects, a gene product is expressed from a polynucleotide comprising a GJB2 gene or characteristic portion thereof. In some aspects, expression of such a polynucleotide can utilize one or more control elements (e.g., promoters, enhancers, splice sites, poly-adenylation sites, translation initiation sites, etc.). Thus, in some aspects, a polynucleotide provided herein can include one or more control elements.

In some aspects, a GJB2 gene is a mammalian GJB2 gene. In some aspects, a GJB2 gene is a murine GJB2 gene. In some aspects, a GJB2 gene is a primate GJB2 gene. In some aspects, a GJB2 gene is a human GJB2 gene. In some aspects, a GJB2 gene is codon optimized. An exemplary human GJB2 coding cDNA sequence is or includes the sequence of SEQ ID NO: 117 or SEQ ID NO: 118. An exemplary human GJB2 spliced cDNA sequence with untranslated regions is or includes the sequence of SEQ ID NO: 119. An alternative transcriptional start site exemplary human GJB2 spliced cDNA sequence with untranslated regions is or includes the sequence of SEQ ID NO: 120. An exemplary human GJB2 genomic DNA sequence can be found in SEQ ID NO: 121. Exemplary codon optimized GJB2 DNA sequences can be found in SEQ ID NOs: 123-126.

Exemplary Human GJB2 cDNA coding Sequence
(SEQ ID NO: 117)
ATGGATTGGGGCACGCTGCAGACGATCCTGGGGGGTGTGAACAAACACTCCA
CCAGCATTGGAAAGATCTGGCTCACCGTCCTCTTCATTTTTCGCATTATGATCC
TCGTTGTGGCTGCAAAGGAGGTGTGGGGAGATGAGCAGGCCGACTTTGTCTG
CAACACCCTGCAGCCAGGCTGCAAGAACGTGTGCTACGATCACTACTTCCCC
ATCTCCCACATCCGGCTATGGGCCCTGCAGCTGATCTTCGTGTCCACGCCAGC
GCTCCTAGTGGCCATGCACGTGGCCTACCGGAGACATGAGAAGAAGAGGAAG
TTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCA
TCTTCTTCCGGGTCATCTTCGAAGCCGCCTTCATGTACGTCTTCTATGTCATGT
ACGACGGCTTCTCCATGCAGCGGCTGGTGAAGTGCAACGCCTGGCCTTGTCCC
AACACTGTGGACTGCTTTGTGTCCCGGCCCACGGAGAAGACTGTCTTCACAGT
GTTCATGATTGCAGTGTCTGGAATTTGCATCCTGCTGAATGTCACTGAATTGT
GTTATTTGCTAATTAGATATTGTTCTGGGAAGTCAAAAAAGCCAGTT
Exemplary Human GJB2 cDNA coding Sequence
(SEQ ID NO: 118)
ATGGATTGGGGCACGCTGCAGACGATCCTGGGGGGTGTGAACAAACACTCCA
CCAGCATTGGAAAGATCTGGCTCACCGTCCTCTTCATTTTTCGCATTATGATCC
TCGTTGTGGCTGCAAAGGAGGTGTGGGGAGATGAGCAGGCCGACTTTGTCTG
CAACACCCTGCAGCCAGGCTGCAAGAACGTGTGCTACGATCACTACTTCCCC
ATCTCCCACATCCGGCTATGGGCCCTGCAGCTGATCTTCGTGTCCACGCCAGC
GCTCCTAGTGGCCATGCACGTGGCCTACCGGAGACATGAGAAGAAGAGGAAG
TTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCA
TCTTCTTCCGGGTCATCTTCGAAGCCGCCTTCATGTACGTCTTCTATGTCATGT
ACGACGGCTTCTCCATGCAGCGGCTGGTGAAGTGCAACGCCTGGCCTTGTCCC
AACACTGTGGACTGCTTTGTGTCCCGGCCCACGGAGAAGACTGTCTTCACAGT
GTTCATGATTGCAGTGTCTGGAATTTGCATCCTGCTGAATGTCACTGAATTGT
GTTATTTGCTAATTAGATATTGTTCTGGGAAGTCAAAAAAGCCAGTTTAA
Exemplary spliced Human GJB2 isoform 1 cDNA including untranslated
regions Sequence
(SEQ ID NO: 119)
GTTGCGGCCCCGCAGCGCCCGCGCGCTCCTCTCCCCGACTCGGAGCCCCTCGG
CGGCGCCCGGCCCAGGACCCGCCTAGGAGCGCAGGAGCCCCAGCGCAGAGA
CCCCAACGCCGAGACCCCCGCCCCGGCCCCGCCGCGCTTCCTCCCGACGCAG
AGCAAACCGCCCAGAGTAGAAGATGGATTGGGGCACGCTGCAGACGATCCTG
GGGGGTGTGAACAAACACTCCACCAGCATTGGAAAGATCTGGCTCACCGTCC
TCTTCATTTTTCGCATTATGATCCTCGTTGTGGCTGCAAAGGAGGTGTGGGGA
GATGAGCAGGCCGACTTTGTCTGCAACACCCTGCAGCCAGGCTGCAAGAACG
TGTGCTACGATCACTACTTCCCCATCTCCCACATCCGGCTATGGGCCCTGCAG
CTGATCTTCGTGTCCACGCCAGCGCTCCTAGTGGCCATGCACGTGGCCTACCG
GAGACATGAGAAGAAGAGGAAGTTCATCAAGGGGGAGATAAAGAGTGAATT
TAAGGACATCGAGGAGATCAAAACCCAGAAGGTCCGCATCGAAGGCTCCCTG
TGGTGGACCTACACAAGCAGCATCTTCTTCCGGGTCATCTTCGAAGCCGCCTT
CATGTACGTCTTCTATGTCATGTACGACGGCTTCTCCATGCAGCGGCTGGTGA
AGTGCAACGCCTGGCCTTGTCCCAACACTGTGGACTGCTTTGTGTCCCGGCCC
ACGGAGAAGACTGTCTTCACAGTGTTCATGATTGCAGTGTCTGGAATTTGCAT
CCTGCTGAATGTCACTGAATTGTGTTATTTGCTAATTAGATATTGTTCTGGGA
AGTCAAAAAAGCCAGTTTAACGCATTGCCCAGTTGTTAGATTAAGAAATAGA
CAGCATGAGAGGGATGAGGCAACCCGTGCTCAGCTGTCAAGGCTCAGTCGCT
AGCATTTCCCAACACAAAGATTCTGACCTTAAATGCAACCATTTGAAACCCCT
GTAGGCCTCAGGTGAAACTCCAGATGCCACAATGGAGCTCTGCTCCCCTAAA
GCCTCAAAACAAAGGCCTAATTCTATGCCTGTCTTAATTTTCTTTCACTTAAGT
TAGTTCCACTGAGACCCCAGGCTGTTAGGGGTTATTGGTGTAAGGTACTTTCA
TATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGT
TCTTTTTGCCAACTTTCCCCACGTTAAAGGTGAACATTGGTTCTTTCATTTGCT
TTGGAAGTTTTAATCTCTAACAGTGGACAAAGTTACCAGTGCCTTAAACTCTG
TTACACTTTTTGGAAGTGAAAACTTTGTAGTATGATAGGTTATTTTGATGTAA
AGATGTTCTGGATACCATTATATGTTCCCCCTGTTTCAGAGGCTCAGATTGTA
ATATGTAAATGGTATGTCATTCGCTACTATGATTTAATTTGAAATATGGTCTTT
TGGTTATGAATACTTTGCAGCACAGCTGAGAGGCTGTCTGTTGTATTCATTGT
GGTCATAGCACCTAACAACATTGTAGCCTCAATCGAGTGAGACAGACTAGAA
GTTCCTAGTGATGGCTTATGATAGCAAATGGCCTCATGTCAAATATTTAGATG
TAATTTTGTGTAAGAAATACAGACTGGATGTACCACCAACTACTACCTGTAAT
GACAGGCCTGTCCAACACATCTCCCTTTTCCATGACTGTGGTAGCCAGCATCG
GAAAGAACGCTGATTTAAAGAGGTCGCTTGGGAATTTTATTGACACAGTACC
ATTTAATGGGGAGGACAAAATGGGGCAGGGGAGGGAGAAGTTTCTGTCGTTA
AAAACAGATTTGGAAAGACTGGACTCTAAAGTCTGTTGATTAAAGATGAGCT
TTGTCTACTTCAAAAGTTTGTTTGCTTACCCCTTCAGCCTCCAATTTTTTAAGT
GAAAATATAGCTAATAACATGTGAAAAGAATAGAAGCTAAGGTTTAGATAAA
TATTGAGCAGATCTATAGGAAGATTGAACCTGAATATTGCCATTATGCTTGAC
ATGGTTTCCAAAAAATGGTACTCCACATATTTCAGTGAGGGTAAGTATTTTCC
TGTTGTCAAGAATAGCATTGTAAAAGCATTTTGTAATAATAAAGAATAGCTTT
AATGATATGCTTGTAACTAAAATAATTTTGTAATGTATCAAATACATTTAAAA
CATTAAAATATAATCTCTATAATAA
Exemplary spliced Human GJB2 isoform X1 cDNA including untranslated
regions Sequence
(SEQ ID NO: 120)
TTTAGGACCCTTGTTCGCGAAGAGGTGGTGTGCGGCTGAGACCCGCGTCCTCA
GGACGGTTCCATCAGTGCCTCGATCCTGCCCCACTGGAGGAGGAAGGCAGCC
CGAACAGCGCTCACCTAACTAACAGCTGCTGAGAGCTGGGTTCCGTGGCCAT
GCACCTGGGACTGCCTTGAGAAGCGTGAGCAAACCGCCCAGAGTAGAAGATG
GATTGGGGCACGCTGCAGACGATCCTGGGGGGTGTGAACAAACACTCCACCA
GCATTGGAAAGATCTGGCTCACCGTCCTCTTCATTTTTCGCATTATGATCCTCG
TTGTGGCTGCAAAGGAGGTGTGGGGAGATGAGCAGGCCGACTTTGTCTGCAA
CACCCTGCAGCCAGGCTGCAAGAACGTGTGCTACGATCACTACTTCCCCATCT
CCCACATCCGGCTATGGGCCCTGCAGCTGATCTTCGTGTCCACGCCAGCGCTC
CTAGTGGCCATGCACGTGGCCTACCGGAGACATGAGAAGAAGAGGAAGTTCA
TCAAGGGGGAGATAAAGAGTGAATTTAAGGACATCGAGGAGATCAAAACCC
AGAAGGTCCGCATCGAAGGCTCCCTGTGGTGGACCTACACAAGCAGCATCTT
CTTCCGGGTCATCTTCGAAGCCGCCTTCATGTACGTCTTCTATGTCATGTACGA
CGGCTTCTCCATGCAGCGGCTGGTGAAGTGCAACGCCTGGCCTTGTCCCAACA
CTGTGGACTGCTTTGTGTCCCGGCCCACGGAGAAGACTGTCTTCACAGTGTTC
ATGATTGCAGTGTCTGGAATTTGCATCCTGCTGAATGTCACTGAATTGTGTTA
TTTGCTAATTAGATATTGTTCTGGGAAGTCAAAAAAGCCAGTTTAACGCATTG
CCCAGTTGTTAGATTAAGAAATAGACAGCATGAGAGGGATGAGGCAACCCGT
GCTCAGCTGTCAAGGCTCAGTCGCTAGCATTTCCCAACACAAAGATTCTGACC
TTAAATGCAACCATTTGAAACCCCTGTAGGCCTCAGGTGAAACTCCAGATGCC
ACAATGGAGCTCTGCTCCCCTAAAGCCTCAAAACAAAGGCCTAATTCTATGCC
TGTCTTAATTTTCTTTCACTTAAGTTAGTTCCACTGAGACCCCAGGCTGTTAGG
GGTTATTGGTGTAAGGTACTTTCATATTTTAAACAGAGGATATCGGCATTTGT
TTCTTTCTCTGAGGACAAGAGAAAAAAGCCAGGTTCCACAGAGGACACAGAG
AAGGTTTGGGTGTCCTCCTGGGGTTCTTTTTGCCAACTTTCCCCACGTTAAAG
GTGAACATTGGTTCTTTCATTTGCTTTGGAAGTTTTAATCTCTAACAGTGGACA
AAGTTACCAGTGCCTTAAACTCTGTTACACTTTTTGGAAGTGAAAACTTTGTA
GTATGATAGGTTATTTTGATGTAAAGATGTTCTGGATACCATTATATGTTCCC
CCTGTTTCAGAGGCTCAGATTGTAATATGTAAATGGTATGTCATTCGCTACTA
TGATTTAATTTGAAATATGGTCTTTTGGTTATGAATACTTTGCAGCACAGCTG
AGAGGCTGTCTGTTGTATTCATTGTGGTCATAGCACCTAACAACATTGTAGCC
TCAATCGAGTGAGACAGACTAGAAGTTCCTAGTGATGGCTTATGATAGCAAA
TGGCCTCATGTCAAATATTTAGATGTAATTTTGTGTAAGAAATACAGACTGGA
TGTACCACCAACTACTACCTGTAATGACAGGCCTGTCCAACACATCTCCCTTT
TCCATGACTGTGGTAGCCAGCATCGGAAAGAACGCTGATTTAAAGAGGTCGC
TTGGGAATTTTATTGACACAGTACCATTTAATGGGGAGGACAAAATGGGGCA
GGGGAGGGAGAAGTTTCTGTCGTTAAAAACAGATTTGGAAAGACTGGACTCT
AAAGTCTGTTGATTAAAGATGAGCTTTGTCTACTTCAAAAGTTTGTTTGCTTA
CCCCTTCAGCCTCCAATTTTTTAAGTGAAAATATAGCTAATAACATGTGAAAA
GAATAGAAGCTAAGGTTTAGATAAATATTGAGCAGATCTATAGGAAGATTGA
ACCTGAATATTGCCATTATGCTTGACATGGTTTCCAAAAAATGGTACTCCACA
TATTTCAGTGAGGGTAAGTATTTTCCTGTTGTCAAGAATAGCATTGTAAAAGC
ATTTTGTAATAATAAAGAATAGCTTTAATGATATGCTTGTAACTAAAATAATT
TTGTAATGTATCAAATACATTTAAAACATTAAAATATAATCTCTATAATAA
Exemplary Human GJB2 Genomic DNA Sequence
(SEQ ID NO: 121)
GTTGCGGCCCCGCAGCGCCCGCGCGCTCCTCTCCCCGACTCGGAGCCCCTCGG
CGGCGCCCGGCCCAGGACCCGCCTAGGAGCGCAGGAGCCCCAGCGCAGAGA
CCCCAACGCCGAGACCCCCGCCCCGGCCCCGCCGCGCTTCCTCCCGACGCAG
GTGAGCCCGCCGGCCCCGGACTGCCCGGCCAGGAACCTGGCGCGGGGAGGG
ACCGCGAGACCCAGAGCGGTTGCCCGGCCGCGTGGGTCTCGGGGAACCGGGG
GGCTGGACCAACACACGTCCTTGGGCCGGGGGGGGGGGGCCGCCTTCTGGAG
CGGGCGTTTCTGCGGCCGAGCTCCGGAGCTGGAATGGGGCGGCCGGGGAAGT
GGACGCGATGGCACCGCCCGGGGTGCGAGTGGGGCCGGGCGCGCGCGGGAG
GGGAAAAAGGCGCGGGCGAGCCGCCAGCGCGAGGTTTGTGGTGTCGCCGATG
TCCCTTCGGGGTACTCTAGCGCAGCCGCCTGGCTACTTGACCCACTGCCACCA
AACGTTTTAAATTCACCGAAAGCTTAGCTTCGAAGCAAAGCTCCGTTTCGCCG
GTGAAGCAGGAAGCCTTCGCTGCAGGAACTGACCTTTACCTCTTGGAGCGGC
TTCTGCAGAAAAATCCCCGGGCAGAGATTTGGGCGGAGTTTGCCTAGAACTA
ACGCGGAGCCAGCCGATCCCGGCCTACCCCGGGGCCAAGATTTCAGTGGCTT
CCCTTTTTCCTAAACACTTCACGAGGGTCTGTTTCCGGGCTGTGCTCCCCGCCT
AGAAGGAAAATTTTTAGGACCCTTGTTCGCGAAGAGGTGGTGTGCGGCTGAG
ACCCGCGTCCTCAGGACGGTTCCATCAGTGCCTCGATCCTGCCCCACTGGAGG
AGGAAGGCAGCCCGAACAGCGCTCACCTAACTAACAGCTGCTGAGAGCTGGG
TTCCGTGGCCATGCACCTGGGACTGCCTTGAGAAGCGTGGTACGGCCGTGTCC
CCATGTGACCTTAGAGTCCCTTTCGAAACTGCTGTGCACAGTCGGTCACAATT
TCAGACACTGGTGAGAAGGGTGGAGGAACCCTCTGGGGACAGCCAGGCAAG
GTCGACCACCCATCACCTAAGGGTGGAGAAATTTAAGGGGTGAAGAGTCCCT
TTTGCCTTTTCTGGATCCTGGTGATTCACCTAGTGTCTTCCCTAAGGAACTGAA
CCAACTCCTCCGCTGGCCTCTGGCAGCCCTCCAGGCGGTGCAGGATGGCGTG
GGCCCGGTAGGAAGCTGCATGTAACCGCCCAGGGTCGGGAGGCCAGGAGGG
CAGCTCCTCCTCTGACTTGAATATTGAAAACAACTTCGTCCTGCTTCTGAGCC
CCTCTTAACCCATGACCCCCTAGCCCATTGGGGAGTAAATCTTAATTTACTCC
TCTTCCTGAAAAAGGATCTTTAAAACAGGTAGCTTCAACTCAAGCTTTATAAA
ATAACAATATAGGGTTTCTCGGAACTGTATTTTTCTCAGCTGATGGTAACTGG
ACAGGTCTGTAGAAGGGTGTATGACCTGGGTTTGGCAGGTGGAAGAGGGCAA
AGGATAAACCCCTCCTCCTGCAGCCCCATATTCTTGGCCAGGTGTATTGTTGT
AAACCAGGAGAGAGTTTACTTCGGGGAGTATCCTGTTTTCCACTCAGTGAGG
GCCAATGAAGAATGTCTAATTCCATAAGATGCTTTTGTTAAAATCGGAATGTT
GCTGTCCTCGGTGGTTCTGCTGTTGGGACGGGACTGGCCTGAGCTGTGGGTGC
TGTAGCAGGACAACCAGCTCACCTAAGGGCCTCCCAGTCTGGATTATCAATG
GGTCAGTGCTGAACCTGGGCTAAAATATTGTTTTTTCCAATGATGTTGTCTTTC
CCAAGCTCAGTGAAGCTAAATGTTTCACAGGCCTATGTCAATCTGATGTAACT
TTCGTGGCCACCTCTCTCCTGTTAGCCTCTGACCAAGGTGGCACTGGATGGTT
TCTGCCTGACCTTGGTGCCCCGTGGCAGCGACTGTGGGTCATGAAAGACATTC
ACTACGAGCCTGCTTCTGGAGTCCATCAGAAAACGGGATGCAACTTGCCTAA
AATGAGGAGAGGAGGATGCTTTTAAGAAAAAGAAGAAGGAGGATTCACTAC
CAGCTCTGAAGGGTGGAAAAGAGATGATTCATCCGGATTGTGGAGAGGGTGG
AATCTTGTTTAGGAGAGCGTTGGTTGTGGCAGGCAGGGTGTAACTATGAATC
AGTGAAGACAATTCACATCCTGGGATGAAAAGAAGGCCATGGGCTCACAGGA
GATTATCCACTGGCCTCTCCACATCCGCTTGCAGTAAGGAGTGTGGGACTCTC
CCAAGCTTCAGCGCTGAACTGCAATGCAGTGACGTCGCTTAGCTGGGCCAGT
AACCGAGGGAGTTGAATTTTCTGTCATTTTAAAATAATGTGTCTTTTAAGAAA
CACTTTGAAATTAAAACCACAGCCCACAATTATAATGCACTGTTGCAGCACTT
ATCAAAACAGATATGCTAACTGAGCCATCAGTGCCAGCCTGACAGTGAGGCC
ACCAAGCCATCCACAAAGCCTACACGAAAGTCTGTGCTCACAGTGGCTTTTCT
CCATGAAGAGGGCATTCCTAACCTCTTCCTTTCACGTAGGAGGAAGCAAGGT
CCTTTGTAAAATTTTAACTCGGGGTGCCTCAAATGTAAACTTAACCACTGGTA
ACAACAGTTTCACTGCTACATGCCACGTCTGTGAAAATTCATTCAAGACATTA
AGGAAAGTGGCTCAGCAGAGAGACTAGACATCTTATCCTCACGGTTCTCCTGT
ACTTGGCCTCTCAGCCTTTGAGCAAGGTTGGCCCAAGCTAGTATCGGCCCCAG
TGGTACAGCCAAAACTTGAGACTGCAAATGGATGCAGCTGTTGAACGCTGAG
TAACTTCTGCAGAGTCAGGAAGACCCAAGGAAGCTCTGCAGAGGATGCAGGG
GTACGGTCAGAACCCCTGAGTGCCTTTCAGCTAACGAGGACTTTATGACACTC
CCCAGCACAGCAAATTTTTATGATGTGTTTAAAGATTGGGTGAATTACTCAGG
TGAACAAGCTACTTTTTATCAGAGAACACCTAAAAACACGTTCAAGAGGGTT
TGGGAACTATACATTTAATCCTATGACAAACTAAGTTGGTTCTGTCTTCACCT
GTTTTGGTGAGGTTGTGTAAGAGTTGGTGTTTGCTCAGGAAGAGATTTAAGCA
TGCTTGCTTACCCAGACTCAGAGAAGTCTCCCTGTTCTGTCCTAGCTAGTGAT
TCCTGTGTTGTGTGCATTCGTCTTTTCCAGAGCAAACCGCCCAGAGTAGAAGA
TGGATTGGGGCACGCTGCAGACGATCCTGGGGGGTGTGAACAAACACTCCAC
CAGCATTGGAAAGATCTGGCTCACCGTCCTCTTCATTTTTCGCATTATGATCCT
CGTTGTGGCTGCAAAGGAGGTGTGGGGAGATGAGCAGGCCGACTTTGTCTGC
AACACCCTGCAGCCAGGCTGCAAGAACGTGTGCTACGATCACTACTTCCCCAT
CTCCCACATCCGGCTATGGGCCCTGCAGCTGATCTTCGTGTCCACGCCAGCGC
TCCTAGTGGCCATGCACGTGGCCTACCGGAGACATGAGAAGAAGAGGAAGTT
CATCAAGGGGGAGATAAAGAGTGAATTTAAGGACATCGAGGAGATCAAAAC
CCAGAAGGTCCGCATCGAAGGCTCCCTGTGGTGGACCTACACAAGCAGCATC
TTCTTCCGGGTCATCTTCGAAGCCGCCTTCATGTACGTCTTCTATGTCATGTAC
GACGGCTTCTCCATGCAGCGGCTGGTGAAGTGCAACGCCTGGCCTTGTCCCAA
CACTGTGGACTGCTTTGTGTCCCGGCCCACGGAGAAGACTGTCTTCACAGTGT
TCATGATTGCAGTGTCTGGAATTTGCATCCTGCTGAATGTCACTGAATTGTGT
TATTTGCTAATTAGATATTGTTCTGGGAAGTCAAAAAAGCCAGTTTAACGCAT
TGCCCAGTTGTTAGATTAAGAAATAGACAGCATGAGAGGGATGAGGCAACCC
GTGCTCAGCTGTCAAGGCTCAGTCGCTAGCATTTCCCAACACAAAGATTCTGA
CCTTAAATGCAACCATTTGAAACCCCTGTAGGCCTCAGGTGAAACTCCAGATG
CCACAATGGAGCTCTGCTCCCCTAAAGCCTCAAAACAAAGGCCTAATTCTATG
CCTGTCTTAATTTTCTTTCACTTAAGTTAGTTCCACTGAGACCCCAGGCTGTTA
GGGGTTATTGGTGTAAGGTACTTTCATATTTTAAACAGAGGATATCGGCATTT
GTTTCTTTCTCTGAGGACAAGAGAAAAAAGCCAGGTTCCACAGAGGACACAG
AGAAGGTTTGGGTGTCCTCCTGGGGTTCTTTTTGCCAACTTTCCCCACGTTAA
AGGTGAACATTGGTTCTTTCATTTGCTTTGGAAGTTTTAATCTCTAACAGTGG
ACAAAGTTACCAGTGCCTTAAACTCTGTTACACTTTTTGGAAGTGAAAACTTT
GTAGTATGATAGGTTATTTTGATGTAAAGATGTTCTGGATACCATTATATGTT
CCCCCTGTTTCAGAGGCTCAGATTGTAATATGTAAATGGTATGTCATTCGCTA
CTATGATTTAATTTGAAATATGGTCTTTTGGTTATGAATACTTTGCAGCACAG
CTGAGAGGCTGTCTGTTGTATTCATTGTGGTCATAGCACCTAACAACATTGTA
GCCTCAATCGAGTGAGACAGACTAGAAGTTCCTAGTGATGGCTTATGATAGC
AAATGGCCTCATGTCAAATATTTAGATGTAATTTTGTGTAAGAAATACAGACT
GGATGTACCACCAACTACTACCTGTAATGACAGGCCTGTCCAACACATCTCCC
TTTTCCATGACTGTGGTAGCCAGCATCGGAAAGAACGCTGATTTAAAGAGGT
CGCTTGGGAATTTTATTGACACAGTACCATTTAATGGGGAGGACAAAATGGG
GCAGGGGAGGGAGAAGTTTCTGTCGTTAAAAACAGATTTGGAAAGACTGGAC
TCTAAAGTCTGTTGATTAAAGATGAGCTTTGTCTACTTCAAAAGTTTGTTTGCT
TACCCCTTCAGCCTCCAATTTTTTAAGTGAAAATATAGCTAATAACATGTGAA
AAGAATAGAAGCTAAGGTTTAGATAAATATTGAGCAGATCTATAGGAAGATT
GAACCTGAATATTGCCATTATGCTTGACATGGTTTCCAAAAAATGGTACTCCA
CATATTTCAGTGAGGGTAAGTATTTTCCTGTTGTCAAGAATAGCATTGTAAAA
GCATTTTGTAATAATAAAGAATAGCTTTAATGATATGCTTGTAACTAAAATAA
TTTTGTAATGTATCAAATACATTTAAAACATTAAAATATAATCTCTATAATAA
Exemplary expanded Human GJB2 Genomic DNA Sequence including certain
regulatory regions
(SEQ ID NO: 122)
GACTGTGAACTTAAGGCACAGCAGAGCTGGGGCTGCTCTTAAGGCCCTGCTG
TCTCTCCTCTTAGTAACAACACCATTTCACATGAAGTGACAGTGGTATCTTTT
GTTGCCCTGGAAATGGAATACAACAATGGCTTTCCAACTTTTCTGTGGCAGAG
ACCTACAGACAGAAGTACATTTTACACTGGATCCAGGACACACATCAGTCTG
AAAACACACACATGAACCAAACGTTTCCTAAAGCATTACTTATCCTTGCTAAT
AGCAACACATTCTCATATTCTTTTATACTTCATTTAATTTCATATAAAAAAGA
AAAGGAAAGGAAAGAAATCTATTTCTCAGCCCATTAATAAGGTCAGGAGCAG
CAACACCAGACTAGAAGAAAAGCTTACCTATAGATTTTTCTGCCACCTCTTGA
GTGCGTCCAGCTTTCCGACAAGTCTCAGTGCCATCTACTGTGCGCTCTGGGTA
TTGCAATTGCTTTTTTTTTTTTTTTTTTTTTTTTTTTAGAATGAGACTAAGTCAG
AGAACACAAAGAACTTCTTTCCCCACAGTGGAGATGGCTCTGAAAGCGTTTA
AGGAATAGCTTAGATGAGTGGCTAACACATTCTCCCGGTTCTGAATTCTAAGA
CCACAGACTCCATGTCCAGTCCCCAAAGAGAGGCTTTGCAAGCTACAGAATA
CCCCTCTGACTGGGACCTCAGGAGCTAAACTGACCACGTAATTGGTTCTAGAA
AGTGAAACGTTTTAATTTGAAACATCCAAATGAGCATTTTGTGAAAAGCTACT
GCCGTCCATCAAATACAACACAGCCAGGGAGTCATCGCTCTATTGCCCTTGTC
AATCCTACATCTATAGTTTTTTTTGCTACAGCAGTTCATGAGTGTTGACTCTAT
TCTAACTTGTTCCAGAAGCCCTTCAAGATGATAGATAGCACAATATTTTTGTA
GCCAGAGCTAGAATGTAGAGCTCTTTTTGGCTTCCTTGTGAATGATCCAGAAT
TTCCATGTTGGCAAGCCACCATAATTTACAGAATTTACTTTTTATATTCAATAG
AAGTAAAAAAAATTTACCTATTTAAGGAGTTATAGCTCTGGATTCATTTCTGA
CCAAAATGTGCTTTTTGACACAAATACAATTGGAAATGTCTTTGTAATTTATC
CACAGTCTGCCTAGATAATCATAAAAGAACTGCATGGATATATTTGTGAGTA
AGAGCACGTGTCCATTCAGCAAAACCAAGGAGATCAACTAATTCTACCATTG
CCTTGAAACGGAGACACATCTAGCAGTTTGAATTTCCCCCAAAAGATTGTATG
TGTGAAAATAAGAATAGAATGAGGAAAATTTAAAAGCCTATATAATAATTTC
AGTCACAACTTGGCAATTAGAATTTTATGAGATGTCTTTAATTTGGAAGCAAA
GAACAATTAAATTATTGAAGGCTGGAATTTTTTTTTAACTCTTTGAATGGAAC
AACAGATTTTCCCCAAAAGATTTGACTTTAACAATTTTCAGAAAACATAAGTC
AGGGTGTGGTTCAATTACACAGAGAGAAATTGTAGTGAAATAGTGTTCCCTG
TAATAATTACCCACAAAGGAGCACAGTGGAGCCACTCCTGCATTAAAATTAC
AGTATCATATGTAAGTTATTATTAATTAACCAGAGATGCCAGGAGCTTGTCAG
TTTCCAACTGCTATTTTGAGGAGAGCTAAAGTTTCTCTTTTTTTGCCAGTTATT
ATTATTATTAATATTTCAACAGCAAGGCAAGAAAAGGGAATGTGGTCCATTA
ACTAATGGCTCTTGAAAAGACACTCAATGAATCCAACTTGCCCTAAAACTGCC
AAGTGGTAGGACAGTCTCTTCGCGTCTTGCATCATTTTCTGCCATCACCTACG
TGTGATTCGTGAGTCGGAAATTCAACCAAGACATGTTTAATGTATATTTAGAG
CATTCTTCCCGGCGGGAATTCACGGTGCCATTCCATCAGGCAGTTGGCAAGCA
GTCACTTGAAATATTAAGAAATATGATTTGTGTCACACTGATTTATTGCAAAA
CAGCAACTTCTTTCTTTTTGGTTCATTTATAAAACAACTGTCAAATTAAAATGC
CAAATAGCTTTAAACATTAGCATTTTCACCTTATAACCTTACAAGTGCATCAC
TTTAAACATCTGAGTAAAAGTTCAGCTCGATGACAATCACCTGGGATTTACCT
GCATGGTACTAAGCATATATGTAAAAATATTACTGATGGGTATCTCTGGCACT
CTGAAGTGACAAAGTGTAGCCTTCACAGATCTTTGTCAGTTAATCATCAATAG
TTACCTGAAAAGTGCCCACTTGCCATCATTCAAGATCAACCAGGCAGACACC
ACAGTGAGTTTTCCATCAAAAAACCTTCTCTATCTGGTCAGTCTCTGCACGTC
AATGAGACAAAGGTGTATGCTGCACGCAGCAGTACTATCCTAAGCTCCCTGT
GTCCTCACCATGGGGCTGGGTGGCTGGGGTGGAGGAACACAGGATTGGGCTT
CAGCTTCTCTAGGGACTGGTACATTAAGAGATGAAGACATAAAAGGTGAGAA
AAACATGGTTTATTTCCAATGTTTCCATTTCTGTTAAAAGTAATGCTTTCAACA
GAAAAAAAATGCAGCAATATAAGTGTGTAATTTACAAAATAATTTCAGGATT
TCTTTAATCATTAATTTGTGGTGTCATCTGTTAACTGGATTTACGTCTAAGCTC
ATTTGTAAATAACTTCAAATATCCAAGCCTTCCCTCACCCTTTTCCCACCTCAC
CTCTCCTCCTTCTCCTCCCCTACACTGGAGGACACTATGTACATGCATATAAT
GTCCTGCCCTAGAGGAGTCCTGAGCCTACTTGGGAAGAAAACACCAACTCAC
AGGAAAACAGCAGAAATCACACAAAACAGAATAAAAGCAAGCGCTGATCTG
TAAGTGAAGACTTAAGTGCTATAGGACTTCCAGCTACAAATCCTGAAAACAC
GGAGTGGCTGTGATAATACGACTAGCCAACATCACACAGTAATTTTGCACAT
AAGGAGAACTAAATCAAAGAAAACAAGGAAAAGAAAGTTGAGCCTATAATC
GTGATACAGGCACTAAAATCTCAGGTGACATTTTTCAATGGGGGAAAGTCAG
TCAACTTCCGATCTCCAAACCATCTTTACTAGCGAGCTTCCCACAATGGTTCT
AGAACCTTCCTTCATTCCAACCCAACCAGGATTCCAACAGACTCATAAACACC
ACAGCCTTTGAGAAATTAAAGGGAGAACCCACCAACCGGCGCCCCACTCCCC
ACCCCAAGTCACCTCTGGCTCAACCAAGATGCGCTCAGGCCAAGAAAGCTGC
CCCACCCCACAGGCTTTGCCTGTCATTTTTAACAAGCCGACTCAGCACATCTC
TCAGATGGGCCATGCAAGGCTTTTCGCAGCTCCTGGGGCTTTGCCTCTTCATG
AGCAGACACTCCCTCTTAGACTAAGACCTGGAGCTGGAAAGTAGGTGGTAAC
CGCGGTACAAAACTCACGCTCGTCCCTGCAGAAACTGCCTAGGTCGGCCCAT
GGCCACGGGGCGCCAATTTTTCAAGGAAAAGTCAATGCTAATAATGGTGGCA
ATCACGGGAAATCCATTCTGAGGCCAGATCTGACTTGTCAGGATTAATCATCA
TTTCCACTTAACTTCGAACTGACCTGGGTAAAAACGTGAGCGCGAGGGGACC
AGGCTGCACCTCTGACCTGGCTCCCCTCTGCAAAAATCGCGAAGTGGGTGCCC
GAGGTGGGGCGGGGGTTGGGGGAGACCTCCCCGGGAGTCCCCACCCAGCCTG
CTCTGCACATCTTAGTCCCTCATCCGCTTGCGCTGTGCAAATCTGTCTTCTGTC
ATTTGTATCGCAAGACATCAAAATCCCCAACCAAATGCAAATACTGAGACCT
CATAATCTGAGACAAAGTTTCACGGTATCCAGAAAGCCCCCAGCAGGTGTGC
AGTGCAGAGCCAGCCCCCCAGCGGTCTTCCGCAGAATCCTATCAGTTTCCCCC
TTTCGTGCTGTGTGCATCGAGCAGGAAGGGGCTTGGCAGGTTTTACCTGCCCT
CTTTCCTTTCTGAAAAGTCTGGGCCTCCTCACCCCGAAAGGAGTCACCTCCTT
GCAGTTCCCCAGTTGCGAAAAGAGGAGGAAGTTGGCTGGGCCGGGGGCCGCG
GGGGGCACCCTCCGCAGATGGCGGGACCCCCCTGCCGGCCATGGCAAAAACG
AGGCTTGTCTCTCCCACCGCCCCCAACCTTAGTCCTTGGCACATTGTTGAAAG
TAATTGAATAAAATCGGAAATTCGAGAAGGCGTTCGTTCGGATTGGTGAGAT
TTTGAGGGGAGAAAGAAGCGGGGACTTCGCCGGCACCAGCGGCGCCCCCTCC
TCGGCCACCGTTAACCCCCATTCCAGAGGGCACTGCCCCGCCACCCAGCCTAG
GTCCCCCTGCGAGAGCCTCGCGGGCCCGCGCAGCCTCCGCGACTCGAACAGA
TCTTCAGTCCTTGGAGGAATGCCTGTTTCTCTAACAATAAAAAATTAAAGAAG
CGCTCATAAATGCCAAGTCCTCTCGCACTATGCGGAGTACAGAGGACAACGA
CCACAGCCATCCCTGAACCCCGCCCACGGCACAGCGCCGGAGCCGGGGTCTG
GGGCGCCGCTTCCTGGGGGGTCCCGACTCTCAGCCGCCCCCGCTTCACCCGGG
CCGCCAAGGGGCTGGGGGAGGCGGCGCTCGGGGTAACCGGGGGAGACTCAG
GGCGCTGGGGGCACTTGGGGAACTCATGGGGGCTCAAAGGAACTAGGAGATC
GGGACCTCGAAGGGGACTTGGGGGGTTCGGGGCTTTCGGGGGCGGTCGGGGG
TTCGCGGACCCGGGAAGCTCTGAGGACCCAGAGGCCGGGCGCGCTCCGCCCG
CGGCGCCGCCCCCTCCGTAACTTTCCCAGTCTCCGAGGGAAGAGGCGGGGTG
TGGGGTGCGGTTAAAAGGCGCCACGGCGGGAGACAGGTGTTGCGGCCCCGCA
GCGCCCGCGCGCTCCTCTCCCCGACTCGGAGCCCCTCGGCGGCGCCCGGCCCA
GGACCCGCCTAGGAGCGCAGGAGCCCCAGCGCAGAGACCCCAACGCCGAGA
CCCCCGCCCCGGCCCCGCCGCGCTTCCTCCCGACGCAGGTGAGCCCGCCGGCC
CCGGACTGCCCGGCCAGGAACCTGGCGCGGGGAGGGACCGCGAGACCCAGA
GCGGTTGCCCGGCCGCGTGGGTCTCGGGGAACCGGGGGGCTGGACCAACACA
CGTCCTTGGGCCGGGGGGGGGGGGCCGCCTTCTGGAGCGGGCGTTTCTGCGG
CCGAGCTCCGGAGCTGGAATGGGGCGGCCGGGGAAGTGGACGCGATGGCAC
CGCCCGGGGTGCGAGTGGGGCCGGGCGCGCGCGGGAGGGGAAAAAGGCGCG
GGCGAGCCGCCAGCGCGAGGTTTGTGGTGTCGCCGATGTCCCTTCGGGGTACT
CTAGCGCAGCCGCCTGGCTACTTGACCCACTGCCACCAAACGTTTTAAATTCA
CCGAAAGCTTAGCTTCGAAGCAAAGCTCCGTTTCGCCGGTGAAGCAGGAAGC
CTTCGCTGCAGGAACTGACCTTTACCTCTTGGAGCGGCTTCTGCAGAAAAATC
CCCGGGCAGAGATTTGGGCGGAGTTTGCCTAGAACTAACGCGGAGCCAGCCG
ATCCCGGCCTACCCCGGGGCCAAGATTTCAGTGGCTTCCCTTTTTCCTAAACA
CTTCACGAGGGTCTGTTTCCGGGCTGTGCTCCCCGCCTAGAAGGAAAATTTTT
AGGACCCTTGTTCGCGAAGAGGTGGTGTGCGGCTGAGACCCGCGTCCTCAGG
ACGGTTCCATCAGTGCCTCGATCCTGCCCCACTGGAGGAGGAAGGCAGCCCG
AACAGCGCTCACCTAACTAACAGCTGCTGAGAGCTGGGTTCCGTGGCCATGC
ACCTGGGACTGCCTTGAGAAGCGTGGTACGGCCGTGTCCCCATGTGACCTTAG
AGTCCCTTTCGAAACTGCTGTGCACAGTCGGTCACAATTTCAGACACTGGTGA
GAAGGGTGGAGGAACCCTCTGGGGACAGCCAGGCAAGGTCGACCACCCATC
ACCTAAGGGTGGAGAAATTTAAGGGGTGAAGAGTCCCTTTTGCCTTTTCTGGA
TCCTGGTGATTCACCTAGTGTCTTCCCTAAGGAACTGAACCAACTCCTCCGCT
GGCCTCTGGCAGCCCTCCAGGCGGTGCAGGATGGCGTGGGCCCGGTAGGAAG
CTGCATGTAACCGCCCAGGGTCGGGAGGCCAGGAGGGCAGCTCCTCCTCTGA
CTTGAATATTGAAAACAACTTCGTCCTGCTTCTGAGCCCCTCTTAACCCATGA
CCCCCTAGCCCATTGGGGAGTAAATCTTAATTTACTCCTCTTCCTGAAAAAGG
ATCTTTAAAACAGGTAGCTTCAACTCAAGCTTTATAAAATAACAATATAGGGT
TTCTCGGAACTGTATTTTTCTCAGCTGATGGTAACTGGACAGGTCTGTAGAAG
GGTGTATGACCTGGGTTTGGCAGGTGGAAGAGGGCAAAGGATAAACCCCTCC
TCCTGCAGCCCCATATTCTTGGCCAGGTGTATTGTTGTAAACCAGGAGAGAGT
TTACTTCGGGGAGTATCCTGTTTTCCACTCAGTGAGGGCCAATGAAGAATGTC
TAATTCCATAAGATGCTTTTGTTAAAATCGGAATGTTGCTGTCCTCGGTGGTT
CTGCTGTTGGGACGGGACTGGCCTGAGCTGTGGGTGCTGTAGCAGGACAACC
AGCTCACCTAAGGGCCTCCCAGTCTGGATTATCAATGGGTCAGTGCTGAACCT
GGGCTAAAATATTGTTTTTTCCAATGATGTTGTCTTTCCCAAGCTCAGTGAAG
CTAAATGTTTCACAGGCCTATGTCAATCTGATGTAACTTTCGTGGCCACCTCT
CTCCTGTTAGCCTCTGACCAAGGTGGCACTGGATGGTTTCTGCCTGACCTTGG
TGCCCCGTGGCAGCGACTGTGGGTCATGAAAGACATTCACTACGAGCCTGCTT
CTGGAGTCCATCAGAAAACGGGATGCAACTTGCCTAAAATGAGGAGAGGAG
GATGCTTTTAAGAAAAAGAAGAAGGAGGATTCACTACCAGCTCTGAAGGGTG
GAAAAGAGATGATTCATCCGGATTGTGGAGAGGGTGGAATCTTGTTTAGGAG
AGCGTTGGTTGTGGCAGGCAGGGTGTAACTATGAATCAGTGAAGACAATTCA
CATCCTGGGATGAAAAGAAGGCCATGGGCTCACAGGAGATTATCCACTGGCC
TCTCCACATCCGCTTGCAGTAAGGAGTGTGGGACTCTCCCAAGCTTCAGCGCT
GAACTGCAATGCAGTGACGTCGCTTAGCTGGGCCAGTAACCGAGGGAGTTGA
ATTTTCTGTCATTTTAAAATAATGTGTCTTTTAAGAAACACTTTGAAATTAAA
ACCACAGCCCACAATTATAATGCACTGTTGCAGCACTTATCAAAACAGATAT
GCTAACTGAGCCATCAGTGCCAGCCTGACAGTGAGGCCACCAAGCCATCCAC
AAAGCCTACACGAAAGTCTGTGCTCACAGTGGCTTTTCTCCATGAAGAGGGC
ATTCCTAACCTCTTCCTTTCACGTAGGAGGAAGCAAGGTCCTTTGTAAAATTT
TAACTCGGGGTGCCTCAAATGTAAACTTAACCACTGGTAACAACAGTTTCACT
GCTACATGCCACGTCTGTGAAAATTCATTCAAGACATTAAGGAAAGTGGCTC
AGCAGAGAGACTAGACATCTTATCCTCACGGTTCTCCTGTACTTGGCCTCTCA
GCCTTTGAGCAAGGTTGGCCCAAGCTAGTATCGGCCCCAGTGGTACAGCCAA
AACTTGAGACTGCAAATGGATGCAGCTGTTGAACGCTGAGTAACTTCTGCAG
AGTCAGGAAGACCCAAGGAAGCTCTGCAGAGGATGCAGGGGTACGGTCAGA
ACCCCTGAGTGCCTTTCAGCTAACGAGGACTTTATGACACTCCCCAGCACAGC
AAATTTTTATGATGTGTTTAAAGATTGGGTGAATTACTCAGGTGAACAAGCTA
CTTTTTATCAGAGAACACCTAAAAACACGTTCAAGAGGGTTTGGGAACTATA
CATTTAATCCTATGACAAACTAAGTTGGTTCTGTCTTCACCTGTTTTGGTGAGG
TTGTGTAAGAGTTGGTGTTTGCTCAGGAAGAGATTTAAGCATGCTTGCTTACC
CAGACTCAGAGAAGTCTCCCTGTTCTGTCCTAGCTAGTGATTCCTGTGTTGTG
TGCATTCGTCTTTTCCAGAGCAAACCGCCCAGAGTAGAAGATGGATTGGGGC
ACGCTGCAGACGATCCTGGGGGGTGTGAACAAACACTCCACCAGCATTGGAA
AGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCA
GCCAGGCTGCAAGAACGTGTGCTACGATCACTACTTCCCCATCTCCCACATCC
GGCTATGGGCCCTGCAGCTGATCTTCGTGTCCACGCCAGCGCTCCTAGTGGCC
ATGCACGTGGCCTACCGGAGACATGAGAAGAAGAGGAAGTTCATCAAGGGG
GAGATAAAGAGTGAATTTAAGGACATCGAGGAGATCAAAACCCAGAAGGTC
CGCATCGAAGGCTCCCTGTGGTGGACCTACACAAGCAGCATCTTCTTCCGGGT
CATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGAC
TGCTTTGTGTCCCGGCCCACGGAGAAGACTGTCTTCACAGTGTTCATGATTGC
AGTGTCTGGAATTTGCATCCTGCTGAATGTCACTGAATTGTGTTATTTGCTAAT
TAGATATTGTTCTGGGAAGTCAAAAAAGCCAGTTTAACGCATTGCCCAGTTGT
TAGATTAAGAAATAGACAGCATGAGAGGGATGAGGCAACCCGTGCTCAGCTG
TCAAGGCTCAGTCGCTAGCATTTCCCAACACAAAGATTCTGACCTTAAATGCA
ACCATTTGAAACCCCTGTAGGCCTCAGGTGAAACTCCAGATGCCACAATGGA
GCTCTGCTCCCCTAAAGCCTCAAAACAAAGGCCTAATTCTATGCCTGTCTTAA
TTTTCTTTCACTTAAGTTAGTTCCACTGAGACCCCAGGCTGTTAGGGGTTATTG
GTGTAAGGTACTTTCATATTTTAAACAGAGGATATCGGCATTTGTTTCTTTCTC
TGAGGACAAGAGAAAAAAGCCAGGTTCCACAGAGGACACAGAGAAGGTTTG
GGTGTCCTCCTGGGGTTCTTTTTGCCAACTTTCCCCACGTTAAAGGTGAACATT
GGTTCTTTCATTTGCTTTGGAAGTTTTAATCTCTAACAGTGGACAAAGTTACC
AGTGCCTTAAACTCTGTTACACTTTTTGGAAGTGAAAACTTTGTAGTATGATA
GGTTATTTTGATGTAAAGATGTTCTGGATACCATTATATGTTCCCCCTGTTTCA
GAGGCTCAGATTGTAATATGTAAATGGTATGTCATTCGCTACTATGATTTAAT
TTGAAATATGGTCTTTTGGTTATGAATACTTTGCAGCACAGCTGAGAGGCTGT
CTGTTGTATTCATTGTGGTCATAGCACCTAACAACATTGTAGCCTCAATCGAG
TGAGACAGACTAGAAGTTCCTAGTGATGGCTTATGATAGCAAATGGCCTCAT
GTCAAATATTTAGATGTAATTTTGTGTAAGAAATACAGACTGGATGTACCACC
AACTACTACCTGTAATGACAGGCCTGTCCAACACATCTCCCTTTTCCATGACT
GTGGTAGCCAGCATCGGAAAGAACGCTGATTTAAAGAGGTCGCTTGGGAATT
TTATTGACACAGTACCATTTAATGGGGAGGACAAAATGGGGCAGGGGAGGGA
GAAGTTTCTGTCGTTAAAAACAGATTTGGAAAGACTGGACTCTAAAGTCTGTT
GATTAAAGATGAGCTTTGTCTACTTCAAAAGTTTGTTTGCTTACCCCTTCAGCC
TCCAATTTTTTAAGTGAAAATATAGCTAATAACATGTGAAAAGAATAGAAGC
TAAGGTTTAGATAAATATTGAGCAGATCTATAGGAAGATTGAACCTGAATAT
TGCCATTATGCTTGACATGGTTTCCAAAAAATGGTACTCCACATATTTCAGTG
AGGGTAAGTATTTTCCTGTTGTCAAGAATAGCATTGTAAAAGCATTTTGTAAT
AATAAAGAATAGCTTTAATGATATGCTTGTAACTAAAATAATTTTGTAATGTA
TCAAATACATTTAAAACATTAAAATATAATCTCTATAATAATTTAAAATCTAA
TATGGTTTTAATAGAACAGCAAATTTTAATTTCATCTATCACTTTTTATATAAA
TACATTAATGTTTTATATTTCATAACACCAATGGGTAAGTTGCCAGAGTGTCT
GACCCCATTCTGCCCCAGTTACAGAAAAGCTTCTGTCACCAGAAAGTTTGGTG
GGGAAGGAAGGGAGGAAGATGATTTCTACCTAACCCCGTGCCCACCTCTACC
AGGTTTTTGAGGCATATCAGTCTATGGACAATGTGGTGTTTGGTCTGGAAACG
TACCTTGGTGAATGCTGAGTTGGCTGGACATGACCCGTTTAGCTCCTGGATGA
ATCCCAGAAGTGGACCTTCAAAATGTTACTCATAGCATGACCTTGGCTCACTG
CAACCTCTGCCTCCCAGGCTCAAGCGATCCTCCCACCTCAGCGTCCCAAGTAG
CTGGGACCACTGGAGTGTGCCACCACACTCCACTAATTTTTTCATTTTTTGTAG
AAACGAGGTCCCACTATATTGCCCAGTCTGGTCTCGAACTCCTGGGCTGAAGG
GATCCCCCTGCCTCAGTCTCCTAAAGTGCAAGGATTACAGGCATGGGCCACC
GCACCTGGCCTGAAACTGCTTTTTATTCCTCAGTGCCCACTTCCATGGGAAAT
AAGCCTGCCAGGTCAGCCTGTCCCCATGGGAGTGACTGCCTGCTACCCCCACA
GGCTTGCCCGGCCCTCGTGAGCCTCTCCCAGAGACACCACCAACAGTTCTGTT
CTTTCATGGTACAAGATTTCCATCCAAGGATTTCAAAGCATTTCACACATCAA
TAATTAGAAGTATTTTCATAGAGGACCATACACTTTTAAAATGGATTTCAAAG
AACAAAAACCAGTCAACTATCACCCAGGTAATAGAAAATGGGAAATGGTTTC
TACCTGACTTCCAAAATGCTCTGCACATAGACTGTGAAAATAGGATTTTTTAA
GCTGGGTGCAGAGGCTTATACCTATAATCCCAACACTTTGGGAGGCTGAGAC
GAGAGGATCACTTGAGCCCAGGAGTTCAAAACCAGCCTGGGCAATATAGGGA
GACATTGTTTCTATAAAAAATAAAAATGTTAGCCAGGCAGGCGTGGTAACAT
GTGCCTGTAGTCTCAGCTACTCAGGAGGCTGAGGTGGGAAGATTGCTTGAAC
CTGGGAGGTCCATGCTGCAGTGAGCTGAGATTGTGCCACTGCACTCCAGCCTA
GGCGACAGCAAGATCCTGTCCCAAACAACAACAACATCAAAAAACACAGAA
CTTTTAAAATAAGTACATTCACTTCTACAAGCTATGTAGATTATTACTCTCAA
GCTATTAAAAGACCAAGCCAAAATAATTATGGGCTACTCTCGACCACTTGTA
GGAATGGATAGAGAGGTCTGGTCACATGCCTGGAAATTAGAGCTTGAGCTCT
GAAAATGATAATCCTGACTATATCTCAAAGCATCAGTCTGCACTTTGTATGGA
GCAAGAAAAAGCCTTGTGGAAGCGGCCTCCCACCCAGCCGAGCCCTCGGCGT
GGACAAGCTCTGCTTTTTATGAGCAGTGGGTGCAGCCTCGCTGCTCCCTCCTC
CTGTCAAAAGACAGTCACAGCTGGGGTGAGCAGATCGGGCCCACTTGGGAGG
CCCCAAGGAATATGCTGCAGGGGTCGGGCCTGAGCCACCCCCACGGGTTGGT
CTTTGACAACTAGAGAGCAGCTGAGAGGTGGGTAAAAGCTCACTCACTTACC
CTGACCTCAGTGTCCTCATCTTAAAATGGGTTTCCTGAATCTTTCCCCGGCTTA
GTGGCAATGAAATAAGATAATTTATGTAAACGTTCTCCACATAGTAAAGCAC
TAAGTAACATATGACTGTCATCTGTTTTCCACTAGACAGATCCCAACCTGGAA
GAGTGACAGATGGTATTTCAGATACAAGTGACTCAAGCAAAGCTTGATAAAC
TGGGGGCTGGAAAAAAATGCACATTTACACAAAGCCTGGAGTAACTGC

In some aspects, the GJB2 gene is codon optimized. In some aspects, the codon optimized GJB2 gene as at least 70%, at least 75%, at least 80%, at least 85%, at least 90%, at least 91%, at least 92%, at least 93%, at least 94%, at least 95%, at least 96%, at least 97%, at least 98%, at least 99%, or 100 identity to any one of SEQ ID NOs: 123-126. In some aspects, the codon optimized GJB2 gene has the sequence of any one of SEQ ID NOs: 123-126.

Exemplary codon optimized Human GJB2 DNA Sequence
(SEQ ID NO: 123)
ATGGACTGGGGCACCCTGCAGACTATCCTGGGGGGCGTCAATAAGCATTC
AACTAGCATCGGAAAGATTTGGCTGACTGTCCTGTTTATCTTTCGGATCA
TGATCCTGGTGGTGGCAGCAAAGGAAGTGTGGGGCGACGAGCAGGCCGAT
TTCGTGTGCAACACACTGCAGCCAGGCTGCAAGAACGTGTGCTACGACCA
CTATTTTCCCATCTCTCACATCAGGCTGTGGGCCCTGCAGCTGATCTTCG
TGAGCACCCCTGCCCTGCTGGTGGCAATGCACGTGGCCTATCGGAGACAC
GAGAAGAAGCGCAAGTTTATCAAGGGCGAGATCAAGAGCGAGTTCAAGGA
TATCGAGGAGATCAAGACACAGAAGGTGAGGATCGAGGGCTCCCTGTGGT
GGACCTACACAAGCTCCATCTTCTTTCGCGTGATCTTCGAGGCCGCCTTT
ATGTACGTGTTCTATGTGATGTACGACGGCTTTTCTATGCAGCGGCTGGT
GAAGTGCAACGCCTGGCCCTGTCCTAATACAGTGGATTGTTTCGTGTCCA
GACCCACCGAGAAGACAGTGTTCACCGTGTTTATGATCGCCGTGTCTGGC
ATCTGCATCCTGCTGAACGTGACCGAGCTGTGCTATCTGCTGATCCGGTA
CTGTAGTGGAAAGAGCAAAAAACCCGTG
Exemplary codon optimized Human GJB2 DNA Sequence
(SEQ ID NO: 124)
ATGGACTGGGGAACATTGCAAACTATTTTGGGAGGAGTCAACAAGCATTC
AACTAGCATCGGGAAGATCTGGCTGACCGTGCTGTTCATCTTTCGCATCA
TGATTCTCGTGGTGGCCGCTAAGGAAGTCTGGGGCGATGAACAGGCCGAC
TTCGTGTGTAACACGCTGCAGCCCGGTTGCAAAAACGTCTGCTACGATCA
CTACTTCCCCATCTCACACATTAGACTGTGGGCGCTGCAGCTGATTTTCG
TGTCCACCCCGGCACTTCTTGTGGCGATGCACGTGGCCTACCGGCGGCAC
GAGAAGAAAAGGAAGTTCATTAAGGGCGAAATCAAGTCCGAGTTCAAGGA
CATCGAAGAAATCAAGACCCAGAAGGTCCGCATTGAGGGCTCCCTCTGGT
GGACCTACACCTCGTCCATCTTCTTCCGGGTCATATTCGAGGCCGCCTTT
ATGTACGTGTTTTACGTGATGTACGACGGTTTCAGCATGCAAAGACTCGT
CAAGTGCAACGCTTGGCCTTGCCCCAATACCGTGGATTGCTTCGTGTCCC
GCCCGACCGAGAAAACTGTGTTCACTGTGTTCATGATCGCCGTGTCCGGC
ATCTGCATCCTGCTGAACGTGACCGAGCTGTGCTATCTCCTGATCCGGTA
CTGTAGCGGAAAGTCGAAGAAGCCTGTG
Exemplary codon optimized Human GJB2 DNA Sequence
(SEQ ID NO: 125)
ATGGATTGGGGGACGCTCCAGACTATACTTGGCGGGGTAAACAAACATTC
CACCTCAATTGGCAAAATCTGGCTCACAGTCCTCTTCATCTTCAGAATAA
TGATACTCGTGGTTGCCGCTAAAGAAGTTTGGGGTGACGAGCAAGCCGAT
TTCGTCTGTAACACCCTCCAACCAGGTTGCAAAAATGTCTGTTACGATCA
CTACTTTCCTATTAGCCATATTAGACTCTGGGCCCTGCAACTTATCTTCG
TTTCCACTCCTGCTCTGCTCGTCGCTATGCACGTTGCCTATCGCCGCCAT
GAAAAAAAACGGAAATTCATTAAGGGAGAGATTAAGAGTGAATTCAAGGA
TATTGAAGAGATTAAAACGCAAAAAGTTAGAATTGAGGGATCACTGTGGT
GGACTTATACCAGTAGCATCTTTTTTAGGGTCATTTTCGAAGCTGCTTTC
ATGTATGTTTTCTATGTAATGTACGACGGTTTCTCCATGCAACGCTTGGT
TAAATGTAACGCCTGGCCATGCCCTAATACGGTTGATTGCTTTGTCTCCC
GCCCTACTGAAAAGACAGTGTTTACCGTTTTCATGATCGCCGTAAGTGGA
ATTTGTATCCTTCTTAACGTGACCGAGTTGTGCTATCTCCTTATTCGCTA
CTGTTCAGGAAAAAGTAAAAAACCAGTA
Exemplary codon optimized Human GJB2 DNA Sequence
(SEQ ID NO: 126)
ATGGACTGGGGCACGCTGCAGACTATCCTGGGGGGTGTCAACAAGCATTC
AACTAGCATCGGAAAGATCTGGCTGACCGTCCTGTTCATCTTTCGCATCA
TGATCCTCGTGGTGGCCGCTAAGGAAGTGTGGGGCGACGAGCAGGCCGAT
TTCGTGTGTAACACCCTGCAGCCAGGTTGCAAAAACGTCTGCTACGATCA
CTACTTTCCCATCTCCCACATTAGACTGTGGGCCCTGCAGCTGATCTTCG
TGTCCACCCCTGCGCTGCTAGTGGCCATGCACGTGGCCTATCGGCGACAC
GAGAAGAAACGGAAGTTCATTAAGGGCGAGATCAAGAGCGAGTTCAAGGA
TATCGAAGAGATCAAGACCCAGAAGGTCCGCATTGAGGGCTCCCTGTGGT
GGACCTACACCAGCTCCATCTTCTTTCGGGTCATCTTCGAGGCCGCCTTT
ATGTACGTGTTCTATGTGATGTACGACGGTTTCTCCATGCAACGGCTGGT
GAAGTGCAACGCCTGGCCTTGCCCTAATACTGTGGATTGCTTCGTGTCCC
GCCCCACCGAGAAGACAGTGTTCACCGTGTTCATGATCGCCGTGTCTGGC
ATCTGCATCCTGCTGAACGTGACCGAGCTGTGCTATCTCCTGATCCGGTA
CTGTAGTGGAAAGTCAAAAAAACCAGTGTAA

The present disclosure recognizes that certain changes to a polynucleotide sequence will not impact its expression or a protein encoded by said polynucleotide. In some aspects, a polynucleotide comprises a GJB2 gene having one or more silent mutations. In some aspects, the disclosure provides a polynucleotide that comprises a GJB2 gene having one or more silent mutations, e.g., a GJB2 gene having a sequence different from SEQ ID NOs: 117-126 but encoding the same amino acid sequence as a functional GJB2 gene. In some aspects, the disclosure provides a polynucleotide that comprises a GJB2 gene having a sequence different from SEQ ID NO: 117-126 that encodes an amino acid sequence including one or more mutations (e.g., a different amino acid sequence when compared to that produced from a functional GJB2 gene), where the one or more mutations are conservative amino acid substitutions.

In some aspects, the disclosure provides a polynucleotide that comprises a GJB2 gene having a sequence different from SEQ ID NO: 117-126 that encodes an amino acid sequence including one or more mutations (e.g., a different amino acid sequence when compared to that produced from a functional GJB2 gene), where the one or more mutations are not within a characteristic portion of a GJB2 gene or an encoded connexin 26 protein. In some aspects, a polynucleotide in accordance with the present disclosure comprises a GJB2 gene that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 117-126. In some aspects, a polynucleotide in accordance with the present disclosure comprises a GJB2 gene that is identical to the sequence of SEQ ID NO: 117-126. As can be appreciated in the art, SEQ ID NO: 117-126 can be optimized (e.g., codon optimized) to achieve increased or optimal expression in an animal, e.g., a mammal, e.g., a human.

Among other things, the present disclosure provides polypeptides encoded by a GJB2 gene or characteristic portion thereof. In some aspects, a GJB2 gene is a mammalian GJB2 gene. In some aspects, a GJB2 gene is a murine GJB2 gene. In some aspects, a GJB2 gene is a primate GJB2 gene. In some aspects, a GJB2 gene is a human GJB2 gene.

In some aspects, a polypeptide comprises a connexin 26 protein or characteristic portion thereof. In some aspects, a connexin 26 protein or characteristic portion thereof is mammalian connexin 26 protein or characteristic portion thereof, e.g., primate connexin 26 protein or characteristic portion thereof. In some aspects, a connexin 26 protein or characteristic portion thereof is a human connexin 26 protein or characteristic portion thereof.

In some aspects, a polypeptide provided herein comprises post-translational modifications. In some aspects, a connexin 26 protein or characteristic portion thereof provided herein comprises post-translational modifications. In some aspects, post-translational modifications can comprise but is not limited to glycosylation (e.g., N-linked glycosylation, O-linked glycosylation), phosphorylation, acetylation, amidation, hydroxylation, methylation, ubiquitylation, sulfation, and/or a combination thereof. An exemplary human connexin 26 protein sequence is or includes the sequence of SEQ ID NO: 127.

Exemplary Human Connexin 26 Protein Sequence
(SEQ ID NO: 127)
MDWGTLQTILGGVNKHSTSIGKIWLTVLFIFRIMILVVAAKEVWGDEQAD
FVCNTLQPGCKNVCYDHYFPISHIRLWALQLIFVSTPALLVAMHVAYRRH
EKKRKFIKGEIKSEFKDIEEIKTQKVRIEGSLWWTYTSSIFFRVIFEAAF
MYVFYVMYDGFSMQRLVKCNAWPCPNTVDCFVSRPTEKTVFTVFMIAVSG
ICILLNVTELCYLLIRYCSGKSKKPV

The present disclosure recognizes that certain mutations in an amino acid sequence of a polypeptide described herein (e.g., including connexin 26 or a characteristic portion thereof) will not impact the expression, folding, or activity of the polypeptide. In some aspects, a polypeptide (e.g., including connexin 26 or a characteristic portion thereof) includes one or more mutations, where the one or more mutations are conservative amino acid substitutions. In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 or a characteristic portion thereof that is identical to the sequence of SEQ ID NO: 127. In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 protein or a characteristic portion thereof that is identical to the sequence of SEQ ID NO: 127.

In some aspects, the polypeptide is a therapeutic polypeptide (e.g., a Connexin 26 polypeptide). In some aspects, the polypeptide is a supporting cell polypeptide (e.g., a Connexin 26 polypeptide). In some aspects, the polypeptide is a reporter polypeptide.

### Supporting Cell Polypeptides

Certain aspects of the disclosure are directed to polynucleotides encoding a supporting cell polypeptide (e.g., a Connexin 26 polypeptide). The polynucleotide can encode a polypeptide that is capable of being expressed in a cell (e.g., an inner ear cell). In some aspects, the supporting cell polypeptide (e.g., a Connexin 26 polypeptide) is a poypeptide that is endogenously expressed in supporting cells of the inner ear. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. The polynucleotide can encode a full length polypeptide or a functional fragment thereof.

Exemplary supporting cell polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary supporting cell polynucleotides encoding polypeptides include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4). In some aspects, the polynucleotide comprises a gap junction protein beta 2 (GJB2) gene. In some aspects, the polynucleotide encodes a gap junction protein beta 2 polypeptide. In some aspects, the polynucleotide encodes a Connexin 26 polypeptide. In some aspects, the supporting cell polypeptide is a gap junction protein beta 2 polypeptide. In some aspects, the supporting cell polypeptide is a Connexin 26 polypeptide.

In some aspects, a polynucleotide in accordance with the present disclosure comprises a GJB2 gene that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 117-126. In some aspects, a polynucleotide in accordance with the present disclosure comprises a GJB2 gene that is identical to the sequence of SEQ ID NO: 117-126. As can be appreciated in the art, SEQ ID NO: 117-126 can be optimized (e.g., codon optimized) to achieve increased or optimal expression in an animal, e.g., a mammal, e.g., a human.

In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 or a characteristic portion thereof that is identical to the sequence of SEQ ID NO: 127. In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 protein or a characteristic portion thereof that is identical to the sequence of SEQ ID NO: 127.

### Therapeutic Polypeptides

Certain aspects of the disclosure are directed to polynucleotides encoding a polypeptide (e.g., a therapeutic polypeptide, a Connexin 26 polypeptide). The polynucleotide can encode a polypeptide that is capable of being expressed in a cell (e.g., an inner ear cell). The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding therapeutic polypeptides (e.g., a Connexin 26 polypeptide) include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4). In some aspects, the polynucleotide comprises a gap junction protein beta 2 (GJB2) gene. In some aspects, the polynucleotide encodes a gap junction protein beta 2 polypeptide. In some aspects, the polynucleotide encodes a Connexin 26 polypeptide. In some aspects, the therapeutic polypeptide is a gap junction protein beta 2 polypeptide. In some aspects, the therapeutic polypeptide is a Connexin 26 polypeptide.

In some aspects, a polynucleotide in accordance with the present disclosure comprises a GJB2 gene that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 117-126. In some aspects, a polynucleotide in accordance with the present disclosure comprises a GJB2 gene that is identical to the sequence of SEQ ID NO: 117-126. As can be appreciated in the art, SEQ ID NO: 117-126 can be optimized (e.g., codon optimized) to achieve increased or optimal expression in an animal, e.g., a mammal, e.g., a human.

In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 or a characteristic portion thereof that is identical to the sequence of SEQ ID NO: 127. In some aspects, a polypeptide in accordance with the present disclosure comprises a connexin 26 or a characteristic portion thereof that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence of SEQ ID NO: 127. In some aspects, a polypeptide in accordance with the present disclosure comprises a connexin 26 protein or a characteristic portion thereof that is identical to the sequence of SEQ ID NO: 127.

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a nucleic acid sequence (e.g., GJB2 gene) or characteristic portion thereof encoding a polypeptide (e.g., Connexin 26). Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series). In some aspects, the construct is a mammalian or a viral vector.

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated in its entirety herein by reference). In some aspects, the construct is a viral vector. In some aspects, the construct is a lentivirus, retrovirus, adenovirus, or adeno-associated virus vector. In some aspects, the construct is an AAV vector. In some aspects, a viral construct is an adenovirus construct. In some aspects, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adeno-associated virus construct and can have a total number of nucleotides of up to 8 kb. In some aspects, an adeno-associated virus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

In some aspects, the construct comprises a polynucleotide encoding a therapeutic polypeptide (e.g., a Connexin 26 polypeptide) operably linked to a promoter which selectively expresses the polynucleotide in an inner ear support cell. In some aspects, the construct comprise a 5′ ITR, a promoter which selectively expresses the polynucleotide in an inner ear support cell, a 5′ UTR, a polynucleotide encoding a therapeutic polypeptide (e.g., a Connexin 26 polypeptide), a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a therapeutic polypeptide (e.g., a Connexin 26 polypeptide), a tag, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a therapeutic polypeptide (e.g., a Connexin 26 polypeptide), a tag, a 3′ UTR, a microRNA regulatory target site, a polyA, and a 3′ ITR.

In some aspects, the construct comprises a polynucleotide encoding a polypeptide operably linked to a promoter which selectively expresses the polynucleotide in an inner ear support cell. In some aspects, the construct comprise a 5′ ITR, a promoter which selectively expresses the polynucleotide in an inner ear support cell, a 5′ UTR, a polynucleotide encoding a polypeptide (e.g., a Connexin 26 polypeptide), a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a polypeptide (e.g., a Connexin 26 polypeptide), a tag, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a polypeptide (e.g., a Connexin 26 polypeptide), a tag, a 3′ UTR, a microRNA regulatory target site, a polyA, and a 3′ ITR.

In some aspects, the construct comprises a polynucleotide encoding a therapeutic polypeptide (e.g., a Connexin 26 polypeptide) operably linked to a promoter, wherein the construct comprises a miRNA regulatory target site for a microRNA expressed in an inner ear cell (e.g., a hair cell). In some aspects, the construct comprises a 5′ ITR, a promoter which selectively expresses the polynucleotide in an inner ear support cell, a 5′ UTR, a polynucleotide encoding a therapeutic polypeptide (e.g., a Connexin 26 polypeptide), a 3′ UTR, a microRNA regulatory target site, a polyA, and a 3′ ITR. In some aspects, the construct comprises a 5′ ITR, a promoter which selectively expresses the polynucleotide in an inner ear support cell, a 5′ UTR, a polynucleotide encoding a therapeutic polypeptide (e.g., a Connexin 26 polypeptide), a tag, a 3′ UTR, a microRNA regulatory target site, a polyA, and a 3′ ITR. In some aspects, the construct comprises a 5′ ITR, a constitutive promoter, a 5′ UTR, a polynucleotide encoding a therapeutic polypeptide (e.g., a Connexin 26 polypeptide), a 3′ UTR, a microRNA regulatory target site, a polyA, and a 3′ ITR. In some aspects, the construct comprises a 5′ ITR, a constitutive promoter, a 5′ UTR, a polynucleotide encoding a therapeutic polypeptide (e.g., a Connexin 26 polypeptide), a tag, a 3′ UTR, a microRNA regulatory target site, a polyA, and a 3′ ITR.

In some aspects, the construct comprises a polynucleotide encoding a polypeptide operably linked to a promoter, wherein the construct comprises a miRNA regulatory target site for a microRNA expressed in an inner ear cell (e.g., a hair cell). In some aspects, the construct comprises a 5′ ITR, a promoter which selectively expresses the polynucleotide in an inner ear support cell, a 5′ UTR, a polynucleotide encoding a polypeptide (e.g., a Connexin 26 polypeptide), a 3′ UTR, a microRNA regulatory target site, a polyA, and a 3′ ITR. In some aspects, the construct comprises a 5′ ITR, a promoter which selectively expresses the polynucleotide in an inner ear support cell, a 5′ UTR, a polynucleotide encoding a polypeptide (e.g., a Connexin 26 polypeptide), a tag, a 3′ UTR, a microRNA regulatory target site, a polyA, and a 3′ ITR. In some aspects, the construct comprises a 5′ ITR, a constitutive promoter, a 5′ UTR, a polynucleotide encoding a polypeptide (e.g., a Connexin 26 polypeptide), a 3′ UTR, a microRNA regulatory target site, a polyA, and a 3′ ITR. In some aspects, the construct comprises a 5′ ITR, a constitutive promoter, a 5′ UTR, a polynucleotide encoding a polypeptide (e.g., a Connexin 26 polypeptide), a tag, a 3′ UTR, a microRNA regulatory target site, a polyA, and a 3′ ITR.

In some aspects, the construct comprises (i) a 5′ inverted terminal repeat (ITR), (ii) a polynucleotide encoding a polypeptide (e.g., a Connexin 26 polypeptide) operably linked to a promoter which expresses the polynucleotide in an inner ear support cell, and (iii) a 3′ ITR, wherein the promoter is heterologous to the polynucleotide.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 95. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98.

In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 95 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter which expresses the polynucleotide in an inner ear support cell, and (iv) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter, and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to a promoter which expresses the polynucleotide in an inner ear support cell, (iii) a 3′ untranslated region (UTR), and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to a promoter, (iii) a 3′ untranslated region (UTR), and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to a promoter which expresses the polynucleotide in an inner ear support cell, (iii) a miRNA regulatory target site (miRTS) for a microRNA expressed in an inner ear cell (e.g., a hair cell), (iv) a 3′ untranslated region (UTR), and (v) the 3′ ITR.

In some aspects, the construct comprises (i) the 5′ inverted terminal repeat (ITR), (ii) the polynucleotide encoding a polypeptide (e.g., a Connexin 26 polypeptide) operably linked to a promoter, (iii) a miRNA regulatory target site (miRTS) for a microRNA expressed in an inner ear cell (e.g., a hair cell), (iv) a 3′ untranslated region (UTR), and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter which expresses the polynucleotide in an inner ear support cell, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter which expresses the polynucleotide in an inner ear support cell, (iii) a miRNA regulatory target site (miRTS) for a microRNA expressed in an inner ear cell (e.g., a hair cell), (v) a 3′ UTR, and (vi) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to a promoter, (iv) a miRNA regulatory target site (miRTS) for a microRNA expressed in an inner ear cell (e.g., a hair cell), (v) a 3′ UTR, and (vi) the 3′ ITR.

In some aspects, the construct comprises a polynucleotide encoding a polypeptide (e.g., a Connexin 26 polypeptide) operably linked to a promoter, wherein the construct comprises a miRNA regulatory target site (miRTS) for a microRNA expressed in an inner ear cell (e.g., a hair cell).

In some aspects, the construct comprises (i) a 5′ inverted terminal repeat (ITR), (ii) the polynucleotide encoding a polypeptide (e.g., a Connexin 26 polypeptide) operably linked to a promoter, (iii) the miRNA regulatory target site (miRTS) for a microRNA expressed in an inner ear cell (e.g., a hair cell), and (iv) a 3′ ITR.

In some aspects, the construct comprises (i) a 5′ inverted terminal repeat (ITR), (ii) a 5′ untranslated region (UTR), (iii) the polynucleotide encoding a polypeptide (e.g., a Connexin 26 polypeptide) operably linked to a promoter, (iv) the miRNA regulatory target site (miRTS) for a microRNA expressed in an inner ear cell (e.g., a hair cell), and (v) a 3′ ITR.

In some aspects, the construct comprises (i) a 5′ inverted terminal repeat (ITR), (ii) the polynucleotide encoding a polypeptide (e.g., a Connexin 26 polypeptide) operably linked to a promoter, (iii) the miRNA regulatory target site (miRTS) for a microRNA expressed in an inner ear cell (e.g., a hair cell), (iv) a 3′ untranslated region (UTR) and (v) a 3′ ITR.

In some aspects, the construct comprises (i) a 5′ inverted terminal repeat (ITR), (ii) a 5′ untranslated region (UTR), (iii) the polynucleotide encoding a polypeptide (e.g., a Connexin 26 polypeptide) operably linked to a promoter, (iv) the miRNA regulatory target site (miRTS) for a microRNA expressed in an inner ear cell (e.g., a hair cell), (v) a 3′ UTR, and (vi) a 3′ ITR.

In some aspects, the construct comprises (i) a 5′ inverted terminal repeat (ITR), (ii) a polynucleotide encoding a polypeptide (e.g., a Connexin 26 polypeptide) operably linked to an inner ear supporting cell selective promoter and a minimal GJB2 promoter which expresses the polynucleotide in an inner ear support cell, and (iii) a 3′ ITR, wherein the inner ear supporting cell selective promoter is heterologous to the polynucleotide.

In some aspects, the construct comprises (i) a 5′ inverted terminal repeat (ITR), (ii) a polynucleotide encoding a polypeptide (e.g., a Connexin 26 polypeptide) operably linked to an inner ear supporting cell selective promoter and a minimal GJB2 promoter, and (iii) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 95. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98. In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter which expresses the polynucleotide in an inner ear support cell, and (iv) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter, and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter which expresses the polynucleotide in an inner ear support cell, (iii) a 3′ untranslated region (UTR), and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter, (iii) a 3′ untranslated region (UTR), and (iv) the 3′ ITR.

In some aspects, the construct comprises (i) the 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter which expresses the polynucleotide in an inner ear support cell, (iii) a miRNA regulatory target site (miRTS) for a microRNA expressed in an inner ear cell (e.g., hair cell), (iv) a 3′ untranslated region (UTR), and (v) the 3′ ITR.

In some aspects, the construct comprises (i) the 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter, (iii) a miRNA regulatory target site (miRTS) for a microRNA expressed in an inner ear cell (e.g., a hair cell), (iv) a 3′ untranslated region (UTR), and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter which expresses the polynucleotide in an inner ear support cell, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter which expresses the polynucleotide in an inner ear support cell, (iii) a miRNA regulatory target site (miRTS) for a microRNA expressed in an inner ear cell (e.g., a hair cell), (v) a 3′ UTR, and (vi) the 3′ ITR.

In some aspects, the construct comprises (i) the 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter, (iv) a miRNA regulatory target site (miRTS) for a microRNA expressed in an inner ear cell (e.g., a hair cell), (v) a 3′ UTR, and (vi) the 3′ ITR.

In some aspects, the construct comprises a polynucleotide encoding a polypeptide (e.g., a Connexin 26 polypeptide) operably linked to an inner ear supporting cell selective promoter and a minimal GJB2 promoter, wherein the construct comprises a miRNA regulatory target site (miRTS) for a microRNA expressed in an inner ear cell (e.g., a hair cell).

In some aspects, the construct comprises (i) a 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter, (iii) the miRNA regulatory target site (miRTS) for a microRNA expressed in an inner ear cell (e.g., a hair cell), and (iv) a 3′ ITR.

In some aspects, the construct comprises (i) a 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter, (iv) the miRNA regulatory target site (miRTS) for a microRNA expressed in an inner ear cell (e.g., a hair cell), and (v) a 3′ ITR.

In some aspects, the construct comprises (i) a 5′ inverted terminal repeat (ITR), (ii) the polynucleotide encoding a polypeptide (e.g., a Connexin 26 polypeptide) operably linked to an inner ear supporting cell selective promoter and a minimal GJB2 promoter, (iii) the miRNA regulatory target site (miRTS) for a microRNA expressed in an inner ear cell (e.g., a hair cell), (iv) a 3′ untranslated region (UTR) and (v) a 3′ ITR.

In some aspects, the construct comprises (i) a 5′ inverted terminal repeat (ITR), (ii) a 5′ untranslated region (UTR), (iii) the polynucleotide encoding a polypeptide (e.g., a Connexin 26 polypeptide) operably linked to an inner ear supporting cell selective promoter and a minimal GJB2 promoter, (iv) the miRNA regulatory target site (miRTS) for a microRNA expressed in an inner ear cell (e.g., a hair cell), (v) a 3′ UTR, and (vi) a 3′ ITR.

In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 95 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86. In some aspects, the construct comprises (i) a 5′ inverted terminal repeat (ITR), (ii) a polynucleotide encoding a polypeptide (e.g., a Connexin 26 polypeptide) operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98 and a minimal GJB2 promoter comprising a nucleic acid sequence at least 80%, at least 85%, at least 90%, at least 95%, at least 96%, at least 97%, at least 98%, at least 99%, or 100% identity to SEQ ID NO: 86.

### AAV Particles

Among other things, the present disclosure provides AAV particles that comprise a construct encoding a therapeutic polypeptide (e.g., a Connexin 26 polypeptide), and a capsid described herein. Among other things, the present disclosure provides AAV particles that comprise a construct comprising a nucleic acid sequence (e.g., a gene) encoding a polypeptide, and a capsid described herein. In some aspects, AAV particles can be described as having a serotype, which is a description of the construct strain and the capsid strain. In some aspects, the AAV particle has an AAV1, AAV2, AAV3 (e.g., AAV3B), AAV4, AAV5, AAV6, AAV7, AAV8, AAV9, AAV10, AAV11, AAV2-tYF, AAV2-P2V2, AAV2-P2V3, AAV2-MeBtYFTV, AAV2-MeB, AAV2-P2V6, AAV2-DGEDF, or an AAV Anc80 serotype. In some aspects, the AAV particle has an AAVAnc80 serotype (including, for example, an AAVAnc80L65). In some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid. AAV Construct

The present disclosure provides constructs that comprise a nucleic acid sequence (e.g., a gene) encoding a polypeptide or characteristic portion thereof. In some aspects described herein, a construct comprising a nucleic acid sequence (e.g., a gene) encoding a polypeptide or characteristic portion thereof can be included in an AAV particle.

The present disclosure provides polynucleotide constructs that comprise a nucleic acid sequence (e.g., a gene) encoding a therapeutic polypeptide (e.g., a Connexin 26 polypeptide) or characteristic portion thereof). In some aspects described herein, a polynucleotide comprising a nucleic acid sequence (e.g., a gene) encoding a therapeutic polypeptide (e.g., a Connexin 26 polypeptide) or characteristic portion thereof can be included in an AAV particle.

In some aspects, a polynucleotide construct comprises one or more components derived from or modified from naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, an AAV2-tYF construct, an AAV2-P2V2 construct, an AAV2-P2V3 construct, an AAV2-MeBtYFTV construct, an AAV2-MeB construct, an AAV2-P2V6 construct, an AAV2-DGEDF construct, or AAV Anc80 construct. In some aspects, the construct is derived from an AAV Anc80 construct (including, for example, an AAVAnc80L65). Additional exemplary AAV constructs that can be used herein are known in the art. See, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference.

In some aspects, provided constructs comprise coding sequence, e.g., a nucleic acid encoding polypeptide (e.g., a therapeutic polypeptide, a Connexin 26 polypeptide), one or more regulatory and/or control sequences, and optionally 5′ and 3′ AAV derived inverted terminal repeats (ITRs). In some aspects wherein a 5′ and 3′ AAV derived ITR is utilized, the polynucleotide construct may be referred to as a recombinant AAV (rAAV) construct. In some aspects, provided rAAV constructs are packaged into an AAV capsid to form an AAV particle. In some aspects, an AAV capsid is an Anc80 capsid (e.g., an Anc80L65 capsid).

In some aspects, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some aspects, at least 75% of a typical ITR sequence (e.g., at least 80%, at least 85%, at least 90%, or at least 95%) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some aspects, a nucleic acid coding sequence is operatively linked to and/or control components in a manner that permits coding sequence transcription, translation, and/or expression in a cell of a target tissue.

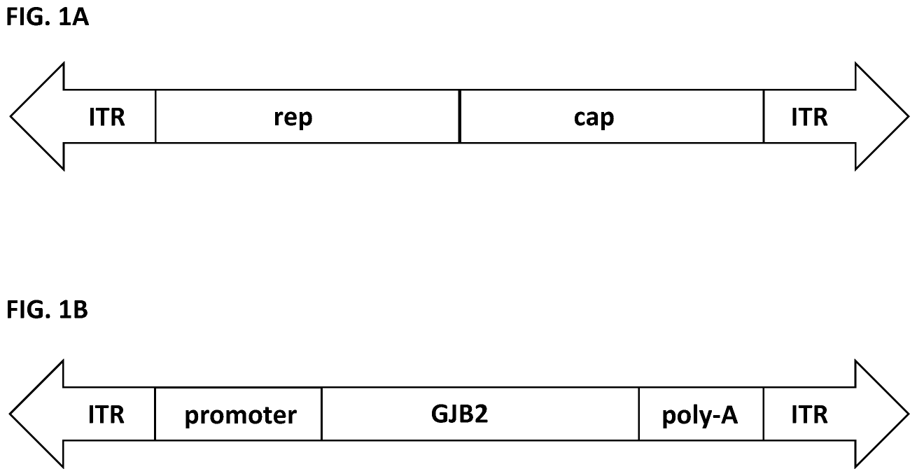

As shown in FIG. 1A, an unmodified AAV endogenous genome includes two open reading frames, “cap” and “rep,” which are flanked by ITRs. As shown in FIG. 1, exemplary rAAV constructs similarly include ITRs flanking a coding region, e.g., a coding sequence (e.g., a polynucleotide encoding a polypeptide (e.g., a therapeutic polypeptide, a Connexin 26 polypeptide)). In some aspects, a rAAV construct also comprises conventional control elements that are operably linked to the coding sequence in a manner that permits its transcription, translation and/or expression in a cell transfected with the plasmid construct or infected with the virus produced by the disclosure. In some aspects, a rAAV construct optionally comprises a promoter (shown in FIG. 1, panel (B)), an enhancer, an untranslated region (e.g., a 5′ UTR, 3′ UTR), a Kozak sequence, an internal ribosomal entry site (IRES), splicing sites (e.g., an acceptor site, a donor site), a polyadenylation site (shown in FIG. 1, panel (B)), or any combination thereof. In some aspects, an rAAV construct comprises a promoter, a 5′ UTR, and a polyadenylation site. In some aspects, an rAAV construct comprises a promoter, a 5′ UTR, a 3′ UTR, and a polyadenylation site. Such additional elements are described further herein.

In some aspects, a construct is an rAAV construct. In some aspects, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein.

In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter which expresses the polynucleotide in an inner ear support cell, and (iii) a 3′ ITR, wherein the promoter is heterologous to the polynucleotide. In some aspects, the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 40, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 90, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 96, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 99, and (iii) a 3′ ITR, wherein the promoter is heterologous to the polynucleotide.

In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 16, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 28, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 57, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 91, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 92, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 93, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 94, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 95, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 97, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 98, and (iii) a 3′ ITR, wherein the promoter is heterologous to the polynucleotide.

In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 40 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 90 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 96 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 99 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide.

In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 16 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 28 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 57 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 91 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 92 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 93 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 94 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 95 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 97 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle (e.g., an Anc80 particle) comprises a construct comprising: (i) a 5′ inverted terminal repeat (ITR), (ii) a polynucleotide encoding a polypeptide (e.g., a Connexin 26 polypeptide) operably linked to a promoter having at least 85%, at least 90%, at least 95%, at least 96%, at least 97%, at least 98%, at least 99%, or 100% identity to any one of SEQ ID NO: 98 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86, and (iii) a 3′ ITR, wherein the promoter is heterologous to the polynucleotide. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g., a Connexin 26 polypeptide) operably linked to a promoter which expresses the polynucleotide in an inner ear support cell, (iv) a 3′ UTR, and (v) a 3′ ITR, wherein the promoter is heterologous to the polynucleotide.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g., a Connexin 26 polypeptide) operably linked to a promoter, (iv) a miRNA regulatory target site (miRTS) for a microRNA expressed in an inner ear cell (e.g., a hair cell), (v) a 3′ UTR, and (vi) a 3′ ITR.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g., a Connexin 26 polypeptide) operably linked to an inner ear supporting cell selective promoter and a minimal GJB2 promoter which expresses the polynucleotide in an inner ear support cell, (iv) a 3′ UTR, and (v) a 3′ ITR, wherein the inner ear supporting cell selective promoter is heterologous to the polynucleotide.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g., a Connexin 26 polypeptide) operably linked to an inner ear supporting cell selective promoter and a minimal GJB2 promoter, (iv) a 3′ UTR, and (v) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, (iv) a miRNA regulatory target site (miRTS) for a microRNA expressed in an inner ear cell, (v) a 3′ UTR, and (vi) a 3′ ITR.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 40 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 90 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 99 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 16 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 28 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 57 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 91 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 92 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 93 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 94 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 96 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 97 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86. In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide (e.g. a Connexin 26 polypeptide) operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NO: 98 and a minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

### Exemplary Construct Components

### Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence, e.g., a polynucleotide encoding a polypeptide (e.g., a therapeutic polypeptide, a Connexin 26 polypeptide) into a genome of a subject's cell. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., a polynucleotide encoding a polypeptide (e.g., a therapeutic polypeptide, a Connexin 26 polypeptide)) and associated elements flanked by a 5′ and a 3′ AAV ITR sequences. In some aspects, an ITR is or comprises about 145 nucleic acids. In some aspects, an ITR is or comprises about 119 nucleic acids. In some aspects, an ITR is or comprises about 130 nucleic acids. In some aspects, all or substantially all of a sequence encoding an ITR is used. An AAV ITR sequence may be obtained from any known AAV, including presently identified mammalian AAV types. In some aspects an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some aspects an ITR (e.g., a 5′ ITR) can have a sequence according to SEQ ID NO: 8. In some aspects, an ITR (e.g., a 3′ ITR) can have a sequence according to SEQ ID NO: 9. In some aspects, an ITR includes one or more modifications, e.g., truncations, deletions, substitutions or insertions, as is known in the art. In some aspects, an ITR comprises fewer than 145 nucleotides, e.g., 119, 127, 130, 134 or 141 nucleotides. For example, in some aspects, an ITR comprises 110, 111, 112, 113, 114, 115, 116, 117, 118, 119, 120, 121, 122, 123,124, 125, 126, 127, 128, 129, 130, 131, 132, 133, 134, 135, 136, 137, 138, 139, 140, 141, 142, 143 144, or 145 nucleotides. In some aspects, the ITR comprises about 119 nucleotides. In some aspects, the ITR comprises about 130 nucleotides. In some aspects an ITR (e.g., a 5′ ITR) can have a sequence according to SEQ ID NO: 52. In some aspects, an ITR (e.g., a 3′ ITR) can have a sequence according to SEQ ID NO: 53.

A non-limiting example of 5′ AAV ITR sequences includes SEQ ID NO: 8 or 52. A non-limiting example of 3′ AAV ITR sequences includes SEQ ID NO: 9 or 53. In some aspects, the 5′ and a 3′ AAV ITRs (e.g., SEQ ID NOs: 8 and 9, or SEQ ID NOs: 52 and 53) flank a portion of a coding sequence, e.g., all or a portion of a polynucleotide encoding a polypeptide (e.g., a therapeutic polypeptide, a Connexin 26 polypeptide). The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some aspects, a 5′ ITR sequence is at least at least 85%, at least 90%, at least 95%, at least 96%, at least 97%, at least 98% or at least 99%, or 100% identical to a 5′ ITR sequence represented by SEQ ID NO: 8. In some aspects, a 3′ ITR sequence is at least at least 85%, at least 90%, at least 95%, at least 96%, at least 97%, at least 98% at least 99%, or 100% identical to a 3′ ITR sequence represented by SEQ ID NO: 9. In some aspects, a 5′ ITR sequence is at least at least 85%, at least 90%, at least 95%, at least 96%, at least 97%, at least 98% at least 99%, or 100% identical to a 5′ ITR sequence represented by SEQ ID NO: 52. In some aspects, a 3′ ITR sequence is at least 85%, at least 90%, at least 95%, at least 96%, at least 97%, at least 98% at least 99%, or 100% identical to a 3′ ITR sequence represented by SEQ ID NO: 53.

In some aspects, a 3′ ITR sequence is at least at least 85%, at least 90%, at least 95%, at least 96%, at least 97%, at least 98% at least 99%, or 100% identical to a 3′ ITR sequence represented by SEQ ID NO: 116. In some aspects, a 3′ ITR sequence is at least 85%, at least 90%, at least 95%, at least 96%, at least 97%, at least 98% at least 99%, or 100% identical to a 3′ ITR sequence represented by SEQ ID NO: 116.

Exemplary 5′ AAV ITR
(SEQ ID NO: 8)
TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT
Exemplary 3′ AAV ITR
(SEQ ID NO: 9)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA
Exemplary 5′ AAV ITR
(SEQ ID NO: 52)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT
Exemplary 3′ AAV ITR
(SEQ ID NO: 53)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG
Exemplary 3′ ITR
(SEQ ID NO: 116)
AGGAACCCCTAGTGATGGAGTTGGCCACTCCCTCTCTGCGCGCTCGCTCG
CTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAA

### Promoters

In some aspects, the disclosure is directed to constructs comprising a cell selective promoter which can be used to regulate (e.g., increase) expression of a polynucleotide encoding a therapeutic polypeptide (e.g., a Connexin 26 polypeptide) in a cell (e.g., an inner ear cell, e.g., a supporting cell). In some aspects, the constructs provide reduced toxicity associated with expression of the therapeutic polypeptide (e.g., a Connexin 26 polypeptide) in some cells (e.g., an inner ear cell, e.g., a hair cell).

In some aspects, the disclosure is directed to constructs comprising a cell selective promoter which can be used to regulate (e.g., increase) expression of a polynucleotide encoding a polypeptide in a cell (e.g., an inner ear cell, e.g., a supporting cell). In some aspects, the constructs provide reduced toxicity associated with expression of the polypeptide in some cells (e.g., an inner ear cell, e.g., a hair cell).

In some aspects, a construct (e.g., an rAAV construct) comprises a promoter. The term “promoter” refers to a DNA sequence recognized by enzymes/proteins that can promote and/or initiate transcription of an operably linked gene (e.g., a polynucleotide encoding a polypeptide (e.g., a therapeutic polypeptide, a Connexin 26 polypeptide)). For example, a promoter typically refers to, e.g., a nucleotide sequence to which an RNA polymerase and/or any associated factor binds and from which it can initiate transcription. Thus, in some aspects, a construct (e.g., an rAAV construct) comprises a polynucleotide operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, a cell-selective promoter or any other type of promoter known in the art. In some aspects, a promoter is a RNA polymerase II promoter, such as a mammalian RNA polymerase II promoter. In some aspects, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some aspects, a promoter is a cochlea-selective promoter or a cochlea-oriented promoter. In some aspects, a promoter is a hair cell selective promoter, or a supporting cell selective promoter. In some aspects, a promoter is an inner ear supporting cell selective promoter.

The term “constitutive” promoter refers to a nucleotide sequence that, when operably linked with a nucleic acid encoding a protein (e.g., a polypeptide (e.g., a therapeutic polypeptide, a Connexn 26 polypeptide)), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated in its entirety herein by reference), the SV40 promoter, the dihydrofolate reductase promoter, the beta-actin promoter, the phosphoglycerol kinase (PGK) promoter, and the EFl-alpha promoter (Invitrogen). In some aspects, the promoter is a constitutive promoter. In some aspects, the constitutive promoter is a CAG promoter, a CBA promoter, a CMV promoter, a CMV/CBA enhancer/promoter, or a CB7 promoter. In some aspects, the a CMV/CBA enhancer/promoter comprises a nucleic acid with at least 85%, at least 90%, at least 95%, at least 96%, at least 97%, at least 98% at least 99%, or 100% identity to SEQ ID NOs: 12 or 13. In some aspects, the CMV/CBA enhancer/promoter comprises a nucleic acid of SEQ ID NO: 12. In some aspects, the CMV/CBA enhancer/promoter comprises a nucleic acid of SEQ ID NO: 13. In some aspects, the CBA promoter comprises a nucleic acid with at least 85%, at least 90%, at least 95%, at least 96%, at least 97%, at least 98% at least 99%, or 100% identity to SEQ ID NOs: 10 or 11. In some aspects, the CBA promoter comprises a nucleic acid of SEQ ID NO: 10. In some aspects, the CBA promoter comprises a nucleic acid of SEQ ID NO: 11.

In some aspects, the CAG promoter comprises a nucleic acid with at least 85%, at least 90%, at least 95%, at least 96%, at least 97%, at least 98% at least 99%, or 100% identity to SEQ ID NOs: 14 or 15. In some aspects, the CAG promoter comprises a nucleic acid of SEQ ID NO: 14. In some aspects, the CAG promoter comprises a nucleic acid of SEQ ID NO: 15.

In some aspects, regulatory and/or control sequences impart cell selective gene expression capabilities. In some cases, cell selective regulatory and/or control sequences bind cell selective transcription factors that induce transcription in a cell selective manner.

In some aspects, a cell selective promoter is an ear cell selective promoter. In some aspects, a cell selective promoter is an inner ear cell selective promoter. In some aspects, a promoter is a characteristic fragment of a cell selective promoter. In some aspects, the promoter is an inner ear supporting cell selective promoter.

In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, inner ear support cell selective promoters are selected from one or more of GJB2, GJB6, IGFBP2, RBP7, GDF6, PARM1, GFAP, BACE2, DBI2, FABP3, KLHL14, MMP15, SPARC, TSPAN8, VIM, derivatives thereof, or fragments thereof.

In some aspects, the inner ear support cell selective promoter is a GDF6 promoter. In some aspects, the inner ear support cell selective promoter is a PARM1 promoter. In some aspects, the inner ear support cell selective promoter is a MMP15 promoter. In some aspects, the inner ear support cell selective promoter is a VIM promoter.

In some aspects, the inner ear support cell selective promoter is a GJB2 promoter. In some aspects, the inner ear support cell selective promoter is a GJB6 promoter. In some aspects, the inner ear support cell selective promoter is a IGFBP2 promoter. In some aspects, the inner ear support cell selective promoter is a RBP7 promoter. In some aspects, the inner ear support cell selective promoter is a GFAP promoter. In some aspects, the inner ear support cell selective promoter is BACE2 promoter. In some aspects, the inner ear support cell selective promoter is a DBI2 promoter. In some aspects, the inner ear support cell selective promoter is a FABP3. In some aspects, the inner ear support cell selective promoter is a KLHL14 promoter. In some aspects, the inner ear support cell selective promoter is a SPARC promoter. In some aspects, the inner ear support cell selective promoter is a TSPAN8 promoter.

In some aspects, derivatives thereof can include a modified parent sequence (e.g., a naturally occuring promoter sequence), one or more portions of a parent sequence, fragments of a parent sequence, and the like.

In some aspects, the promoter is an inner ear medial support cell selective promoter. In some aspects, inner ear medial support cells are selected from one or more of lateral greater epithelial ridge cells and inner sulcus cells. In some aspects, inner ear medial support cell selective promoters are selected from one or more of GJB6, IGFBP2, GDF6, PARM1, derivatives thereof, or fragments thereof. In some aspects, the inner ear medial support cell selective promoter is a GDF6 promoter. In some aspects, the inner ear medial support cell selective promoter is a PARM1 promoter. In some aspects, the inner ear medial support cell selective promoter is a IGFBP2 promoter. In some aspects, the inner ear medial support cell selective promoter is a GJB6 promoter.

In some aspects, the promoter is an inner ear sensory epithelial support cell selective promoter. In some aspects, sensory epithelial support cells are selected from one or more of inner pillar cells, outer pillar cells, dieter cells, and inner phalangeal cells. In some aspects, a inner ear sensory epithelial support cell selective promoters are selected from one or more of GJB6, IGFBP2, RBP7, GDF6, PARM1, FABP3, BACE2 derivatives thereof, or fragments thereof.

In some aspects, a inner ear sensory epithelial support cell selective promoter is a GDF6 promoter. In some aspects, a inner ear sensory epithelial support cell selective promoter is a PARM1 promoter. In some aspects, a inner ear sensory epithelial support cell selective promoter is a GJB6 promoter. In some aspects, a inner ear sensory epithelial support cell selective promoter is a IGFBP2 promoter. In some aspects, a inner ear sensory epithelial support cell selective promoter is a RBP7 promoter. In some aspects, a inner ear sensory epithelial support cell selective promoter is a FABP3 promoter. In some aspects, a inner ear sensory epithelial support cell selective promoter is a BACE2 promoter.

In some aspects, the promoter is an inner phalangeal cell selective promoter. In some aspects, the inner phalangeal cell selective promoters are selected from one or more of IGFBP2, GDF6, FABP3, BACE2, derivatives thereof, or fragments thereof. In some aspects, the inner phalangeal cell selective promoter is a IGFBP2 promoter. In some aspects, the inner phalangeal cell selective promoter is a GDF6 promoter. In some aspects, the inner phalangeal cell selective promoter is a FABP3 promoter. In some aspects, the inner phalangeal cell selective promoter is a BACE2 promoter.

In some aspects, the promoter is an interdental cell selective promoter. In some aspects, the interdental cell promoter is IGFBP2, derivative thereof, or fragment thereof.

In some aspects, the inner ear supporting cell selective promoter is a GJB2 promoter. In some aspects, the GJB2 enhancer comprises a nucleic acid sequence with at least 85%, at least 90%, at least 95%, at least 96%, at least 97%, at least 98% at least 99%, or 100% identity to SEQ ID NO: 65. In some aspects, the GJB2 enhancer comprises the nucleic acid sequence of SEQ ID NO: 65 In some aspects, the GJB2 minimal promoter comprises a nucleic acid sequence with at least 85%, at least 90%, at least 95%, at least 96%, at least 97%, at least 98% at least 99%, or 100% identity to SEQ ID NO: 86. In some aspects, the GJB2 minimal promoter comprises the nucleic acid sequence of SEQ ID NO: 86.

In some aspects, the promoter is derived from a GJB2 promoter and has a length of 1000-1050 nucleotides. In some aspects, the inner ear supporting cell selective promoter is a GJB6 promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16. In some aspects, the GJB6 promoter comprises the nucleic acid sequence of SEQ ID NO: 16. In some aspects, the promoter is derived from a GJB6 promoter and has a length of 700-750 nucleotides.

In some aspects, the inner ear supporting cell selective promoter is an IGFBP2 promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 57. In some aspects, the IGFBP2 promoter comprises the nucleic acid sequence of SEQ ID NO: 57. In some aspects, the promoter is derived from an IGFBP2 promoter and has a length of 1500-1550 nucleotides.

In some aspects, the inner ear supporting cell selective promoter is a RBP7 promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 28. In some aspects, the RBP7 promoter comprises the nucleic acid sequence of SEQ ID NO: 28. In some aspects, the promoter is derived from a RBP7 promoter and has a length of 1050-1100 nucleotides.

In some aspects, the inner ear supporting cell selective promoter is a GDF6 promoter. In some aspects, the GDF6 promoter comprises a nucleic acid sequence with at least 85%, at least 90%, at least 95%, at least 96%, at least 97%, at least 98% at least 99%, or 100% identity to SEQ ID NO: 90. In some aspects, the GDF6 promoter comprises the nucleic acid sequence of SEQ ID NO: 90. In some aspects, the promoter is derived from a GDF6 promoter and has a length of 1150-1200 nucleotides.

In some aspects, the inner ear supporting cell selective promoter is a PARM1 promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 40. In some aspects, the PARM1 promoter comprises the nucleic acid sequence of SEQ ID NO: 40. In some aspects, the promoter is derived from a PARM1 promoter and has a length of 1300-1350 nucleotides.

In some aspects, the construct comprises two or more promoters. In some aspects, the first promoter is selected from a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, and any combination thereof. In some aspects, the second promoter is selected from a GJB2 promoter or a minimal GJB2 promoter.

In some aspects, the first promoter is a GDF6 promoter and the second promoter is a minimal GJB2 promoter. In some aspects, the first promoter is a PARM1 promoter and the second promoter is a minimal GJB2 promoter. In some aspects, the first promoter is a MMP15 promoter and the second promoter is a minimal GJB2 promoter. In some aspects, the first promoter is a VIM promoter and the second promoter is a minimal GJB2 promoter.

In some aspects, the first promoter is a GJB6 promoter and the second promoter is a minimal GJB2 promoter. In some aspects, the first promoter is a IGFBP2 promoter and the second promoter is a minimal GJB2 promoter. In some aspects, the first promoter is a GDF6 promoter and the second promoter is a minimal RBP7 promoter. In some aspects, the first promoter is a GFAP promoter and the second promoter is a minimal GJB2 promoter. In some aspects, the first promoter is a BACE2 promoter and the second promoter is a minimal GJB2 promoter. In some aspects, the first promoter is a DBI2 promoter and the second promoter is a minimal GJB2 promoter. In some aspects, the first promoter is a FABP3 promoter and the second promoter is a minimal GJB2 promoter. In some aspects, the first promoter is a KLHL14 promoter and the second promoter is a minimal GJB2 promoter. In some aspects, the first promoter is a SPARC promoter and the second promoter is a minimal GJB2 promoter. In some aspects, the first promoter is a TSPAN8 promoter and the second promoter is a minimal GJB2 promoter.In some aspects, the inner ear supporting cell selective promoter comprises a GJB6 and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the GJB6 has the nucleic acid sequence of SEQ ID NO: 16 and the hGJB2 minimal promoter has the nucleic acid sequence of SEQ ID NO: 86.

In some aspects, the inner ear supporting cell selective promoter comprises a IGFBP2 promoter and a hGJB2 minimal promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 57 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the IGFBP2 has the nucleic acid sequence of SEQ ID NO: 57 and the hGJB2 minimal promoter has the nucleic acid sequence of SEQ ID NO: 86.

In some aspects, the inner ear supporting cell selective promoter comprises a RBP7 promoter and a hGJB2 minimal promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 28 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the RBP7 has the nucleic acid sequence of SEQ ID NO: 28 and the hGJB2 minimal promoter has the nucleic acid sequence of SEQ ID NO: 86.

In some aspects, the inner ear supporting cell selective promoter comprises a GJB6 promoter and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the GJB6 has the nucleic acid sequence of SEQ ID NO: 16 and the hGJB2 minimal promoter has the nucleic acid sequence of SEQ ID NO: 86.

In some aspects, the inner ear supporting cell selective promoter comprises a PARM1 promoter and a hGJB2 minimal promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 40 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the PARM1 has the nucleic acid sequence of SEQ ID NO: 40 and the hGJB2 minimal promoter has the nucleic acid sequence of SEQ ID NO: 86.

In some aspects, the inner ear supporting cell selective promoter is a BACE2 promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92. In some aspects, the BACE2 promoter comprises the nucleic acid sequence of SEQ ID NO: 92. In some aspects, the promoter is derived from a BACE2 promoter and has a length of 1400-1450 nucleotides.

In some aspects, the inner ear supporting cell selective promoter is a DBI2 promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93. In some aspects, the DBI2 promoter comprises the nucleic acid sequence of SEQ ID NO: 93. In some aspects, the promoter is derived from a DBI2 promoter and has a length of 1450-1500 nucleotides.

In some aspects, the inner ear supporting cell selective promoter is a FABP3 promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94. In some aspects, the FABP3 promoter comprises the nucleic acid sequence of SEQ ID NO: 94. In some aspects, the promoter is derived from a FABP3 promoter and has a length of 1750-1800 nucleotides.

In some aspects, the inner ear supporting cell selective promoter is a KLHL14 promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95. In some aspects, the KLHL14 promoter comprises the nucleic acid sequence of SEQ ID NO: 95. In some aspects, the promoter is derived from a KLHL14 promoter and has a length of 1250-1300 nucleotides.

In some aspects, the inner ear supporting cell selective promoter is a MMP15 promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96. In some aspects, the MMP15 promoter comprises the nucleic acid sequence of SEQ ID NO: 96. In some aspects, the promoter is derived from a MMP15 promoter and has a length of 1000-1050 nucleotides.

In some aspects, the inner ear supporting cell selective promoter is a SPARC promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97. In some aspects, the SPARC promoter comprises the nucleic acid sequence of SEQ ID NO: 97. In some aspects, the promoter is derived from a SPARC promoter and has a length of 1000-1050 nucleotides.

In some aspects, the inner ear supporting cell selective promoter is a TSPAN8 promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98. In some aspects, the TSPAN8 promoter comprises the nucleic acid sequence of SEQ ID NO: 98. In some aspects, the promoter is derived from a TSPAN8 promoter and has a length of 1200-1250 nucleotides.

In some aspects, the inner ear supporting cell selective promoter is a GFAP promoter. In some aspects, the GFAP promoter comprises a nucleic acid sequence with at least 85%, at least 90%, at least 95%, at least 96%, at least 97%, at least 98% at least 99%, or 100% identity to SEQ ID NO: 91. In some aspects, the GFAP promoter comprises the nucleic acid sequence of SEQ ID NO: 91. In some aspects, the promoter is derived from a GFAP promoter and has a length of 650-700 nucleotides.

In some aspects, the inner ear supporting cell selective promoter is a VIM promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99. In some aspects, the VIM promoter comprises the nucleic acid sequence of SEQ ID NO: 99. In some aspects, the promoter is derived from a VIM promoter and has a length of 1050-1100 nucleotides.

In some aspects, the inner ear supporting cell selective promoter comprises a BACE2 promoter and a hGJB2 minimal promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the BACE2 promoter comprises the nucleic acid sequence of SEQ ID NO: 92 and the hGJB2 minimal promoter has the nucleic acid sequence of SEQ ID NO: 86.

In some aspects, the inner ear supporting cell selective promoter comprises a DBI2 promoter and a hGJB2 minimal promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the DBI2 promoter comprises the nucleic acid sequence of SEQ ID NO: 93 and the hGJB2 minimal promoter has the nucleic acid sequence of SEQ ID NO: 86.

In some aspects, the inner ear supporting cell selective promoter comprises a FABP3 promoter and a hGJB2 minimal promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the FABP3 promoter comprises the nucleic acid sequence of SEQ ID NO: 94 and the hGJB2 minimal promoter has the nucleic acid sequence of SEQ ID NO: 86.

In some aspects, the inner ear supporting cell selective promoter comprises a KLHL14 promoter and a hGJB2 minimal promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the KLHL14 promoter comprises the nucleic acid sequence of SEQ ID NO: 95 and the hGJB2 minimal promoter has the nucleic acid sequence of SEQ ID NO: 86.

In some aspects, the inner ear supporting cell selective promoter comprises a MMP15 promoter and a hGJB2 minimal promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the MMP15 promoter comprises the nucleic acid sequence of SEQ ID NO: 96 and the hGJB2 minimal promoter has the nucleic acid sequence of SEQ ID NO: 86.

In some aspects, the inner ear supporting cell selective promoter comprises a SPARC promoter and a hGJB2 minimal promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the SPARC promoter comprises the nucleic acid sequence of SEQ ID NO: 97 and the hGJB2 minimal promoter has the nucleic acid sequence of SEQ ID NO: 86.

In some aspects, the inner ear supporting cell selective promoter comprises a TSPAN8 promoter and a hGJB2 minimal promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the TSPAN8 promoter comprises the nucleic acid sequence of SEQ ID NO: 98 and the hGJB2 minimal promoter has the nucleic acid sequence of SEQ ID NO: 86.

In some aspects, the inner ear supporting cell selective promoter comprises a VIM promoter and a hGJB2 minimal promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the VIM promoter comprises the nucleic acid sequence of SEQ ID NO: 99 and the hGJB2 minimal promoter has the nucleic acid sequence of SEQ ID NO: 86.

Exemplary CBA promoter
(SEQ ID NO: 10)
GTCGAGGTGAGCCCCACGTTCTGCTTCACTCTCCCCATCTCCCCCCCCTCCCCACCCCCAATTTT
GTATTTATTTATTTTTTAATTATTTTGTGCAGCGATGGGGGGGGGGGGGGGGGGGGCGCGCGCCA
GGCGGGGGGGGGCGGGGCGAGGGGCGGGGGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAGC
GAAGCGCGCGGCGGGCG
Exemplary CBA promoter
(SEQ ID NO: 11)
GTCGAGGTGAGCCCCACGTTCTGCTTCACTCTCCCCATCTCCCCCCCCTCCCCACCCCCAATTTT
GTATTTATTTATTTTTTAATTATTTTGTGCAGCGATGGGGGGGGGGGGGGGGGGGGGGGGCGCGC
CAGGCGGGGGGGGGGGGGGCGAGGGGGGGGGGGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAAA
GCGAAGCGCGCGGCGGGCG
Exemplary CMV/CBA enhancer/promoter
(SEQ ID NO: 12)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGCGCGCGCCAGGCGGGGCG
GGGGGGGGCGAGGGGCGGGGGGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGC
GGCGGGCG
Exemplary CMV/CBA enhancer/promoter
(SEQ ID NO: 13)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCGCGCGCCAGGCGGGG
CGGGGCGGGGCGAGGGGCGGGGGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCG
Exemplary CAG enhancer/promoter
(SEQ ID NO: 14)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGGGGGGGGGGGGGGGGCGCGCGCCAGGCGGGGCG
GGGGGGGGCGAGGGGGGGGGGGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCCGCCTCGCGCCGCCCG
CCCCGGCTCTGACTGACCGCGTTACTCCCACAGGTGAGCGGGCGGGACGGCCCTTCTCCTCCGGG
CTGTAATTAGCGCTTGGTTTAATGACGGCTCGTTTCTTTTCTGTGGCTGCGTGAAAGCCTTAAAG
GGCTCCGGGAGGGCCCTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGGGCGCGGCGCGGGGCT
TTGTGCGCTCCGCGTGTGCGCGAGGGGAGCGCGGCCGGGGGGGGTGCCCCGCGGTGCGGGGGGGC
TGCGAGGGGAACAAAGGCTGCGTGCGGGGTGTGTGCGTGGGGGGGTGAGCAGGGGGTGTGGGCGC
GGCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGGGGGGGGTGGCGGCAGGT
GGGGGTGCCGGGGGGGGGGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTTTTATGGTAATCGTGC
GAGAGGGCGCAGGGACTTCCTTTGTCCCAAATCTGTGCGGAGCCGAAATCTGGGAGGCGCCGCCG
CACCCCCTCTAGCGGGCGCGGGGCGAAGCGGTGCGGCGCCGGCAGGAAGGAAATGGGGGGGGAGG
GCCTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAG
Exemplary CAG enhancer/promoter
(SEQ ID NO: 15)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGGGGGGGGGGGGGGGGGGGGCGCGCCAGGCGGGG
CGGGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCGGGAGTCGCTGCGTTGCCTTCGCCCCGTGCCCCGCTCCGCGCCGCCTCGCGCCGCC
CGCCCCGGCTCTGACTGACCGCGTTACTCCCACAGGTGAGCGGGCGGGACGGCCCTTCTCCTCCG
GGCTGTAATTAGCGCTTGGTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGTGCGTGCGTGTGTGTGTGC
GTGGGGAGCGCCGCGTGCGGCCCGCGCTGCCCGGCGGCTGTGAGCGCTGCGGGCGCGGCGCGGGG
CTTTGTGCGCTCCGCGTGTGCGCGAGGGGAGCGCGGCCGGGGGCGGTGCCCCGCGGTGCGGGGGG
GCTGCGAGGGGAACAAAGGCTGCGTGCGGGGTGTGTGCGTGGGGGGGTGAGCAGGGGGTGTGGGC
GCGGCGGTCGGGCTGTAACCCCCCCCTGCACCCCCCTCCCCGAGTTGCTGAGCACGGCCCGGCTT
CGGGTGCGGGGCTCCGTGCGGGGCGTGGCGCGGGGCTCGCCGTGCCGGGCGGGGGGTGGCGGCAG
GTGGGGGTGCCGGGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCGT
GCGAGAGGGCGCAGGGACTTCCTTTGTCCCAAATCTGTGCGGAGCCGAAATCTGGGAGGCGCCGC
CGCACCCCCTCTAGCGGGCGCGGGGCGAAGCGGTGCGGCGCCGGCAGGAAGGAAATGGGGGGGGA
GGGCCTTCGTGCGTCGCCGCGCCGCCGTCCCCTTCTCCCTCTCCAGCCTCGGGGCTGTCCGCGGG
GGGACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAG

In some aspects, the promoter is a GJB2 minimal promoter as set forth in SEQ ID NO: 86. In some aspects, a promoter is at least 85%, at least 90%, at least 95%, at least 96%, at least 97%, at least 98%, at least 99%, or 100% identical to SEQ ID NO: 86.

Exemplary Human GJB2 minimal promoter
(SEQ ID NO: 86)
AAGCTCTGAGGACCCAGAGGCCGGGCGCGCTCCGCCCGCGGCGCCGCCCC
CTCCGTAACTTTCCCAGTCTCCGAGGGAAGAGGCGGGGTGTGGGGTGCGG
TTAAAAGGCGCCACGGCGGGAGACAGGT

In certain aspects, the promoter is a GDF6 promoter as set forth in SEQ ID NO: 90. In some aspects, an promoter sequence is at least 85%, at least 90%, at least 95%, at least 96%, at least 97%, at least 98%, at least 99%, or 100% identical to a promoter sequence represented by SEQ ID NO: 90. In some aspects, the promoter is a GDF6 promoter sequence comprising the sequence of SEQ ID NO: 90.

Exemplary Human GDF6 promoter
(SEQ ID NO: 90)
CCACAGGTAACTCCGTCGGCGTCCACAGGGGGGCAGGAGATACCATACTGCACAGTTGTACGTCT
TCCATCTGTTTGGTGTAGAAAAATCTAACCACTACAAGAATGCCACGGGCACTGTGGCAGACAGA
AGCAGCGCTACGCCGCATCGCCTTTCAGCGTGCAGGCCCAGGAATGAGCGAGGCAGTGGGGGGGG
AAGACAGGCACGGGGAATCTGGGGACAGATAAAGGAAACTCGTGATGGGGCGAGGCTGGGCTGAA
GAGAAACAGATTGGGGTAGAGCTGCAAAGGGAGGGGTCCACTGGAAGGCGAGGGGGGAGGCCGGG
AAGAGAGAGGGTGGGAAGGCAGTGTGAGATGGGAGGGCAGTGTGAGAAGAAAAGCAGGCTGGGGA
AGAGGGATTGGAATGCAGAAGGAACTTGGGGAAGGAGGAAGTCCTGCAGGCGGGAGGGAAAGAAG
AGAGGGGGAGCAGCTAAAGTCTGCGTCAGAAGAGGTTGGGGACTGCGAGAGGAGAGGCTGGGGCC
TGCAGGGGAGCGCAGCAGCTTTTAGCATCGATCCAAACTCTAAAGACTCGTGGCCTTTGCCTGAC
CTCGAGGGTCGGGAATAGACGCCTGTCTTTGTGGAGAGCGATACCCAACCGAGAAAATGGGGCTG
TTCCGAGCTGGGCCCTGCGCCTGGCCCAGGGCGAGGCTTCTCTGGCTCCGGGCTGGCCCCTGAGG
GGCAGCACGCAGCCTGCAGCAGAGGCGCCTGCTCCAAGCTGTCTCTTGGGGGCGCCGCCGCCGCT
TCCCTCCTCCGGGGCCGCTCGCTCCCAGGAAAGTGGAGGCGGCTGGCGAGGACCGAGAGCCGGGG
CCGCGCTGCGGAGGGACCACACCTCCGGGAGTTCGAGGGGGACCCTGGCGCGGCGGGCCAGCCTT
TCGGGCCGGCAGCGCCCGCCTTCCCCCGGTCAGCGCTTGCGGCCCGCGCCGCGCGCACCGCCCGG
CAACCCCGCGCGCGTCCCGCGGGGGCGCTGCGTCTTCCTGCCACACCGGCGCACCGCGGCCCCTC
TCCCCCACACCTCCGGCCCGCACCACCCGGCTCTCCTCCCACCCTCCCCACCCCTCCTCTGCCCT
CCCTCCCCATTCCTCCCCTCCCGGCGAGGGGGGGGAGGGGGCGTGGCGGGGCCGGGGTTTGTGTG
GCTGGGACCCGGCTCCTC

In certain aspects, the promoter is a human IGFBP2 promoter as set forth in SEQ ID NO: 57. In some aspects, an promoter sequence is at least 85%, at least 90%, at least 95%, at least 96%, at least 97%, at least 98%, at least 99%, or 100% identical to a promoter sequence represented by SEQ ID NO: 57. In some aspects, the promoter is a human IGFBP2 promoter sequence comprised within SEQ ID NO: 57.

Exemplary Human IGFBP2 promoter
(SEQ ID NO: 57)
AAGAAACTTGCCCGAGTTTACACAGCTAGTAAATGGTTGCATTAGTCAGGACAGCTAGCCTATAT
TACAATAACAACCCTCTCAAATCCTAATGGCTTAAAACAACAGAGGTTTAATTTATACTCATTAG
CTGTTCAAGGCAGGAGGCTCTATTCTCTAATCCATACAGTCACTCAGGATCCAGGCTGGTGGAGA
CCCTGCCATATTGTAGCCTCACCATTTAAAACATGAAGAAGATAGAAAGTGAGGAGTCATGTAGG
TTTTGTTCCGTTGCCTCAGGCTAGGAGTGACAGGTCACTTCATCTCACTCACAGCTCACTGCCCA
CAACTAGTCACTTGTGACTGTGCGAGTTAAGCTTCTGTGTGTGAAGGAAGGAAAAGAGAATGGGA
TAAAGGTGAACATCAGCAGGCTCTACCACAGTAGTTTGAACCAAGACTTGAGCCTAGGTCATGTG
GCTTCAGAATCTTTGCTCTTAATCACACTAAACAGCCTCTGTAAGTCATCTTTCCTTCATCCAGT
GCCTAAGAACATGCAGTCCAATGCCCTCATCCTTCAGAAGAACTTGAGTGAACTCAGAGAAATTG
AGTAGAGTGCCACAGCATGCCCAAGGCCACACACCCTGAGGTTGGCAGTAGGTCCTGAGTTAGAG
TTGTCATTTCTTGGCTCCCCTGGTAGTAGTGGAAAGGTAAGGTTTTGACATACTAGTTGGATGAC
CACGGGCAGGTCACTTAAATTGTCTAAGCATCGTTTGACCCTTGTAAGAATTAAATGAAATAGCA
CCTGTAAAAGTGTCTGCACGGACTTACTGCTGTTAGTTTTGTTCCTTTCTTCCTGTTGTCACTGC
ACTTCCCTGCCTGTTACCCAGGCCATGCAGACCAGCCAGGCCTTCGACTTACAGTGCGGATAAGA
TTCCAAATCTCCACGGCTGGTTTCCATGCTTTCTTCCAGGCTTCTGAGGACCCTGTGCTCTGGTT
TCTTCTATTTCTTTTCTATTACTTTTCTGTTACTCTTGAGCACACTTGCTGGAAGCAATATGCAT
CCAGTTCTCCCTCTCTTGCCTCATTACACTTTGCAGAACAACTCCAATCCCTTCCAACCAAGTAG
TCCCTTTGAATTTCTTGTCACCCAAGGAATCTCTCTGACAGGGGTCTTTGTTAGGGTCACACCCC
AGGAGATGGTTGATTATGGCTGAGTCCAGCCTGGAATGATGGGGGTTGGGGGCAGCTTGGGTAGA
TGACTCAGTAAATCAAACAGAACAATGAAAGGAGGTCATGCTTGTCCATCTGCATTATTGAAGAC
AGCCATAAATGGCCTTACCCCAGAGCGGGTCTGTCACACCTGGAGAGCTGATCTGACCTCTCCAA
GACCCCTGCAACTGAGTGTTCTGGGATCTGTCCTGCAACAAGTGCCTCGAGATTTGTAGGTGGGG
GCCCAGAGGGAGGGGGTCTGCAGACGAAGGGGGCAGGTTTTGCGGGGCACTTAGGGTTCTCATAG
GTTGTAGTCACGAGCTCC

In certain aspects, the promoter is a human RBP7 promoter as set forth in SEQ ID NO: 28. In some aspects, an promoter sequence is at least 85%, at least 90%, at least 95%, at least 96%, at least 97%, at least 98% at least 99%, or 100% identical to a promoter sequence represented by SEQ ID NO: 28. In some aspects, the promoter is a human RBP7 promoter sequence comprised within SEQ ID NO: 28.

Exemplary Human RBP7 promoter
(SEQ ID NO: 28)
CCCATGGCTCTGTTAAAATCAAAGAAACATCTTTTCCAACAGCCCTTTCAAACTCCTCATCGCAT
CTCACTGGCTGATTCAGTCATTTAAACCTGCTTCTCCCTAAAGCTGATCACTGGCTAAGCTAATA
GGGTTTCCGGGATTGGTTTAGCCTGATACTAATCCAGGTCTACCTTCAGGAGCCAGACCAAACTG
CCTATTGGCATTGCATTCTTGCAGTAGGGAGGGGAGGTATGGATGGTGTGGAGTCCACCACAAGG
TCCATGCCAGTCTTTGCTGAACCAGCATCAGACTCCATCAAGCAACAGATGAGAGGTTCCATGAT
AAAGTGGCCCTCAGCAATCCCCATCCATTGCTGTCTAGGAAGAACAGTGCTTGTACACAGGTTTA
GGACCTCAGTCTTGGCTGTAATCTTCTGGTTTACTTTGCCAGCACCAAACAGAAGGAAAGAAAGG
GCTCAAATTTGACCAAATAAATTATGCTTCTCCTTCCAGAGATAACCTTGAGTCCTGTCTAGGAA
GATATTAGAATTGTAAAGAAAAAAAAAATTACTCCTTATCCTATGGCAAGTGGAGTCTATGTCTA
CTTCAGCTGAAATTAAATCCTGTCCATAATAGATGACCCTTGCTCAAGCTGGCCAGAAGCCATAC
CAACCAGCACGAAGGTTAAAACTATTATTAGTTTTTTCTGTGATTTTCATTTTCAGGCCAAGTTT
TAGAACAATAAGATTTTAAGAATAGGAAGTAAGTAAGATTTCTGCATATCCTGTTCTCTTAGTCA
GCTGAATTTTTTTTTTTTTTTTTTTAGTCCTAACTCAGCCTCCCAAAGTGCTGGGATTACAGGCG
TGAGCCACCGCACCAAGCCTGGAATCTATGTCTTACAGTTATGAGAATCAACAGCTAGCTCATTA
TGGGCAAGGTGATGTCACTCTGGCTTCTCAATGAAAATGGCATTTCTCCCTTGGAAAAGGTCATA
GCCAGTCAGTCAGTCAGTCACGGGAGCGCAGCGGCTTCTAGGGGTGAGTGGGACCCACGCGGCCC
CACCTGCTCCTCCCGCGCGCGGCCCCACCCCCCTGCCCCGCCCCGCCTGGTTTATAG

In certain aspects, the promoter is a human GJB6 promoter as set forth in SEQ ID NO: 16. In some aspects, an promoter sequence is at least 85%, at least 90%, at least 95%, at least 96%, at least 97%, at least 98% at least 99%, or 100% identical to promoter sequence represented by SEQ ID NO: 16. In some aspects, the promoter is a human GJB6 promoter sequence comprised within SEQ ID NO: 16.

Exemplary Human GJB6 promoter
(SEQ ID NO: 16)
AAATAGCTTCCAACGTTTCCACCCCACCAGCCCTTGCACCACTCCCTGTACTGGCCCTGAGCTTT
CTAGTCTTGACTGAAAAGCGGGGAGGCAATGTGGTCTCTCCTGGTGCACTGTCCCGAGGAAGGCC
TGCTCCGCTTCCCCGGAGGAGTCTTCAAAGGATGGAGGTAATTAATAAAAACAACCCCTGTACCT
CCTCTAAGTGGTCATTAATTAATAAAGAACCTCCAGGCTCCTATAGGAGAGGTCTGTGCACCCCG
CGGGCTATGAGAAGGCTGGATCACCCAGAAAGACTGAGGATGTGTCCTGGCAAAAACACAGCCTG
CCCCTCACACTGCTCCCCACGGGTGCACTAGGGAGGAAGAGTTCCCTCGAGGGCCTGAGCAGGCG
CCCCACACCTGCACCCGTGCAGAGGGGGCTGGGCCCGCCCTCTGCGCTCCCGAGGGAGAGCCCTA
CCCCCTGCATCCCCGGTACCCCGTTCCCTCCAAGGGCCGGAAAGAGGGCCCCGCGCACTGTGCAC
TTCTTAGGGGTCCCCCACCCTGCGCCCCCGCCACGGGAAAAAGGTCCCCGCTCTGCGCATCCGGC
CCCGGAGGGACAGCCCCGGTCCTGCACTCCTTGCTCCTCAGGGGGACGGTCCGCGCCCAGCGGCT
AGTGCGCCCCGGGTAGGTGGGGGGGGGGGGCTCGTCGAGTGACAGCGCTCGCCTCCCGCAGCCCG
CCCGAGCCGCGTCAGGGCAG

In certain aspects, the promoter is a human PARM1 promoter as set forth in SEQ ID NO: 40. In some aspects, a promoter sequence is at least 85%, at least 90%, at least 95%, at least 96%, at least 97%, at least 98% at least 99%, or 100% identical to promoter sequence represented by SEQ ID NO: 40. In some aspects, the promoter is a human PARM1 promoter sequence comprised within SEQ ID NO: 40.

Exemplary Human PARMI promoter
(SEQ ID NO: 40)
TGTACAGGAGATAGTCAGGGAATTAGTAATTTTCAAAGAGGTGACTTTGAATTCAAACTTAAATA
TCATCTTCAGCTGAAACAAAGAAGGGGTGCAGTTATGAGGAAGTGACCAGGTAAAGCATGGCAAA
CAAAGGTAAAGTTTGTTATGCGTATTTAAGTCAGAGCCCTCTCCATTGATAAGAGTTTCCAGTAA
TTTAGTGCCATCCTTTTCTTGCTATAGAGTTCTCGTCTCTATCTGAGCACGCAAAAATAACATGC
TTTCTTGCTTTCTTGAAGTTGGGCATGGCCATTGACTTGCCTTAGCCCATATTTTTCTGTGAAGT
GGTCTTCAAAAACCTATATTTCTGCCATAGAGTCACTTACTTAACCTGCCCTATTTAAAGGGGCT
AATGCCTGATAGAATGTCGCTGCATAACTCCATCTGTGTGTGGTCCCTGCATCCATGACAACCAA
AACCCAGATGCAGAAATTGTTCCTAATCACATAGATTACCCTAGAAACCGGAAGGGCCTTGAAGT
CAAAAGCATTCAGAGAACATGCTGAACAAATTGAATTTGCAGTTTATCTGGCCAGGGAGGATGGA
GAGGGGATGGGCACTTGGTCTGAGTATTTTTTGTTTCTCATTCCAACAGAAATTACTAGATTTAC
CAAAAAATCTACAAGTGGTAGTGTGATAGAGTCAGGCAGAGGAATTGACCATAGATAAGGTGCTC
AGGACTCCTAGAGTCAGCTTCTGGTATGTGAGAAAGAAGTGAGAACAGAGCCCATGGCATATGAA
GAAGATATTACAGAAAAAAGAAAGCTGCCTTCCACGCAAATCATTTCTTTACAAAGGCTTGTTAA
CTCCTGCAGTGCCAAGAAGCTGAATGCAGCGGCAGACATCCTGGTTCGGGCCCCAGGAAGCTCAG
CCGGGTTTAATGTGGATGAGGGTTTAATGATGTACACGCAGAAGTGTTTTGACAAATGAAGAAGG
TCCTCATTCTTGGAACATGTGCCGGTTCTCCGAGGGAACTCCTAAAAGGCTGTAAGCTCATGTAG
GAAAAGCTGAGCTAGATTCCTAAGGGCAGAGATGTGCTCACATTTCTTTGCATCCCTAGTTCCCA
GCACAGTGCAAGGCGCTGCAAACATTTGCTGAACCCAGGGTCTCGTGTCTTGACTGTCCAGCAGA
GGCCGCTCTGGGCCGGGGCTCTCGGGACCTGAGGGCTGAGAGAAGGAAGGCCAGGGGGTGGCCCA
GTCATCGCCGCGGGGCCCGGGTGGGAGGGGTTTGGCAGCGGCAGGCGCGGCGGCGGCGGCGGAGG
CGGAGGCGGCCCCGGG

In some aspects, the inner ear supporting cell selective promoter is a BACE2 promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92. In some aspects, the BACE2 promoter comprises the nucleic acid sequence of SEQ ID NO: 92.

Exemplary BACE2 promoter
(SEQ ID NO: 92)
TGTGCTGCGAGGGCTTCATCTCCTAAGCACTAAATGCTAAATTCCCCCTCCCACGCCC
ATCGCCACTGTCCTCACGGATCCTCGCAGCAGCTTCCCAATCGGTCTCCCTGTCTCCA
GCCTCACCACCCCCAACTAAGACCATTCATGAAAACAGAGACAACCAAGGAGACAG
TCACCCAATGCTGTCCCTTCAGCTTGCATTATTTTCTGACAAGACAGCTCTGCCATCC
ATGGAAGCCTGTGTTTGAAGATCTCTGACATAAAGGTCCCTTGCAGAGCTAGACGTG
ATTCTAAAATTGGGAACACAGGAATAAAAATCAAATCTTGAGTAGAAGTAGCTGAA
AATTGCAGTGATTCGGGGAAGCTTGGCTTCTAACTCCCCACTGTTTGAAGATGGGCT
TGTTTGTTTTTTAAAACAGCCAACATAATTCAGCTGGAGGAGGTACAAAGAATTTTC
TATTCCTTGTTTCTGTAGAAATCGATGGACTTTAGCTTGTCTAATTGTCCCCCCTGCCT
TTAGTATCTAAAATAAAATAACCCTCGTTGCTTGCATTACTCAACGCATTTCTGCGTC
TTGGCGTCTATGGCTAAACGAGTATTAATTAGACAGTCCGCAGAGAGCTGGCTGGGG
ATAGAAGGGGAGGTGGGGGAGAAGGGCAGGGATCACAGCAGGGTGGACTCGTGGC
CCTGATTTGGGATCCTGACAGCAACTTACTAGGTGGCCTGAGGGCTGGGTGCCAGGG
GAGGCAGCGGGTTCCAGTAGCATCTGACCTGCATTAGGGACAGGGGCGCGGCGGAG
GGGGCGAAGGGGGCGGGGGTGGGGGGAAGGTGGCTGGGGTGAAGCCCAGCTTCGC
AGCTAGCTGTGGGCAACAGAGGGAGTAAGGGGGGGCAATGAGGCTGGGGCCAGGC
GCCAGCAGCAGCCACGCCCCCCACCTCCCCCGATTTTTAGGGAAAATTCTCCAAAGC
TCTCGCATCCTCCTCTGCCTCCTTCCACCCTCCACCCTCCCAGCCTCCACTGAGACCT
CTTTAAAACCACCCAGGGGCCGCCGGGGGATGAGGCCGGGGAACGGGCTGGACTGA
GGGCGGGGGCTCGGGGGCAGCGGACGGGAAACGCCTCGAAAGCAGCCAGACCCGG
CGACTGAAATGAGGCGGAGGAGCTTGGCGAGGGGAGGCGCAGGCTCGGAAAGGCG
CGCGAGGCTCCAGGCTCCTTCCCGATCCACCGCTCTCCTCGCTGACCTCCGAGTCACC
CCCGGAAGCTCCCGCCACTGCCGGGCGAATAGACCCCCGCGGACCCCCAAGCGCGC
GGGGCCGGGGCCCTAGTTCAGGCCCTCGCTGCCCCTTTAAGGGTTCTCGAAACTTTC
CCCCCGGTATCAGATGAGCCTCGTCACATCCGTTGGCCGTGGC

In some aspects, the inner ear supporting cell selective promoter is a DBI2 promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93. In some aspects, the DBI2 promoter comprises the nucleic acid sequence of SEQ ID NO: 93.

Exemplary DBI2 promoter
(SEQ ID NO: 93)
GAAGAAACCTGCATTTCTTACACTTCAGTGTACTTTCCCCATATTTAACTCCAAGATT
TTTGTTAATTTGTTTGGTTTTCCTTTCTCAAACAAAATTATGCTCAGACTGAAAACCC
TAGATTTGTTCCCTATTGCATCTTCATTTCTTCCCAAACATTCCATAAAACGTGACCT
ACATTAAGTTAGCAAGTTAAGTCTGAAAGCGTCTACCTTCCCTGGGGAGGGGGAAG
GTGTAGGCAGGGCAGAGATTTGTAGTCCAGCCCTCTTGCCACAAATTATGAATTAGA
GAGGAATGACTTTGCTTTTTTAATGATCTCCAGAGAATTTTCCATCATTTCCCTCTCTT
CACCCAGCTCCTTTGCAACCACTGCCAGAGAAGTCTTCCTTTAGCTTCTTAAACATCG
ATCCTAAAACACTTCCAGACACCTGTGCTGCTCCTTTCAGTTCCCATGGAGATTAGGC
TGTGTAACAATCTCGCAAAGACGTTCCCCTCCGTCTCCTCATCCTCTTTTCAAACCCT
TTTACGATTTCCCATCTCACTCAGCATGACAGTCAAAGTCCCTGTGATGGCCAACTTC
TGCATCACCTAGCCAGTCTGCCACCGCCAAAACTCTCCAGCCTCATCTTACACTTGTT
CTCTGCTTGGAATCTTCCCTCCCCTCCTTGAGGAACTTTCTCAAATGTCACCTTCCCTC
AATACTCCCCCTCCTCCATTTAAAACTATAAACTTCCAACTCTCTAAGCCCCTAAAGT
ACTCTATATTTAACTTATTGTATAAACTACTGTCCCTACTTGTAAGTTCCAAGATTGC
AGGGATTCACCCGCTTTGTTCACTGCTGTCTGCCAAGGTCTAGAACAGTGCAAGTTA
CCCAACAGGAGTTCAATAAACAGCCATTCATTTAACAAATATTTGCTGAGCACTTCG
TCCCGTCCAAGTTTGTTAAATCAAGACAAATAAGACACCGTCCCTGCCTTTAACGCA
CCAGATGGAGAAATGCACCACAGACATAAATGTGCAATACAGGCCTGACACTACGG
CCACAAGCAAGTCAAAGAACGTGCCAAAAGTTCAGAGGAAGAAGCCTCGGCTTCGC
CTTTCGGGAGACCAGTCCAGCTTTCCACCATCACGCTGCTCATCAGGGACCATCTCC
GGGGGTCTCCTCTAGACCCCAAGGGAGGAGCGGGTCCCGCCCGCCATTCCCAGGTCT
CAGAGTTTACTTGTCCAGAGATGCAACTTCCGGCCTCTTCAGGCCGGGCAAGATTTA
AGGAAAGAAAAGAAACATAAGGACCTCCGTTCTTCGGTCTCCGTCCCCTCCCCTTCC
CCCGCGTGCCCCACCTGTTCCCGGCGTCCCCTTCGGCTACTCCCGGCGTTTGCGCAAG
CGGTCCCACGTGGGCTCGGGCGGGGCTAGCGCCGCGGCGGGGGCTGGGCACGCCCC
TAGCGCATAGCTGGCTTCTGATTGGCTTTCC

In some aspects, the inner ear supporting cell selective promoter is a FABP3 promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94. In some aspects, the FABP3 promoter comprises the nucleic acid sequence of SEQ ID NO: 94.

Exemplary FABP3 promoter
(SEQ ID NO: 94)
TACCATTCTGCCTTTCACCTGATGTTGCTATCCTCCTCCCTCTTGTTTCCTTCCACCCA
TCCTTTCCCTCCCACATTACTCTCTTATCCCACCCTATTTTACAACCAGTAGCCTAGG
GAAAAGAGCATAGCTCAAATGAGGAAGAAGGCAGGACAGGCAGTCATGGCTTAGCT
GGACTGAGCTGCAGTGCTTCTCCTTCTGGGGAAGGGGGTGCACTGTCATCTGCTACT
GACACATCCCTCCAAGGCACTCAGCCCTGCAGGGAGCAACCTGATTCTATGACTGAC
ATCTAATCTTCACATTCACCTTGCAGGAAGGCAAGAAGTGATCCCAGCCTCCAGATG
GAAAGATCAAGGCCCAGAGAAGGTCAGTGGTGGTTGGAGGCCTGAGGTCACACAGC
AGCCAAGTCTGGAGTCACTAGTCAAGGTGACCTTGACTAGCCACCCCACCTCCCCTT
CCCTGCCCCACCATGGCCCTGGGAGATCTGTTGTCCTGTGAGGGAAAGGGGCTCCAG
GCTGGGCTGCATCTGAAGCCCCTAGATCCAGAGACTTCATTTCTTAGGCTATCTATA
AAATCCACCTTCCTTTCTTTTCCCAGGACCCCCATACCCTGCTCCCAGCATCGTCTGC
CTCAGCTAAGCCATGGGGATTGAGAGACCAGGCCTGGTGCCCAGATAAACTGACCC
TGGGTGAGGGGACAGGGGCCCAGAATGGGCAGGTAGAGACTGAATACTGAAGAAG
AATCCTCTGGAGTCTGTTAGCAGAAGCAGATGGGCCTTGCCTGACTATTGGCAGGCG
GACCTGGTGGTCAGACCTCAGTGATCCTCAGGGACCAGTGAATATTTCAGGCTGGGG
CTGAGCATCACCTGCTCCCTTGGCCCCACTTATAGGGCAAAGGGGAGTCTACCAGCC
TACTCACTGATGACAAACTGGAAAAGTTTGTCCTGTCTCTGCTCTGGCCCCACCTCGC
CCTCTCCCCTACTTGGAAGTTCCTTTCCTGAACCACTGACTGCCAAAGCTTGAGGGAT
TAAATAAATCATCTGGCCCAACCTCCTACCATAGAGTTGGGAACACTGAAGAAAAG
AGACTGGCCCAAGGTCACAGAGAAGGCAGGGTGAACACTGTCACAGGGAGAGCCA
GTGTAGAATAATGGTTAAGCCACGCAAGCTCTAGAACCACTCTATCTGAGTGCAAAT
CCTGGCTGTCATCTGGTACTTGCTTCCTGGAACACATCTGGCCTCAGACTCCTGAGGC
CAAGACACACTCCCTGCCCTAAGACTTGCTGGTTCTATGGCAGGCAGAGGCAGAAA
GAGCCCCACCATCATTCCCAGCAAATGGGAAAAGTTCCCAGTTGCAGATATTAGGGG
TGGGATGGGGCGGGGGTAGTCAGCAACCATAGACTTAGACCCTGAAGAGGCAAAAA
AGGAGGGCCATGTTCTTGGGTCAGCAGAGCTTCTACTCAGCTTCTTCAGCCTCTAGCT
CTTTCCTGGTGCTAGTAGCACATTCTCTAGTGGAGGCATCCAGATGGCAGGGAGGGT
CCAGGAAACAGCTGAACATGCTGAGCAGGCCTCCCTTGTCCCCGCTCCCCATGGCCC
CATGGATCATCCGGTGCTGCAGCTCATCTCATTGGCTGGCTTCTGGTTACTCATCTCT
CCTCTTCTCCATCTTCCCAGCCTGTGGTTGCCGTGGAAACATAGAACAGTGACCTCAC
CATAGGATGAGGGCTGGGGAGATGCTGTTCTTGGCAGGCGCT

In some aspects, the inner ear supporting cell selective promoter is a KLHL14 promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95. In some aspects, the KLHL14 promoter comprises the nucleic acid sequence of SEQ ID NO: 95.

Exemplary KLHL14 promoter
(SEQ ID NO: 95)
GAAACAGCAGCCATTGATGTAGCTCAGGGTTCTGTGGATCTGTCATTTGGAGCATGT
TGGTTCTCCTGTCTCAGCTGGGCTCATTCATGCATCTGAGTTCAGCTATTGGGCAATC
TGGGGAATGTTTTGTCCATGTGATGTGTCATCTTCTACCAGGCTAGCCTGGGCTTCAT
CACATGGTATCTGGCAGGGCTCTAAGAGGGAGAGTTGAAACACACAAGGCCTCTTG
AAGCTTAGACTCAGAATTGGCACAAGGTCGCTTCTGGCACATTCCATTGGTCAAAGC
AAGTTACAAGGCCAGCTCACATTCAAGGATTAGGTAAGTCGATTCCACTCTTGATGA
GAAGTCTGAAGGATTTGGAACAGTGTCCACCATGCAGTAATAAACTCAATAAGTAGT
AGCCATTATTATTCTGTTAGAGGTTGCCAGGAAAAGTTTTATAGTGGAAAGAAATCT
GAGTTTACTCTTGAGAGGTAAGTGGAATTTCTATTTGTAGAGAATGAAGGCCTCTCA
AAAAGACACAGCCTAACAATAGGTGCTGCAGTTTAACAGTGGAGCGTGTCCAGAAC
AGGCTGCCCTTTTAGGCAAGGGCTAGTGTCTTTCAGGACAGACCCAAACCCCAAATA
CCAAAACAGAATAAAGTAGTGTCTTAGCATACTTTGAGATCAGACTGTTTCTGCATT
TCACAGTGCTGGGGGTGGGGGGGAGGTGTGGGGGGAAGGGAAAAGCAGCATACCA
ATGTAGTGAAATCTGGAAACAACAGCCAAAAAAAGTTTGCATATTGCACAGAGCAC
TTGAAGATCATAAATCTATGCATGAGAAAGATGTAGTGGAAATTTTGGGGGGGATTA
GAGTTTATTTTTGTCATCTCTGTGAGACAGCTACTCATTCATCCAGATCACAGCTAAG
AAAAAAGCTGGTCACAGAAATTAGCAGTTTCAGCTCAGCAGCGAAGTCGCCAGCCT
GTGAAGGCAGAGAGAAATTGACTAATTAGCAATGCGCACTAAAACTTGACGGTTCTT
TATAGAGAGAGAGAAGAGAGAGGGAGAGAGAGGGAGAGGGAGGGAGGGGGGGCT
CGCTTTTTCCCCTTCTTTCTTCCAAAGATGTTTGAAATCGCAGTCATTTACGCTCGAC
AATTTTTACAATAGCCTTGAGCCATAATTTTGCGAGTCTCTCCAGCATCCATCCCCCT
GTATGGTCTCTCTCTACTGGCCAAGCACGACCGTTTCTCTCCCCAACCGTGGATTTCC
TATT

In some aspects, the inner ear supporting cell selective promoter is a MMP15 promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96. In some aspects, the MMP15 promoter comprises the nucleic acid sequence of SEQ ID NO: 96.

Exemplary MMP15 promoter
(SEQ ID NO: 96)
CCTTCCTCCTCCAGGGCCCTCTGCAGACCAGGCTGAGATGGAGGAACCTGCTAAAAT
CGATGGAGATGCTTCTAGCCTCCCAGTAGGAGGCCCCAGCCATGCCTTCAACCTGGC
AGGAGGTGTAGCCACTCCTCATCCTTGGGTTGCAGGTTGGGTGCTGCTGTTGTGGTC
CTTCCCAGAAACTGCCAGTAGAGGGCAGCCTGGGCATCCTAATGCTTACTCTGGTTG
TTACACAAAGAAAATATTGGGGTCACTGGCGAGCCCACCCACACTCACCAGAATCTC
CACTGTAGTCCCCCTAACAAACAGCCCTTCACTTCCTCTCCCACTTCAGCAATTTGTA
TTTTGATGCCATTGGCCTCAGATCAGAGTGTTTTAAATCATCACGCCCTGGCTTATCC
CTGGTCGAGCCAGGACACGGGGTGCTTCAGTGGGTCTGTCACCCTCTCTCCTTGAAG
CATGTTGCTTTTATTTATTTACTTTTACTCTCACCCTGCTCCTGTACCAGCAGGGGCCA
CTTCAAAGCCAAGGTACAGGGTGATAACTTGTGGTCCAGCATCAGTTTTCTCCACTT
CTTTCTCCCACTCACCCCCAGCAAGGTGCCTGGGGAGACTTGAGCAGATGTTTCATTT
TGGCCTGGCCAGTGGCTGAAAGCCAGGCCTCCAATGCACTGTGACCTCTGGCTTCCC
CAGCAGCTTTCCCAGAGAGGCAGAGGGAGTCTTCATTCTTCCCAGGGGGGAGACC
ACGCCTTCCCTGCCTCCTCCCTCCGCGGGGGGTCGCGTTGGAGGTCACCCCCGCCCC
CTAGGCGCTGGGTTGGGAGTGACGCGGGGTGGGCTGGAGAGGTTTCCTGCCGTCTGG
GAAGCGTAAACGGACCGCCCACCTGTCGGGCCTCGGCCGCCCGCACCTGCTTGTGAG
AAGCCTGCGGCTGGGGCACCGCCCCCGGTCCCCGCCCGGGTCCGCGCATTGGGAGC
ACACTGGCCCTTTAAGAGCGCGGCGGCCGCGGCGCGCGGG

In some aspects, the inner ear supporting cell selective promoter is a SPARC promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97. In some aspects, the SPARC promoter comprises the nucleic acid sequence of SEQ ID NO: 97.

Exemplary SPARC promoter
(SEQ ID NO: 97)
CAGGCTACCTCTCAGGCTGACTGAGTCATGCAGCATAGGCTGCCACGTCTCTGGGCT
GGCGGGGCCGTCATTATTCCTGGCCTCACTGCAGCTAAATTGAAGAAACGTTTGGTT
TGTGGGCCACGTCAAGGAATGTGTAAGAGCTGCCACGTTGTCGGGTCTGGGTTATTG
GGCTTTTCCCCTCCTTCAGAGAAGATTTCCAGGCGTGTGGGTGGGGTTTCAGAAGAA
AATTGATGCCTGCGTGTGAGTGTTCCCTGGACCTGGACCAGCAGCGGCAATATTACA
GACCCGGGGGTTGGGGCAGACTGAGCCAATCTCTGCACCGTCAAAGTTATGGATAC
AGAGCCCTGGAAAAAGGCTGAAGGATAAGATAGCTGACATTTATGAAGTGCTTCAT
TCATGTAGCAGTGGGCCAAATGCGTACTTTACACTTGAGGAAGCTGAGGCTGGAGGT
TGATAACATGCCTCAAGTCTTCTAGAGTTAAATAACTTTGACCCAGGACCCAAGCCC
AGAGTTCTGACTCAAAAACTAGGCCTCCTAAACATCCTCTTATATGAGGTTAAATTT
CATCTTCCTCTGTTTGGCCTTGGCCTGGTTGGTGGATGCTCTGCTTCGGGGACCCAGG
GCCAGATGACAATGGGTTCTTTGTGCCCTTCAGACAATGGGAAGGGCTGCCTGGGGA
AAGATACAGTAACAAGGCAACAGGCTGAGTCAGCCTCCAATGTGCTTGAACCTTCTT
AGCTTGGCAGCCTTGACATTCAGCCAGCCACACAAAGGGTATATCAAGGATGATACC
ACTAGTAGCAGCTTGTCTTGTCTGTACCTCTGAACAAGAAAGAGGCTGTTCTGGGTC
ATCCCTCCAGGCCTGTCCAGCCCTGGCACTCTGTGAGTCGGTTTAGGCAGCAGCCCC
GGAACAGATGAGGCAGGCAGGGTTGGGACGTTTGGTCAGGACAGCCCACCAGGAGG
AAAGAAATGAAAGACAGAGACAGCTTTGGCTATGGGAGAAGGAGGAGGCCGGGGG
AAGGAGGAGACAGGAGGAGGAGGGACCACGGGGTGGAGGGGAGATAGACCCAGCC
CA

In some aspects, the inner ear supporting cell selective promoter is a TSPAN8 promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98. In some aspects, the TSPAN8 promoter comprises the nucleic acid sequence of SEQ ID NO: 98.

Exemplary TSPAN8 promoter
(SEQ ID NO: 98)
CCAAGGACTCTTTTTTCTAAACTTCCCTTCATCTTCTAGTTTGACGCCCTTGGTGGGA
AAAGTGTCTGAGATAAGGAAAAGGCATCCTTTCAGTTCTCTGATACTATCTTGAAGC
GAGGGATGGAGAAAGGCAAAGAGAGACACAGGAGAAGCGTATCCCCTGGGAACAG
GTGTCTAGTGGAGTCCAGTAACTCACAGTCTCTCAGTTCCGTCAGCACTGTCCCTTGG
GTCGCAAATTTCTTCCATTAGCCCTTCCACCAGCTGTATTTCAAATGGGGCTGGACAA
TAATTGTGGCCAGTGGCCTTGTGTTGTTTGTACTTGCGGACTAGTAGTTCTCACCTGT
CTTTCTCTGACTCCTATTAGCCACTGGGATTTCAGCAGCTGGTTCAGCCAATTCTACT
CAATTCAACATTAAGTTGCAGTGGGCTAGAACTCATGGGCCGATTTAACAAGTGAAA
TTCTACCAAGATACATCAAAAATAGCAACAGGACTAGATACTCAGCTCATTTTGTTT
TATTTGTAATATACCAGTTGTGGCTTTAGTGCCAGTCTGATTCATCTCTCTACTACAA
AATGAGGCTCTATAAAGGAAAATATTGCAACTGGAGTGAGGAATTTGAATCTTATAG
GAAGGAATTTGTCTTCTCATGAAGACTTCAGTTTACCAGAAGTATCTATTGAGGAAG
TGTTTACAAGAAAATGTGCCATTTAGCTTTATTCTAAATTTGCATAATAACTGAACCA
AACAAAAAAATATAGATAGATAGATTGTTCTATCTATAGATAGATAGGGAACATTG
GCAGTAGGTGGCAGTAAGTTCCCCTGAGCACATGGAGGACACAGTTAAATGCATTTG
AGGTATGTGGGAAATGGTTTAAAGCAGAATTTTATGCCAACTTTTAGTAACGGAAGC
CTAACAAATGTTTGTTCTTTCAAGTGAGAGAAGCAAGCAATCTGGAACTATTCATAA
GCTTATTTTCTGTATCCTTAAACATATTTTATAATGAATGTATGATTTAAATAGTAAG
TTAAGTGTCTGGGGGTACTGCACACCTCCCTTGCATACAGTCAAACTTCTTCAGGGT
GATGGGGAAGAGGAGTTATAGGCTGCCAAGCAAAATTGCCAAACTGGTCTCAGAAA
TTCACTGCATTGGAGAGCGCGGGATCCTTGCAACACTGACTTTAGCAGTTAAACTAG
AGTGGTTGGGGATGAGTATTCT

In some aspects, the inner ear supporting cell selective promoter is a VIM promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99. In some aspects, the VIM promoter comprises the nucleic acid sequence of SEQ ID NO: 99.

Exemplary VIM promoter
(SEQ ID NO: 99)
ATTCACAATGCATTCCCTCTGCCCACCACATTAATTATCAACTCCTTTTCCTGGCATT
TACTCATCCAACGCATGGCCCCACGTTAACTTTCAGTTCCCTTTCTCCCCTACAAATA
CTCCATAATCCAGCAACCCTGGGATCCCTGAGATGATGAAGAGGACCAGTGCCCATT
CCAGGAGACATCACCGCAGCCCTGAGGAATCGGCTATGGGCACCAGCAGGGCACAG
TGCCACACCTCGCCAATGCCTTGTCCTCCTTTTCCATAGTGAGTCAGTCAGCAAGCGT
GTAGAAGTGAGTTCCACACTCTCTTCCTCCCATAGGGAGATCACTTTTCTCATTCTAA
GGGTTCCAGGCACACTCACAATGGTGGCATTTGCTGAGCAGTGGCTTGAATAAAGGG
CTCTCAGAAAGCAAGATGTAACTCAGAGCATAGGCTAGCCCCAGGAATGCTCTTGG
GGAATGACCTGCAGCCTCCCAGTGAAAGAGAGAATAAAAGAAAGCCCCAGCAGGCG
AGCTGGGCAGTAGAGAGTCCTGTAATTCCACCTTGGCAAGCACCATTTGCAAGAACG
AACTGGGATAAGGTAAACAAAATATTGCCTAAAAGAGGCTTGTCCAAAGAAGTCAG
AATACGCTCTTCATTTACCTCTAAATTTCAGTACACCATAAATCTAAATACTCAAAAA
AACCTGTGCCTTTTCAATCAAGGTCAATTGCACGAATTCTTTTGGAAAACAGGACCT
ATGGCATTTCCCAGACAAATCACCGTGAACCCTGTACTGTGCATTGCTGTCCTAAAA
TCCAAAGATTCTGTCATTTGTGTTACATAATTGCCTTTCATTTGAACTCATTAATCAA
ATTGGGGTTTTTAAGCAACACCTAATTAATTCTTTAACTGGCTCATCCACTGATCACT
GAGTTCTATTTTGAAACTACGGACGTCGAGTTTCCTCTTTCACCCAGAATTTTCAGAT
CTTGTTTAAAAAGTTGGGTGTGGTTTCATGGGGGGAGGGGGAAGAGCGAGAGGAGA
CCAGAGGGACGGGGGCGGGGACTCTGCAAGAAAAACCTTCCCGGTGCAATCGTGAT
CT

In some aspects, the inner ear supporting cell selective promoter is a GFAP promoter. In some aspects, the GFAP promoter comprises a nucleic acid sequence with at least 85%, at least 90%, at least 95%, at least 96%, at least 97%, at least 98% at least 99%, or 100% identity to SEQ ID NO: 91. In some aspects, the GFAP promoter comprises the nucleic acid sequence of SEQ ID NO: 91.

Exemplary GFAP promoter
(SEQ ID NO: 91)
GAACATATCCTGGTGTGGAGTAGGGGACGCTGCTCTGACAGAGGCTCGG
GGGCCTGAGCTGGCTCTGTGAGCTGGGGAGGAGGCAGACAGCCAGGCCT
TGTCTGCAAGCAGACCTGGCAGCATTGGGCTGGCCGCCCCCCAGGGCCT
CCTCTTCATGCCCAGTGAATGACTCACCTTGGCACAGACACAATGTTCG
GGGTGGGCACAGTGCCTGCTTCCCGCCGCACCCCAGCCCCCCTCAAATG
CCTTCCGAGAAGCCCATTGAGCAGGGGGCTTGCATTGCACCCCAGCCTG
ACAGCCTGGCATCTTGGGATAAAAGCAGCACAGCCCCCTAGGGGCTGCC
CTTGCTGTGTGGCGCCACCGGCGGTGGAGAACAAGGCTCTATTCAGCCT
GTGCCCAGGAAAGGGGATCAGGGGATGCCCAGGCATGGACAGTGGGTGG
CAGGGGGGGAGAGGAGGGCTGTCTGCTTCCCAGAAGTCCAAGGACACAA
ATGGGTGAGGGGAGCTCTCCCCATAGCTGGGCTGCGGCCCAACCCCACC
CCCTCAGGCTATGCCAGGGGGTGTTGCCAGGGGCACCCGGGCATCGCCA
GTCTAGCCCACTCCTTCATAAAGCCCTCGCATCCCAGGAGCGAGCAGAG
CCAGAGCAGGTTGGAGAGGAGACGCATCACCTCCGCTGCTCGC

TABLE 2
Exemplary Promoters

| Promoter | SEQ ID NO |
|---|---|
| PARM1 derivative | 40 |
| GJB6 derivative | 16 |
| RBP7 derivative | 28 |
| IGFBP2 derivative | 57 |
| GDF6 derivative | 90 |
| GFAP derivative | 91 |
| BACE2 derivative | 92 |
| DBI2 derivative | 93 |
| FABP3 derivative | 94 |
| KLHL14 derivative | 95 |
| MMP15 derivative | 96 |
| SPARC derivative | 97 |
| TSPAN8 derivative | 98 |
| VIM derivative | 99 |
| GJB2 minimal promoter | 86 |
| 1. CAG enhancer/promoter | 15 |
| 2. CAG enhancer/promoter | 14 |
| 1. CMV/CBA enhancer/promoter | 13 |
| 2. CMV/CBA enhancer/promoter | 12 |
| 1. CBA promoter | 11 |
| 2. CBA promoter | 10 |

### Enhancers

In some instances, a construct can include an enhancer sequence. The term “enhancer” refers to a nucleotide sequence that can increase the level of transcription of a nucleic acid encoding a protein of interest (e.g., a polypeptide (e.g., a therapeutic polypeptide, a Connexin 26 polypeptide)). Enhancer sequences (generally 50-1500 bp in length) generally increase the level of transcription by providing additional binding sites for transcription-associated proteins (e.g., transcription factors). In some aspects, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some aspects, a construct comprises a CMV enhancer exemplified by SEQ ID NO: 18. In some aspects, a construct comprises a CMV enhancer exemplified by SEQ ID NO: 63. In some aspects, a construct comprises a chimeric intron enhancer exemplified by SEQ ID NO: 64. In some aspects, an enhancer sequence is at least 85%, at least 90%, at least 95%, at least 96%, at least 97%, at least 98% at least 99%, or 100% identical to the enhancer sequence represented by SEQ ID NO: 18. In some aspects, an enhancer sequence is at least 85%, at least 90%, at least 95%, at least 96%, at least 97%, at least 98% at least 99%, or 100% identical to the enhancer sequence represented by SEQ ID NO: 63. In some aspects, an enhancer sequence is at least 85%, at least 90%, at least 95%, at least 96%, at least 97%, at least 98% at least 99%, or 100% identical to the enhancer sequence represented by SEQ ID NO: 64. In some aspects, an enhancer sequence is at least 85%, at least 90%, at least 95% at least 96%, at least 97%, at least 98% at least 99%, or 100% identical to the enhancer sequence represented by SEQ ID NO: 65. In some aspects, an SV-40 derived enhancer is the SV-40 T intron sequence, which is exemplified by SEQ ID NO: 19. In some aspects, an enhancer sequence is at least 85%, at least 90%, at least 95%, at least 96%, at least 97%, at least 98% at least 99%, or 100% identical to the enhancer sequence represented by SEQ ID NO: 19.

In some instances, the construct does not include an enhancer sequence.

Exemplary CMV enhancer
(SEQ ID NO: 18)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGG
Exemplary CMV enhancer
(SEQ ID NO: 63)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGT
Exemplary SV-40 synthetic intron
(SEQ ID NO: 19)
GGAGTCGCTGCGTTGCCTTCGCCCCGTGCCCCGCTCCGCGCCGCCTCGCGCCGCCCGCCCCGGCT
CTGACTGACCGCGTTACTCCCACAGGTGAGCGGGCGGGACGGCCCTTCTCCTCCGGGCTGTAATT
AGCGCTTGGTTTAATGACGGCTCGTTTCTTTTCTGTGGCTGCGTGAAAGCCTTAAAGGGCTCCGG
GAGGGCCCTTTGTGCGGGGGGGAGCGGCTCGGGGGGTGCGTGCGTGTGTGTGTGCGTGGGGAGCG
CCGCGTGCGGCCCGCGCTGCCCGGCGGCTGTGAGCGCTGCGGGCGCGGCGCGGGGCTTTGTGCGC
TCCGCGTGTGCGCGAGGGGAGCGCGGCCGGGGGGGGTGCCCCGCGGTGCGGGGGGGCTGCGAGGG
GAACAAAGGCTGCGTGCGGGGTGTGTGCGTGGGGGGGTGAGCAGGGGGTGTGGGCGCGGCGGTCG
GGCTGTAACCCCCCCCTGCACCCCCCTCCCCGAGTTGCTGAGCACGGCCCGGCTTCGGGTGCGGG
GCTCCGTGCGGGGCGTGGCGCGGGGCTCGCCGTGCCGGGCGGGGGGTGGCGGCAGGTGGGGGTGC
CGGGCGGGGGGGGGCCGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTAATCGTGCGAGAGGGC
GCAGGGACTTCCTTTGTCCCAAATCTGTGCGGAGCCGAAATCTGGGAGGCGCCGCCGCACCCCCT
CTAGCGGGCGCGGGGCGAAGCGGTGCGGCGCCGGCAGGAAGGAAATGGGGGGGGAGGGCCTTCGT
GCGTCGCCGCGCCGCCGTCCCCTTCTCCCTCTCCAGCCTCGGGGCTGTCCGCGGGGGGACGGCTG
CCTTCGGGGGGGACGGGGCAGGGCGGGGTTCGGCTTCTGGCGTGTGACCGGCGGCTCTAGAGCCT
CTGCTAACCATGTTCATGCCTTCTTCTTTTTCCTACAG
Exemplary chimeric intron
(SEQ ID NO: 64)
GGAGTCGCTGCGTTGCCTTCGCCCCGTGCCCCGCTCCGCGCCGCCTCGCGCCGCCCGCCCCGGCT
CTGACTGACCGCGTTACTCCCACAGGTGAGCGGGCGGGACGGCCCTTCTCCTCCGGGCTGTAATT
AGCGCTTGGTTTAATGACGGCTTGTTTCTTTTCTGTGGCTGCGTGAAAGCCTTGAGGGGCTCCGG
GAGCTAGAGCCTCTGCTAACCATGTTCATGCCTTCTTCTTTTTCCTACAG

### Flanking Untranslated Regions, 5′ UTRs and 3′ UTRs

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a polypeptide (e.g., a therapeutic polypeptide, a Connexin 26 polypeptide).

Natural 5′ UTRs include a sequence that plays a role in translation initiation, in some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some aspects, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

In some aspects, a 5′ UTR from an mRNA that is transcribed by a cell in the cochlea can be included in any of the constructs, compositions, kits, and methods described herein. In some aspects, a 5′ UTR is derived from the endogenous GJB2 gene loci and may include all or part of the endogenous sequence exemplified by SEQ ID NO: 20, SEQ ID NO: 21, or SEQ ID NO: 66. In some aspects, a 5′ UTR sequence is at least 85%, at least 90%, at least 95%, at least 96%, at least 97%, at least 98% at least 99%, or 100% identical to the 5′ UTR sequence represented by SEQ ID NO: 20, SEQ ID NO: 21, or SEQ ID NO: 66.

3′ UTRs are found immediately 3′ to the stop codon of the gene of interest. In some aspects, a 3′ UTR from an mRNA that is transcribed by a cell in the cochlea can be included in any of the constructs, compositions, kits, and methods described herein. In some aspects, a 3′ UTR is derived from the endogenous GJB2 gene loci and may include all or part of the endogenous sequence exemplified by SEQ ID NO: 22. In some aspects, a 3′ UTR sequence is at least 85%, at least 90%, at least 95%, at least 96%, at least 97%, at least 98% at least 99%, or 100% identical to the 3′ UTR sequence represented by SEQ ID NO: 22. In some aspects, a 3′ UTR is derived from the endogenous GJB2 gene loci and may include all or part of the endogenous sequence exemplified by SEQ ID NO: 67, or SEQ ID NO: 68. In some aspects, a 3′ UTR sequence is at least 85%, at least 90%, at least 95%, at least 96%, at least 97%, at least 98% at least 99%, or 100% identical to the 3′ UTR sequence represented by SEQ ID NO: 67, or SEQ ID NO: 68.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some aspects, the introduction, removal, or modification of 3′ UTR AREs can be used to modulate the stability of an mRNA encoding a polypeptide (e.g., a therapeutic polypeptide, a Connexin 26 polypeptide). In other aspects, AREs can be removed or mutated to increase the intracellular stability and thus increase translation and production of a polypeptide (e.g., a therapeutic polypeptide, a Connexin 26 polypeptide).

In other aspects, non-ARE sequences may be incorporated into the 5′ or 3′ UTRs. In some aspects, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 20)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAG
Exemplary 5′ UTR Sequence
(SEQ ID NO: 21)
TTTAGGACCCTTGTTCGCGAAGAGGTGGTGTGCGGCTGAGACCCGCGTCCTCAGGACGGTTCCAT
CAGTGCCTCGATCCTGCCCCACTGGAGGAGGAAGGCAGCCCGAACAGCGCTCACCTAACTAACAG
CTGCTGAGAGCTGGGTTCCGTGGCCATGCACCTGGGACTGCCTTGAGAAGCGTGAGCAAACCGCC
CAGAGTAGAAG
Exemplary 5′ UTR Sequence
(SEQ ID NO: 66)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGTTTAGGACCCTTGTTCGCGAAGAGGTGGTGTGCGGCTGA
GACCCGCGTCCTCAGGACGGTTCCATCAGTGCCTCGATCCTGCCCCACTGGAGGAGGAAGGCAGC
CCGAACAGCGCTCACCTAACTAACAGCTGCTGAGAGCTGGGTTCCGTGGCCATGCACCTGGGACT
GCCTTGAGAAGCGTGAGCAAACCGCCCAGAGTAGAAG
Exemplary 3′ UTR Sequence
(SEQ ID NO: 22)
CGCATTGCCCAGTTGTTAGATTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAACCATTTGA
AACCCCTGTAGGCCTCAGGTGAAACTCCAGATGCCACAATGGAGCCTCTGCTCCCCTAAAGCCTC
AAAACAAAGGCCTAATTCTATGCCTGTCTTAATTTTCTTTCACTTAAGTTAGTTCCACTGAGACC
CCAGGCTGTTAGGGGTTATTGGTGTAAGGTACTTTCATATTTTAAACAGAGGATATCGGCATTTG
TTTCTTTCTCTGAGGACAAGAGAAAAAAGCCAGGTTCCACAGAGGACACAGAGAAGGTTTGGGTG
TCCTCCTGGGGTTCTTTTTGCCAACTTTCCCCACGTTAAAGGTGAACATTGGTTCTTTCATTTGC
TTTGGAAGTTTTAATCTCTAACAGTGGACAAAGTTACCAGTGCCTTAAACTCTGTTACACTTTTT
GGAAGTGAAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACTATGATTT
AATTTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTTCCT
AGTGATGGCTTATGATAGCAAATGGCCTCATGTCAAATATTTAGATGTAATTTTGTGTAAGAAAT
ACAGACTGGATGTACCACCAACTACTACCTGTAATGACAGGCCTGTCCAACACATCTCCCTTTTC
CATGACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGAAAA
GAATAGAAGCTAAGGTTTAGATAAATATTGAGCAGATCTATAGGAAGATTGAACCTGAATATTGC
CATTATGCTTGACATGGTTTCCAAAAAATGGTACTCCACATATTTCAGTGAGGGTAAGTATTTTC
CTGTTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTATAAT
AA
Exemplary 3′ UTR Sequence
(SEQ ID NO: 67)
GAAATAGACAGCATGAGAGGGATGAGGCAACCCGTGCTCAGCTGTCAAGGCTCAGTCGCTAGCAT
TTCCCAACACAAAGATTCTGACCTTAAATGCAACCATTTGAAACCCCTGTAGGCCTCAGGTGAAA
CTCCAGATGCCACAATGGAGCTCTGCTCCCCTAAAGCCTCAAAACAAAGGCCTAATTCTATGCCT
GTCTTAATTTTCTTTCACTTAAGTTAGTTCCACTGAGACCCCAGGCTGTTAGGGGTTATTGGTGT
AAGGTACTTTCATATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGTTCTTTTTGCCAAC
TTTCCCCACGTTAAAGGTGAACATTGGTTCTTTCATTTGCTTTGGAAGTTTTAATCTCTAACAGT
GGACAAAGTTACCAGTGCCTTAAACTCTGTTACACTTTTTGGAAGTGAAAACTTTGTAGTATGAT
AGGTTATTTTGATGTAAAGATGTTCTGGATACCATTATATGTTCCCCCTGTTTCAGAGGCTCAGA
TTGTAATATGTAAATGGTATGTCATTCGCTACTATGATTTAATTTGAAATATGGTCTTTTGGTTA
TGAATACTTTGCAGCACAGCTGAGAGGCTGTCTGTTGTATTCATTGTGGTCATAGCACCTAACAA
CATTGTAGCCTCAATCGAGTGAGACAGACTAGAAGTTCCTAGTGATGGCTTATGATAGCAAATGG
CCTCATGTCAAATATTTAGATGTAATTTTGTGTAAGAAATACAGACTGGATGTACCACCAACTAC
TACCTGTAATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAATGGGGAGGA
CAAAATGGGGCAGGGGAGGGAGAAGTTTCTGTCGTTAAAAACAGATTTGGAAAGACTGGACTCTA
AAGTCTGTTGATTAAAGATGAGCTTTGTCTACTTCAAAAGTTTGTTTGCTTACCCCTTCAGCCTC
CAATTTTTTAAGTGAAAATATAGCTAATAACATGTGAAAAGAATAGAAGCTAAGGTTTAGATAAA
TATTGAGCAGATCTATAGGAAGATTGAACCTGAATATTGCCATTATGCTTGACATGGTTTCCAAA
AAATGGTACTCCACATATTTCAGTGAGGGTAAGTATTTTCCTGTTGTCAAGAATAGCATTGTAAA
AGCATTTTGTAATAATAAAGAATAGCTTTAATGATATGCTTGTAACTAAAATAATTTTGTAATGT
ATCAAATACATTTAAAACATTAAAATATAATCTCTATAATAA
Exemplary 3′ UTR Sequence
(SEQ ID NO: 68)
CGCATTGCCCAGTTGTTAGATTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAACCATTTGA
AACCCCTGTAGGCCTCAGGTGAAACTCCAGATGCCACAATGGAGCCTCTGCTCCCCTAAAGCCTC
AAAACAAAGGCCTAATTCTATGCCTGTCTTAATTTTCTTTCACTTAAGTTAGTTCCACTGAGACC
CCAGGCTGTTAGGGGTTATTGGTGTAAGGTACTTTCATATTTTAAACAGAGGATATCGGCATTTG
TTTCTTTCTCTGAGGACAAGAGAAAAAAGCCAGGTTCCACAGAGGACACAGAGAAGGTTTGGGTG
TCCTCCTGGGGTTCTTTTTGCCAACTTTCCCCACGTTAAAGGTGAACATTGGTTCTTTCATTTGC
TTTGGAAGTTTTAATCTCTAACAGTGGACAAAGTTACCAGTGCCTTAAACTCTGTTACACTTTTT
GGAAGTGAAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACTATGATTT
AATTTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTTCCT
AGTGATGGCTTATGATAGCAAATGGCCTCATGTCAAATATTTAGATGTAATTTTGTGTAAGAAAT
ACAGACTGGATGTACCACCAACTACTACCTGTAATGACAGGCCTGTCCAACACATCTCCCTTTTC
CATGACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGAAAA
GAATAGAAGCTAAGGTTTAGATAAATATTGAGCAGATCTATAGGAAGATTGAACCTGAATATTGC
CATTATGCTTGAC
Exemplary 3′ UTR Sequence
(SEQ ID NO: 69)
GAGCTCAGTGTGAGTTCTACCATTGCCAAACTCGAGCAGTGAATTCTACCAGTGCCATAGGATCC
AGTGTGAGTTCTACCATTGCCAAAGGTACCCAGTGAATTCTACCAGTGCCATAGTTAACCGCATT
GCCCAGTTGTTAGATTAAGAAATAGACAGCATGAGAGGGATGAGGCAACCCGTGCTCAGCTGTCA
AGGCTCAGTCGCTAGCATTTCCCAACACAAAGATTCTGACCTTAAATGCAACCATTTGAAACCCC
TGTAGGCCTCAGGTGAAACTCCAGATGCCACAATGGAGCCTCTGCTCCCCTAAAGCCTCAAAACA
AAGGCCTAATTCTATGCCTGTCTTAATTTTCTTTCACTTAAGTTAGTTCCACTGAGACCCCAGGC
TGTTAGGGGTTATTGGTGTAAGGTACTTTCATATTTTAAACAGAGGATATCGGCATTTGTTTCTT
TCTCTGAGGACAAGAGAAAAAAGCCAGGTTCCACAGAGGACACAGAGAAGGTTTGGGTGTCCTCC
TGGGGTTCTTTTTGCCAACTTTCCCCACGTTAAAGGTGAACATTGGTTCTTTCATTTGCTTTGGA
AGTTTTAATCTCTAACAGTGGACAAAGTTACCAGTGCCTTAAACTCTGTTACACTTTTTGGAAGT
GAAAACTTTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATTTAATTTG
AAATATGGTCTTTTGGTTATGAATACTTTGCAGCACAGCTGAGAGGCTGTCTGTTGTATTCATTG
TGGTCATAGCACCTAACAACATTGTAGCCTCAATCGAGTGAGACAGACTAGAAGTTCCTAGTGAT
GGCTTATGATAGCAAATGGCCTCATGTCAAATATTTAGATGTAATTTTGTGTAAGAAATACAGAC
TGGATGTACCACCAACTACTACCTGTAATGACAGGCCTGTCCAACACATCTCCCTTTTCCATGAC
TGTGGTAGCCAGCATCGGAAAGAACGCTGATTTAAAGAGGTCGCTTGGGAATTTTATTGACACAG
TACCATTTAATGGGGAGGACAAAATGGGGCAGGGGAGGGAGAAGTTTCTGTCGTTAAAAACAGAT
TTGGAAAGACTGGACTCTAAAGTCTGTTGATTAAAGATGAGCTTTGTCTACTTCAAAAGTTTGTT
TGCTTACCCCTTCAGCCTCCAATTTTTTAAGTGAAAATATAGCTAATAACATGTGAAAAGAATAG
AAGCTAAGGTTTAGATAAATATTGAGCAGATCTATAGGAAGATTGAACCTGAATATTGCCATTAT
GCTTGAC

microRNA Regulatory Target Sites (miRTS)

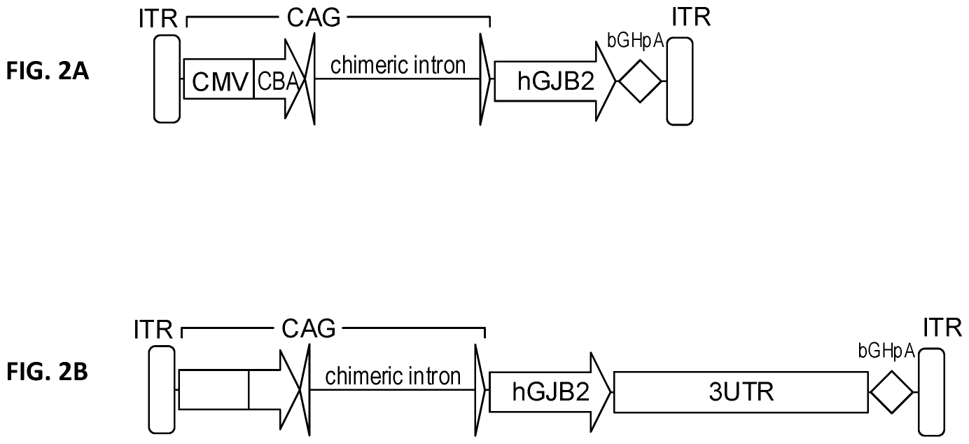

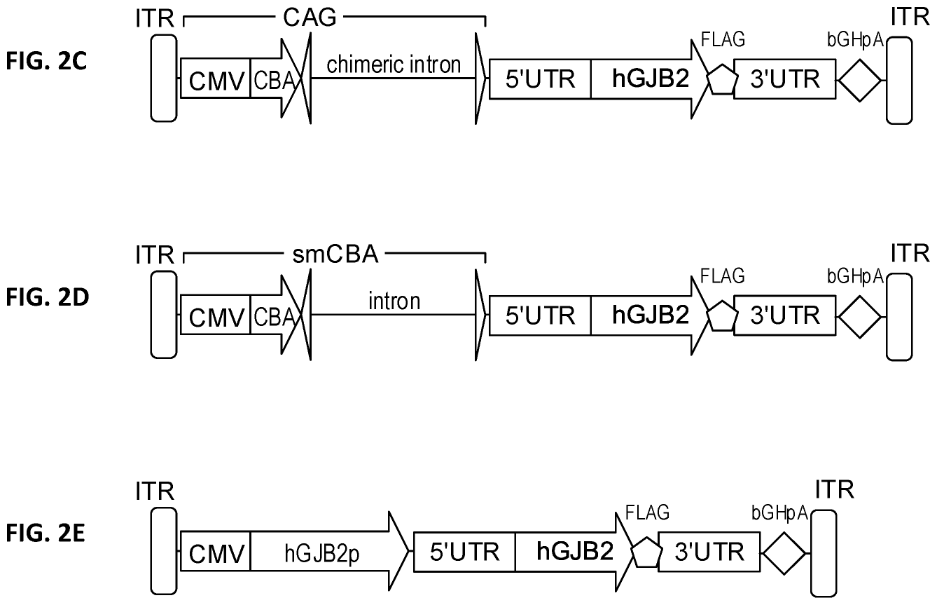

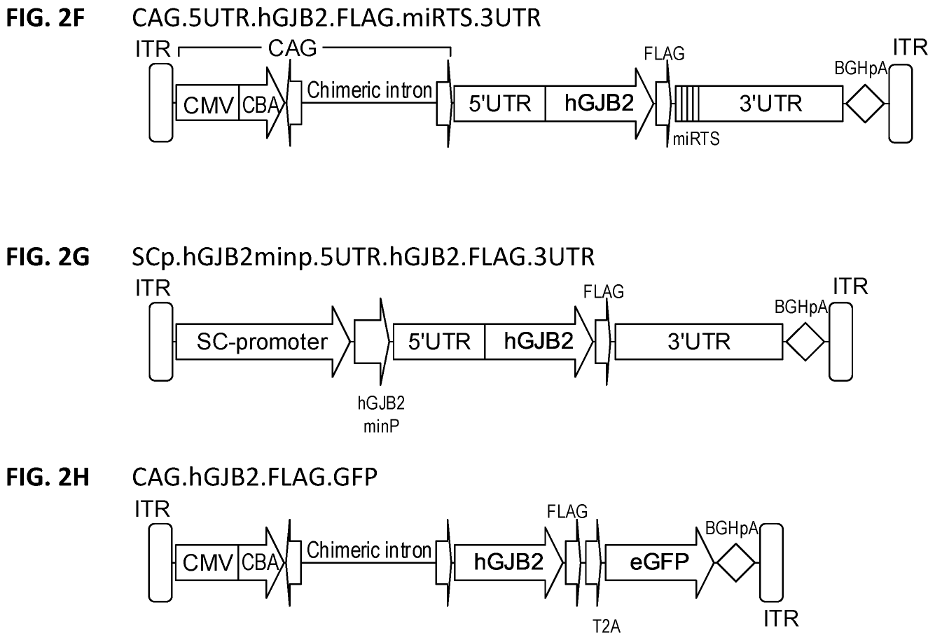

In some aspects, the disclosure is directed to constructs comprising microRNA regulatory target site (miRTS) which can be used to regulate (e.g., reduce) expression of a polynucleotide encoding a therapeutic polypeptide (e.g., a Connexin 26 polypeptide) in a cell (e.g., an inner ear cell, e.g., a hair cell). In some aspects, the constructs provide reduced toxicity associated with expression of the therapeutic polypeptide (e.g., a Connexin 26 polypeptide) in some cells (e.g., an inner ear cell, e.g., a hair cell). In some aspects, the construct comprising a polynucleotide encoding a therapeutic polypeptide (e.g., a Connexin 26 polypeptide) comprises a microRNA regulatory target site (miRTS). An exemplary polynucleotide construct comprising a miRTS is provided in FIG. 2F. In some aspects, a UTR may comprise miRTS. In some aspects, a 3′ UTR may comprise a miRTS. In some aspects, a 5′ UTR may comprise a miRTS.

In some aspects, expression of the therapeutic polypeptide (e.g., a Connexin 26 polypeptide) is reduced, suppressed, inhibited, or eliminated in cells that express the microRNA. In some aspects, the therapeutic polypeptide is predominately expressed in cells that do not express the microRNA. In some aspects, toxicity associated with the expression of the therapeutic polypeptide (e.g., a Connexin 26 polypeptide) is reduced, suppressed, inhibited, or eliminated in cells that express the microRNA.

In some aspects, the disclosure is directed to constructs comprising microRNA regulatory target site (miRTS) which can be used to regulate (e.g., reduce) expression of a polynucleotide encoding a polypeptide in a cell (e.g., an inner ear cell, e.g., a hair cell). In some aspects, the constructs provide reduced toxicity associated with expression of the polypeptide in some cells (e.g., an inner ear cell, e.g., a hair cell). In some aspects, the construct comprising a polynucleotide encoding a polypeptide comprises a microRNA regulatory target site (miRTS). An exemplary polynucleotide construct comprising a miRTS is provided in FIG. 2F. In some aspects, a UTR may comprise miRTS. In some aspects, a 3′ UTR may comprise a miRTS. In some aspects, a 5′ UTR may comprise a miRTS.

In some aspects, expression of the polypeptide is reduced, suppressed, inhibited, or eliminated in cells that express the microRNA (e.g., hair cells). In some aspects, the polypeptide is predominately expressed in cells that do not express the microRNA (e.g., supporting cells). In some aspects, toxicity associated with the expression of the polypeptide is reduced, suppressed, inhibited, or eliminated in cells that express the microRNA.

In some aspects, the miRTS is specific microRNAs expressed in inner ear hair cells, spiral ganglion cells, lateral supporting cells, basilar membrane cells, medial supporting cells, spiral limbus cells, or any combination thereof. In some aspects, the miRTS is specific microRNAs expressed in inner ear hair cells. In some aspects, the miRTS is specific microRNAs expressed in spiral ganglion cells. In some aspects, the miRTS is specific microRNAs expressed in lateral supporting cells. In some aspects, the miRTS is specific microRNAs expressed in basilar membrane cells. In some aspects, the miRTS is specific microRNAs expressed in medial supporting cells. In some aspects, the miRTS is specific microRNAs expressed in spiral limbus cells.

In some aspects, the miRTS may be a human miRNA-182, miRNA-183, miRNA-194, miRNA-140, miRNA-18a, miRNA-99a, miRNA-30b, miRNA-15a target sequence. In some aspects, a miRTS may be a human miRNA-182 target sequence. In some aspects, a UTR may include all or part of the miRNA-182 target sequence. In some aspects, a UTR may contain more than one miRNA-182 target sequence. In some aspects, more than one miRNA-182 target sequences may be dispersed at multiple locations in a UTR. In some aspects, the 3′ UTR may include all or part of the miRNA-182 target sequence. In some aspects, the 3′ UTR may contain more than one miRNA-182 target sequence. In some aspects, more than one miRNA-182 target sequences may be dispersed at multiple locations in the 3′ UTR. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78.

In some aspects, a miRTS may be a human miRNA-183 target sequence. In some aspects, a UTR may include all or part of the miRNA-183 target sequence. In some aspects, a UTR may contain more than one miRNA-183 target sequence. In some aspects, more than one miRNA-183 target sequences may be dispersed at multiple locations in a UTR. In some aspects, the 3′ UTR may include all or part of the miRNA-183 target sequence. In some aspects, the 3′ UTR may contain more than one miRNA-183 target sequence. In some aspects, more than one miRNA-183 target sequences may be dispersed at multiple locations in the 3′ UTR. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, a miRTS may be a human miRNA-194 target sequence. In some aspects, a UTR may include all or part of the miRNA-194 target sequence. In some aspects, a UTR may contain more than one miRNA-194 target sequence. In some aspects, more than one miRNA-194 target sequences may be dispersed at multiple locations in a UTR. In some aspects, the 3′ UTR may include all or part of the miRNA-194 target sequence. In some aspects, the 3′ UTR may contain more than one miRNA-194 target sequence. In some aspects, more than one miRNA-194 target sequences may be dispersed at multiple locations in the 3′ UTR. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1.

In some aspects, a miRTS may be a human miRNA-140 target sequence. In some aspects, a UTR may include all or part of the miRNA-140 target sequence. In some aspects, a UTR may contain more than one miRNA-140 target sequence. In some aspects, more than one miRNA-140 target sequences may be dispersed at multiple locations in a UTR. In some aspects, the 3′ UTR may include all or part of the miRNA-140 target sequence. In some aspects, the 3′ UTR may contain more than one miRNA-140 target sequence. In some aspects, more than one miRNA-140 target sequences may be dispersed at multiple locations in the 3′ UTR. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 2. In some aspects, the miRNA-140 target sequence comprises the nucleic acid sequence of SEQ ID NO: 2.

In some aspects, a miRTS may be a human miRNA-18a target sequence. In some aspects, a UTR may include all or part of the miRNA-18a target sequence. In some aspects, a UTR may contain more than one miRNA-18a target sequence. In some aspects, more than one miRNA-18a target sequences may be dispersed at multiple locations in a UTR. In some aspects, the 3′ UTR may include all or part of the miRNA-18a target sequence. In some aspects, the 3′ UTR may contain more than one miRNA-18a target sequence. In some aspects, more than one miRNA-18a target sequences may be dispersed at multiple locations in the 3′ UTR. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3.

In some aspects, a miRTS may be a human miRNA-99a target sequence. In some aspects, a UTR may include all or part of the miRNA-99a target sequence. In some aspects, a UTR may contain more than one miRNA-99a target sequence. In some aspects, more than one miRNA-99a target sequences may be dispersed at multiple locations in a UTR. In some aspects, the 3′ UTR may include all or part of the miRNA-99a target sequence. In some aspects, the 3′ UTR may contain more than one miRNA-99a target sequence. In some aspects, more than one miRNA-99a target sequences may be dispersed at multiple locations in the 3′ UTR. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4.

In some aspects, a miRTS may be a human miRNA-30b target sequence. In some aspects, a UTR may include all or part of the miRNA-30b target sequence. In some aspects, a UTR may contain more than one miRNA-30b target sequence. In some aspects, more than one miRNA-30b target sequences may be dispersed at multiple locations in a UTR. In some aspects, the 3′ UTR may include all or part of the miRNA-30b target sequence. In some aspects, the 3′ UTR may contain more than one miRNA-30b target sequence. In some aspects, more than one miRNA-30b target sequences may be dispersed at multiple locations in the 3′ UTR. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5.

In some aspects, a miRTS may be a human miRNA-15a target sequence. In some aspects, a UTR may include all or part of the miRNA-15a target sequence. In some aspects, a UTR may contain more than one miRNA-15a target sequence. In some aspects, more than one miRNA-15a target sequences may be dispersed at multiple locations in a UTR. In some aspects, the 3′ UTR may include all or part of the miRNA-15a target sequence. In some aspects, the 3′ UTR may contain more than one miRNA-15a target sequence. In some aspects, more than one miRNA-15a target sequences may be dispersed at multiple locations in the 3′ UTR. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in specific cells of the inner ear. In some aspects, the miRTS may be a target sequence for a miRNA that is expressed in inner ear hair cells, spiral ganglion cells, lateral supporting cells, basilar membrane cells, medial supporting cells, spiral limbus cells, or any combination thereof.

In some aspects, the miRTS may be a target sequence for a miRNA that is expressed in inner ear hair cells. In some aspects, the miRNA that is expressed in inner ear hair cells reduces, suppresses, inhibits, or eliminates expression of the polypeptide (e.g., a therapeutic polypeptide, a Connexin 26 polypeptide) in the inner ear hair cells. In some aspects, miRNAs that are expressed in inner ear hair cells are miR-194, miR-140, miR-18a, miR-99a, miR-30b, miR-15a, miR182, or miR-183. In some aspects, the miRNA that is expressed in inner ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1. In some aspects, the miRNA that is expressed in inner ear hair cells is miR-140. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 2. In some aspects, the miRNA-140 target sequence comprises the nucleic acid sequence of SEQ ID NO: 2. In some aspects, the miRNA that is expressed in inner ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3. In some aspects, the miRNA that is expressed in inner ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in inner ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in inner ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6. In some aspects, the miRNA that is expressed in inner ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in inner ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in spiral ganglion cells. In some aspects, the miRNA that is expressed in spiral ganglion cells reduces, suppresses, inhibits, or eliminates expression of the polypeptide (e.g., a therapeutic polypeptide, a Connexin 26 polypeptide) in the spiral ganglion cells. In some aspects, miRNAs that are expressed in the spiral ganglion cells are miR-194, miR-18a, miR-99a, miR-30b, miR-15a, miR182, or miR-183. In some aspects, the miRNA that is expressed in ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1. In some aspects, the miRNA that is expressed in ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in basilar membrane cells. In some aspects, the miRNA that is expressed in basilar membrane cells reduces, suppresses, inhibits, or eliminates expression of the polypeptide (e.g., a therapeutic polypeptide, a Connexin 26 polypeptide) in the basilar membrane cells. In some aspects, miRNAs that are expressed in basilar membrane cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in lateral supporting cells. In some aspects, the miRNA that is expressed in lateral supporting cells reduces, suppresses, inhibits, or eliminates expression of the polypeptide (e.g., a therapeutic polypeptide, a Connexin 26 polypeptide) in the lateral supporting cells. In some aspects, miRNAs that are expressed in lateral supporting cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in medial supporting cells. In some aspects, the miRNA that is expressed in medial supporting cells reduces, suppresses, inhibits, or eliminates expression of the polypeptide (e.g., a therapeutic polypeptide, a Connexin 26 polypeptide) in the medial supporting cells. In some aspects, miRNAs that are expressed in medial supporting cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in spiral limbus cells. In some aspects, the miRNA that is expressed in spiral limbus cells reduces, suppresses, inhibits, or eliminates expression of the polypeptide (e.g., a therapeutic polypeptide, a Connexin 26 polypeptide) in the spiral limbus cells. In some aspects, miRNAs that are expressed in spiral limbus cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, a non-endogenous regulatory region included in a UTR may comprise multiple miRNA regulatory target sites (miRTS). In some aspects, a UTR may comprise at least one miRNA-182 target site and at least one miRNA-183 target site. In some aspects, a non-endogenous regulatory region included in a UTR is a destabilizing domain, and is exemplified by SEQ ID NO: 80. In some aspects, a UTR may include a sequence that is at least 85%, at least 90%, at least 95%, at least 96%, at least 97%, at least 98% at least 99%, or 100% identical to a non-endogenous regulatory region exemplified by SEQ ID NO: 80.

miRNA-182 target sequence
(SEQ ID NO: 78)
AGTGTGAGTTCTACCATTGCCAAA
miRNA-183 target sequence
(SEQ ID NO: 79)
AGTGAATTCTACCAGTGCCATA
miRNA-194 target sequence
(SEQ ID NO: 1)
TCCACATGGAGTTGCTGTTACA
miRNA-140 target sequence
(SEQ ID NO: 2)
CCGTGGTTCTACCCTGTGGTA
miRNA-18a target sequence
(SEQ ID NO: 3)
CTATCTGCACTAGATGCACCTTA
miRNA-99a target sequence
(SEQ ID NO: 4)
CACAAGATCGGATCTACGGGTT
miRNA-30b target sequence
(SEQ ID NO: 5)
CTGAGTGTAGGATGTTTACA
miRNA-15a target sequence
(SEQ ID NO: 6)
CACAAACCATTATGTGCTGCTA
Exemplary mRNA destabilizing domain Sequence
(SEQ ID NO: 80)
GAGCTCAGTGTGAGTTCTACCATTGCCAAACTCGAGCAGTGAATTCTAC
CAGTGCCATAGGATCCAGTGTGAGTTCTACCATTGCCAAAGGTACCCAG
TGAATTCTACCAGTGCCATAGTTAAC

TABLE 3
Exemplary microRNA regulatory target sites

| microRNA regulatory target sites | SEQ ID NO |
|---|---|
| miRNA-182 target sequence | 78 |
| miRNA-183 target sequence | 79 |
| miRNA-194 target sequence | 1 |
| miRNA-140 target sequence | 2 |
| miRNA-18a target sequence | 3 |
| miRNA-99a target sequence | 4 |
| miRNA-30b target sequence | 5 |
| miRNA-15a target sequence | 6 |

### Internal Ribosome Entry Sites (IRES)

In some aspects, a construct encoding a polypeptide (e.g., a therapeutic polypeptide, a Connexin 26 polypeptide) can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001, each of which is incorporated in its entirety herein by reference.

In some aspects, the IRES sequence that is incorporated into a construct that encodes a polypeptide (e.g., a therapeutic polypeptide, a Connexin 26 polypeptide) is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11: 1921-1931, 2000; and Klump et al., Gene Therapy 8:811-817, 2001, each of which is incorporated in its entirety herein by reference).

### Splice Sites

In some aspects, any of the constructs provided herein can include splice donor and/or splice acceptor sequences, which are functional during RNA processing occurring during transcription. In some aspects, splice sites are involved in trans-splicing.

Exemplary splice donor intron
(SEQ ID NO: SEQ ID NO: 23)
GTAAGTATCAAGGTTACAAGACAGGTTTAAGGAGACCAATAGAAACTGG
GCTTGTCGAGACAGAGAAGACTCTTGCGTTTCT
Exemplary splice acceptor intron
(SEQ ID NO: SEQ ID NO: 24)
GATAGGCACCTATTGGTCTTACTGACATCCACTTTGCCTTTCTCTCCAC
AG

### Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some aspects, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. USA. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71 (2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some aspects, a poly(A) signal sequence can be a synthetic polyadenylation site (see, e.g., the pCl-neo expression construct of Promega that is based on Levitt el al., Genes Dev. 3(7):1019-1025, 1989, which is incorporated herein by reference in its entirety). In some aspects, a poly(A) signal sequence is the polyadenylation signal of soluble neuropilin-1 (sNRP) (AAATAAAATACGAAATG; SEQ ID NO: 89) (see, e.g., WO 05/073384, which is incorporated herein by reference in its entirety). In some aspects, a poly(A) signal sequence comprises or consists of the SV40 poly(A) site. In some aspects, a poly(A) signal comprises or consists of SEQ ID NO: 25. In some aspects, a poly(A) signal sequence comprises or consists of bGHpA. In some aspects, a poly(A) signal comprises or consists of SEQ ID NO: 26. Additional examples of poly(A) signal sequences are known in the art. In some aspects, a poly(A) sequence is at least 85%, at least 90%, at least 95%, at least 96%, at least 97%, at least 98% at least 99%, or 100% identical to the poly(A) sequence represented by SEQ ID NO: 25.

Exemplary bGH poly(A) signal sequence
(SEQ ID NO: 25)
CTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAAT
GAGGAAATTGCATCGCATTGTCTGAGTAGGTGTCATTCTATTCTGGGGG
GTGGGGTGGGGCAGGACAGCAAGGGGGAGGATTGGGAAGACAATAGCAG
GCATGCTGGGGATGCGGTGGGCTCTATGG
Exemplary SV40 poly(A) signal sequence
(SEQ ID NO: 26)
AACTTGTTTATTGCAGCTTATAATGGTTACAAATAAAGCAATAGCATCA
CAAATTTCACAAATAAAGCATTTTTTTCACTGCATTCTAGTTGTGGTTT
GTCCAAACTCATCAATGTATCTTA

### Additional Sequences

In some aspects, constructs of the present disclosure may include one or more filler sequences. In some aspects, filler sequences may function as regulatory elements, altering construct expression. In some such aspects, filler sequences may not be fully removed prior to manufacturing for administration to a subject. In some aspects, filler sequences may have functional roles including as linker sequences, as regulatory regions, or as stabilizing regions. As will be appreciated by those skilled in the art, filler sequences may vary significantly in primary sequence while retaining their desired function. In some aspects, constructs may contain any combination of filler sequences, exemplary filler sequences which may function as regulatory sequences are represented by SEQ ID NO: 128 or 129.

In some aspects, constructs of the present disclosure may comprise a T2A element or sequence. In some aspects, constructs of the present disclosure may include one or more cloning sites. In some such aspects, cloning sites may not be fully removed prior to manufacturing for administration to a subject. In some aspects, cloning sites may have functional roles including as linker sequences, portions of a Kozak site, or as sites encoding a stop codon. As will be appreciated by those skilled in the art, cloning sites may vary significantly in primary sequence while retaining their desired function. In some aspects, constructs may contain any combination of cloning sites, exemplary cloning sites are represented by SEQ ID NO: 29, 30, 31, 32, 33, 34, 35, 36, 37, or 85. In some aspects, constructs may contain additional cloning sites less than five nucleotides in length.

Exemplary Regulatory sequence C3
(SEQ ID NO: 128)
CTTCTTCTGGAGTCTTTTCTGGAATAATTCTGGGAGTGGGCTCAGCCTGCGGGAGAGTAACATTT
TTATAACTTGATAGATGTAGCTGAGATGCCTCCCAGAGGGGAGACCCGCCTCTCCTCCGGCAGCT
GTGCACGTAGGCTTGTTCCCAGCAGCCTGGCCAGGGTGGTCCACCTGGTGTTTCTCATCTTCTTT
CCCCGGAGCGCTGACTCCTGCGCGTCCTCTTGGAAGACTCTTGACAGGACGGGTGTTTTATGGGT
GTGATTCAGTGTCCTCTTGCATCAGTTCAATGTGGTGGTGTTCAATCAACCCTTGTAGCGTTAGC
AAAATTTGCTCAAGTCATTCCGCAGGAATGTCTGTGTCTTGCTTCCAAGAAAGCTTGTAAGTGCC
GGCAACAGGCCAAGCAGCTCACAAACCTGACCACAAGCCTGTGAGTAATTGTGGGGCAGCACTTA
GCAGTCTTTTATTTTCGACTTATTAAAGTCTCATCTTGGCCTCACCTTCTCCCTGGAAGGTGGCG
TGGGTGGGAACCACTGGGTCAGATCTTTTTCACCCTTGCCGTGGAGCCAGTTTCCTGTTGCATGT
GGGGGAAGCAACATGTGGTGAAGAGTATAGAAAACGAAAACATGTGGGTACAGTATGTATAAGTG
GAGGGAACAAACTCATAATTCCAACTAGTTTCTCATGAGAGACTCATGAATCATTGTGGTAGTTC
TCAATATAAACTTAATCTAGGCCGGATGTGGTGGCTCACACCTGTAATCTCAGCACTCTGGGTGG
ATCACTTGAGGTCAGGAGTTTGAGACCAGTCTGACCAACATGGAGAAACCCCATCGCTACTAAAA
ATACAAAATTATCCAGATGTGGTGGCTCACACCTGTAATCCCAGCACTTTGGGAGGCTGAGGCGG
GTGGATCACTTGAGGTCAGGAGTTTGAGACCAGCCTGACCAACATGGAGAAACTGTGTCTCTACT
AAAAATACAAAATTAGCTGGGCGTGGTGACGCATGCCTGTAATCCCAGCTATTTGGAGGCCGAAG
CAGG
Exemplary Regulatory sequence D7
(SEQ ID NO: 129)
CTTCTTCTGGAGTCTTTTCTGGAATAATTCTGGGAGTGGGCTCAGCCTGCGGGAGAGTAACATTT
TTATAACTTGATAGATGTAGCTGAGATGCCTCCCAGAGGGGAGACCCGCCTCTCCTCCGGCAGCT
GTGCACGTAGGCTTGTTCCCAGCAGCCTGGCCAGGGTGGTCCACCTGGTGTTTCTCATCTTCTTT
CCCCGGAGCGCTGACTCCTGCGCGTCCTCTTGGAAGACTCTTGACAGGACGGGTGTTTTATGGGT
GTGATTCAGTGTCCTCTTGCATCAGTTCAATGTGGTGGTGTTCAATCAACCCTTGTAGCGTTAGC
AAAATTTGCTCAAGTCATTCCGCAGGAATGTCTGTGTCTTGCTTCCAAGAAAGCTTGTAAGTGCC
GGCAACAGGCCAAGCAGCTCACAAACCTGACCACAAGCCTGTGAGTAATTGTGGGGCAGCACTTA
GCAGTCTTTTATTTTCGACTTATTAAAGTCTCATCTTGGCCTCACCTTCTCCCTGGAAGGTGGCG
TGGGTGGGAACCACTGGGTCAGATCTTTTTCACCCTTGCCGTGGAGCCAGTTTCCTGTTGCATGT
GGGGGAAGCAACATGTGGTGAAGAGTATAGAAAACGAAAACATGTGGGTACAGTATGTATAAGTG
GAGGGAACAAACTCATAATTCCAACTAGTTTCTCATGAGAGACTCATGAATCATTGTGGTAGTTC
TCAATATAAACTTAATCTAGGCCGGATGTGGTGGCTCACACCTGTAATCTCAGCACTCTGGGTGG
ATCACTTGAGGTCAGGAGTTTGAGACCAGTCTGACCAACATGGAGAAACCCCATCGCTACTAAAA
ATACAAAATTATCCAGATGTGGTGGCTCACACCTGTAATCCCAGCACTTTGGGAGGCTGAGGCGG
GTGGATCACTTGAGGTCAGGAGTTTGAGACCAGCCTGACCAACATGGAGAAACTGTGTCTCTACT
AAAAATACAAAATTAGCTGGGCGTGGTGACGCATGCCTGTAATCCCAGCTATTTGGAGGCCGAAG
CAGG
Exemplary cloning site A
(SEQ ID NO: 29)
TTGTCGACGCGGCCGCACGCGT
Exemplary cloning site B
(SEQ ID NO: 30)
CTCCTGGGCAACGTGCTGGTTATTGTGACCGGTGCCACC
Exemplary cloning site C
(SEQ ID NO: 31)
TAAGAGCTCGCTGATCAGCCTCGA
Exemplary cloning site D
(SEQ ID NO: 32)
AAGCTTGAATTCAGCTGACGTGCCTCGGACCGCCTAGG
Exemplary cloning site E
(SEQ ID NO: 33)
TAAGAGCTC
Exemplary cloning site F
(SEQ ID NO: 34)
GCTGATCAGCCTCGA
Exemplary cloning site G
(SEQ ID NO: 35)
GGCATTCCGGTACTGTTGGTAAAGCCACCAGCAAACCGCCCAGAGTAGAAGACCGGTGGCCACC
Exemplary cloning site H
(SEQ ID NO: 36)
AAGCTTGAATTC
Exemplary cloning site I
(SEQ ID NO: 37)
AGCTGACGTGCCTCGGACCGCCTAGG
Exemplary cloning site J
(SEQ ID NO: 70)
GCGGCCGCACGCGT
Exemplary cloning site K
(SEQ ID NO: 71)
GCGGCCGCACGCGTGGT
Exemplary cloning site L
(SEQ ID NO: 72)
CTCCTGGGCAACGTGCTGGTTATTGTGACCGGT
Exemplary cloning site M
(SEQ ID NO: 73)
CGCTAGCCACC
Exemplary cloning site N
(SEQ ID NO: 74)
ACCGGTCGCTAGCCACC
Exemplary cloning site O
(SEQ ID NO: 75)
GAGCTCGCTGATCAGCCTCGA
Exemplary cloning site P
(SEQ ID NO: 76)
AAGCTTGAATTCAGCTGACGTGCCTCGGACCGCT
Exemplary cloning site Q
(SEQ ID NO: 85)
CTCACCGGT
Exemplary linker sequence
(SEQ ID NO: 77)
GGATCCCGGGCT

### Reporter Sequences, Elements, or Reporter Polypeptides

In some aspects, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), FLAG, and a luciferase. Additional examples of reporter sequences are known in the art. Non-limiting examples of reporter polypeptides include a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), FLAG, and a luciferase. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some aspects, a reporter sequence is the LacZ gene, and the presence of a construct carrying the LacZ gene in a mammalian cell (e.g., a cochlear hair cell) is detected by assays for beta-galactosidase activity. When the reporter polypeptide is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some aspects, a reporter sequence can be used to verify the tissue-specific targeting capabilities and tissue-specific promoter regulatory and/or control activity of any of the constructs described herein. In some aspects, a reporter polypeptide can be used to verify the tissue-specific targeting capabilities and tissue-specific promoter regulatory and/or control activity of any of the constructs described herein.

In some aspects, a reporter sequence is a FLAG tag (e.g., a 3×FLAG tag), and the presence of a construct carrying the FLAG tag in a mammalian cell (e.g., an inner ear cell, e.g., a cochlear hair or supporting cell) is detected by protein binding or detection assays (e.g., Western blots, immunohistochemistry, radioimmunoassay (RIA), mass spectrometry). An exemplary 3×FLAG tag sequence is provided as SEQ ID NO: 42.

Exemplary 3xFLAG tag sequence
(SEQ ID NO: 42)
GGATCCCGGGCTGACTACAAAGACCATGACGGTGATTATAAAGATCATG
ACATCGACTACAAGGATGACGATGACAAG
Exemplary 3xFLAG tag sequence with stop codon
(SEQ ID NO: 81)
GACTACAAAGACCATGACGGTGATTATAAAGATCATGACATCGACTACA
AGGATGACGATGACAAGTAA
Exemplary barcode tag
(SEQ ID NO: 62)
GTGTCACC
Exemplary barcode tag
(SEQ ID NO: 55)
CACAACCT
Exemplary barcode tag
(SEQ ID NO: 27)
CGTGTGTT
Exemplary barcode tag
(SEQ ID NO: 41)
TCGTGGGT
Exemplary barcode tag
(SEQ ID NO: 39)
GCAAACTG
Exemplary barcode tag
(SEQ ID NO: 108)
CCTACGCT
Exemplary barcode tag
(SEQ ID NO: 109)
GCCAAAGC
Exemplary barcode tag
(SEQ ID NO: 110)
CCATCCAC
Exemplary barcode tag
(SEQ ID NO: 111)
CCCGTTCT
Exemplary barcode tag
(SEQ ID NO: 112)
TTCACTGG
Exemplary barcode tag
(SEQ ID NO: 113)
ATACTCTC
Exemplary barcode tag
(SEQ ID NO: 114)
GGCACTTC
Exemplary barcode tag
(SEQ ID NO: 115)
TTTCAGGT

### AAV Capsids

The present disclosure provides one or more polynucleotide constructs packaged into an AAV capsid. In some aspects, an AAV capsid is from or derived from an AAV capsid of an AAV2, 3, 4, 5, 6, 7, 8, 9, 10, rh8, rh10, rh39, rh43, AAV2-tYF, AAV2-P2V2, AAV2-P2V3, AAV2-MeBtYFTV, AAV2-MeB, AAV2-P2V6, AAV2-DGEDF, or Anc80 serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some aspects, an AAV capsid is an Anc80 capsid (e.g., an Anc80L65 capsid). In some aspects, an AAV capsid is created using a template nucleotide coding sequence comprising SEQ ID NO: 43. In some aspects, the capsid comprises a polypeptide represented by SEQ ID NO: 44. In some aspects, the capsid comprises a polypeptide with at least 85%, at least 90%, at least 95%, at least 96%, at least 97%, at least 98% at least 99%, or 100% identical to the polypeptide represented by SEQ ID NO: 44.

As provided herein, any combination of AAV capsids and AAV constructs (e.g., comprising AAV ITRs) may be used in recombinant AAV (rAAV) particles of the present disclosure. For example, wild-type or variant AAV2 ITRs and Anc80 capsid (e.g., an Anc80L65 capsid), wild-type or variant AAV2 ITRs and AAV6 capsid, etc. In some aspects of the present disclosure, an AAV particle is wholly comprised of AAV2 components (e.g., capsid and ITRs are AAV2 serotype). In some aspects, an AAV particle is an AAV2/6, AAV2/8 or AAV2/9 particle (e.g., an AAV6, AAV8 or AAV9 capsid with an AAV construct having AAV2 ITRs). In some aspects of the present disclosure, an AAV particle is an AAV2/Anc80 particle that comprises an Anc80 capsid (e.g., comprising a polypeptide of SEQ ID NO: 44) that encapsidates an AAV construct with AAV2 ITRs (e.g., SEQ ID NOs: 8 and 9) flanking a portion of a coding sequence, for example, a nucleic acid encoding a polypeptide (e.g., a therapeutic polypeptide, a Connexin 26 polypeptide). Other AAV particles are known in the art and are described in, e.g., Sharma et al., Brain Res Bull. 2010 Feb. 15; 81(2-3): 273, which is incorporated in its entirety herein by reference. In some aspects, a capsid sequence is at least 85%, at least 90%, at least 95%, at least 96%, at least 97%, at least 98% at least 99%, or 100% identical to a capsid nucleotide or amino acid sequence represented by SEQ ID NO: 43 or 44, respectively.

Exemplary AAV Anc80 Capsid DNA Sequence
(SEQ ID NO: 43)
ATGGCTGCCGATGGTTATCTTCCAGATTGGCTCGAGGACAACCTCTCTGAGGGCATTCGCGAGTG
GTGGGACTTGAAACCTGGAGCCCCGAAACCCAAAGCCAACCAGCAAAAGCAGGACGACGGCCGGG
GTCTGGTGCTTCCTGGCTACAAGTACCTCGGACCCTTCAACGGACTCGACAAGGGGGAGCCCGTC
AACGCGGCGGACGCAGCGGCCCTCGAGCACGACAAGGCCTACGACCAGCAGCTCAAAGCGGGTGA
CAATCCGTACCTGCGGTATAACCACGCCGACGCCGAGTTTCAGGAGCGTCTGCAAGAAGATACGT
CTTTTGGGGGCAACCTCGGGCGAGCAGTCTTCCAGGCCAAGAAGCGGGTTCTCGAACCTCTCGGT
CTGGTTGAGGAAGGCGCTAAGACGGCTCCTGGAAAGAAGAGACCGGTAGAGCAATCACCCCAGGA
ACCAGACTCCTCTTCGGGCATCGGCAAGAAAGGCCAGCAGCCCGCGAAGAAGAGACTCAACTTTG
GGCAGACAGGCGACTCAGAGTCAGTGCCCGACCCTCAACCACTCGGAGAACCCCCCGCAGCCCCC
TCTGGTGTGGGATCTAATACAATGGCAGCAGGCGGTGGCGCTCCAATGGCAGACAATAACGAAGG
CGCCGACGGAGTGGGTAACGCCTCAGGAAATTGGCATTGCGATTCCACATGGCTGGGCGACAGAG
TCATCACCACCAGCACCCGAACCTGGGCCCTCCCCACCTACAACAACCACCTCTACAAGCAAATC
TCCAGCCAATCGGGAGCAAGCACCAACGACAACACCTACTTCGGCTACAGCACCCCCTGGGGGTA
TTTTGACTTTAACAGATTCCACTGCCACTTCTCACCACGTGACTGGCAGCGACTCATCAACAACA
ACTGGGGATTCCGGCCCAAGAGACTCAACTTCAAGCTCTTCAACATCCAGGTCAAGGAGGTCACG
ACGAATGATGGCACCACGACCATCGCCAATAACCTTACCAGCACGGTTCAGGTCTTTACGGACTC
GGAATACCAGCTCCCGTACGTCCTCGGCTCTGCGCACCAGGGCTGCCTGCCTCCGTTCCCGGCGG
ACGTCTTCATGATTCCTCAGTACGGGTACCTGACTCTGAACAATGGCAGTCAGGCCGTGGGCCGT
TCCTCCTTCTACTGCCTGGAGTACTTTCCTTCTCAAATGCTGAGAACGGGCAACAACTTTGAGTT
CAGCTACACGTTTGAGGACGTGCCTTTTCACAGCAGCTACGCGCACAGCCAAAGCCTGGACCGGC
TGATGAACCCCCTCATCGACCAGTACCTGTACTACCTGTCTCGGACTCAGACCACGAGTGGTACC
GCAGGAAATCGGACGTTGCAATTTTCTCAGGCCGGGCCTAGTAGCATGGCGAATCAGGCCAAAAA
CTGGCTACCCGGGCCCTGCTACCGGCAGCAACGCGTCTCCAAGACAGCGAATCAAAATAACAACA
GCAACTTTGCCTGGACCGGTGCCACCAAGTATCATCTGAATGGCAGAGACTCTCTGGTAAATCCC
GGTCCCGCTATGGCAACCCACAAGGACGACGAAGACAAATTTTTTCCGATGAGCGGAGTCTTAAT
ATTTGGGAAACAGGGAGCTGGAAATAGCAACGTGGACCTTGACAACGTTATGATAACCAGTGAGG
AAGAAATTAAAACCACCAACCCAGTGGCCACAGAACAGTACGGCACGGTGGCCACTAACCTGCAA
TCGTCAAACACCGCTCCTGCTACAGGGACCGTCAACAGTCAAGGAGCCTTACCTGGCATGGTCTG
GCAGAACCGGGACGTGTACCTGCAGGGTCCTATCTGGGCCAAGATTCCTCACACGGACGGACACT
TTCATCCCTCGCCGCTGATGGGAGGCTTTGGACTGAAACACCCGCCTCCTCAGATCCTGATTAAG
AATACACCTGTTCCCGCGAATCCTCCAACTACCTTCAGTCCAGCTAAGTTTGCGTCGTTCATCAC
GCAGTACAGCACCGGACAGGTCAGCGTGGAAATTGAATGGGAGCTGCAGAAAGAAAACAGCAAAC
GCTGGAACCCAGAGATTCAATACACTTCCAACTACAACAAATCTACAAATGTGGACTTTGCTGTT
GACACAAATGGCGTTTATTCTGAGCCTCGCCCCATCGGCACCCGTTACCTCACCCGTAATCTG
Exemplary AAV Anc80 Capsid Amino Acid Sequence
(SEQ ID NO: 44)
MAADGYLPDWLEDNLSEGIREWWDLKPGAPKPKANQQKQDDGRGLVLPGYKYLGPFNGLDKGEPV
NAADAAALEHDKAYDQQLKAGDNPYLRYNHADAEFQERLQEDTSFGGNLGRAVFQAKKRVLEPLG
LVEEGAKTAPGKKRPVEQSPQEPDSSSGIGKKGQQPAKKRLNFGQTGDSESVPDPQPLGEPPAAP
SGVGSNTMAAGGGAPMADNNEGADGVGNASGNWHCDSTWLGDRVITTSTRTWALPTYNNHLYKQI
SSQSGASTNDNTYFGYSTPWGYFDFNRFHCHFSPRDWQRLINNNWGFRPKRLNFKLFNIQVKEVT
TNDGTTTIANNLTSTVQVFTDSEYQLPYVLGSAHQGCLPPFPADVFMIPQYGYLTLNNGSQAVGR
SSFYCLEYFPSQMLRTGNNFEFSYTFEDVPFHSSYAHSQSLDRLMNPLIDQYLYYLSRTQTTSGT
AGNRTLQFSQAGPSSMANQAKNWLPGPCYRQQRVSKTANQNNNSNFAWTGATKYHLNGRDSLVNP
GPAMATHKDDEDKFFPMSGVLIFGKQGAGNSNVDLDNVMITSEEEIKTTNPVATEQYGTVATNLQ
SSNTAPATGTVNSQGALPGMVWQNRDVYLQGPIWAKIPHTDGHFHPSPLMGGFGLKHPPPQILIK
NTPVPANPPTTFSPAKFASFITQYSTGQVSVEIEWELQKENSKRWNPEIQYTSNYNKSTNVDFAV
DTNGVYSEPRPIGTRYLTRNL

### Compositions

Among other things, the present disclosure provides compositions. In some aspects, a composition comprises a construct as described herein. In some aspects, a composition comprises one or more constructs as described herein. In some aspects, a composition comprises a plurality of constructs as described herein. In some aspects, when more than one construct is included in the composition, the constructs are each different.

In some aspects, a composition comprises an AAV particle as described herein. In some aspects, a composition comprises one or more AAV particles as described herein. In some aspects, a composition comprises a plurality of AAV particles. In come aspects, when more than one AAV particle is included in the composition, the AAV particles are each different.

In some aspects, a composition comprises a vector as described herein.

In some aspects, a composition comprises a cell.

In some aspects, a composition is or comprises a pharmaceutical composition. In some aspects, the pharmaceutic composition comprises a pharmaceutically acceptable carrier. In some aspects, a composition is or comprises a synthetic perilymph solution. In some aspects, a synthetic perilymph solution comprises 20-200 mM NaCl; 1-5 mM KCl; 0.1-10 mM CaCl2); 1-10 mM glucose; and 2-50 mM HEPES, with a pH between about 6 and about 9.

### Dosing and Volume of Administration

In some aspects, a composition disclosed herein, e.g., one or a plurality of AAV vectors disclosed herein, is administered as a single dose or as a plurality of doses.

In some aspects, a composition disclosed herein is administered as a single dose. In some aspects, a composition disclosed herein is administered as a plurality of doses, e.g., 2, 3, 4, 5, 6, 7, 8, 9 or 10 doses.

In some aspects, a composition disclosed herein (e.g., a composition comprising one or a plurality of rAAV constructs disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some aspects, a composition disclosed herein is administered at a volume of about 0.01 mL. In some aspects, a composition disclosed herein is administered at a volume of about 0.02 mL. In some aspects, a composition disclosed herein is administered at a volume of about 0.03 mL. In some aspects, a composition disclosed herein is administered at a volume of about 0.04 mL. In some aspects, a composition disclosed herein is administered at a volume of about 0.05 mL. In some aspects, a composition disclosed herein is administered at a volume of about 0.06 mL. In some aspects, a composition disclosed herein is administered at a volume of about 0.07 mL. In some aspects, a composition disclosed herein is administered at a volume of about 0.08 mL. In some aspects, a composition disclosed herein is administered at a volume of about 0.09 mL. In some aspects, a composition disclosed herein is administered at a volume of about 1.00 mL. In some aspects, a composition disclosed herein is administered at a volume of about 1.10 mL. In some aspects, a composition disclosed herein is administered at a volume of about 1.20 mL. In some aspects, a composition disclosed herein is administered at a volume of about 1.30 mL. In some aspects, a composition disclosed herein is administered at a volume of about 1.40 mL. In some aspects, a composition disclosed herein is administered at a volume of about 1.50 mL. In some aspects, a composition disclosed herein is administered at a volume of about 1.60 mL. In some aspects, a composition disclosed herein is administered at a volume of about 1.70 mL. In some aspects, a composition disclosed herein is administered at a volume of about 1.80 mL. In some aspects, a composition disclosed herein is administered at a volume of about 1.90 mL. In some aspects, a composition disclosed herein is administered at a volume of about 2.00 mL.

In some aspects, a composition disclosed herein (e.g., a composition comprising one or a plurality of rAAV constructs disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some aspects a composition disclosed herein (e.g., a composition comprising one or a plurality of rAAV constructs disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

In some aspects, a dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some aspects, a dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some aspects, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

### Single AAV Construct Compositions

In some aspects, the present disclosure provides compositions or systems comprising AAV particles comprised of a single construct. In some such aspects, a single construct may deliver a polynucleotide that encodes a functional (e.g., wild-type or otherwise functional, e.g., codon optimized) polypeptide (e.g., a therapeutic polypeptide, a Connexin 26 polypeptide). In some aspects, a construct is or comprises an rAAV construct. In some aspects described herein, a single rAAV construct is capable of expressing a polypeptide (e.g., a therapeutic polypeptide, a Connexin 26 polypeptide) thereof in a target cell (e.g., an inner ear supporting cell).

In some aspects, a single construct composition or system may comprise any or all of the exemplary construct components described herein. In some aspects, the, the construct comprises a nucleic acid sequence with at least 85%, at least 90%, at least 95%, at least 96%, at least 97%, at least 98% at least 99%, or 100% identity to SEQ ID NOs: 45-51, 82-84, 88, or 100-107. In some aspects, the, the construct comprises a nucleic acid sequence with at least 85%, at least 90%, at least 95%, at least 96%, at least 97%, at least 98% at least 99%, or 100% identity to SEQ ID NO: 82. In some aspects, an exemplary single construct is represented by SEQ ID NO: 82. In some aspects, the, the construct comprises a nucleic acid sequence with at least 85%, at least 90%, at least 95%, at least 96%, at least 97%, at least 98% at least 99%, or 100% identity to SEQ ID NO: 83. In some aspects, an exemplary single construct is represented by SEQ ID NO: 83. In some aspects, the, the construct comprises a nucleic acid sequence with at least 85%, at least 90%, at least 95%, at least 96%, at least 97%, at least 98% at least 99%, or 100% identity to SEQ ID NO: 84. In some aspects, an exemplary single construct is represented by SEQ ID NO: 84. In some aspects, the, the construct comprises a nucleic acid sequence with at least 85%, at least 90%, at least 95%, at least 96%, at least 97%, at least 98% at least 99%, or 100% identity to SEQ ID NO: 87. In some aspects, an exemplary single construct is represented by SEQ ID NO: 87.

In some aspects, the construct comprises the nucleic acid sequence of SEQ ID NO: 54. In some aspects, the, the construct comprises a nucleic acid sequence with at least 85%, at least 90%, at least 95%, at least 96%, at least 97%, at least 98% at least 99%, or 100% identity to SEQ ID NO: 54. In some aspects, the construct comprises the nucleic acid sequence of nucleotides 12-4754 of SEQ ID NO: 54. In some aspects, the, the construct comprises a nucleic acid sequence with at least 85%, at least 90%, at least 95%, at least 96%, at least 97%, at least 98% at least 99%, or 100% identity to 12-4754 of SEQ ID NO: 54.

In some aspects, the construct comprises the nucleic acid sequence of SEQ ID NO: 17. In some aspects, the, the construct comprises a nucleic acid sequence with at least 85%, at least 90%, at least 95%, at least 96%, at least 97%, at least 98% at least 99%, or 100% identity to SEQ ID NO: 17. In some aspects, the construct comprises nucleotides 12-4338 of SEQ ID NO: 17. In some aspects, the, the construct comprises a nucleic acid sequence with at least 85%, at least 90%, at least 95%, at least 96%, at least 97%, at least 98% at least 99%, or 100% identity to nucleotides 12-4338 of SEQ ID NO: 17.

In some aspects, the construct comprises the nucleic acid sequence of SEQ ID NO: 7. In some aspects, the, the construct comprises a nucleic acid sequence with at least 85%, at least 90%, at least 95%, at least 96%, at least 97%, at least 98% at least 99%, or 100% identity to SEQ ID NO: 7.

In some aspects, the construct comprises nucleotides 12-4557 of SEQ ID NO: 7. In some aspects, the, the construct comprises a nucleic acid sequence with at least 85%, at least 90%, at least 95%, at least 96%, at least 97%, at least 98% at least 99%, or 100% identity nucleotides 12-4557 of SEQ ID NO: 7.

In some aspects, the construct comprises a nucleic acid sequence with at least 85%, at least 90%, at least 95%, at least 96%, at least 97%, at least 98% at least 99%, or 100% identity to SEQ ID NO: 61. In some aspects, the construct comprises the nucleic acid sequence of SEQ ID NO: 61. In some aspects, the construct comprises a nucleic acid sequence with at least 85%, at least 90%, at least 95%, at least 96%, at least 97%, at least 98% at least 99%, or 100% identity to nucleotides 12-4429 of SEQ ID NO: 61. In some aspects, the construct comprises the nucleic acid sequence of nucleotides 12-4429 of SEQ ID NO: 61.

In some aspects, the construct comprises a nucleic acid sequence with at least 85%, at least 90%, at least 95%, at least 96%, at least 97%, at least 98% at least 99%, or 100% identity to SEQ ID NO: 38. In some aspects, the construct comprises the nucleic acid sequence of SEQ ID NO: 38. In some aspects, the construct comprises a nucleic acid sequence with at least 85%, at least 90%, at least 95%, at least 96%, at least 97%, at least 98% at least 99%, or 100% identity to nucleotides 12-3976 of SEQ ID NO: 38. In some aspects, the construct comprises the nucleic acid sequence of nucleotides 12-3976 of SEQ ID NO: 38.

In some aspects, the construct comprises a nucleic acid sequence with at least 85%, at least 90%, at least 95%, at least 96%, at least 97%, at least 98% at least 99%, or 100% identity to SEQ ID NO: 100. In some aspects, the construct comprises SEQ ID NO: 100. In some aspects, the construct comprises a nucleic acid sequence with at least 85%, at least 90%, at least 95%, at least 96%, at least 97%, at least 98% at least 99%, or 100% identity to nucleotides 12-4645 of SEQ ID NO: 100. In some aspects, the construct comprises nucleotides 12-4645 of SEQ ID NO: 100.

In some aspects, the construct comprises a nucleic acid sequence with at least 85%, at least 90%, at least 95%, at least 96%, at least 97%, at least 98% at least 99%, or 100% identity to SEQ ID NO: 101. In some aspects, the construct comprises SEQ ID NO: 101. In some aspects, the construct comprises a nucleic acid sequence with at least 85%, at least 90%, at least 95%, at least 96%, at least 97%, at least 98% at least 99%, or 100% identity to nucleotides 12-4708 of SEQ ID NO: 101. In some aspects, the construct comprises nucleotides 12-4708 SEQ ID NO: 101.

In some aspects, the construct comprises a nucleic acid sequence with at least 85%, at least 90%, at least 95%, at least 96%, at least 97%, at least 98% at least 99%, or 100% identity to SEQ ID NO: 102. In some aspects, the construct comprises SEQ ID NO: 102. In some aspects, the construct comprises a nucleic acid sequence with at least 85%, at least 90%, at least 95%, at least 96%, at least 97%, at least 98% at least 99%, or 100% identity to nucleotides 12-4993 of SEQ ID NO: 102. In some aspects, the construct comprises nucleotides 12-4993 of SEQ ID NO: 102.

In some aspects, the construct comprises a nucleic acid sequence with at least 85%, at least 90%, at least 95%, at least 96%, at least 97%, at least 98% at least 99%, or 100% identity to SEQ ID NO: 103. In some aspects, the construct comprises SEQ ID NO: 103. In some aspects, the construct comprises a nucleic acid sequence with at least 85%, at least 90%, at least 95%, at least 96%, at least 97%, at least 98% at least 99%, or 100% identity to nucleotides 12-4496 of SEQ ID NO: 103. In some aspects, the construct comprises nucleotides 12-4496 of SEQ ID NO: 103.

In some aspects, the construct comprises a nucleic acid sequence with at least 85%, at least 90%, at least 95%, at least 96%, at least 97%, at least 98% at least 99%, or 100% identity to SEQ ID NO: 104 In some aspects, the construct comprises SEQ ID NO: 104. In some aspects, the construct comprises a nucleic acid sequence with at least 85%, at least 90%, at least 95%, at least 96%, at least 97%, at least 98% at least 99%, or 100% identity to nucleotides 12-4253 of SEQ ID NO: 104 In some aspects, the construct comprises nucleotides 12-4253 of SEQ ID NO: 104.

In some aspects, the construct comprises a nucleic acid sequence with at least 85%, at least 90%, at least 95%, at least 96%, at least 97%, at least 98% at least 99%, or 100% identity to SEQ ID NO: 105. In some aspects, the construct comprises SEQ ID NO: 105. In some aspects, the construct comprises a nucleic acid sequence with at least 85%, at least 90%, at least 95%, at least 96%, at least 97%, at least 98% at least 99%, or 100% identity to nucleotides 12-4320 of SEQ ID NO: 105. In some aspects, the construct comprises nucleotides 12-4320 SEQ ID NO: 105.

In some aspects, the construct comprises a nucleic acid sequence with at least 85%, at least 90%, at least 95%, at least 96%, at least 97%, at least 98% at least 99%, or 100% identity to SEQ ID NO: 106. In some aspects, the construct comprises SEQ ID NO: 106. In some aspects, the construct comprises a nucleic acid sequence with at least 85%, at least 90%, at least 95%, at least 96%, at least 97%, at least 98% at least 99%, or 100% identity to nucleotides 12-4464 of SEQ ID NO: 106. In some aspects, the construct comprises nucleotides 12-4464 of SEQ ID NO: 106.

In some aspects, the construct comprises a nucleic acid sequence with at least 85%, at least 90%, at least 95%, at least 96%, at least 97%, at least 98% at least 99%, or 100% identity to SEQ ID NO: 107 In some aspects, the construct comprises SEQ ID NO: 107. In some aspects, the construct comprises a nucleic acid sequence with at least 85%, at least 90%, at least 95%, at least 96%, at least 97%, at least 98% at least 99%, or 100% identity to nucleotides 12-4328 of SEQ ID NO: 107 In some aspects, the construct comprises nucleotides 12-4328 of SEQ ID NO: 107.

One skilled in the art would recognize that constructs may undergo additional modifications including codon-optimization, introduction of novel but functionally equivalent (e.g., silent mutations), addition of reporter sequences, and/or other routine modification.

In some aspects, an exemplary rAAVAnc80 particle comprises a construct represented by SEQ ID NO: 82.

In one aspect, an exemplary construct comprises: a 5′ ITR exemplified by SEQ ID NO: 52, optionally a cloning site exemplified by SEQ ID NO: 70, a CAG enhancer/promoter exemplified by SEQ ID NO: 14, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 83.

In one aspect, an exemplary construct comprises: a 5′ ITR exemplified by SEQ ID NO: 52, optionally a cloning site exemplified by SEQ ID NO: 70, a CMV/CBA enhancer/promoter exemplified by SEQ ID NO: 12, a chimeric intron exemplified by SEQ ID NO: 64, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 84.

In one aspect, an exemplary construct comprises: a 5′ ITR exemplified by SEQ ID NO: 52, optionally a cloning site exemplified by SEQ ID NO: 70, a CMV enhancer exemplified by SEQ ID NO: 63, a human GJB2 promoter exemplified by SEQ ID NO: 61, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 87.

In one aspect, an exemplary construct comprises: a 5′ ITR exemplified by SEQ ID NO: 52, optionally a cloning site exemplified by SEQ ID NO: 70, a human GFAP enhancer-promoter exemplified by SEQ ID NO: 91, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a destabilization domain exemplified by SEQ ID NO: 80, a 3′ UTR exemplified by SEQ ID NO: 68, optionally a cloning site exemplified by SEQ ID NO: 34, a poly(A) site exemplified by SEQ ID NO: 25, optionally a cloning site exemplified by SEQ ID NO: 76, and a 3′ ITR exemplified by SEQ ID NO: 53.

In some aspects, the rAAVAnc80 particle comprises a construct comprising the nucleic acid sequence of SEQ ID NO: 61.

In one aspect, the construct comprises a 5′ ITR comprising the nucleic acid sequence of SEQ ID NO: 52, optionally a cloning site comprising the nucleic acid sequence of SEQ ID NO: 71, a GDF6 promoter sequence comprising the nucleic acid sequence of SEQ ID NO: 90; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 62; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 54.

In one aspect, the construct comprises a 5′ ITR comprising the nucleic acid sequence of SEQ ID NO: 52, optionally a cloning site comprising the nucleic acid sequence of SEQ ID NO: 71, a IGFBP2 promoter sequence comprising the nucleic acid sequence of SEQ ID NO: 57; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 55; a 5′UTR sequence comprising the nucleic acid sequence of SEQ ID NO: 66, a GJB2 coding region comp, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 17.

In one aspect, the construct comprises a 5′ ITR comprising the nucleic acid sequence of SEQ ID NO: 52, optionally a cloning site comprising the nucleic acid sequence of SEQ ID NO: 71, a RBP7 promoter sequence comprising the nucleic acid sequence of SEQ ID NO: 28; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 27; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 17.

In one aspect, the construct comprises a 5′ ITR comprising the nucleic acid sequence of SEQ ID NO: 52, optionally a cloning site comprising the nucleic acid sequence of SEQ ID NO: 71, a GJB6 promoter sequence comprising the nucleic acid sequence of SEQ ID NO: 16; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 41; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 7.

In one aspect, the construct comprises a 5′ ITR comprising the nucleic acid sequence of SEQ ID NO: 52, optionally a cloning site comprising the nucleic acid sequence of SEQ ID NO: 71, a PARM1 promoter sequence comprising the nucleic acid sequence of SEQ ID NO: 40; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 39; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 100.

In one aspect, the construct comprises a 5′ ITR comprising the nucleic acid sequence of SEQ ID NO: 52, optionally a cloning site comprising the nucleic acid sequence of SEQ ID NO: 71, a BACE2 promoter sequence comprising the nucleic acid sequence of SEQ ID NO: 92; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 108; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 101.

In one aspect, the construct comprises a 5′ ITR comprising the nucleic acid sequence of SEQ ID NO: 52, optionally a cloning site comprising the nucleic acid sequence of SEQ ID NO: 71, a DBI2 promoter sequence comprising the nucleic acid sequence of SEQ ID NO: 93; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 109; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 102.

In one aspect, the construct comprises a 5′ ITR comprising the nucleic acid sequence of SEQ ID NO: 52, optionally a cloning site comprising the nucleic acid sequence of SEQ ID NO: 71, a FABP3 promoter sequence comprising the nucleic acid sequence of SEQ ID NO: 94; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 110; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 103.

In one aspect, the construct comprises a 5′ ITR comprising the nucleic acid sequence of SEQ ID NO: 52, optionally a cloning site comprising the nucleic acid sequence of SEQ ID NO: 71, a KLHL14 promoter sequence comprising the nucleic acid sequence of SEQ ID NO: 95; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 111; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 104.

In one aspect, the construct comprises a 5′ ITR comprising the nucleic acid sequence of SEQ ID NO: 52, optionally a cloning site comprising the nucleic acid sequence of SEQ ID NO: 71, a MMP15 promoter sequence comprising the nucleic acid sequence of SEQ ID NO: 96; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 112; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 105.

In one aspect, the construct comprises a 5′ ITR comprising the nucleic acid sequence of SEQ ID NO: 52, optionally a cloning site comprising the nucleic acid sequence of SEQ ID NO: 71, a SPARC promoter sequence comprising the nucleic acid sequence of SEQ ID NO: 97; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 113; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 106.

In one aspect, the construct comprises a 5′ ITR comprising the nucleic acid sequence of SEQ ID NO: 52, optionally a cloning site comprising the nucleic acid sequence of SEQ ID NO: 71, a TSPAN8 promoter sequence comprising the nucleic acid sequence of SEQ ID NO: 98; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 124; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 107.

In one aspect, the construct comprises a 5′ ITR comprising the nucleic acid sequence of SEQ ID NO: 52, optionally a cloning site comprising the nucleic acid sequence of SEQ ID NO: 71, a VIM promoter sequence comprising the nucleic acid sequence of SEQ ID NO: 99; a hGJB2 minimal promoter comprising the nucleic acid sequence of SEQ ID NO: 91, optionally a cloning site comprising the nucleic acid sequence of SEQ ID NO: 85, optionally a synthetic barcode comprising the nucleic acid sequence of SEQ ID NO: 114; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

Exemplary Construct sequence
(SEQ ID NO: 82)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGGGGGGGGGGGGGGGGCGCGCGCCAGGCGGG
GCGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTCGTTTCTTTTCTGTGGCTGCGTGAAAGCCTTA
AAGGGCTCCGGGAGGGCCCTTTGTGCGGGGGGGAGCGGCTCGGGGGGTGCGTGCGTGTGTGTGTG
CGTGGGGAGCGCCGCGTGCGGCCCGCGCTGCCCGGCGGCTGTGAGCGCTGCGGGCGCGGCGCGGG
GCTTTGTGCGCTCCGCGTGTGCGCGAGGGGAGCGCGGCCGGGGGCGGTGCCCCGCGGTGCGGGGG
GGCTGCGAGGGGAACAAAGGCTGCGTGCGGGGTGTGTGCGTGGGGGGGTGAGCAGGGGGTGTGGG
CGCGGCGGTCGGGCTGTAACCCCCCCCTGCACCCCCCTCCCCGAGTTGCTGAGCACGGCCCGGCT
TCGGGTGCGGGGCTCCGTGCGGGGCGTGGCGCGGGGCTCGCCGTGCCGGGCGGGGGGTGGCGGCA
GGTGGGGGTGCCGGGGGGGGGGGGGCCGCCTCGGGCCGGGGAGGGCTCGGGGGAGGGGCGCGGCG
GCCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCCG
CCGCACCCCCTCTAGCGGGCGCGGGGCGAAGCGGTGCGGCGCCGGCAGGAAGGAAATGGGGGGGG
AGGGCCTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGGGGGGTTCGGCTTCTGGCGTGTGACCGGCGG
CTCTAGAGCCTCTGCTAACCATGTTCATGCCTTCTTCTTTTTCCTACAGCTCCTGGGCAACGTGC
TGGTTATTGTGACCGGTGTTGCGGCCCCGCAGCGCCCGCGCGCTCCTCTCCCCGACTCGGAGCCC
CTCGGCGGCGCCCGGCCCAGGACCCGCCTAGGAGCGCAGGAGCCCCAGCGCAGAGACCCCAACGC
CGAGACCCCCGCCCCGGCCCCGCCGCGCTTCCTCCCGACGCAGTTTAGGACCCTTGTTCGCGAAG
AGGTGGTGTGCGGCTGAGACCCGCGTCCTCAGGACGGTTCCATCAGTGCCTCGATCCTGCCCCAC
TGGAGGAGGAAGGCAGCCCGAACAGCGCTCACCTAACTAACAGCTGCTGAGAGCTGGGTTCCGTG
GCCATGCACCTGGGACTGCCTTGAGAAGCGTGAGCAAACCGCCCAGAGTAGAAGCGCTAGCCACC
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTTGGATCCCGGGCTGACTACAAAGACCATGACGGTGATT
ATAAAGATCATGACATCGACTACAAGGATGACGATGACAAGTAAGAAATAGACAGCATGAGAGGG
ATGAGGCAACCCGTGCTCAGCTGTCAAGGCTCAGTCGCTAGCATTTCCCAACACAAAGATTCTGA
CCTTAAATGCAACCATTTGAAACCCCTGTAGGCCTCAGGTGAAACTCCAGATGCCACAATGGAGC
TCTGCTCCCCTAAAGCCTCAAAACAAAGGCCTAATTCTATGCCTGTCTTAATTTTCTTTCACTTA
AGTTAGTTCCACTGAGACCCCAGGCTGTTAGGGGTTATTGGTGTAAGGTACTTTCATATTTTAAA
CAGAGGATATCGGCATTTGTTTCTTTCTCTGAGGACAAGAGAAAAAAGCCAGGTTCCACAGAGGA
CACAGAGAAGGTTTGGGTGTCCTCCTGGGGTTCTTTTTGCCAACTTTCCCCACGTTAAAGGTGAA
CATTGGTTCTTTCATTTGCTTTGGAAGTTTTAATCTCTAACAGTGGACAAAGTTACCAGTGCCTT
AAACTCTGTTACACTTTTTGGAAGTGAAAACTTTGTAGTATGATAGGTTATTTTGATGTAAAGAT
GTTCTGGATACCATTATATGTTCCCCCTGTTTCAGAGGCTCAGATTGTAATATGTAAATGGTATG
TCATTCGCTACTATGATTTAATTTGAAATATGGTCTTTTGGTTATGAATACTTTGCAGCACAGCT
GAGAGGCTGTCTGTTGTATTCATTGTGGTCATAGCACCTAACAACATTGTAGCCTCAATCGAGTG
AGACAGACTAGAAGTTCCTAGTGATGGCTTATGATAGCAAATGGCCTCATGTCAAATATTTAGAT
GTAATTTTGTGTAAGAAATACAGACTGGATGTACCACCAACTACTACCTGTAATGACAGGCCTGT
CCAACACATCTCCCTTTTCCATGACTGTGGTAGCCAGCATCGGAAAGAACGCTGATTTAAAGAGG
TCGCTTGGGAATTTTATTGACACAGTACCATTTAATGGGGAGGACAAAATGGGGCAGGGGAGGGA
GAAGTTTCTGTCGTTAAAAACAGATTTGGAAAGACTGGACTCTAAAGTCTGTTGATTAAAGATGA
GCTTTGTCTACTTCAAAAGTTTGTTTGCTTACCCCTTCAGCCTCCAATTTTTTAAGTGAAAATAT
AGCTAATAACATGTGAAAAGAATAGAAGCTAAGGTTTAGATAAATATTGAGCAGATCTATAGGAA
GATTGAACCTGAATATTGCCATTATGCTTGACATGGTTTCCAAAAAATGGTACTCCACATATTTC
AGTGAGGGTAAGTATTTTCCTGTTGTCAAGAATAGCATTGTAAAAGCATTTTGTAATAATAAAGA
ATAGCTTTAATGATATGCTTGTAACTAAAATAATTTTGTAATGTATCAAATACATTTAAAACATT
AAAATATAATCTCTATAATAAGAGCTCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGGGATGCGGTGGGC
TCTATGGAAGCTTGAATTCAGCTGACGTGCCTCGGACCGCTAGGAACCCCTAGTGATGGAGTTGG
CCACTCCCTCTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAG
Exemplary Construct sequence
(SEQ ID NO: 83)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGGGGGGGGGGGGGGGGCGCGCGCCAGGCGGG
GCGGGGCGGGGCGAGGGGGGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTTGTTTCTTTTCTGTGGCTGCGTGAAAGCCTTG
AGGGGCTCCGGGAGCTAGAGCCTCTGCTAACCATGTTCATGCCTTCTTCTTTTTCCTACAGCTCC
TGGGCAACGTGCTGGTTATTGTGACCGGTGTTGCGGCCCCGCAGCGCCCGCGCGCTCCTCTCCCC
GACTCGGAGCCCCTCGGCGGCGCCCGGCCCAGGACCCGCCTAGGAGCGCAGGAGCCCCAGCGCAG
AGACCCCAACGCCGAGACCCCCGCCCCGGCCCCGCCGCGCTTCCTCCCGACGCAGTTTAGGACCC
TTGTTCGCGAAGAGGTGGTGTGCGGCTGAGACCCGCGTCCTCAGGACGGTTCCATCAGTGCCTCG
ATCCTGCCCCACTGGAGGAGGAAGGCAGCCCGAACAGCGCTCACCTAACTAACAGCTGCTGAGAG
CTGGGTTCCGTGGCCATGCACCTGGGACTGCCTTGAGAAGCGTGAGCAAACCGCCCAGAGTAGAA
GCGCTAGCCACCATGGATTGGGGCACGCTGCAGACGATCCTGGGGGGTGTGAACAAACACTCCAC
CAGCATTGGAAAGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCAGCCAGGCTGCAAG
AACGTGTGCTACGATCACTACTTCCCCATCTCCCACATCCGGCTATGGGCCCTGCAGCTGATCTT
CGTGTCCACGCCAGCGCTCCTAGTGGCCATGCACGTGGCCTACCGGAGACATGAGAAGAAGAGGA
AGTTCATCAAGGGGGAGATAAAGAGTGAATTTAAGGACATCGAGGAGATCAAAACCCAGAAGGTC
CGCATCGAAGGCTCCCTGTGGTGGACCTACACAAGCAGCATCTTCTTCCGGGTCATCTTCGAAGC
CGCCTTCATGTACGTCTTCTATGTCATGTACGACGGCTTCTCCATGCAGCGGCTGGTGAAGTGCA
ACGCCTGGCCTTGTCCCAACACTGTGGACTGCTTTGTGTCCCGGCCCACGGAGAAGACTGTCTTC
ACAGTGTTCATGATTGCAGTGTCTGGAATTTGCATCCTGCTGAATGTCACTGAATTGTGTTATTT
GCTAATTAGATATTGTTCTGGGAAGTCAAAAAAGCCAGTTGGATCCCGGGCTGACTACAAAGACC
ATGACGGTGATTATAAAGATCATGACATCGACTACAAGGATGACGATGACAAGTAAGAAATAGAC
AGCATGAGAGGGATGAGGCAACCCGTGCTCAGCTGTCAAGGCTCAGTCGCTAGCATTTCCCAACA
CAAAGATTCTGACCTTAAATGCAACCATTTGAAACCCCTGTAGGCCTCAGGTGAAACTCCAGATG
CCACAATGGAGCTCTGCTCCCCTAAAGCCTCAAAACAAAGGCCTAATTCTATGCCTGTCTTAATT
TTCTTTCACTTAAGTTAGTTCCACTGAGACCCCAGGCTGTTAGGGGTTATTGGTGTAAGGTACTT
TCATATTTTAAACAGAGGATATCGGCATTTGTTTCTTTCTCTGAGGACAAGAGAAAAAAGCCAGG
TTCCACAGAGGACACAGAGAAGGTTTGGGTGTCCTCCTGGGGTTCTTTTTGCCAACTTTCCCCAC
GTTAAAGGTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAGGTTATTT
TGATGTAAAGATGTTCTGGATACCATTATATGTTCCCCCTGTTTCAGAGGCTCAGATTGTAATAT
GTAAATGGTATGTCATTCGCTACTATGATTTAATTTGAAATATGGTCTTTTGGTTATGAATACTT
TGCAGCACAGCTGAGAGGCTGTCTGTTGTATTCATTGTGGTCATAGCACCTAACAACATTGTAGC
CTCAATCGAGTGAGACAGACTAGAAGTTCCTAGTGATGGCTTATGATAGCAAATGGCCTCATGTC
AAATATTTAGATGTAATTTTGTGTAAGAAATACAGACTGGATGTACCACCAACTACTACCTGTAA
TGACAGGCCTGTCCAACACATCTCCCTTTTCCATGACTGTGGTAGCCAGCATCGGAAAGAACGCT
GATTTAAAGAGGTCGCTTGGGAATTTTATTGACACAGTACCATTTAATGGGGAGGACAAAATGGG
GCAGGGGAGGGAGAAGTTTCTGTCGTTAAAAACAGATTTGGAAAGACTGGACTCTAAAGTCTGTT
GATTAAAGATGAGCTTTGTCTACTTCAAAAGTTTGTTTGCTTACCCCTTCAGCCTCCAATTTTTT
AAGTGAAAATATAGCTAATAACATGTGAAAAGAATAGAAGCTAAGGTTTAGATAAATATTGAGCA
GATCTATAGGAAGATTGAACCTGAATATTGCCATTATGCTTGACATGGTTTCCAAAAAATGGTAC
TCCACATATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTATCAAATAC
ATTTAAAACATTAAAATATAATCTCTATAATAAGAGCTCGCTGATCAGCCTCGACTGTGCCTTCT
AGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAGGCATGCTGGG
GATGCGGTGGGCTCTATGGAAGCTTGAATTCAGCTGACGTGCCTCGGACCGCTAGGAACCCCTAG
TGATGGAGTTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAG
Exemplary Construct sequence
(SEQ ID NO: 84)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTAAGC
TTCCGCAGAATCCTATCAGTTTCCCCCTTTCGTGCTGTGTGCATCGAGCAGGAAGGGGCTTGGCA
GGTTTTACCTGCCCTCTTTCCTTTCTGAAAAGTCTGGGCCTCCTCACCCCGAAAGGAGTCACCTC
CTTGCAGTTCCCCAGTTGCGAAAAGAGGAGGAAGTTGGCTGGGCCGGGGGCCGCGGGGGGCACCC
TCCGCAGATGGCGGGACCCCCCTGCCGGCCATGGCAAAAACGAGGCTTGTCTCTCCCACCGCCCC
CAACCTTAGTCCTTGGCACATTGTTGAAAGTAATTGAATAAAATCGGAAATTCGAGAAGGCGTTC
GTTCGGATTGGTGAGATTTTGAGGGGAGAAAGAAGCGGGGACTTCGCCGGCACCAGCGGCGCCCC
CTCCTCGGCCACCGTTAACCCCCATTCCAGAGGGCACTGCCCCGCCACCCAGCCTAGGTCCCCCT
GCGAGAGCCTCGCGGGCCCGCGCAGCCTCCGCGACTCGAACAGATCTTCAGTCCTTGGAGGAATG
CCTGTTTCTCTAACAATAAAAAATTAAAGAAGCGCTCATAAATGCCAAGTCCTCTCGCACTATGC
GGAGTACAGAGGACAACGACCACAGCCATCCCTGAACCCCGCCCACGGCACAGCGCCGGAGCCGG
GGTCTGGGGCGCCGCTTCCTGGGGGGTCCCGACTCTCAGCCGCCCCCGCTTCACCCGGGCCGCCA
AGGGGCTGGGGGAGGCGGCGCTCGGGGTAACCGGGGGAGACTCAGGGCGCTGGGGGCACTTGGGG
AACTCATGGGGGCTCAAAGGAACTAGGAGATCGGGACCTCGAAGGGGACTTGGGGGGTTCGGGGC
TTTCGGGGGCGGTCGGGGGTTCGCGGACCCGGGAAGCTCTGAGGACCCAGAGGCCGGGCGCGCTC
CGCCCGCGGCGCCGCCCCCTCCGTAACTTTCCCAGTCTCCGAGGGAAGAGGCGGGGTGTGGGGTG
CGGTTAAAAGGCGCCACGGCGGGAGACAGGTCTCCTGGGCAACGTGCTGGTTATTGTGACCGGTG
TTGCGGCCCCGCAGCGCCCGCGCGCTCCTCTCCCCGACTCGGAGCCCCTCGGCGGCGCCCGGCCC
AGGACCCGCCTAGGAGCGCAGGAGCCCCAGCGCAGAGACCCCAACGCCGAGACCCCCGCCCCGGC
CCCGCCGCGCTTCCTCCCGACGCAGTTTAGGACCCTTGTTCGCGAAGAGGTGGTGTGCGGCTGAG
ACCCGCGTCCTCAGGACGGTTCCATCAGTGCCTCGATCCTGCCCCACTGGAGGAGGAAGGCAGCC
CGAACAGCGCTCACCTAACTAACAGCTGCTGAGAGCTGGGTTCCGTGGCCATGCACCTGGGACTG
CCTTGAGAAGCGTGAGCAAACCGCCCAGAGTAGAAGCGCTAGCCACCATGGATTGGGGCACGCTG
CAGACGATCCTGGGGGGTGTGAACAAACACTCCACCAGCATTGGAAAGATCTGGCTCACCGTCCT
CTTCATTTTTCGCATTATGATCCTCGTTGTGGCTGCAAAGGAGGTGTGGGGAGATGAGCAGGCCG
ACTTTGTCTGCAACACCCTGCAGCCAGGCTGCAAGAACGTGTGCTACGATCACTACTTCCCCATC
TCCCACATCCGGCTATGGGCCCTGCAGCTGATCTTCGTGTCCACGCCAGCGCTCCTAGTGGCCAT
GCACGTGGCCTACCGGAGACATGAGAAGAAGAGGAAGTTCATCAAGGGGGAGATAAAGAGTGAAT
TTAAGGACATCGAGGAGATCAAAACCCAGAAGGTCCGCATCGAAGGCTCCCTGTGGTGGACCTAC
ACAAGCAGCATCTTCTTCCGGGTCATCTTCGAAGCCGCCTTCATGTACGTCTTCTATGTCATGTA
CGACGGCTTCTCCATGCAGCGGCTGGTGAAGTGCAACGCCTGGCCTTGTCCCAACACTGTGGACT
GCTTTGTGTCCCGGCCCACGGAGAAGACTGTCTTCACAGTGTTCATGATTGCAGTGTCTGGAATT
TGCATCCTGCTGAATGTCACTGAATTGTGTTATTTGCTAATTAGATATTGTTCTGGGAAGTCAAA
AAAGCCAGTTGGATCCCGGGCTGACTACAAAGACCATGACGGTGATTATAAAGATCATGACATCG
ACTACAAGGATGACGATGACAAGTAAGAAATAGACAGCATGAGAGGGATGAGGCAACCCGTGCTC
AGCTGTCAAGGCTCAGTCGCTAGCATTTCCCAACACAAAGATTCTGACCTTAAATGCAACCATTT
GAAACCCCTGTAGGCCTCAGGTGAAACTCCAGATGCCACAATGGAGCTCTGCTCCCCTAAAGCCT
CAAAACAAAGGCCTAATTCTATGCCTGTCTTAATTTTCTTTCACTTAAGTTAGTTCCACTGAGAC
CCCAGGCTGTTAGGGGTTATTGGTGTAAGGTACTTTCATATTTTAAACAGAGGATATCGGCATTT
GTTTCTTTCTCTGAGGACAAGAGAAAAAAGCCAGGTTCCACAGAGGACACAGAGAAGGTTTGGGT
GTCCTCCTGGGGTTCTTTTTGCCAACTTTCCCCACGTTAAAGGTGAACATTGGTTCTTTCATTTG
CTTTGGAAGTTTTAATCTCTAACAGTGGACAAAGTTACCAGTGCCTTAAACTCTGTTACACTTTT
TGGAAGTGAAAACTTTGTAGTATGATAGGTTATTTTGATGTAAAGATGTTCTGGATACCATTATA
TGTTCCCCCTGTTTCAGAGGCTCAGATTGTAATATGTAAATGGTATGTCATTCGCTACTATGATT
TAATTTGAAATATGGTCTTTTGGTTATGAATACTTTGCAGCACAGCTGAGAGGCTGTCTGTTGTA
TTCATTGTGGTCATAGCACCTAACAACATTGTAGCCTCAATCGAGTGAGACAGACTAGAAGTTCC
TAGTGATGGCTTATGATAGCAAATGGCCTCATGTCAAATATTTAGATGTAATTTTGTGTAAGAAA
TACAGACTGGATGTACCACCAACTACTACCTGTAATGACAGGCCTGTCCAACACATCTCCCTTTT
CCATGACTGTGGTAGCCAGCATCGGAAAGAACGCTGATTTAAAGAGGTCGCTTGGGAATTTTATT
GACACAGTACCATTTAATGGGGAGGACAAAATGGGGCAGGGGAGGGAGAAGTTTCTGTCGTTAAA
AACAGATTTGGAAAGACTGGACTCTAAAGTCTGTTGATTAAAGATGAGCTTTGTCTACTTCAAAA
GTTTGTTTGCTTACCCCTTCAGCCTCCAATTTTTTAAGTGAAAATATAGCTAATAACATGTGAAA
AGAATAGAAGCTAAGGTTTAGATAAATATTGAGCAGATCTATAGGAAGATTGAACCTGAATATTG
CCATTATGCTTGACATGGTTTCCAAAAAATGGTACTCCACATATTTCAGTGAGGGTAAGTATTTT
CCTGTTGTCAAGAATAGCATTGTAAAAGCATTTTGTAATAATAAAGAATAGCTTTAATGATATGC
TTGTAACTAAAATAATTTTGTAATGTATCAAATACATTTAAAACATTAAAATATAATCTCTATAA
TAAGAGCTCGCTGATCAGCCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGAAGCTTGAATT
CAGCTGACGTGCCTCGGACCGCTAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCC
TCAGTGAGCGAGCGAGCGCGCAG
Exemplary Construct sequence
(SEQ ID NO: 87)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGGGGGGGGGGGGGGGGCGCGCGCCAGGCGGG
GCGGGGCGGGGCGAGGGGGGGGGGGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTCGTTTCTTTTCTGTGGCTGCGTGAAAGCCTTA
AAGGGCTCCGGGAGGGCCCTTTGTGCGGGGGGGAGCGGCTCGGGGGGTGCGTGCGTGTGTGTGTG
CGTGGGGAGCGCCGCGTGCGGCCCGCGCTGCCCGGCGGCTGTGAGCGCTGCGGGCGCGGCGCGGG
GCTTTGTGCGCTCCGCGTGTGCGCGAGGGGAGCGCGGCCGGGGGGGGTGCCCCGCGGTGCGGGGG
GGCTGCGAGGGGAACAAAGGCTGCGTGCGGGGTGTGTGCGTGGGGGGGTGAGCAGGGGGTGTGGG
CGCGGCGGTCGGGCTGTAACCCCCCCCTGCACCCCCCTCCCCGAGTTGCTGAGCACGGCCCGGCT
TCGGGTGCGGGGCTCCGTGCGGGGCGTGGCGCGGGGCTCGCCGTGCCGGGGGGGGGGTGGCGGCA
GGTGGGGGTGCCGGGCGGGGGGGGGCCGCCTCGGGCCGGGGAGGGCTCGGGGGAGGGGCGCGGCG
GCCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCCG
CCGCACCCCCTCTAGCGGGCGCGGGGCGAAGCGGTGCGGCGCCGGCAGGAAGGAAATGGGGGGGG
AGGGCCTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGGGGGGTTCGGCTTCTGGCGTGTGACCGGCGG
CTCTAGAGCCTCTGCTAACCATGTTCATGCCTTCTTCTTTTTCCTACAGCTCCTGGGCAACGTGC
TGGTTATTGTGACCGGTGTTGCGGCCCCGCAGCGCCCGCGCGCTCCTCTCCCCGACTCGGAGCCC
CTCGGCGGCGCCCGGCCCAGGACCCGCCTAGGAGCGCAGGAGCCCCAGCGCAGAGACCCCAACGC
CGAGACCCCCGCCCCGGCCCCGCCGCGCTTCCTCCCGACGCAGTTTAGGACCCTTGTTCGCGAAG
AGGTGGTGTGCGGCTGAGACCCGCGTCCTCAGGACGGTTCCATCAGTGCCTCGATCCTGCCCCAC
TGGAGGAGGAAGGCAGCCCGAACAGCGCTCACCTAACTAACAGCTGCTGAGAGCTGGGTTCCGTG
GCCATGCACCTGGGACTGCCTTGAGAAGCGTGAGCAAACCGCCCAGAGTAGAAGCGCTAGCCACC
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTTGGATCCCGGGCTGACTACAAAGACCATGACGGTGATT
ATAAAGATCATGACATCGACTACAAGGATGACGATGACAAGTAAGAGCTCAGTGTGAGTTCTACC
ATTGCCAAACTCGAGCAGTGAATTCTACCAGTGCCATAGGATCCAGTGTGAGTTCTACCATTGCC
AAAGGTACCCAGTGAATTCTACCAGTGCCATAGTTAACCGCATTGCCCAGTTGTTAGATTAAGAA
ATAGACAGCATGAGAGGGATGAGGCAACCCGTGCTCAGCTGTCAAGGCTCAGTCGCTAGCATTTC
CCAACACAAAGATTCTGACCTTAAATGCAACCATTTGAAACCCCTGTAGGCCTCAGGTGAAACTC
CAGATGCCACAATGGAGCCTCTGCTCCCCTAAAGCCTCAAAACAAAGGCCTAATTCTATGCCTGT
CTTAATTTTCTTTCACTTAAGTTAGTTCCACTGAGACCCCAGGCTGTTAGGGGTTATTGGTGTAA
GGTACTTTCATATTTTAAACAGAGGATATCGGCATTTGTTTCTTTCTCTGAGGACAAGAGAAAAA
AGCCAGGTTCCACAGAGGACACAGAGAAGGTTTGGGTGTCCTCCTGGGGTTCTTTTTGCCAACTT
TCCCCACGTTAAAGGTGAACATTGGTTCTTTCATTTGCTTTGGAAGTTTTAATCTCTAACAGTGG
ACAAAGTTACCAGTGCCTTAAACTCTGTTACACTTTTTGGAAGTGAAAACTTTGTAGTATGATAG
GTTATTTTGATGTAAAGATGTTCTGGATACCATTATATGTTCCCCCTGTTTCAGAGGCTCAGATT
GTAATATGTAAATGGTATGTCATTCGCTACTATGATTTAATTTGAAATATGGTCTTTTGGTTATG
AATACTTTGCAGCACAGCTGAGAGGCTGTCTGTTGTATTCATTGTGGTCATAGCACCTAACAACA
TTGTAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTACTA
CCTGTAATGACAGGCCTGTCCAACACATCTCCCTTTTCCATGACTGTGGTAGCCAGCATCGGAAA
GAACGCTGATTTAAAGAGGTCGCTTGGGAATTTTATTGACACAGTACCATTTAATGGGGAGGACA
AAATGGGGCAGGGGAGGGAGAAGTTTCTGTCGTTAAAAACAGATTTGGAAAGACTGGACTCTAAA
GTCTGTTGATTAAAGATGAGCTTTGTCTACTTCAAAAGTTTGTTTGCTTACCCCTTCAGCCTCCA
ATTTTTTAAGTGAAAATATAGCTAATAACATGTGAAAAGAATAGAAGCTAAGGTTTAGATAAATA
TTGAGCAGATCTATAGGAAGATTGAACCTGAATATTGCCATTATGCTTGACGCTGATCAGCCTCG
ACTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGGAAGACAATAGC
AGGCATGCTGGGGATGCGGTGGGCTCTATGGAAGCTTGAATTCAGCTGACGTGCCTCGGACCGCT
AGGAACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCGCAG
Exemplary Construct sequence
(SEQ ID NO: 61)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCCACAGGTAACTCCGTCGGCGTC
CACAGGGGGGCAGGAGATACCATACTGCACAGTTGTACGTCTTCCATCTGTTTGGTG
TAGAAAAATCTAACCACTACAAGAATGCCACGGGCACTGTGGCAGACAGAAGCAGC
GCTACGCCGCATCGCCTTTCAGCGTGCAGGCCCAGGAATGAGCGAGGCAGTGGGCG
GGGAAGACAGGCACGGGGAATCTGGGGACAGATAAAGGAAACTCGTGATGGGGCG
AGGCTGGGCTGAAGAGAAACAGATTGGGGTAGAGCTGCAAAGGGAGGGGTCCACTG
GAAGGCGAGGGGGGAGGCCGGGAAGAGAGAGGGTGGGAAGGCAGTGTGAGATGGG
AGGGCAGTGTGAGAAGAAAAGCAGGCTGGGGAAGAGGGATTGGAATGCAGAAGGA
ACTTGGGGAAGGAGGAAGTCCTGCAGGCGGGAGGGAAAGAAGAGAGGGGGAGCAG
CTAAAGTCTGCGTCAGAAGAGGTTGGGGACTGCGAGAGGAGAGGCTGGGGCCTGCA
GGGGAGCGCAGCAGCTTTTAGCATCGATCCAAACTCTAAAGACTCGTGGCCTTTGCC
TGACCTCGAGGGTCGGGAATAGACGCCTGTCTTTGTGGAGAGCGATACCCAACCGA
GAAAATGGGGCTGTTCCGAGCTGGGCCCTGCGCCTGGCCCAGGGCGAGGCTTCTCTG
GCTCCGGGCTGGCCCCTGAGGGGCAGCACGCAGCCTGCAGCAGAGGCGCCTGCTCC
AAGCTGTCTCTTGGGGGCGCCGCCGCCGCTTCCCTCCTCCGGGGCCGCTCGCTCCCA
GGAAAGTGGAGGCGGCTGGCGAGGACCGAGAGCCGGGGCCGCGCTGCGGAGGGAC
CACACCTCCGGGAGTTCGAGGGGGACCCTGGCGCGGCGGGCCAGCCTTTCGGGCCG
GCAGCGCCCGCCTTCCCCCGGTCAGCGCTTGCGGCCCGCGCCGCGCGCACCGCCCGG
CAACCCCGCGCGCGTCCCGCGGGGGCGCTGCGTCTTCCTGCCACACCGGCGCACCGC
GGCCCCTCTCCCCCACACCTCCGGCCCGCACCACCCGGCTCTCCTCCCACCCTCCCCA
CCCCTCCTCTGCCCTCCCTCCCCATTCCTCCCCTCCCGGCGAGGGGGGGGAGGGGGC
GTGGCGGGGCCGGGGTTTGTGTGGCTGGGACCCGGCTCCTCAAGCTCTGAGGACCCA
GAGGCCGGGCGCGCTCCGCCCGCGGCGCCGCCCCCTCCGTAACTTTCCCAGTCTCCG
AGGGAAGAGGCGGGGTGTGGGGTGCGGTTAAAAGGCGCCACGGCGGGAGACAGGT
CTCACCGGTGTGTCACCGTTGCGGCCCCGCAGCGCCCGCGCGCTCCTCTCCCCGACT
CGGAGCCCCTCGGCGGCGCCCGGCCCAGGACCCGCCTAGGAGCGCAGGAGCCCCAG
CGCAGAGACCCCAACGCCGAGACCCCCGCCCCGGCCCCGCCGCGCTTCCTCCCGACG
CAGTTTAGGACCCTTGTTCGCGAAGAGGTGGTGTGCGGCTGAGACCCGCGTCCTCAG
GACGGTTCCATCAGTGCCTCGATCCTGCCCCACTGGAGGAGGAAGGCAGCCCGAAC
AGCGCTCACCTAACTAACAGCTGCTGAGAGCTGGGTTCCGTGGCCATGCACCTGGGA
CTGCCTTGAGAAGCGTGAGCAAACCGCCCAGAGTAGAAGCGCTAGCCACCATGGAT
TGGGGCACGCTGCAGACGATCCTGGGGGGTGTGAACAAACACTCCACCAGCATTGG
AAAGATCTGGCTCACCGTCCTCTTCATTTTTCGCATTATGATCCTCGTTGTGGCTGCA
AAGGAGGTGTGGGGAGATGAGCAGGCCGACTTTGTCTGCAACACCCTGCAGCCAGG
CTGCAAGAACGTGTGCTACGATCACTACTTCCCCATCTCCCACATCCGGCTATGGGC
CCTGCAGCTGATCTTCGTGTCCACGCCAGCGCTCCTAGTGGCCATGCACGTGGCCTA
CCGGAGACATGAGAAGAAGAGGAAGTTCATCAAGGGGGAGATAAAGAGTGAATTT
AAGGACATCGAGGAGATCAAAACCCAGAAGGTCCGCATCGAAGGCTCCCTGTGGTG
GACCTACACAAGCAGCATCTTCTTCCGGGTCATCTTCGAAGCCGCCTTCATGTACGTC
TTCTATGTCATGTACGACGGCTTCTCCATGCAGCGGCTGGTGAAGTGCAACGCCTGG
CCTTGTCCCAACACTGTGGACTGCTTTGTGTCCCGGCCCACGGAGAAGACTGTCTTC
ACAGTGTTCATGATTGCAGTGTCTGGAATTTGCATCCTGCTGAATGTCACTGAATTGT
GTTATTTGCTAATTAGATATTGTTCTGGGAAGTCAAAAAAGCCAGTTGGATCCCGGG
CTGACTACAAAGACCATGACGGTGATTATAAAGATCATGACATCGACTACAAGGAT
GACGATGACAAGTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAA
CCATTTGAAACCCCTGTAGGCCTCAGGTGAAACTCCAGATGCCACAATGGAGCTCTG
CTCCCCTAAAGCCTCAAAACAAAGGCCTAATTCTATGCCTGTCTTAATTTTCTTTCAC
TTAAGTTAGTTCCACTGAGACCCCAGGCTGTTAGGGGTTATTGGTGTAAGGTACTTTC
ATATTTTAAACAGAGGATATCGGCATTTGTTTCTTTCTCTGAGGACAAGAGAAAAAA
GCCAGGTTCCACAGAGGACACAGAGAAGGTTTGGGTGTCCTCCTGGGGTTCTTTTTG
CCAACTTTCCCCACGTTAAAGGTGAACATTGGTTCTTTCATTTGCTTTGGAAGTTTTA
ATCTCTAACAGTGGACAAAGTTACCAGTGCCTTAAACTCTGTTACACTTTTTGGAAGT
GAAAACTTTGTAGTATGATAGGTTATTTTGATGTAAAGATGTTCTGGATACCATTATA
TGTTCCCCCTGTTTCAGAGGCTCAGATTGTAATATGTAAATGGTATGTCATTCGCTAC
TATGATTTAATTTGAAATATGGTCTTTTGGTTATGAATACTTTGCAGCACAGCTGAGA
GGCTGTCTGTTGTATTCATTGTGGTCATAGCACCTAACAACATTGTAGCCTCAATCGA
GTGAGACAGACTAGAAGTTCCTAGTGATGGCTTATGATAGCAAATGGCCTCATGTCA
AATATTTAGATGTAATTTTGTGTAAGAAATACAGACTGGATGTACCACCAACTACTA
CCTGTAATGACAGGCCTGTCCAACACATCTCCCTTTTCCATGACTGTGGTAGCCAGC
ATCGGAAAGAACGCTGATTTAAAGAGGTCGCTTGGGAATTTTATTGACACAGTACCA
TTTAATGGGGAGGACAAAATGGGGCAGGGGAGGGAGAAGTTTCTGTCGTTAAAAAC
AGATTTGGAAAGACTGGACTCTAAAGTCTGTTGATTAAAGATGAGCTTTGTCTACTT
CAAAAGTTTGTTTGCTTACCCCTTCAGCCTCCAATTTTTTAAGTGAAAATATAGCTAA
TAACATGTGAAAAGAATAGAAGCTAAGGTTTAGATAAATATTGAGCAGATCTATAG
GAAGATTGAACCTGAATATTGCCATTATGCTTGACATGGTTTCCAAAAAATGGTACT
CCACATATTTCAGTGAGGGTAAGTATTTTCCTGTTGTCAAGAATAGCATTGTAAAAG
CATTTTGTAATAATAAAGAATAGCTTTAATGATATGCTTGTAACTAAAATAATTTTGT
AATGTATCAAATACATTTAAAACATTAAAATATAATCTCTATAATAAGAGCTCGCTG
ATCAGCCTCGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGCGGTGG
GCTCTATGGAAGCTTGAATTCAGCTGACGTGCCTCGGACCGCTAGGAACCCCTAGTG
ATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGTATT
TCACACCGCATACGTCAAAGCAACCATAGTACGCGCCCTGTAGCGGCGCATTAAGCG
CGGCGGGTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCACCTCGACC
CCAAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAAAC
TGGAACAACACTCAACCCTATCTCGGGCTATTCTTTTGATTTATAAGGGATTTTGCCG
ATTTCGGCCTATTGGTTAAAAAATGAGCTGATTTAACAAAAATTTAACGCGAATTTT
AACAAAATATTAACGTTTACAATTTTATGGTGCACTCTCAGTACAATCTGCTCTGATG
CCGCATAGTTAAGCCAGCCCCGACACCCGCCAACACCCGCTGACGCGCCCTGACGG
GCTTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCGTG
ATACGCCTATTTTTATAGGTTAATGTCATGAACAATAAAACTGTCTGCTTACATAAAC
AGTAATACAAGGGGTGTTATGAGCCATATTCAACGGGAAACGTCGAGGCCGCGATT
AAATTCCAACATGGATGCTGATTTATATGGGTATAAATGGGCTCGCGATAATGTCGG
GCAATCAGGTGCGACAATCTATCGCTTGTATGGGAAGCCCGATGCGCCAGAGTTGTT
TCTGAAACATGGCAAAGGTAGCGTTGCCAATGATGTTACAGATGAGATGGTCAGACT
AAACTGGCTGACGGAATTTATGCCTCTTCCGACCATCAAGCATTTTATCCGTACTCCT
GATGATGCATGGTTACTCACCACTGCGATCCCCGGAAAAACAGCATTCCAGGTATTA
GAAGAATATCCTGATTCAGGTGAAAATATTGTTGATGCGCTGGCAGTGTTCCTGCGC
CGGTTGCATTCGATTCCTGTTTGTAATTGTCCTTTTAACAGCGATCGCGTATTTCGTCT
CGCTCAGGCGCAATCACGAATGAATAACGGTTTGGTTGATGCGAGTGATTTTGATGA
CGAGCGTAATGGCTGGCCTGTTGAACAAGTCTGGAAAGAAATGCATAAACTTTTGCC
ATTCTCACCGGATTCAGTCGTCACTCATGGTGATTTCTCACTTGATAACCTTATTTTT
GACGAGGGGAAATTAATAGGTTGTATTGATGTTGGACGAGTCGGAATCGCAGACCG
ATACCAGGATCTTGCCATCCTATGGAACTGCCTCGGTGAGTTTTCTCCTTCATTACAG
AAACGGCTTTTTCAAAAATATGGTATTGATAATCCTGATATGAATAAATTGCAGTTT
CATTTGATGCTCGATGAGTTTTTCTAATCTCATGACCAAAATCCCTTAACGTGAGTTT
TCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCA
GAGCGCAGATACCAAATACTGTCCTTCTAGTGTAGCCGTAGTTAGGCCACCACTTCA
AGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCT
TTTGCTCACATGT

TABLE 4
Components of Construct Sequence (SEQ ID NO: 61)

| Components | Position in construct |
|---|---|
| 5′ITR | 12-130 |
| Cloning site | 131-147 |
| GDF6 promoter | 148-1335 |
| hGJB2 minimal promoter | 1336-1463 |
| Cloning site | 1464-1472 |
| Synthetic barcode | 1473-1480 |
| 5′UTR | 1481-1842 |
| GJB2 (exon2) | 1854-2531 |
| 3xFLAG | 2544-2609 |
| 3′UTR (exon2) | 2613-4019 |
| bGHpA | 4041-4265 |
| Cloning site | 4266-4299 |
| 3′ITR | 4300-4429 |

Exemplary Construct sequence
(SEQ ID NO: 54)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTAAGAAACTTGCCCGAGTTTACAC
AGCTAGTAAATGGTTGCATTAGTCAGGACAGCTAGCCTATATTACAATAACAACCCT
CTCAAATCCTAATGGCTTAAAACAACAGAGGTTTAATTTATACTCATTAGCTGTTCA
AGGCAGGAGGCTCTATTCTCTAATCCATACAGTCACTCAGGATCCAGGCTGGTGGAG
ACCCTGCCATATTGTAGCCTCACCATTTAAAACATGAAGAAGATAGAAAGTGAGGA
GTCATGTAGGTTTTGTTCCGTTGCCTCAGGCTAGGAGTGACAGGTCACTTCATCTCAC
TCACAGCTCACTGCCCACAACTAGTCACTTGTGACTGTGCGAGTTAAGCTTCTGTGTG
TGAAGGAAGGAAAAGAGAATGGGATAAAGGTGAACATCAGCAGGCTCTACCACAGT
AGTTTGAACCAAGACTTGAGCCTAGGTCATGTGGCTTCAGAATCTTTGCTCTTAATCA
CACTAAACAGCCTCTGTAAGTCATCTTTCCTTCATCCAGTGCCTAAGAACATGCAGTC
CAATGCCCTCATCCTTCAGAAGAACTTGAGTGAACTCAGAGAAATTGAGTAGAGTGC
CACAGCATGCCCAAGGCCACACACCCTGAGGTTGGCAGTAGGTCCTGAGTTAGAGTT
GTCATTTCTTGGCTCCCCTGGTAGTAGTGGAAAGGTAAGGTTTTGACATACTAGTTG
GATGACCACGGGCAGGTCACTTAAATTGTCTAAGCATCGTTTGACCCTTGTAAGAAT
TAAATGAAATAGCACCTGTAAAAGTGTCTGCACGGACTTACTGCTGTTAGTTTTGTTC
CTTTCTTCCTGTTGTCACTGCACTTCCCTGCCTGTTACCCAGGCCATGCAGACCAGCC
AGGCCTTCGACTTACAGTGCGGATAAGATTCCAAATCTCCACGGCTGGTTTCCATGC
TTTCTTCCAGGCTTCTGAGGACCCTGTGCTCTGGTTTCTTCTATTTCTTTTCTATTACT
TTTCTGTTACTCTTGAGCACACTTGCTGGAAGCAATATGCATCCAGTTCTCCCTCTCT
TGCCTCATTACACTTTGCAGAACAACTCCAATCCCTTCCAACCAAGTAGTCCCTTTGA
ATTTCTTGTCACCCAAGGAATCTCTCTGACAGGGGTCTTTGTTAGGGTCACACCCCAG
GAGATGGTTGATTATGGCTGAGTCCAGCCTGGAATGATGGGGGTTGGGGGCAGCTTG
GGTAGATGACTCAGTAAATCAAACAGAACAATGAAAGGAGGTCATGCTTGTCCATC
TGCATTATTGAAGACAGCCATAAATGGCCTTACCCCAGAGCGGGTCTGTCACACCTG
GAGAGCTGATCTGACCTCTCCAAGACCCCTGCAACTGAGTGTTCTGGGATCTGTCCT
GCAACAAGTGCCTCGAGATTTGTAGGTGGGGGCCCAGAGGGAGGGGGTCTGCAGAC
GAAGGGGGCAGGTTTTGCGGGGCACTTAGGGTTCTCATAGGTTGTAGTCACGAGCTC
CAAGCTCTGAGGACCCAGAGGCCGGGCGCGCTCCGCCCGCGGCGCCGCCCCCTCCGT
AACTTTCCCAGTCTCCGAGGGAAGAGGCGGGGTGTGGGGTGCGGTTAAAAGGCGCC
ACGGCGGGAGACAGGTCTCACCGGTCACAACCTGTTGCGGCCCCGCAGCGCCCGCG
CGCTCCTCTCCCCGACTCGGAGCCCCTCGGCGGCGCCCGGCCCAGGACCCGCCTAGG
AGCGCAGGAGCCCCAGCGCAGAGACCCCAACGCCGAGACCCCCGCCCCGGCCCCGC
CGCGCTTCCTCCCGACGCAGTTTAGGACCCTTGTTCGCGAAGAGGTGGTGTGCGGCT
GAGACCCGCGTCCTCAGGACGGTTCCATCAGTGCCTCGATCCTGCCCCACTGGAGGA
GGAAGGCAGCCCGAACAGCGCTCACCTAACTAACAGCTGCTGAGAGCTGGGTTCCG
TGGCCATGCACCTGGGACTGCCTTGAGAAGCGTGAGCAAACCGCCCAGAGTAGAAG
CGCTAGCCACCATGGATTGGGGCACGCTGCAGACGATCCTGGGGGGTGTGAACAAA
CACTCCACCAGCATTGGAAAGATCTGGCTCACCGTCCTCTTCATTTTTCGCATTATGA
TCCTCGTTGTGGCTGCAAAGGAGGTGTGGGGAGATGAGCAGGCCGACTTTGTCTGCA
ACACCCTGCAGCCAGGCTGCAAGAACGTGTGCTACGATCACTACTTCCCCATCTCCC
ACATCCGGCTATGGGCCCTGCAGCTGATCTTCGTGTCCACGCCAGCGCTCCTAGTGG
CCATGCACGTGGCCTACCGGAGACATGAGAAGAAGAGGAAGTTCATCAAGGGGGAG
ATAAAGAGTGAATTTAAGGACATCGAGGAGATCAAAACCCAGAAGGTCCGCATCGA
AGGCTCCCTGTGGTGGACCTACACAAGCAGCATCTTCTTCCGGGTCATCTTCGAAGC
CGCCTTCATGTACGTCTTCTATGTCATGTACGACGGCTTCTCCATGCAGCGGCTGGTG
AAGTGCAACGCCTGGCCTTGTCCCAACACTGTGGACTGCTTTGTGTCCCGGCCCACG
GAGAAGACTGTCTTCACAGTGTTCATGATTGCAGTGTCTGGAATTTGCATCCTGCTG
AATGTCACTGAATTGTGTTATTTGCTAATTAGATATTGTTCTGGGAAGTCAAAAAAG
CCAGTTGGATCCCGGGCTGACTACAAAGACCATGACGGTGATTATAAAGATCATGAC
ATCGACTACAAGGATGACGATGACAAGTAAGAAATAGACAGCATGAGAGGGATGA
GGCAACCCGTGCTCAGCTGTCAAGGCTCAGTCGCTAGCATTTCCCAACACAAAGATT
CTGACCTTAAATGCAACCATTTGAAACCCCTGTAGGCCTCAGGTGAAACTCCAGATG
CCACAATGGAGCTCTGCTCCCCTAAAGCCTCAAAACAAAGGCCTAATTCTATGCCTG
TCTTAATTTTCTTTCACTTAAGTTAGTTCCACTGAGACCCCAGGCTGTTAGGGGTTAT
TGGTGTAAGGTACTTTCATATTTTAAACAGAGGATATCGGCATTTGTTTCTTTCTCTG
AGGACAAGAGAAAAAAGCCAGGTTCCACAGAGGACACAGAGAAGGTTTGGGTGTCC
TCCTGGGGTTCTTTTTGCCAACTTTCCCCACGTTAAAGGTGAACATTGGTTCTTTCAT
TTGCTTTGGAAGTTTTAATCTCTAACAGTGGACAAAGTTACCAGTGCCTTAAACTCTG
TTACACTTTTTGGAAGTGAAAACTTTGTAGTATGATAGGTTATTTTGATGTAAAGATG
TTCTGGATACCATTATATGTTCCCCCTGTTTCAGAGGCTCAGATTGTAATATGTAAAT
GGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAA
CATTGTAGCCTCAATCGAGTGAGACAGACTAGAAGTTCCTAGTGATGGCTTATGATA
GCAAATGGCCTCATGTCAAATATTTAGATGTAATTTTGTGTAAGAAATACAGACTGG
ATGTACCACCAACTACTACCTGTAATGACAGGCCTGTCCAACACATCTCCCTTTTCCA
TGACTGTGGTAGCCAGCATCGGAAAGAACGCTGATTTAAAGAGGTCGCTTGGGAATT
TTATTGACACAGTACCATTTAATGGGGAGGACAAAATGGGGCAGGGGAGGGAGAAG
TTTCTGTCGTTAAAAACAGATTTGGAAAGACTGGACTCTAAAGTCTGTTGATTAAAG
ATGAGCTTTGTCTACTTCAAAAGTTTGTTTGCTTACCCCTTCAGCCTCCAATTTTTTAA
GTGAAAATATAGCTAATAACATGTGAAAAGAATAGAAGCTAAGGTTTAGATAAATA
TTGAGCAGATCTATAGGAAGATTGAACCTGAATATTGCCATTATGCTTGACATGGTT
TCCAAAAAATGGTACTCCACATATTTCAGTGAGGGTAAGTATTTTCCTGTTGTCAAG
AATAGCATTGTAAAAGCATTTTGTAATAATAAAGAATAGCTTTAATGATATGCTTGT
AACTAAAATAATTTTGTAATGTATCAAATACATTTAAAACATTAAAATATAATCTCT
ATAATAAGAGCTCGCTGATCAGCCTCGACTGTGCCTTCTAGTTGCCAGCCATCTGTTG
TTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCAT
GCTGGGGATGCGGTGGGCTCTATGGAAGCTTGAATTCAGCTGACGTGCCTCGGACCG
CTAGGAACCCCTAGTGATGGAGTTGGCCACTCCCTCTCTGCGCGCTCGCTCGCTCACT
GAGGCCGGGCGACCAAAGGTCGCCCGACGCCCGGGCTTTGCCCGGGCGGCCTCAGT
GAGCGAGCGAGCGCGCAGCTGCCTGCAGGGGCGCCTGATGCGGTATTTTCTCCTTAC
GCATCTGTGCGGTATTTCACACCGCATACGTCAAAGCAACCATAGTACGCGCCCTGT
AGCGGCGCATTAAGCGCGGCGGGTGTGGTGGTTACGCGCAGCGTGACCGCTACACTT
GCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCA
TCGCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTTTTGATTT
ATAAGGGATTTTGCCGATTTCGGCCTATTGGTTAAAAAATGAGCTGATTTAACAAAA
ATTTAACGCGAATTTTAACAAAATATTAACGTTTACAATTTTATGGTGCACTCTCAGT
ACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACC
GTCTCCGGGAGCTGCATGTGTCAGAGGTTTTCACCGTCATCACCGAAACGCGCGAGA
CGAAAGGGCCTCGTGATACGCCTATTTTTATAGGTTAATGTCATGAACAATAAAACT
GTCTGCTTACATAAACAGTAATACAAGGGGTGTTATGAGCCATATTCAACGGGAAAC
GTCGAGGCCGCGATTAAATTCCAACATGGATGCTGATTTATATGGGTATAAATGGGC
TCGCGATAATGTCGGGCAATCAGGTGCGACAATCTATCGCTTGTATGGGAAGCCCGA
TGCGCCAGAGTTGTTTCTGAAACATGGCAAAGGTAGCGTTGCCAATGATGTTACAGA
TGAGATGGTCAGACTAAACTGGCTGACGGAATTTATGCCTCTTCCGACCATCAAGCA
TTTTATCCGTACTCCTGATGATGCATGGTTACTCACCACTGCGATCCCCGGAAAAAC
AGCATTCCAGGTATTAGAAGAATATCCTGATTCAGGTGAAAATATTGTTGATGCGCT
GGCAGTGTTCCTGCGCCGGTTGCATTCGATTCCTGTTTGTAATTGTCCTTTTAACAGC
GATCGCGTATTTCGTCTCGCTCAGGCGCAATCACGAATGAATAACGGTTTGGTTGAT
GCGAGTGATTTTGATGACGAGCGTAATGGCTGGCCTGTTGAACAAGTCTGGAAAGA
AATGCATAAACTTTTGCCATTCTCACCGGATTCAGTCGTCACTCATGGTGATTTCTCA
CTTGATAACCTTATTTTTGACGAGGGGAAATTAATAGGTTGTATTGATGTTGGACGA
GTCGGAATCGCAGACCGATACCAGGATCTTGCCATCCTATGGAACTGCCTCGGTGAG
TTTTCTCCTTCATTACAGAAACGGCTTTTTCAAAAATATGGTATTGATAATCCTGATA
TGAATAAATTGCAGTTTCATTTGATGCTCGATGAGTTTTTCTAATCTCATGACCAAAA
TCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAA
GGTAACTGGCTTCAGCAGAGCGCAGATACCAAATACTGTCCTTCTAGTGTAGCCGTA
GTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAG
CGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGAT
GCTCGTCAGGGGGGCGGAGCCTATGGAAAAACGCCAGCAACGCGGCCTTTTTACGG
TTCCTGGCCTTTTGCTGGCCTTTTGCTCACATGT

TABLE 5
Components of Construct Sequence (SEQ ID NO: 54)

| Components | Position in construct |
|---|---|
| 5′ITR | 12-130 |
| Cloning site | 131-147 |
| IGFBP2 promoter | 148-1660 |
| hGJB2 minimal promoter | 1661-1788 |
| Cloning site | 1789-1797 |
| Synthetic barcode | 1798-1805 |
| 5′UTR | 1806-2167 |
| GJB2 (exon2) | 2179-2856 |
| 3xFLAG | 2869-2934 |
| 3′UTR (exon2) | 2938-4344 |
| bGHpA | 4366-4590 |
| Cloning site | 4591-4624 |
| 3′ITR | 4625-4754 |

Exemplary Construct sequence
(SEQ ID NO: 17)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCCCATGGCTCTGTTAAAATCAAA
GAAACATCTTTTCCAACAGCCCTTTCAAACTCCTCATCGCATCTCACTGGCTGATTCA
GTCATTTAAACCTGCTTCTCCCTAAAGCTGATCACTGGCTAAGCTAATAGGGTTTCCG
GGATTGGTTTAGCCTGATACTAATCCAGGTCTACCTTCAGGAGCCAGACCAAACTGC
CTATTGGCATTGCATTCTTGCAGTAGGGAGGGGAGGTATGGATGGTGTGGAGTCCAC
CACAAGGTCCATGCCAGTCTTTGCTGAACCAGCATCAGACTCCATCAAGCAACAGAT
GAGAGGTTCCATGATAAAGTGGCCCTCAGCAATCCCCATCCATTGCTGTCTAGGAAG
AACAGTGCTTGTACACAGGTTTAGGACCTCAGTCTTGGCTGTAATCTTCTGGTTTACT
TTGCCAGCACCAAACAGAAGGAAAGAAAGGGCTCAAATTTGACCAAATAAATTATG
CTTCTCCTTCCAGAGATAACCTTGAGTCCTGTCTAGGAAGATATTAGAATTGTAAAG
AAAAAAAAAATTACTCCTTATCCTATGGCAAGTGGAGTCTATGTCTACTTCAGCTGA
AATTAAATCCTGTCCATAATAGATGACCCTTGCTCAAGCTGGCCAGAAGCCATACCA
ACCAGCACGAAGGTTAAAACTATTATTAGTTTTTTCTGTGATTTTCATTTTCAGGCCA
AGTTTTAGAACAATAAGATTTTAAGAATAGGAAGTAAGTAAGATTTCTGCATATCCT
GTTCTCTTAGTCAGCTGAATTTTTTTTTTTTTTTTTTTAGTCCTAACTCAGCCTCCCAA
AGTGCTGGGATTACAGGCGTGAGCCACCGCACCAAGCCTGGAATCTATGTCTTACAG
TTATGAGAATCAACAGCTAGCTCATTATGGGCAAGGTGATGTCACTCTGGCTTCTCA
ATGAAAATGGCATTTCTCCCTTGGAAAAGGTCATAGCCAGTCAGTCAGTCAGTCACG
GGAGCGCAGCGGCTTCTAGGGGTGAGTGGGACCCACGCGGCCCCACCTGCTCCTCCC
GCGCGCGGCCCCACCCCCCTGCCCCGCCCCGCCTGGTTTATAGAAGCTCTGAGGACC
CAGAGGCCGGGCGCGCTCCGCCCGCGGCGCCGCCCCCTCCGTAACTTTCCCAGTCTC
CGAGGGAAGAGGCGGGGTGTGGGGTGCGGTTAAAAGGCGCCACGGCGGGAGACAG
GTCTCACCGGTCGTGTGTTGTTGCGGCCCCGCAGCGCCCGCGCGCTCCTCTCCCCGAC
TCGGAGCCCCTCGGCGGCGCCCGGCCCAGGACCCGCCTAGGAGCGCAGGAGCCCCA
GCGCAGAGACCCCAACGCCGAGACCCCCGCCCCGGCCCCGCCGCGCTTCCTCCCGAC
GCAGTTTAGGACCCTTGTTCGCGAAGAGGTGGTGTGCGGCTGAGACCCGCGTCCTCA
GGACGGTTCCATCAGTGCCTCGATCCTGCCCCACTGGAGGAGGAAGGCAGCCCGAA
CAGCGCTCACCTAACTAACAGCTGCTGAGAGCTGGGTTCCGTGGCCATGCACCTGGG
ACTGCCTTGAGAAGCGTGAGCAAACCGCCCAGAGTAGAAGCGCTAGCCACCATGGA
TTGGGGCACGCTGCAGACGATCCTGGGGGGTGTGAACAAACACTCCACCAGCATTG
GAAAGATCTGGCTCACCGTCCTCTTCATTTTTCGCATTATGATCCTCGTTGTGGCTGC
AAAGGAGGTGTGGGGAGATGAGCAGGCCGACTTTGTCTGCAACACCCTGCAGCCAG
GCTGCAAGAACGTGTGCTACGATCACTACTTCCCCATCTCCCACATCCGGCTATGGG
CCCTGCAGCTGATCTTCGTGTCCACGCCAGCGCTCCTAGTGGCCATGCACGTGGCCT
ACCGGAGACATGAGAAGAAGAGGAAGTTCATCAAGGGGGAGATAAAGAGTGAATT
TAAGGACATCGAGGAGATCAAAACCCAGAAGGTCCGCATCGAAGGCTCCCTGTGGT
GGACCTACACAAGCAGCATCTTCTTCCGGGTCATCTTCGAAGCCGCCTTCATGTACG
TCTTCTATGTCATGTACGACGGCTTCTCCATGCAGCGGCTGGTGAAGTGCAACGCCT
GGCCTTGTCCCAACACTGTGGACTGCTTTGTGTCCCGGCCCACGGAGAAGACTGTCT
TCACAGTGTTCATGATTGCAGTGTCTGGAATTTGCATCCTGCTGAATGTCACTGAATT
GTGTTATTTGCTAATTAGATATTGTTCTGGGAAGTCAAAAAAGCCAGTTGGATCCCG
GGCTGACTACAAAGACCATGACGGTGATTATAAAGATCATGACATCGACTACAAGG
ATGACGATGACAAGTAAGAAATAGACAGCATGAGAGGGATGAGGCAACCCGTGCTC
AGCTGTCAAGGCTCAGTCGCTAGCATTTCCCAACACAAAGATTCTGACCTTAAATGC
AACCATTTGAAACCCCTGTAGGCCTCAGGTGAAACTCCAGATGCCACAATGGAGCTC
TGCTCCCCTAAAGCCTCAAAACAAAGGCCTAATTCTATGCCTGTCTTAATTTTCTTTC
ACTTAAGTTAGTTCCACTGAGACCCCAGGCTGTTAGGGGTTATTGGTGTAAGGTACT
TTCATATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGTTCTTT
TTGCCAACTTTCCCCACGTTAAAGGTGAACATTGGTTCTTTCATTTGCTTTGGAAGTT
TTAATCTCTAACAGTGGACAAAGTTACCAGTGCCTTAAACTCTGTTACACTTTTTGGA
AGTGAAAACTTTGTAGTATGATAGGTTATTTTGATGTAAAGATGTTCTGGATACCATT
ATATGTTCCCCCTGTTTCAGAGGCTCAGATTGTAATATGTAAATGGTATGTCATTCGC
TACTATGATTTAATTTGAAATATGGTCTTTTGGTTATGAATACTTTGCAGCACAGCTG
AGAGGCTGTCTGTTGTATTCATTGTGGTCATAGCACCTAACAACATTGTAGCCTCAAT
CGAGTGAGACAGACTAGAAGTTCCTAGTGATGGCTTATGATAGCAAATGGCCTCATG
TCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCA
GCATCGGAAAGAACGCTGATTTAAAGAGGTCGCTTGGGAATTTTATTGACACAGTAC
CATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTAC
TTCAAAAGTTTGTTTGCTTACCCCTTCAGCCTCCAATTTTTTAAGTGAAAATATAGCT
AATAACATGTGAAAAGAATAGAAGCTAAGGTTTAGATAAATATTGAGCAGATCTAT
AGGAAGATTGAACCTGAATATTGCCATTATGCTTGACATGGTTTCCAAAAAATGGTA
CTCCACATATTTCAGTGAGGGTAAGTATTTTCCTGTTGTCAAGAATAGCATTGTAAA
AGCATTTTGTAATAATAAAGAATAGCTTTAATGATATGCTTGTAACTAAAATAATTTT
GTAATGTATCAAATACATTTAAAACATTAAAATATAATCTCTATAATAAGAGCTCGC
TGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGGGGGCA
GGACAGCAAGGGGGAGGATTGGGAAGACAATAGCAGGCATGCTGGGGATGCGGTG
GGCTCTATGGAAGCTTGAATTCAGCTGACGTGCCTCGGACCGCTAGGAACCCCTAGT
GATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGC
GCAGCTGCCTGCAGGGGCGCCTGATGCGGTATTTTCTCCTTACGCATCTGTGCGGTAT
TTCACACCGCATACGTCAAAGCAACCATAGTACGCGCCCTGTAGCGGCGCATTAAGC
GCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGAC
CCCAAAAAACTTGATTTGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTGCC
GATTTCGGCCTATTGGTTAAAAAATGAGCTGATTTAACAAAAATTTAACGCGAATTT
TAACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGACG
GGCTTGTCTGCTCCCGGCATCCGCTTACAGACAAGCTGTGACCGTCTCCGGGAGCTG
CATGTGTCAGAGGTTTTCACCGTCATCACCGAAACGCGCGAGACGAAAGGGCCTCGT
GATACGCCTATTTTTATAGGTTAATGTCATGAACAATAAAACTGTCTGCTTACATAA
ACAGTAATACAAGGGGTGTTATGAGCCATATTCAACGGGAAACGTCGAGGCCGCGA
TTAAATTCCAACATGGATGCTGATTTATATGGGTATAAATGGGCTCGCGATAATGTC
GGGCAATCAGGTGCGACAATCTATCGCTTGTATGGGAAGCCCGATGCGCCAGAGTTG
TTTCTGAAACATGGCAAAGGTAGCGTTGCCAATGATGTTACAGATGAGATGGTCAGA
CTAAACTGGCTGACGGAATTTATGCCTCTTCCGACCATCAAGCATTTTATCCGTACTC
CTGATGATGCATGGTTACTCACCACTGCGATCCCCGGAAAAACAGCATTCCAGGTAT
TAGAAGAATATCCTGATTCAGGTGAAAATATTGTTGATGCGCTGGCAGTGTTCCTGC
GCCGGTTGCATTCGATTCCTGTTTGTAATTGTCCTTTTAACAGCGATCGCGTATTTCG
TCTCGCTCAGGCGCAATCACGAATGAATAACGGTTTGGTTGATGCGAGTGATTTTGA
TGACGAGCGTAATGGCTGGCCTGTTGAACAAGTCTGGAAAGAAATGCATAAACTTTT
GCCATTCTCACCGGATTCAGTCGTCACTCATGGTGATTTCTCACTTGATAACCTTATT
TTTGACGAGGGGAAATTAATAGGTTGTATTGATGTTGGACGAGTCGGAATCGCAGAC
CGATACCAGGATCTTGCCATCCTATGGAACTGCCTCGGTGAGTTTTCTCCTTCATTAC
AGAAACGGCTTTTTCAAAAATATGGTATTGATAATCCTGATATGAATAAATTGCAGT
TTCATTTGATGCTCGATGAGTTTTTCTAATCTCATGACCAAAATCCCTTAACGTGAGT
TTTCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTCAG
CAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGG
CCTTTTGCTCACATGT

TABLE 6
Components of Construct Sequence (SEQ ID NO: 17)

| Components | Position in construct |
|---|---|
| 5′ITR | 12-130 |
| Cloning site | 131-147 |
| RBP7 promoter | 148-1244 |
| hGJB2 minimal promoter | 1245-1372 |
| Cloning site | 1373-1381 |
| Synthetic barcode | 1382-1389 |
| 5′UTR | 1390-1751 |
| GJB2 (exon2) | 1763-2440 |
| 3xFLAG | 2453-2518 |
| 3′UTR (exon2) | 2522-3928 |
| bGHpA | 3950-4174 |
| Cloning site | 4175-4208 |
| 3′ITR | 4209-4338 |

Exemplary Construct sequence
(SEQ ID NO: 38)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTAAATAGCTTCCAACGTTTCCACC
CCACCAGCCCTTGCACCACTCCCTGTACTGGCCCTGAGCTTTCTAGTCTTGACTGAAA
AGCGGGGAGGCAATGTGGTCTCTCCTGGTGCACTGTCCCGAGGAAGGCCTGCTCCGC
TTCCCCGGAGGAGTCTTCAAAGGATGGAGGTAATTAATAAAAACAACCCCTGTACCT
CCTCTAAGTGGTCATTAATTAATAAAGAACCTCCAGGCTCCTATAGGAGAGGTCTGT
GCACCCCGCGGGCTATGAGAAGGCTGGATCACCCAGAAAGACTGAGGATGTGTCCT
GGCAAAAACACAGCCTGCCCCTCACACTGCTCCCCACGGGTGCACTAGGGAGGAAG
AGTTCCCTCGAGGGCCTGAGCAGGCGCCCCACACCTGCACCCGTGCAGAGGGGGCT
GGGCCCGCCCTCTGCGCTCCCGAGGGAGAGCCCTACCCCCTGCATCCCCGGTACCCC
GTTCCCTCCAAGGGCCGGAAAGAGGGCCCCGCGCACTGTGCACTTCTTAGGGGTCCC
CCACCCTGCGCCCCCGCCACGGGAAAAAGGTCCCCGCTCTGCGCATCCGGCCCCGGA
GGGACAGCCCCGGTCCTGCACTCCTTGCTCCTCAGGGGGACGGTCCGCGCCCAGCGG
CTAGTGCGCCCCGGGTAGGTGGGGGCGGGGGGCTCGTCGAGTGACAGCGCTCGCCT
CCCGCAGCCCGCCCGAGCCGCGTCAGGGCAGAAGCTCTGAGGACCCAGAGGCCGGG
CGCGCTCCGCCCGCGGCGCCGCCCCCTCCGTAACTTTCCCAGTCTCCGAGGGAAGAG
GCGGGGTGTGGGGTGCGGTTAAAAGGCGCCACGGCGGGAGACAGGTCTCACCGGTT
CGTGGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACC
CCAACGCCGAGACCCCCGCCCCGGCCCCGCCGCGCTTCCTCCCGACGCAGTTTAGGA
CCCTTGTTCGCGAAGAGGTGGTGTGCGGCTGAGACCCGCGTCCTCAGGACGGTTCCA
TCAGTGCCTCGATCCTGCCCCACTGGAGGAGGAAGGCAGCCCGAACAGCGCTCACCT
AACTAACAGCTGCTGAGAGCTGGGTTCCGTGGCCATGCACCTGGGACTGCCTTGAGA
AGCGTGAGCAAACCGCCCAGAGTAGAAGCGCTAGCCACCATGGATTGGGGCACGCT
GCAGACGATCCTGGGGGGTGTGAACAAACACTCCACCAGCATTGGAAAGATCTGGC
TCACCGTCCTCTTCATTTTTCGCATTATGATCCTCGTTGTGGCTGCAAAGGAGGTGTG
GGGAGATGAGCAGGCCGACTTTGTCTGCAACACCCTGCAGCCAGGCTGCAAGAACG
TGTGCTACGATCACTACTTCCCCATCTCCCACATCCGGCTATGGGCCCTGCAGCTGAT
CTTCGTGTCCACGCCAGCGCTCCTAGTGGCCATGCACGTGGCCTACCGGAGACATGA
GAAGAAGAGGAAGTTCATCAAGGGGGAGATAAAGAGTGAATTTAAGGACATCGAG
GAGATCAAAACCCAGAAGGTCCGCATCGAAGGCTCCCTGTGGTGGACCTACACAAG
CAGCATCTTCTTCCGGGTCATCTTCGAAGCCGCCTTCATGTACGTCTTCTATGTCATG
TACGACGGCTTCTCCATGCAGCGGCTGGTGAAGTGCAACGCCTGGCCTTGTCCCAAC
ACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAA
TTAGATATTGTTCTGGGAAGTCAAAAAAGCCAGTTGGATCCCGGGCTGACTACAAAG
ACCATGACGGTGATTATAAAGATCATGACATCGACTACAAGGATGACGATGACAAG
TAAGAAATAGACAGCATGAGAGGGATGAGGCAACCCGTGCTCAGCTGTCAAGGCTC
AGTCGCTAGCATTTCCCAACACAAAGATTCTGACCTTAAATGCAACCATTTGAAACC
CCTGTAGGCCTCAGGTGAAACTCCAGATGCCACAATGGAGCTCTGCTCCCCTAAAGC
CTCAAAACAAAGGCCTAATTCTATGCCTGTCTTAATTTTCTTTCACTTAAGTTAGTTC
CACTGAGACCCCAGGCTGTTAGGGGTTATTGGTGTAAGGTACTTTCATATTTTAAAC
AGAGGATATCGGCATTTGTTTCTTTCTCTGAGGACAAGAGAAAAAAGCCAGGTTCCA
CAGAGGACACAGAGAAGGTTTGGGTGTCCTCCTGGGGTTCTTTTTGCCAACTTTCCC
CACGTTAAAGGTGAACATTGGTTCTTTCATTTGCTTTGGAAGTTTTAATCTCTAACAG
TGGACAAAGTTACCAGTGCCTTAAACTCTGTTACACTTTTTGGAAGTGAAAACTTTGT
AGTATGATAGGTTATTTTGATGTAAAGATGTTCTGGATACCATTATATGTTCCCCCTG
TTTCAGAGGCTCAGATTGTAATATGTAAATGGTATGTCATTCGCTACTATGATTTAAT
TTGAAATATGGTCTTTTGGTTATGAATACTTTGCAGCACAGCTGAGAGGCTGTCTGTT
GTATTCATTGTGGTCATAGCACCTAACAACATTGTAGCCTCAATCGAGTGAGACAGA
CTAGAAGTTCCTAGTGATGGCTTATGATAGCAAATGGCCTCATGTCAAATATTTAGA
TGTAATTTTGTGTAAGAAATACAGACTGGATGTACCACCAACTACTACCTGTAATGA
CAGGCCTGTCCAACACATCTCCCTTTTCCATGACTGTGGTAGCCAGCATCGGAAAGA
ACGCTGATTTAAAGAGGTCGCTTGGGAATTTTATTGACACAGTACCATTTAATGGGG
AGGACAAAATGGGGCAGGGGAGGGAGAAGTTTCTGTCGTTAAAAACAGATTTGGAA
AGACTGGACTCTAAAGTCTGTTGATTAAAGATGAGCTTTGTCTACTTCAAAAGTTTGT
TTGCTTACCCCTTCAGCCTCCAATTTTTTAAGTGAAAATATAGCTAATAACATGTGAA
AAGAATAGAAGCTAAGGTTTAGATAAATATTGAGCAGATCTATAGGAAGATTGAAC
CTGAATATTGCCATTATGCTTGACATGGTTTCCAAAAAATGGTACTCCACATATTTCA
GTGAGGGTAAGTATTTTCCTGTTGTCAAGAATAGCATTGTAAAAGCATTTTGTAATA
ATAAAGAATAGCTTTAATGATATGCTTGTAACTAAAATAATTTTGTAATGTATCAAA
TACATTTAAAACATTAAAATATAATCTCTATAATAAGAGCTCGCTGATCAGCCTCGA
CTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGAAG
CTTGAATTCAGCTGACGTGCCTCGGACCGCTAGGAACCCCTAGTGATGGAGTTGGCC
ACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGCTGCCTGCA
GGGGCGCCTGATGCGGTATTTTCTCCTTACGCATCTGTGCGGTATTTCACACCGCATA
CGTCAAAGCAACCATAGTACGCGCCCTGTAGCGGCGCATTAAGCGCGGCGGGTGTG
GTGGTTACGCGCAGCGTGACCGCTACACTTGCCAGCGCCCTAGCGCCCGCTCCTTTC
GCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACT
TGATTTGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACA
CTCAACCCTATCTCGGGCTATTCTTTTGATTTATAAGGGATTTTGCCGATTTCGGCCT
ATTGGTTAAAAAATGAGCTGATTTAACAAAAATTTAACGCGAATTTTAACAAAATAT
TAACGTTTACAATTTTATGGTGCACTCTCAGTACAATCTGCTCTGATGCCGCATAGTT
AAGCCAGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATT
TTTATAGGTTAATGTCATGAACAATAAAACTGTCTGCTTACATAAACAGTAATACAA
GGGGTGTTATGAGCCATATTCAACGGGAAACGTCGAGGCCGCGATTAAATTCCAAC
ATGGATGCTGATTTATATGGGTATAAATGGGCTCGCGATAATGTCGGGCAATCAGGT
GCGACAATCTATCGCTTGTATGGGAAGCCCGATGCGCCAGAGTTGTTTCTGAAACAT
GGCAAAGGTAGCGTTGCCAATGATGTTACAGATGAGATGGTCAGACTAAACTGGCT
GACGGAATTTATGCCTCTTCCGACCATCAAGCATTTTATCCGTACTCCTGATGATGCA
TGGTTACTCACCACTGCGATCCCCGGAAAAACAGCATTCCAGGTATTAGAAGAATAT
CCTGATTCAGGTGAAAATATTGTTGATGCGCTGGCAGTGTTCCTGCGCCGGTTGCATT
CGATTCCTGTTTGTAATTGTCCTTTTAACAGCGATCGCGTATTTCGTCTCGCTCAGGC
GCAATCACGAATGAATAACGGTTTGGTTGATGCGAGTGATTTTGATGACGAGCGTAA
TGGCTGGCCTGTTGAACAAGTCTGGAAAGAAATGCATAAACTTTTGCCATTCTCACC
GGATTCAGTCGTCACTCATGGTGATTTCTCACTTGATAACCTTATTTTTGACGAGGGG
AAATTAATAGGTTGTATTGATGTTGGACGAGTCGGAATCGCAGACCGATACCAGGAT
CTTGCCATCCTATGGAACTGCCTCGGTGAGTTTTCTCCTTCATTACAGAAACGGCTTT
TTCAAAAATATGGTATTGATAATCCTGATATGAATAAATTGCAGTTTCATTTGATGCT
CGATGAGTTTTTCTAATCTCATGACCAAAATCCCTTAACGTGAGTTTTCGTTCCACTG
AGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGAT
ACCAAATACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCT
ACACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCA
CATGT

TABLE 7
Components of Construct Sequence (SEQ ID NO: 38)

| Components | Position in construct |
|---|---|
| 5′ITR | 12-130 |
| Cloning site | 131-147 |
| GJB6 promoter | 148-882 |
| hGJB2 minimal promoter | 883-1010 |
| Cloning site | 1011-1019 |
| Synthetic barcode | 1020-1027 |
| 5′UTR | 1028-1389 |
| GJB2 (exon2) | 1401-2078 |
| 3xFLAG | 2091-2156 |
| 3′UTR (exon2) | 2160-3566 |
| bGHpA | 3588-3812 |
| Cloning site | 3813-3846 |
| 3′ITR | 3847-3976 |

Exemplary Construct sequence
(SEQ ID NO: 7)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTTGTACAGGAGATAGTCAGGGAAT
TAGTAATTTTCAAAGAGGTGACTTTGAATTCAAACTTAAATATCATCTTCAGCTGAA
ACAAAGAAGGGGTGCAGTTATGAGGAAGTGACCAGGTAAAGCATGGCAAACAAAG
GTAAAGTTTGTTATGCGTATTTAAGTCAGAGCCCTCTCCATTGATAAGAGTTTCCAGT
AATTTAGTGCCATCCTTTTCTTGCTATAGAGTTCTCGTCTCTATCTGAGCACGCAAAA
ATAACATGCTTTCTTGCTTTCTTGAAGTTGGGCATGGCCATTGACTTGCCTTAGCCCA
TATTTTTCTGTGAAGTGGTCTTCAAAAACCTATATTTCTGCCATAGAGTCACTTACTT
AACCTGCCCTATTTAAAGGGGCTAATGCCTGATAGAATGTCGCTGCATAACTCCATC
TGTGTGTGGTCCCTGCATCCATGACAACCAAAACCCAGATGCAGAAATTGTTCCTAA
TCACATAGATTACCCTAGAAACCGGAAGGGCCTTGAAGTCAAAAGCATTCAGAGAA
CATGCTGAACAAATTGAATTTGCAGTTTATCTGGCCAGGGAGGATGGAGAGGGGAT
GGGCACTTGGTCTGAGTATTTTTTGTTTCTCATTCCAACAGAAATTACTAGATTTACC
AAAAAATCTACAAGTGGTAGTGTGATAGAGTCAGGCAGAGGAATTGACCATAGATA
AGGTGCTCAGGACTCCTAGAGTCAGCTTCTGGTATGTGAGAAAGAAGTGAGAACAG
AGCCCATGGCATATGAAGAAGATATTACAGAAAAAAGAAAGCTGCCTTCCACGCAA
ATCATTTCTTTACAAAGGCTTGTTAACTCCTGCAGTGCCAAGAAGCTGAATGCAGCG
GCAGACATCCTGGTTCGGGCCCCAGGAAGCTCAGCCGGGTTTAATGTGGATGAGGGT
TTAATGATGTACACGCAGAAGTGTTTTGACAAATGAAGAAGGTCCTCATTCTTGGAA
CATGTGCCGGTTCTCCGAGGGAACTCCTAAAAGGCTGTAAGCTCATGTAGGAAAAGC
TGAGCTAGATTCCTAAGGGCAGAGATGTGCTCACATTTCTTTGCATCCCTAGTTCCCA
GCACAGTGCAAGGCGCTGCAAACATTTGCTGAACCCAGGGTCTCGTGTCTT7GACTG
TCCAGCAGAGGCCGCTCTGGGCCGGGGCTCTCGGGACCTGAGGGCTGAGAGAAGGA
AGGCCAGGGGGTGGCCCAGTCATCGCCGCGGGGCCCGGGTGGGAGGGGTTTGGCAG
CGGCAGGCGCGGCGGCGGCGGCGGAGGCGGAGGCGGCCCCGGGAAGCTCTGAGGA
CCCAGAGGCCGGGCGCGCTCCGCCCGCGGCGCCGCCCCCTCCGTAACTTTCCCAGTC
TCCGAGGGAAGAGGCGGGGTGTGGGGTGCGGTTAAAAGGCGCCACGGCGGGAGAC
AGGTCTCACCGGTGCAAACTGGTTGCGGCCCCGCAGCGCCCGCGCGCTCCTCTCCCC
GACTCGGAGCCCCTCGGCGGCGCCCGGCCCAGGACCCGCCTAGGAGCGCAGGAGCC
CCAGCGCAGAGACCCCAACGCCGAGACCCCCGCCCCGGCCCCGCCGCGCTTCCTCCC
GACGCAGTTTAGGACCCTTGTTCGCGAAGAGGTGGTGTGCGGCTGAGACCCGCGTCC
TCAGGACGGTTCCATCAGTGCCTCGATCCTGCCCCACTGGAGGAGGAAGGCAGCCCG
AACAGCGCTCACCTAACTAACAGCTGCTGAGAGCTGGGTTCCGTGGCCATGCACCTG
GGACTGCCTTGAGAAGCGTGAGCAAACCGCCCAGAGTAGAAGCGCTAGCCACCATG
GATTGGGGCACGCTGCAGACGATCCTGGGGGGTGTGAACAAACACTCCACCAGCAT
TGGAAAGATCTGGCTCACCGTCCTCTTCATTTTTCGCATTATGATCCTCGTTGTGGCT
GCAAAGGAGGTGTGGGGAGATGAGCAGGCCGACTTTGTCTGCAACACCCTGCAGCC
AGGCTGCAAGAACGTGTGCTACGATCACTACTTCCCCATCTCCCACATCCGGCTATG
GGCCCTGCAGCTGATCTTCGTGTCCACGCCAGCGCTCCTAGTGGCCATGCACGTGGC
CTACCGGAGACATGAGAAGAAGAGGAAGTTCATCAAGGGGGAGATAAAGAGTGAA
TTTAAGGACATCGAGGAGATCAAAACCCAGAAGGTCCGCATCGAAGGCTCCCTGTG
GTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGC
CTGGCCTTGTCCCAACACTGTGGACTGCTTTGTGTCCCGGCCCACGGAGAAGACTGT
CTTCACAGTGTTCATGATTGCAGTGTCTGGAATTTGCATCCTGCTGAATGTCACTGAA
TTGTGTTATTTGCTAATTAGATATTGTTCTGGGAAGTCAAAAAAGCCAGTTGGATCCC
GGGCTGACTACAAAGACCATGACGGTGATTATAAAGATCATGACATCGACTACAAG
GATGACGATGACAAGTAAGAAATAGACAGCATGAGAGGGATGAGGCAACCCGTGCT
CAGCTGTCAAGGCTCAGTCGCTAGCATTTCCCAACACAAAGATTCTGACCTTAAATG
CAACCATTTGAAACCCCTGTAGGCCTCAGGTGAAACTCCAGATGCCACAATGGAGCT
CTGCTCCCCTAAAGCCTCAAAACAAAGGCCTAATTCTATGCCTGTCTTAATTTTCTTT
CACTTAAGTTAGTTCCACTGAGACCCCAGGCTGTTAGGGGTTATTGGTGTAAGGTAC
TTTCATATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGTTCTTT
TTGCCAACTTTCCCCACGTTAAAGGTGAACATTGGTTCTTTCATTTGCTTTGGAAGTT
TTAATCTCTAACAGTGGACAAAGTTACCAGTGCCTTAAACTCTGTTACACTTTTTGGA
AGTGAAAACTTTGTAGTATGATAGGTTATTTTGATGTAAAGATGTTCTGGATACCATT
ATATGTTCCCCCTGTTTCAGAGGCTCAGATTGTAATATGTAAATGGTATGTCATTCGC
TACTATGATTTAATTTGAAATATGGTCTTTTGGTTATGAATACTTTGCAGCACAGCTG
AGAGGCTGTCTGTTGTATTCATTGTGGTCATAGCACCTAACAACATTGTAGCCTCAAT
CGAGTGAGACAGACTAGAAGTTCCTAGTGATGGCTTATGATAGCAAATGGCCTCATG
TCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCA
GCATCGGAAAGAACGCTGATTTAAAGAGGTCGCTTGGGAATTTTATTGACACAGTAC
CATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTAC
TTCAAAAGTTTGTTTGCTTACCCCTTCAGCCTCCAATTTTTTAAGTGAAAATATAGCT
AATAACATGTGAAAAGAATAGAAGCTAAGGTTTAGATAAATATTGAGCAGATCTAT
AGGAAGATTGAACCTGAATATTGCCATTATGCTTGACATGGTTTCCAAAAAATGGTA
CTCCACATATTTCAGTGAGGGTAAGTATTTTCCTGTTGTCAAGAATAGCATTGTAAA
AGCATTTTGTAATAATAAAGAATAGCTTTAATGATATGCTTGTAACTAAAATAATTTT
GTAATGTATCAAATACATTTAAAACATTAAAATATAATCTCTATAATAAGAGCTCGC
TGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGGGGGCA
GGACAGCAAGGGGGAGGATTGGGAAGACAATAGCAGGCATGCTGGGGATGCGGTG
GGCTCTATGGAAGCTTGAATTCAGCTGACGTGCCTCGGACCGCTAGGAACCCCTAGT
GATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGC
GCAGCTGCCTGCAGGGGCGCCTGATGCGGTATTTTCTCCTTACGCATCTGTGCGGTAT
TTCACACCGCATACGTCAAAGCAACCATAGTACGCGCCCTGTAGCGGCGCATTAAGC
GCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGAC
CCCAAAAAACTTGATTTGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTGCC
GATTTCGGCCTATTGGTTAAAAAATGAGCTGATTTAACAAAAATTTAACGCGAATTT
TAACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGACG
GGCTTGTCTGCTCCCGGCATCCGCTTACAGACAAGCTGTGACCGTCTCCGGGAGCTG
CATGTGTCAGAGGTTTTCACCGTCATCACCGAAACGCGCGAGACGAAAGGGCCTCGT
GATACGCCTATTTTTATAGGTTAATGTCATGAACAATAAAACTGTCTGCTTACATAA
ACAGTAATACAAGGGGTGTTATGAGCCATATTCAACGGGAAACGTCGAGGCCGCGA
TTAAATTCCAACATGGATGCTGATTTATATGGGTATAAATGGGCTCGCGATAATGTC
GGGCAATCAGGTGCGACAATCTATCGCTTGTATGGGAAGCCCGATGCGCCAGAGTTG
TTTCTGAAACATGGCAAAGGTAGCGTTGCCAATGATGTTACAGATGAGATGGTCAGA
CTAAACTGGCTGACGGAATTTATGCCTCTTCCGACCATCAAGCATTTTATCCGTACTC
CTGATGATGCATGGTTACTCACCACTGCGATCCCCGGAAAAACAGCATTCCAGGTAT
TAGAAGAATATCCTGATTCAGGTGAAAATATTGTTGATGCGCTGGCAGTGTTCCTGC
GCCGGTTGCATTCGATTCCTGTTTGTAATTGTCCTTTTAACAGCGATCGCGTATTTCG
TCTCGCTCAGGCGCAATCACGAATGAATAACGGTTTGGTTGATGCGAGTGATTTTGA
TGACGAGCGTAATGGCTGGCCTGTTGAACAAGTCTGGAAAGAAATGCATAAACTTTT
GCCATTCTCACCGGATTCAGTCGTCACTCATGGTGATTTCTCACTTGATAACCTTATT
TTTGACGAGGGGAAATTAATAGGTTGTATTGATGTTGGACGAGTCGGAATCGCAGAC
CGATACCAGGATCTTGCCATCCTATGGAACTGCCTCGGTGAGTTTTCTCCTTCATTAC
AGAAACGGCTTTTTCAAAAATATGGTATTGATAATCCTGATATGAATAAATTGCAGT
TTCATTTGATGCTCGATGAGTTTTTCTAATCTCATGACCAAAATCCCTTAACGTGAGT
TTTCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTCAG
CAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGG
CCTTTTGCTCACATGT

TABLE 8
Components of Construct Sequence (SEQ ID NO: 7)

| Components | Position in construct |
|---|---|
| 5′ITR | 12-130 |
| Cloning site | 131-147 |
| PARM1 promoter | 148-1463 |
| hGJB2 minimal promoter | 1464-1591 |
| Cloning site | 1592-1600 |
| Synthetic barcode | 1601-1608 |
| 5′UTR | 1609-1970 |
| GJB2 (exon2) | 1982-2659 |
| 3xFLAG | 2672-2737 |
| 3′UTR (exon2) | 2741-4147 |
| bGHpA | 4169-4393 |
| Cloning site | 4394-4427 |
| 3′ITR | 4428-4557 |

Exemplary Construct sequence
(SEQ ID NO: 100)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTTGTGCTGCGAGGGCTTCATCTCC
TAAGCACTAAATGCTAAATTCCCCCTCCCACGCCCATCGCCACTGTCCTCACGGATC
CTCGCAGCAGCTTCCCAATCGGTCTCCCTGTCTCCAGCCTCACCACCCCCAACTAAG
ACCATTCATGAAAACAGAGACAACCAAGGAGACAGTCACCCAATGCTGTCCCTTCA
GCTTGCATTATTTTCTGACAAGACAGCTCTGCCATCCATGGAAGCCTGTGTTTGAAG
ATCTCTGACATAAAGGTCCCTTGCAGAGCTAGACGTGATTCTAAAATTGGGAACACA
GGAATAAAAATCAAATCTTGAGTAGAAGTAGCTGAAAATTGCAGTGATTCGGGGAA
GCTTGGCTTCTAACTCCCCACTGTTTGAAGATGGGCTTGTTTGTTTTTTAAAACAGCC
AACATAATTCAGCTGGAGGAGGTACAAAGAATTTTCTATTCCTTGTTTCTGTAGAAA
TCGATGGACTTTAGCTTGTCTAATTGTCCCCCCTGCCTTTAGTATCTAAAATAAAATA
ACCCTCGTTGCTTGCATTACTCAACGCATTTCTGCGTCTTGGCGTCTATGGCTAAACG
AGTATTAATTAGACAGTCCGCAGAGAGCTGGCTGGGGATAGAAGGGGAGGTGGGGG
AGAAGGGCAGGGATCACAGCAGGGTGGACTCGTGGCCCTGATTTGGGATCCTGACA
GCAACTTACTAGGTGGCCTGAGGGCTGGGTGCCAGGGGAGGCAGCGGGTTCCAGTA
GCATCTGACCTGCATTAGGGACAGGGGCGCGGCGGAGGGGGCGAAGGGGGCGGGG
GTGGGGGGAAGGTGGCTGGGGTGAAGCCCAGCTTCGCAGCTAGCTGTGGGCAACAG
AGGGAGTAAGGGGGGGCAATGAGGCTGGGGCCAGGCGCCAGCAGCAGCCACGCCC
CCCACCTCCCCCGATTTTTAGGGAAAATTCTCCAAAGCTCTCGCATCCTCCTCTGCCT
CCTTCCACCCTCCACCCTCCCAGCCTCCACTGAGACCTCTTTAAAACCACCCAGGGG
CCGCCGGGGGATGAGGCCGGGGAACGGGCTGGACTGAGGGCGGGGGCTCGGGGGC
AGCGGACGGGAAACGCCTCGAAAGCAGCCAGACCCGGCGACTGAAATGAGGCGGA
GGAGCTTGGCGAGGGGAGGCGCAGGCTCGGAAAGGCGCGCGAGGCTCCAGGCTCCT
TCCCGATCCACCGCTCTCCTCGCTGACCTCCGAGTCACCCCCGGAAGCTCCCGCCACT
GCCGGGCGAATAGACCCCCGCGGACCCCCAAGCGCGCGGGGCCGGGGCCCTAGTTC
AGGCCCTCGCTGCCCCTTTAAGGGTTCTCGAAACTTTCCCCCCGGTATCAGATGAGC
CTCGTCACATCCGTTGGCCGTGGCAAGCTCTGAGGACCCAGAGGCCGGGCGCGCTCC
GCCCGCGGCGCCGCCCCCTCCGTAACTTTCCCAGTCTCCGAGGGAAGAGGCGGGGTG
TGGGGTGCGGTTAAAAGGCGCCACGGCGGGAGACAGGTCTCACCGGTCCTACGCTG
TTGCGGCCCCGCAGCGCCCGCGCGCTCCTCTCCCCGACTCGGAGCCCCTCGGCGGCG
CCCGGCCCAGGACCCGCCTAGGAGCGCAGGAGCCCCAGCGCAGAGACCCCAACGCC
GAGACCCCCGCCCCGGCCCCGCCGCGCTTCCTCCCGACGCAGTTTAGGACCCTTGTT
CGCGAAGAGGTGGTGTGCGGCTGAGACCCGCGTCCTCAGGACGGTTCCATCAGTGCC
TCGATCCTGCCCCACTGGAGGAGGAAGGCAGCCCGAACAGCGCTCACCTAACTAAC
AGCTGCTGAGAGCTGGGTTCCGTGGCCATGCACCTGGGACTGCCTTGAGAAGCGTGA
GCAAACCGCCCAGAGTAGAAGCGCTAGCCACCATGGATTGGGGCACGCTGCAGACG
ATCCTGGGGGGTGTGAACAAACACTCCACCAGCATTGGAAAGATCTGGCTCACCGTC
CTCTTCATTTTTCGCATTATGATCCTCGTTGTGGCTGCAAAGGAGGTGTGGGGAGATG
AGCAGGCCGACTTTGTCTGCAACACCCTGCAGCCAGGCTGCAAGAACGTGTGCTACG
ATCACTACTTCCCCATCTCCCACATCCGGCTATGGGCCCTGCAGCTGATCTTCGTGTC
CACGCCAGCGCTCCTAGTGGCCATGCACGTGGCCTACCGGAGACATGAGAAGAAGA
GGAAGTTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCATCTTC
TTCCGGGTCATCTTCGAAGCCGCCTTCATGTACGTCTTCTATGTCATGTACGACGGCT
TCTCCATGCAGCGGCTGGTGAAGTGCAACGCCTGGCCTTGTCCCAACACTGTGGACT
GCTTTGTGTCCCGGCCCACGGAGAAGACTGTCTTCACAGTGTTCATGATTGCAGTGT
CTGGAATTTGCATCCTGCTGAATGTCACTGAATTGTGTTATTTGCTAATTAGATATTG
TTCTGGGAAGTCAAAAAAGCCAGTTGGATCCCGGGCTGACTACAAAGACCATGACG
GTGATTATAAAGATCATGACATCGACTACAAGGATGACGATGACAAGTAAGAAATA
GACAGCATGAGAGGGATGAGGCAACCCGTGCTCAGCTGTCAAGGCTCAGTCGCTAG
CATTTCCCAACACAAAGATTCTGACCTTAAATGCAACCATTTGAAACCCCTGTAGGC
CTCAGGTGAAACTCCAGATGCCACAATGGAGCTCTGCTCCCCTAAAGCCTCAAAACA
AAGGCCTAATTCTATGCCTGTCTTAATTTTCTTTCACTTAAGTTAGTTCCACTGAGAC
CCCAGGCTGTTAGGGGTTATTGGTGTAAGGTACTTTCATATTTTAAACAGAGGATAT
CGGCATTTGTTTCTTTCTCTGAGGACAAGAGAAAAAAGCCAGGTTCCACAGAGGACA
CAGAGAAGGTTTGGGTGTCCTCCTGGGGTTCTTTTTGCCAACTTTCCCCACGTTAAAG
GTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAG
GTTATTTTGATGTAAAGATGTTCTGGATACCATTATATGTTCCCCCTGTTTCAGAGGC
TCAGATTGTAATATGTAAATGGTATGTCATTCGCTACTATGATTTAATTTGAAATATG
GTCTTTTGGTTATGAATACTTTGCAGCACAGCTGAGAGGCTGTCTGTTGTATTCATTG
TGGTCATAGCACCTAACAACATTGTAGCCTCAATCGAGTGAGACAGACTAGAAGTTC
CTAGTGATGGCTTATGATAGCAAATGGCCTCATGTCAAATATTTAGATGTAATTTTGT
GTAAGAAATACAGACTGGATGTACCACCAACTACTACCTGTAATGACAGGCCTGTCC
AACACATCTCCCTTTTCCATGACTGTGGTAGCCAGCATCGGAAAGAACGCTGATTTA
AAGAGGTCGCTTGGGAATTTTATTGACACAGTACCATTTAATGGGGAGGACAAAATG
GGGCAGGGGAGGGAGAAGTTTCTGTCGTTAAAAACAGATTTGGAAAGACTGGACTC
TAAAGTCTGTTGATTAAAGATGAGCTTTGTCTACTTCAAAAGTTTGTTTGCTTACCCC
TTCAGCCTCCAATTTTTTAAGTGAAAATATAGCTAATAACATGTGAAAAGAATAGAA
GCTAAGGTTTAGATAAATATTGAGCAGATCTATAGGAAGATTGAACCTGAATATTGC
CATTATGCTTGACATGGTTTCCAAAAAATGGTACTCCACATATTTCAGTGAGGGTAA
GTATTTTCCTGTTGTCAAGAATAGCATTGTAAAAGCATTTTGTAATAATAAAGAATA
GCTTTAATGATATGCTTGTAACTAAAATAATTTTGTAATGTATCAAATACATTTAAAA
CATTAAAATATAATCTCTATAATAAGAGCTCGCTGATCAGCCTCGACTGTGCCTTCTA
GTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGG
AAGACAATAGCAGGCATGCTGGGGATGCGGTGGGCTCTATGGAAGCTTGAATTCAG
CTGACGTGCCTCGGACCGCTAGGAACCCCTAGTGATGGAGTTGGCCACTCCCTCTCT
GCGCGCTCGCTCGCTCACTGAGGCCGGGCGACCAAAGGTCGCCCGACGCCCGGGCTT
TGCCCGGGCGGCCTCAGTGAGCGAGCGAGCGCGCAGCTGCCTGCAGGGGCGCCTGA
TGCGGTATTTTCTCCTTACGCATCTGTGCGGTATTTCACACCGCATACGTCAAAGCAA
CCATAGTACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTTGGGTGA
TGGTTCACGTAGTGGGCCATCGCCCTGATAGACGGTTTTTCGCCCTTTGACGTTGGAG
TCCACGTTCTTTAATAGTGGACTCTTGTTCCAAACTGGAACAACACTCAACCCTATCT
CGGGCTATTCTTTTGATTTATAAGGGATTTTGCCGATTTCGGCCTATTGGTTAAAAAA
TGAGCTGATTTAACAAAAATTTAACGCGAATTTTAACAAAATATTAACGTTTACAAT
TTTATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCCGC
TTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAA
TGTCATGAACAATAAAACTGTCTGCTTACATAAACAGTAATACAAGGGGTGTTATGA
GCCATATTCAACGGGAAACGTCGAGGCCGCGATTAAATTCCAACATGGATGCTGATT
TATATGGGTATAAATGGGCTCGCGATAATGTCGGGCAATCAGGTGCGACAATCTATC
GCTTGTATGGGAAGCCCGATGCGCCAGAGTTGTTTCTGAAACATGGCAAAGGTAGCG
TTGCCAATGATGTTACAGATGAGATGGTCAGACTAAACTGGCTGACGGAATTTATGC
CTCTTCCGACCATCAAGCATTTTATCCGTACTCCTGATGATGCATGGTTACTCACCAC
TGCGATCCCCGGAAAAACAGCATTCCAGGTATTAGAAGAATATCCTGATTCAGGTGA
AAATATTGTTGATGCGCTGGCAGTGTTCCTGCGCCGGTTGCATTCGATTCCTGTTTGT
AATTGTCCTTTTAACAGCGATCGCGTATTTCGTCTCGCTCAGGCGCAATCACGAATG
AATAACGGTTTGGTTGATGCGAGTGATTTTGATGACGAGCGTAATGGCTGGCCTGTT
GAACAAGTCTGGAAAGAAATGCATAAACTTTTGCCATTCTCACCGGATTCAGTCGTC
ACTCATGGTGATTTCTCACTTGATAACCTTATTTTTGACGAGGGGAAATTAATAGGTT
GTATTGATGTTGGACGAGTCGGAATCGCAGACCGATACCAGGATCTTGCCATCCTAT
GGAACTGCCTCGGTGAGTTTTCTCCTTCATTACAGAAACGGCTTTTTCAAAAATATGG
TATTGATAATCCTGATATGAATAAATTGCAGTTTCATTTGATGCTCGATGAGTTTTTC
TAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTAC
CAACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGAGA
TACCTACAGCGTGAGCTATGAGAAAGCGCCACGCTTCCCGAAGGGAGAAAGGCGGA
CAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAAC
GCGGCCTTTTTACGGTTCCTGGCCTTTTGCTGGCCTTTTGCTCACATGT

TABLE 9
Components of Construct Sequence (SEQ ID NO: 100)

| Components | Position in construct |
|---|---|
| 5′ITR | 12-130 |
| Cloning site | 121-147 |
| BACE2 promoter | 148-1551 |
| hGJB2 minimal promoter | 1552-1679 |
| Cloning site | 1680-1688 |
| Synthetic barcode | 1689-1696 |
| 5′UTR | 1697-2058 |
| GJB2 (exon2) | 2070-2747 |
| 3xFLAG | 2760-2825 |
| 3′UTR (exon2) | 2829-4235 |
| bGHpA | 4255-4481 |
| Cloning site | 4482-4515 |
| 3′ITR | 4516-4645 |

Exemplary Construct sequence
(SEQ ID NO: 101)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTGAAGAAACCTGCATTTCTTACAC
TTCAGTGTACTTTCCCCATATTTAACTCCAAGATTTTTGTTAATTTGTTTGGTTTTCCT
TTCTCAAACAAAATTATGCTCAGACTGAAAACCCTAGATTTGTTCCCTATTGCATCTT
CATTTCTTCCCAAACATTCCATAAAACGTGACCTACATTAAGTTAGCAAGTTAAGTCT
GAAAGCGTCTACCTTCCCTGGGGAGGGGGAAGGTGTAGGCAGGGCAGAGATTTGTA
GTCCAGCCCTCTTGCCACAAATTATGAATTAGAGAGGAATGACTTTGCTTTTTTAATG
ATCTCCAGAGAATTTTCCATCATTTCCCTCTCTTCACCCAGCTCCTTTGCAACCACTG
CCAGAGAAGTCTTCCTTTAGCTTCTTAAACATCGATCCTAAAACACTTCCAGACACCT
GTGCTGCTCCTTTCAGTTCCCATGGAGATTAGGCTGTGTAACAATCTCGCAAAGACG
TTCCCCTCCGTCTCCTCATCCTCTTTTCAAACCCTTTTACGATTTCCCATCTCACTCAG
CATGACAGTCAAAGTCCCTGTGATGGCCAACTTCTGCATCACCTAGCCAGTCTGCCA
CCGCCAAAACTCTCCAGCCTCATCTTACACTTGTTCTCTGCTTGGAATCTTCCCTCCC
CTCCTTGAGGAACTTTCTCAAATGTCACCTTCCCTCAATACTCCCCCTCCTCCATTTA
AAACTATAAACTTCCAACTCTCTAAGCCCCTAAAGTACTCTATATTTAACTTATTGTA
TAAACTACTGTCCCTACTTGTAAGTTCCAAGATTGCAGGGATTCACCCGCTTTGTTCA
CTGCTGTCTGCCAAGGTCTAGAACAGTGCAAGTTACCCAACAGGAGTTCAATAAACA
GCCATTCATTTAACAAATATTTGCTGAGCACTTCGTCCCGTCCAAGTTTGTTAAATCA
AGACAAATAAGACACCGTCCCTGCCTTTAACGCACCAGATGGAGAAATGCACCACA
GACATAAATGTGCAATACAGGCCTGACACTACGGCCACAAGCAAGTCAAAGAACGT
GCCAAAAGTTCAGAGGAAGAAGCCTCGGCTTCGCCTTTCGGGAGACCAGTCCAGCTT
TCCACCATCACGCTGCTCATCAGGGACCATCTCCGGGGGTCTCCTCTAGACCCCAAG
GGAGGAGCGGGTCCCGCCCGCCATTCCCAGGTCTCAGAGTTTACTTGTCCAGAGATG
CAACTTCCGGCCTCTTCAGGCCGGGCAAGATTTAAGGAAAGAAAAGAAACATAAGG
ACCTCCGTTCTTCGGTCTCCGTCCCCTCCCCTTCCCCCGCGTGCCCCACCTGTTCCCG
GCGTCCCCTTCGGCTACTCCCGGCGTTTGCGCAAGCGGTCCCACGTGGGCTCGGGCG
GGGCTAGCGCCGCGGCGGGGGCTGGGCACGCCCCTAGCGCATAGCTGGCTTCTGATT
GGCTTTCCAAGCTCTGAGGACCCAGAGGCCGGGCGCGCTCCGCCCGCGGCGCCGCCC
CCTCCGTAACTTTCCCAGTCTCCGAGGGAAGAGGCGGGGTGTGGGGTGCGGTTAAAA
GGCGCCACGGCGGGAGACAGGTCTCACCGGTGCCAAAGCGTTGCGGCCCCGCAGCG
CCCGCGCGCTCCTCTCCCCGACTCGGAGCCCCTCGGCGGCGCCCGGCCCAGGACCCG
CCTAGGAGCGCAGGAGCCCCAGCGCAGAGACCCCAACGCCGAGACCCCCGCCCCGG
CCCCGCCGCGCTTCCTCCCGACGCAGTTTAGGACCCTTGTTCGCGAAGAGGTGGTGT
GCGGCTGAGACCCGCGTCCTCAGGACGGTTCCATCAGTGCCTCGATCCTGCCCCACT
GGAGGAGGAAGGCAGCCCGAACAGCGCTCACCTAACTAACAGCTGCTGAGAGCTGG
GTTCCGTGGCCATGCACCTGGGACTGCCTTGAGAAGCGTGAGCAAACCGCCCAGAGT
AGAAGCGCTAGCCACCATGGATTGGGGCACGCTGCAGACGATCCTGGGGGGTGTGA
ACAAACACTCCACCAGCATTGGAAAGATCTGGCTCACCGTCCTCTTCATTTTTCGCAT
TATGATCCTCGTTGTGGCTGCAAAGGAGGTGTGGGGAGATGAGCAGGCCGACTTTGT
CTGCAACACCCTGCAGCCAGGCTGCAAGAACGTGTGCTACGATCACTACTTCCCCAT
CTCCCACATCCGGCTATGGGCCCTGCAGCTGATCTTCGTGTCCACGCCAGCGCTCCTA
GTGGCCATGCACGTGGCCTACCGGAGACATGAGAAGAAGAGGAAGTTCATCAAGGG
GGAGATAAAGAGTGAATTTAAGGACATCGAGGAGATCAAAACCCAGAAGGTCCGCA
TCGAAGGCTCCCTGTGGTGGACCTACACAAGCAGCATCTTCTTCCGGGTCATCTTCG
AAGCCGCCTTCATGTACGTCTTCTATGTCATGTACGACGGCTTCTCCATGCAGCGGCT
GGTGAAGTGCAACGCCTGGCCTTGTCCCAACACTGTGGACTGCTTTGTGTCCCGGCC
CACGGAGAAGACTGTCTTCACAGTGTTCATGATTGCAGTGTCTGGAATTTGCATCCT
GCTGAATGTCACTGAATTGTGTTATTTGCTAATTAGATATTGTTCTGGGAAGTCAAAA
AAGCCAGTTGGATCCCGGGCTGACTACAAAGACCATGACGGTGATTATAAAGATCA
TGACATCGACTACAAGGATGACGATGACAAGTAAGAAATAGACAGCATGAGAGGGA
TGAGGCAACCCGTGCTCAGCTGTCAAGGCTCAGTCGCTAGCATTTCCCAACACAAAG
ATTCTGACCTTAAATGCAACCATTTGAAACCCCTGTAGGCCTCAGGTGAAACTCCAG
ATGCCACAATGGAGCTCTGCTCCCCTAAAGCCTCAAAACAAAGGCCTAATTCTATGC
CTGTCTTAATTTTCTTTCACTTAAGTTAGTTCCACTGAGACCCCAGGCTGTTAGGGGT
TATTGGTGTAAGGTACTTTCATATTTTAAACAGAGGATATCGGCATTTGTTTCTTTCT
CTGAGGACAAGAGAAAAAAGCCAGGTTCCACAGAGGACACAGAGAAGGTTTGGGT
GTCCTCCTGGGGTTCTTTTTGCCAACTTTCCCCACGTTAAAGGTGAACATTGGTTCTT
TCATTTGCTTTGGAAGTTTTAATCTCTAACAGTGGACAAAGTTACCAGTGCCTTAAAC
TCTGTTACACTTTTTGGAAGTGAAAACTTTGTAGTATGATAGGTTATTTTGATGTAAA
GATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAA
TACTTTGCAGCACAGCTGAGAGGCTGTCTGTTGTATTCATTGTGGTCATAGCACCTAA
CAACATTGTAGCCTCAATCGAGTGAGACAGACTAGAAGTTCCTAGTGATGGCTTATG
ATAGCAAATGGCCTCATGTCAAATATTTAGATGTAATTTTGTGTAAGAAATACAGAC
TGGATGTACCACCAACTACTACCTGTAATGACAGGCCTGTCCAACACATCTCCCTTTT
CCATGACTGTGGTAGCCAGCATCGGAAAGAACGCTGATTTAAAGAGGTCGCTTGGG
AATTTTATTGACACAGTACCATTTAATGGGGAGGACAAAATGGGGCAGGGGAGGGA
GAAGTTTCTGTCGTTAAAAACAGATTTGGAAAGACTGGACTCTAAAGTCTGTTGATT
AAAGATGAGCTTTGTCTACTTCAAAAGTTTGTTTGCTTACCCCTTCAGCCTCCAATTT
TTTAAGTGAAAATATAGCTAATAACATGTGAAAAGAATAGAAGCTAAGGTTTAGAT
AAATATTGAGCAGATCTATAGGAAGATTGAACCTGAATATTGCCATTATGCTTGACA
TGGTTTCCAAAAAATGGTACTCCACATATTTCAGTGAGGGTAAGTATTTTCCTGTTGT
CAAGAATAGCATTGTAAAAGCATTTTGTAATAATAAAGAATAGCTTTAATGATATGC
TTGTAACTAAAATAATTTTGTAATGTATCAAATACATTTAAAACATTAAAATATAAT
CTCTATAATAAGAGCTCGCTGATCAGCCTCGACTGTGCCTTCTAGTTGCCAGCCATCT
GTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAGG
CATGCTGGGGATGCGGTGGGCTCTATGGAAGCTTGAATTCAGCTGACGTGCCTCGGA
CCGCTAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCT
CAGTGAGCGAGCGAGCGCGCAGCTGCCTGCAGGGGCGCCTGATGCGGTATTTTCTCC
TTACGCATCTGTGCGGTATTTCACACCGCATACGTCAAAGCAACCATAGTACGCGCC
CTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTCCGATTTAG
TGCTTTACGGCACCTCGACCCCAAAAAACTTGATTTGGGTGATGGTTCACGTAGTGG
GCCATCGCCCTGATAGACGGTTTTTCGCCCTTTGACGTTGGAGTCCACGTTCTTTAAT
AGTGGACTCTTGTTCCAAACTGGAACAACACTCAACCCTATCTCGGGCTATTCTTTTG
ATTTATAAGGGATTTTGCCGATTTCGGCCTATTGGTTAAAAAATGAGCTGATTTAAC
AAAAATTTAACGCGAATTTTAACAAAATATTAACGTTTACAATTTTATGGTGCACTCT
CAGTACAATCTGCTCTGATGCCGCATAGTTAAGCCAGCCCCGACACCCGCCAACACC
CGCTGACGCGCCCTGACGGGCTTGTCTGCTCCCGGCATCCGCTTACAGACAAGCTGT
GACCGTCTCCGGGAGCTGCATGTGTCAGAGGTTTTCACCGTCATCACCGAAACGCGC
GAGACGAAAGGGCCTCGTGATACGCCTATTTTTATAGGTTAATGTCATGAACAATAA
AACTGTCTGCTTACATAAACAGTAATACAAGGGGTGTTATGAGCCATATTCAACGGG
AAACGTCGAGGCCGCGATTAAATTCCAACATGGATGCTGATTTATATGGGTATAAAT
GGGCTCGCGATAATGTCGGGCAATCAGGTGCGACAATCTATCGCTTGTATGGGAAGC
CCGATGCGCCAGAGTTGTTTCTGAAACATGGCAAAGGTAGCGTTGCCAATGATGTTA
CAGATGAGATGGTCAGACTAAACTGGCTGACGGAATTTATGCCTCTTCCGACCATCA
AGCATTTTATCCGTACTCCTGATGATGCATGGTTACTCACCACTGCGATCCCCGGAA
AAACAGCATTCCAGGTATTAGAAGAATATCCTGATTCAGGTGAAAATATTGTTGATG
CGCTGGCAGTGTTCCTGCGCCGGTTGCATTCGATTCCTGTTTGTAATTGTCCTTTTAA
CAGCGATCGCGTATTTCGTCTCGCTCAGGCGCAATCACGAATGAATAACGGTTTGGT
TGATGCGAGTGATTTTGATGACGAGCGTAATGGCTGGCCTGTTGAACAAGTCTGGAA
AGAAATGCATAAACTTTTGCCATTCTCACCGGATTCAGTCGTCACTCATGGTGATTTC
TCACTTGATAACCTTATTTTTGACGAGGGGAAATTAATAGGTTGTATTGATGTTGGAC
GAGTCGGAATCGCAGACCGATACCAGGATCTTGCCATCCTATGGAACTGCCTCGGTG
AGTTTTCTCCTTCATTACAGAAACGGCTTTTTCAAAAATATGGTATTGATAATCCTGA
TATGAATAAATTGCAGTTTCATTTGATGCTCGATGAGTTTTTCTAATCTCATGACCAA
AATCCCTTAACGTGAGTTTTCGTTCCACTGAGCGTCAGACCCCGTAGAAAAGATCAA
AGGATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCGT
AGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTTACG
GTTCCTGGCCTTTTGCTGGCCTTTTGCTCACATGT

TABLE 10
Components of Construct Sequence (SEQ ID NO: 101)

| Components | Position in construct |
|---|---|
| 5′ITR | 12-130 |
| Cloning site | 131-147 |
| DBI2 promoter | 148-1614 |
| hGJB2 minimal promoter | 1615-1742 |
| Cloning site | 1743-1751 |
| Synthetic barcode | 1752-1759 |
| 5′UTR | 1760-2121 |
| GJB2 (exon2) | 2133-2810 |
| 3xFLAG | 2823-2888 |
| 3′UTR (exon2) | 2892-4298 |
| bGHpA | 4320-4544 |
| Cloning site | 4545-4578 |
| 3′ITR | 4579-4708 |

Exemplary Construct sequence
(SEQ ID NO: 102)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTTACCATTCTGCCTTTCACCTGATG
TTGCTATCCTCCTCCCTCTTGTTTCCTTCCACCCATCCTTTCCCTCCCACATTACTCTCT
TATCCCACCCTATTTTACAACCAGTAGCCTAGGGAAAAGAGCATAGCTCAAATGAGG
AAGAAGGCAGGACAGGCAGTCATGGCTTAGCTGGACTGAGCTGCAGTGCTTCTCCTT
CTGGGGAAGGGGGTGCACTGTCATCTGCTACTGACACATCCCTCCAAGGCACTCAGC
CCTGCAGGGAGCAACCTGATTCTATGACTGACATCTAATCTTCACATTCACCTTGCA
GGAAGGCAAGAAGTGATCCCAGCCTCCAGATGGAAAGATCAAGGCCCAGAGAAGGT
CAGTGGTGGTTGGAGGCCTGAGGTCACACAGCAGCCAAGTCTGGAGTCACTAGTCA
AGGTGACCTTGACTAGCCACCCCACCTCCCCTTCCCTGCCCCACCATGGCCCTGGGA
GATCTGTTGTCCTGTGAGGGAAAGGGGCTCCAGGCTGGGCTGCATCTGAAGCCCCTA
GATCCAGAGACTTCATTTCTTAGGCTATCTATAAAATCCACCTTCCTTTCTTTTCCCA
GGACCCCCATACCCTGCTCCCAGCATCGTCTGCCTCAGCTAAGCCATGGGGATTGAG
AGACCAGGCCTGGTGCCCAGATAAACTGACCCTGGGTGAGGGGACAGGGGCCCAGA
ATGGGCAGGTAGAGACTGAATACTGAAGAAGAATCCTCTGGAGTCTGTTAGCAGAA
GCAGATGGGCCTTGCCTGACTATTGGCAGGCGGACCTGGTGGTCAGACCTCAGTGAT
CCTCAGGGACCAGTGAATATTTCAGGCTGGGGCTGAGCATCACCTGCTCCCTTGGCC
CCACTTATAGGGCAAAGGGGAGTCTACCAGCCTACTCACTGATGACAAACTGGAAA
AGTTTGTCCTGTCTCTGCTCTGGCCCCACCTCGCCCTCTCCCCTACTTGGAAGTTCCTT
TCCTGAACCACTGACTGCCAAAGCTTGAGGGATTAAATAAATCATCTGGCCCAACCT
CCTACCATAGAGTTGGGAACACTGAAGAAAAGAGACTGGCCCAAGGTCACAGAGAA
GGCAGGGTGAACACTGTCACAGGGAGAGCCAGTGTAGAATAATGGTTAAGCCACGC
AAGCTCTAGAACCACTCTATCTGAGTGCAAATCCTGGCTGTCATCTGGTACTTGCTTC
CTGGAACACATCTGGCCTCAGACTCCTGAGGCCAAGACACACTCCCTGCCCTAAGAC
TTGCTGGTTCTATGGCAGGCAGAGGCAGAAAGAGCCCCACCATCATTCCCAGCAAAT
GGGAAAAGTTCCCAGTTGCAGATATTAGGGGTGGGATGGGGCGGGGGTAGTCAGCA
ACCATAGACTTAGACCCTGAAGAGGCAAAAAAGGAGGGCCATGTTCTTGGGTCAGC
AGAGCTTCTACTCAGCTTCTTCAGCCTCTAGCTCTTTCCTGGTGCTAGTAGCACATTC
TCTAGTGGAGGCATCCAGATGGCAGGGAGGGTCCAGGAAACAGCTGAACATGCTGA
GCAGGCCTCCCTTGTCCCCGCTCCCCATGGCCCCATGGATCATCCGGTGCTGCAGCTC
ATCTCATTGGCTGGCTTCTGGTTACTCATCTCTCCTCTTCTCCATCTTCCCAGCCTGTG
GTTGCCGTGGAAACATAGAACAGTGACCTCACCATAGGATGAGGGCTGGGGAGATG
CTGTTCTTGGCAGGCGCTAAGCTCTGAGGACCCAGAGGCCGGGCGCGCTCCGCCCGC
GGCGCCGCCCCCTCCGTAACTTTCCCAGTCTCCGAGGGAAGAGGCGGGGTGTGGGGT
GCGGTTAAAAGGCGCCACGGCGGGAGACAGGTCTCACCGGTCCATCCACGTTGCGG
CCCCGCAGCGCCCGCGCGCTCCTCTCCCCGACTCGGAGCCCCTCGGCGGCGCCCGGC
CCAGGACCCGCCTAGGAGCGCAGGAGCCCCAGCGCAGAGACCCCAACGCCGAGACC
CCCGCCCCGGCCCCGCCGCGCTTCCTCCCGACGCAGTTTAGGACCCTTGTTCGCGAA
GAGGTGGTGTGCGGCTGAGACCCGCGTCCTCAGGACGGTTCCATCAGTGCCTCGATC
CTGCCCCACTGGAGGAGGAAGGCAGCCCGAACAGCGCTCACCTAACTAACAGCTGC
TGAGAGCTGGGTTCCGTGGCCATGCACCTGGGACTGCCTTGAGAAGCGTGAGCAAA
CCGCCCAGAGTAGAAGCGCTAGCCACCATGGATTGGGGCACGCTGCAGACGATCCT
GGGGGGTGTGAACAAACACTCCACCAGCATTGGAAAGATCTGGCTCACCGTCCTCTT
CATTTTTCGCATTATGATCCTCGTTGTGGCTGCAAAGGAGGTGTGGGGAGATGAGCA
GGCCGACTTTGTCTGCAACACCCTGCAGCCAGGCTGCAAGAACGTGTGCTACGATCA
CTACTTCCCCATCTCCCACATCCGGCTATGGGCCCTGCAGCTGATCTTCGTGTCCACG
CCAGCGCTCCTAGTGGCCATGCACGTGGCCTACCGGAGACATGAGAAGAAGAGGAA
GTTCATCAAGGGGGAGATAAAGAGTGAATTTAAGGACATCGAGGAGATCAAAACCC
AGAAGGTCCGCATCGAAGGCTCCCTGTGGTGGACCTACACAAGCAGCATCTTCTTCC
GGGTCATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGACTGCTT
TGTGTCCCGGCCCACGGAGAAGACTGTCTTCACAGTGTTCATGATTGCAGTGTCTGG
AATTTGCATCCTGCTGAATGTCACTGAATTGTGTTATTTGCTAATTAGATATTGTTCT
GGGAAGTCAAAAAAGCCAGTTGGATCCCGGGCTGACTACAAAGACCATGACGGTGA
TTATAAAGATCATGACATCGACTACAAGGATGACGATGACAAGTAAGAAATAGACA
GCATGAGAGGGATGAGGCAACCCGTGCTCAGCTGTCAAGGCTCAGTCGCTAGCATTT
CCCAACACAAAGATTCTGACCTTAAATGCAACCATTTGAAACCCCTGTAGGCCTCAG
GTGAAACTCCAGATGCCACAATGGAGCTCTGCTCCCCTAAAGCCTCAAAACAAAGG
CCTAATTCTATGCCTGTCTTAATTTTCTTTCACTTAAGTTAGTTCCACTGAGACCCCA
GGCTGTTAGGGGTTATTGGTGTAAGGTACTTTCATATTTTAAACAGAGGATATCGGC
ATTTGTTTCTTTCTCTGAGGACAAGAGAAAAAAGCCAGGTTCCACAGAGGACACAGA
GAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACC
AGTGCCTTAAACTCTGTTACACTTTTTGGAAGTGAAAACTTTGTAGTATGATAGGTTA
TTTTGATGTAAAGATGTTCTGGATACCATTATATGTTCCCCCTGTTTCAGAGGCTCAG
ATTGTAATATGTAAATGGTATGTCATTCGCTACTATGATTTAATTTGAAATATGGTCT
TTTGGTTATGAATACTTTGCAGCACAGCTGAGAGGCTGTCTGTTGTATTCATTGTGGT
CATAGCACCTAACAACATTGTAGCCTCAATCGAGTGAGACAGACTAGAAGTTCCTAG
TGATGGCTTATGATAGCAAATGGCCTCATGTCAAATATTTAGATGTAATTTTGTGTAA
GAAATACAGACTGGATGTACCACCAACTACTACCTGTAATGACAGGCCTGTCCAACA
CATCTCCCTTTTCCATGACTGTGGTAGCCAGCATCGGAAAGAACGCTGATTTAAAGA
GGTCGCTTGGGAATTTTATTGACACAGTACCATTTAATGGGGAGGACAAAATGGGGC
AGGGGAGGGAGAAGTTTCTGTCGTTAAAAACAGATTTGGAAAGACTGGACTCTAAA
GTCTGTTGATTAAAGATGAGCTTTGTCTACTTCAAAAGTTTGTTTGCTTACCCCTTCA
GCCTCCAATTTTTTAAGTGAAAATATAGCTAATAACATGTGAAAAGAATAGAAGCTA
AGGTTTAGATAAATATTGAGCAGATCTATAGGAAGATTGAACCTGAATATTGCCATT
ATGCTTGACATGGTTTCCAAAAAATGGTACTCCACATATTTCAGTGAGGGTAAGTAT
TTTCCTGTTGTCAAGAATAGCATTGTAAAAGCATTTTGTAATAATAAAGAATAGCTTT
AATGATATGCTTGTAACTAAAATAATTTTGTAATGTATCAAATACATTTAAAACATT
AAAATATAATCTCTATAATAAGAGCTCGCTGATCAGCCTCGACTGTGCCTTCTAGTT
GCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGA
CAATAGCAGGCATGCTGGGGATGCGGTGGGCTCTATGGAAGCTTGAATTCAGCTGAC
GTGCCTCGGACCGCTAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAGCAACCATA
GTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTTGGGTGATGGTTC
ACGTAGTGGGCCATCGCCCTGATAGACGGTTTTTCGCCCTTTGACGTTGGAGTCCAC
GTTCTTTAATAGTGGACTCTTGTTCCAAACTGGAACAACACTCAACCCTATCTCGGGC
TATTCTTTTGATTTATAAGGGATTTTGCCGATTTCGGCCTATTGGTTAAAAAATGAGC
TGATTTAACAAAAATTTAACGCGAATTTTAACAAAATATTAACGTTTACAATTTTATG
GTGCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTCAT
GAACAATAAAACTGTCTGCTTACATAAACAGTAATACAAGGGGTGTTATGAGCCATA
TTCAACGGGAAACGTCGAGGCCGCGATTAAATTCCAACATGGATGCTGATTTATATG
GGTATAAATGGGCTCGCGATAATGTCGGGCAATCAGGTGCGACAATCTATCGCTTGT
ATGGGAAGCCCGATGCGCCAGAGTTGTTTCTGAAACATGGCAAAGGTAGCGTTGCC
AATGATGTTACAGATGAGATGGTCAGACTAAACTGGCTGACGGAATTTATGCCTCTT
CCGACCATCAAGCATTTTATCCGTACTCCTGATGATGCATGGTTACTCACCACTGCGA
TCCCCGGAAAAACAGCATTCCAGGTATTAGAAGAATATCCTGATTCAGGTGAAAATA
TTGTTGATGCGCTGGCAGTGTTCCTGCGCCGGTTGCATTCGATTCCTGTTTGTAATTG
TCCTTTTAACAGCGATCGCGTATTTCGTCTCGCTCAGGCGCAATCACGAATGAATAA
CGGTTTGGTTGATGCGAGTGATTTTGATGACGAGCGTAATGGCTGGCCTGTTGAACA
AGTCTGGAAAGAAATGCATAAACTTTTGCCATTCTCACCGGATTCAGTCGTCACTCA
TGGTGATTTCTCACTTGATAACCTTATTTTTGACGAGGGGAAATTAATAGGTTGTATT
GATGTTGGACGAGTCGGAATCGCAGACCGATACCAGGATCTTGCCATCCTATGGAAC
TGCCTCGGTGAGTTTTCTCCTTCATTACAGAAACGGCTTTTTCAAAAATATGGTATTG
ATAATCCTGATATGAATAAATTGCAGTTTCATTTGATGCTCGATGAGTTTTTCTAATC
TCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCT
AGTGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGT

TABLE 11
Components of Construct Sequence (SEQ ID NO: 102)

| Components | Position in construct |
|---|---|
| 5′ITR | 12-130 |
| Cloning site | 131-147 |
| FABP3 promoter | 148-1899 |
| hGJB2 minimal promoter | 1900-2027 |
| Cloning site | 2028-2036 |
| Synthetic barcode | 2037-2044 |
| 5′UTR | 2045-2406 |
| GJB2 (exon2) | 2418-3095 |
| 3xFLAG | 3108-3173 |
| 3′UTR (exon2) | 3177-4583 |
| bGHpA | 4605-4829 |
| Cloning site | 4830-4863 |
| 3′ITR | 4864-4993 |

Exemplary Construct sequence
(SEQ ID NO: 103)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTGAAACAGCAGCCATTGATGTAGC
TCAGGGTTCTGTGGATCTGTCATTTGGAGCATGTTGGTTCTCCTGTCTCAGCTGGGCT
CATTCATGCATCTGAGTTCAGCTATTGGGCAATCTGGGGAATGTTTTGTCCATGTGAT
GTGTCATCTTCTACCAGGCTAGCCTGGGCTTCATCACATGGTATCTGGCAGGGCTCTA
AGAGGGAGAGTTGAAACACACAAGGCCTCTTGAAGCTTAGACTCAGAATTGGCACA
AGGTCGCTTCTGGCACATTCCATTGGTCAAAGCAAGTTACAAGGCCAGCTCACATTC
AAGGATTAGGTAAGTCGATTCCACTCTTGATGAGAAGTCTGAAGGATTTGGAACAGT
GTCCACCATGCAGTAATAAACTCAATAAGTAGTAGCCATTATTATTCTGTTAGAGGT
TGCCAGGAAAAGTTTTATAGTGGAAAGAAATCTGAGTTTACTCTTGAGAGGTAAGTG
GAATTTCTATTTGTAGAGAATGAAGGCCTCTCAAAAAGACACAGCCTAACAATAGGT
GCTGCAGTTTAACAGTGGAGCGTGTCCAGAACAGGCTGCCCTTTTAGGCAAGGGCTA
GTGTCTTTCAGGACAGACCCAAACCCCAAATACCAAAACAGAATAAAGTAGTGTCTT
AGCATACTTTGAGATCAGACTGTTTCTGCATTTCACAGTGCTGGGGGGGGGGGGAG
GTGTGGGGGGAAGGGAAAAGCAGCATACCAATGTAGTGAAATCTGGAAACAACAGC
CAAAAAAAGTTTGCATATTGCACAGAGCACTTGAAGATCATAAATCTATGCATGAGA
AAGATGTAGTGGAAATTTTGGGGGGGATTAGAGTTTATTTTTGTCATCTCTGTGAGA
CAGCTACTCATTCATCCAGATCACAGCTAAGAAAAAAGCTGGTCACAGAAATTAGC
AGTTTCAGCTCAGCAGCGAAGTCGCCAGCCTGTGAAGGCAGAGAGAAATTGACTAA
TTAGCAATGCGCACTAAAACTTGACGGTTCTTTATAGAGAGAGAGAAGAGAGAGGG
AGAGAGAGGGAGAGGGAGGGAGGGGGGGCTCGCTTTTTCCCCTTCTTTCTTCCAAAG
ATGTTTGAAATCGCAGTCATTTACGCTCGACAATTTTTACAATAGCCTTGAGCCATAA
TTTTGCGAGTCTCTCCAGCATCCATCCCCCTGTATGGTCTCTCTCTACTGGCCAAGCA
CGACCGTTTCTCTCCCCAACCGTGGATTTCCTATTAAGCTCTGAGGACCCAGAGGCC
GGGCGCGCTCCGCCCGCGGCGCCGCCCCCTCCGTAACTTTCCCAGTCTCCGAGGGAA
GAGGCGGGGTGTGGGGTGCGGTTAAAAGGCGCCACGGCGGGAGACAGGTCTCACCG
GTCCCGTTCTGTTGCGGCCCCGCAGCGCCCGCGCGCTCCTCTCCCCGACTCGGAGCC
CCTCGGCGGCGCCCGGCCCAGGACCCGCCTAGGAGCGCAGGAGCCCCAGCGCAGAG
ACCCCAACGCCGAGACCCCCGCCCCGGCCCCGCCGCGCTTCCTCCCGACGCAGTTTA
GGACCCTTGTTCGCGAAGAGGTGGTGTGCGGCTGAGACCCGCGTCCTCAGGACGGTT
CCATCAGTGCCTCGATCCTGCCCCACTGGAGGAGGAAGGCAGCCCGAACAGCGCTC
ACCTAACTAACAGCTGCTGAGAGCTGGGTTCCGTGGCCATGCACCTGGGACTGCCTT
GAGAAGCGTGAGCAAACCGCCCAGAGTAGAAGCGCTAGCCACCATGGATTGGGGCA
CGCTGCAGACGATCCTGGGGGGTGTGAACAAACACTCCACCAGCATTGGAAAGATC
TGGCTCACCGTCCTCTTCATTTTTCGCATTATGATCCTCGTTGTGGCTGCAAAGGAGG
TGTGGGGAGATGAGCAGGCCGACTTTGTCTGCAACACCCTGCAGCCAGGCTGCAAG
AACGTGTGCTACGATCACTACTTCCCCATCTCCCACATCCGGCTATGGGCCCTGCAG
CTGATCTTCGTGTCCACGCCAGCGCTCCTAGTGGCCATGCACGTGGCCTACCGGAGA
CATGAGAAGAAGAGGAAGTTCATCAAGGGGGAGATAAAGAGTGAATTTAAGGACAT
CGAGGAGATCAAAACCCAGAAGGTCCGCATCGAAGGCTCCCTGTGGTGGACCTACA
CAAGCAGCATCTTCTTCCGGGTCATCTTCGAAGCCGCCTTCATGTACGTCTTCTATGT
CATGTACGACGGCTTCTCCATGCAGCGGCTGGTGAAGTGCAACGCCTGGCCTTGTCC
CAACACTGTGGACTGCTTTGTGTCCCGGCCCACGGAGAAGACTGTCTTCACAGTGTT
CATGATTGCAGTGTCTGGAATTTGCATCCTGCTGAATGTCACTGAATTGTGTTATTTG
CTAATTAGATATTGTTCTGGGAAGTCAAAAAAGCCAGTTGGATCCCGGGCTGACTAC
AAAGACCATGACGGTGATTATAAAGATCATGACATCGACTACAAGGATGACGATGA
CAAGTAAGAAATAGACAGCATGAGAGGGATGAGGCAACCCGTGCTCAGCTGTCAAG
GCTCAGTCGCTAGCATTTCCCAACACAAAGATTCTGACCTTAAATGCAACCATTTGA
AACCCCTGTAGGCCTCAGGTGAAACTCCAGATGCCACAATGGAGCTCTGCTCCCCTA
AAGCCTCAAAACAAAGGCCTAATTCTATGCCTGTCTTAATTTTCTTTCACTTAAGTTA
GTTCCACTGAGACCCCAGGCTGTTAGGGGTTATTGGTGTAAGGTACTTTCATATTTTA
AACAGAGGATATCGGCATTTGTTTCTTTCTCTGAGGACAAGAGAAAAAAGCCAGGTT
CCACAGAGGACACAGAGAAGGTTTGGGTGTCCTCCTGGGGTTCTTTTTGCCAACTTT
CCCCACGTTAAAGGTGAACATTGGTTCTTTCATTTGCTTTGGAAGTTTTAATCTCTAA
CAGTGGACAAAGTTACCAGTGCCTTAAACTCTGTTACACTTTTTGGAAGTGAAAACT
TTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATT
TAATTTGAAATATGGTCTTTTGGTTATGAATACTTTGCAGCACAGCTGAGAGGCTGTC
TGTTGTATTCATTGTGGTCATAGCACCTAACAACATTGTAGCCTCAATCGAGTGAGA
CAGACTAGAAGTTCCTAGTGATGGCTTATGATAGCAAATGGCCTCATGTCAAATATT
TAGATGTAATTTTGTGTAAGAAATACAGACTGGATGTACCACCAACTACTACCTGTA
ATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAAT
GGGGAGGACAAAATGGGGCAGGGGAGGGAGAAGTTTCTGTCGTTAAAAACAGATTT
GGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACAT
GTGAAAAGAATAGAAGCTAAGGTTTAGATAAATATTGAGCAGATCTATAGGAAGAT
TGAACCTGAATATTGCCATTATGCTTGACATGGTTTCCAAAAAATGGTACTCCACAT
ATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTA
TCAAATACATTTAAAACATTAAAATATAATCTCTATAATAAGAGCTCGCTGATCAGC
CTCGACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATG
GAAGCTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGCTGCC
TGCAGGGGCGCCTGATGCGGTATTTTCTCCTTACGCATCTGTGCGGTATTTCACACCG
CATACGTCAAAGCAACCATAGTACGCGCCCTGTAGCGGCGCATTAAGCGCGGCGGG
TGTGGTGGTTACGCGCAGCGTGACCGCTACACTTGCCAGCGCCCTAGCGCCCGCTCC
TTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAA
AACTTGATTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAAC
AACACTCAACCCTATCTCGGGCTATTCTTTTGATTTATAAGGGATTTTGCCGATTTCG
GCCTATTGGTTAAAAAATGAGCTGATTTAACAAAAATTTAACGCGAATTTTAACAAA
ATATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTC
AGAGGTTTTCACCGTCATCACCGAAACGCGCGAGACGAAAGGGCCTCGTGATACGC
CTATTTTTATAGGTTAATGTCATGAACAATAAAACTGTCTGCTTACATAAACAGTAAT
ACAAGGGGTGTTATGAGCCATATTCAACGGGAAACGTCGAGGCCGCGATTAAATTC
CAACATGGATGCTGATTTATATGGGTATAAATGGGCTCGCGATAATGTCGGGCAATC
AGGTGCGACAATCTATCGCTTGTATGGGAAGCCCGATGCGCCAGAGTTGTTTCTGAA
ACATGGCAAAGGTAGCGTTGCCAATGATGTTACAGATGAGATGGTCAGACTAAACT
GGCTGACGGAATTTATGCCTCTTCCGACCATCAAGCATTTTATCCGTACTCCTGATGA
TGCATGGTTACTCACCACTGCGATCCCCGGAAAAACAGCATTCCAGGTATTAGAAGA
ATATCCTGATTCAGGTGAAAATATTGTTGATGCGCTGGCAGTGTTCCTGCGCCGGTT
GCATTCGATTCCTGTTTGTAATTGTCCTTTTAACAGCGATCGCGTATTTCGTCTCGCTC
AGGCGCAATCACGAATGAATAACGGTTTGGTTGATGCGAGTGATTTTGATGACGAGC
GTAATGGCTGGCCTGTTGAACAAGTCTGGAAAGAAATGCATAAACTTTTGCCATTCT
CACCGGATTCAGTCGTCACTCATGGTGATTTCTCACTTGATAACCTTATTTTTGACGA
GGGGAAATTAATAGGTTGTATTGATGTTGGACGAGTCGGAATCGCAGACCGATACC
AGGATCTTGCCATCCTATGGAACTGCCTCGGTGAGTTTTCTCCTTCATTACAGAAACG
GCTTTTTCAAAAATATGGTATTGATAATCCTGATATGAATAAATTGCAGTTTCATTTG
ATGCTCGATGAGTTTTTCTAATCTCATGACCAAAATCCCTTAACGTGAGTTTTCGTTC
CACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGAAC
TCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGC
TCACATGT

TABLE 12
Components of Construct Sequence (SEQ ID NO: 103)

| Components | Position in construct |
|---|---|
| 5′ITR | 12-130 |
| Cloning site | 131-147 |
| KLHL14 promoter | 148-1402 |
| hGJB2 minimal promoter | 1403-1530 |
| Cloning site | 1531-1539 |
| Synthetic barcode | 1540-1547 |
| 5′UTR | 1548-1909 |
| GJB2 (exon2) | 1921-2598 |
| 3xFLAG | 2611-2676 |
| 3′UTR (exon2) | 2680-4086 |
| bGHpA | 4108-4332 |
| Cloning site | 4333-4366 |
| 3′ITR | 4367-4496 |

Exemplary Construct sequence
(SEQ ID NO: 104)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCCTTCCTCCTCCAGGGCCCTCTGC
AGACCAGGCTGAGATGGAGGAACCTGCTAAAATCGATGGAGATGCTTCTAGCCTCC
CAGTAGGAGGCCCCAGCCATGCCTTCAACCTGGCAGGAGGTGTAGCCACTCCTCATC
CTTGGGTTGCAGGTTGGGTGCTGCTGTTGTGGTCCTTCCCAGAAACTGCCAGTAGAG
GGCAGCCTGGGCATCCTAATGCTTACTCTGGTTGTTACACAAAGAAAATATTGGGGT
CACTGGCGAGCCCACCCACACTCACCAGAATCTCCACTGTAGTCCCCCTAACAAACA
GCCCTTCACTTCCTCTCCCACTTCAGCAATTTGTATTTTGATGCCATTGGCCTCAGAT
CAGAGTGTTTTAAATCATCACGCCCTGGCTTATCCCTGGTCGAGCCAGGACACGGGG
TGCTTCAGTGGGTCTGTCACCCTCTCTCCTTGAAGCATGTTGCTTTTATTTATTTACTT
TTACTCTCACCCTGCTCCTGTACCAGCAGGGGCCACTTCAAAGCCAAGGTACAGGGT
GATAACTTGTGGTCCAGCATCAGTTTTCTCCACTTCTTTCTCCCACTCACCCCCAGCA
AGGTGCCTGGGGAGACTTGAGCAGATGTTTCATTTTGGCCTGGCCAGTGGCTGAAAG
CCAGGCCTCCAATGCACTGTGACCTCTGGCTTCCCCAGCAGCTTTCCCAGAGAGGCA
GAGGGAGTCTTCATTCTTCCCAGGCGGGGAGACCACGCCTTCCCTGCCTCCTCCCTCC
GCGGGGGGTCGCGTTGGAGGTCACCCCCGCCCCCTAGGCGCTGGGTTGGGAGTGAC
GCGGGGTGGGCTGGAGAGGTTTCCTGCCGTCTGGGAAGCGTAAACGGACCGCCCAC
CTGTCGGGCCTCGGCCGCCCGCACCTGCTTGTGAGAAGCCTGCGGCTGGGGCACCGC
CCCCGGTCCCCGCCCGGGTCCGCGCATTGGGAGCACACTGGCCCTTTAAGAGCGCGG
CGGCCGCGGCGCGCGGGAAGCTCTGAGGACCCAGAGGCCGGGCGCGCTCCGCCCGC
GGCGCCGCCCCCTCCGTAACTTTCCCAGTCTCCGAGGGAAGAGGCGGGGTGTGGGGT
GCGGTTAAAAGGCGCCACGGCGGGAGACAGGTCTCACCGGTTTCACTGGGTTGCGG
CCCCGCAGCGCCCGCGCGCTCCTCTCCCCGACTCGGAGCCCCTCGGCGGCGCCCGGC
CCAGGACCCGCCTAGGAGCGCAGGAGCCCCAGCGCAGAGACCCCAACGCCGAGACC
CCCGCCCCGGCCCCGCCGCGCTTCCTCCCGACGCAGTTTAGGACCCTTGTTCGCGAA
GAGGTGGTGTGCGGCTGAGACCCGCGTCCTCAGGACGGTTCCATCAGTGCCTCGATC
CTGCCCCACTGGAGGAGGAAGGCAGCCCGAACAGCGCTCACCTAACTAACAGCTGC
TGAGAGCTGGGTTCCGTGGCCATGCACCTGGGACTGCCTTGAGAAGCGTGAGCAAA
CCGCCCAGAGTAGAAGCGCTAGCCACCATGGATTGGGGCACGCTGCAGACGATCCT
GGGGGGTGTGAACAAACACTCCACCAGCATTGGAAAGATCTGGCTCACCGTCCTCTT
CATTTTTCGCATTATGATCCTCGTTGTGGCTGCAAAGGAGGTGTGGGGAGATGAGCA
GGCCGACTTTGTCTGCAACACCCTGCAGCCAGGCTGCAAGAACGTGTGCTACGATCA
CTACTTCCCCATCTCCCACATCCGGCTATGGGCCCTGCAGCTGATCTTCGTGTCCACG
CCAGCGCTCCTAGTGGCCATGCACGTGGCCTACCGGAGACATGAGAAGAAGAGGAA
GTTCATCAAGGGGGAGATAAAGAGTGAATTTAAGGACATCGAGGAGATCAAAACCC
AGAAGGTCCGCATCGAAGGCTCCCTGTGGTGGACCTACACAAGCAGCATCTTCTTCC
GGGTCATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGACTGCTT
TGTGTCCCGGCCCACGGAGAAGACTGTCTTCACAGTGTTCATGATTGCAGTGTCTGG
AATTTGCATCCTGCTGAATGTCACTGAATTGTGTTATTTGCTAATTAGATATTGTTCT
GGGAAGTCAAAAAAGCCAGTTGGATCCCGGGCTGACTACAAAGACCATGACGGTGA
TTATAAAGATCATGACATCGACTACAAGGATGACGATGACAAGTAAGAAATAGACA
GCATGAGAGGGATGAGGCAACCCGTGCTCAGCTGTCAAGGCTCAGTCGCTAGCATTT
CCCAACACAAAGATTCTGACCTTAAATGCAACCATTTGAAACCCCTGTAGGCCTCAG
GTGAAACTCCAGATGCCACAATGGAGCTCTGCTCCCCTAAAGCCTCAAAACAAAGG
CCTAATTCTATGCCTGTCTTAATTTTCTTTCACTTAAGTTAGTTCCACTGAGACCCCA
GGCTGTTAGGGGTTATTGGTGTAAGGTACTTTCATATTTTAAACAGAGGATATCGGC
ATTTGTTTCTTTCTCTGAGGACAAGAGAAAAAAGCCAGGTTCCACAGAGGACACAGA
GAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACC
AGTGCCTTAAACTCTGTTACACTTTTTGGAAGTGAAAACTTTGTAGTATGATAGGTTA
TTTTGATGTAAAGATGTTCTGGATACCATTATATGTTCCCCCTGTTTCAGAGGCTCAG
ATTGTAATATGTAAATGGTATGTCATTCGCTACTATGATTTAATTTGAAATATGGTCT
TTTGGTTATGAATACTTTGCAGCACAGCTGAGAGGCTGTCTGTTGTATTCATTGTGGT
CATAGCACCTAACAACATTGTAGCCTCAATCGAGTGAGACAGACTAGAAGTTCCTAG
TGATGGCTTATGATAGCAAATGGCCTCATGTCAAATATTTAGATGTAATTTTGTGTAA
GAAATACAGACTGGATGTACCACCAACTACTACCTGTAATGACAGGCCTGTCCAACA
CATCTCCCTTTTCCATGACTGTGGTAGCCAGCATCGGAAAGAACGCTGATTTAAAGA
GGTCGCTTGGGAATTTTATTGACACAGTACCATTTAATGGGGAGGACAAAATGGGGC
AGGGGAGGGAGAAGTTTCTGTCGTTAAAAACAGATTTGGAAAGACTGGACTCTAAA
GTCTGTTGATTAAAGATGAGCTTTGTCTACTTCAAAAGTTTGTTTGCTTACCCCTTCA
GCCTCCAATTTTTTAAGTGAAAATATAGCTAATAACATGTGAAAAGAATAGAAGCTA
AGGTTTAGATAAATATTGAGCAGATCTATAGGAAGATTGAACCTGAATATTGCCATT
ATGCTTGACATGGTTTCCAAAAAATGGTACTCCACATATTTCAGTGAGGGTAAGTAT
TTTCCTGTTGTCAAGAATAGCATTGTAAAAGCATTTTGTAATAATAAAGAATAGCTTT
AATGATATGCTTGTAACTAAAATAATTTTGTAATGTATCAAATACATTTAAAACATT
AAAATATAATCTCTATAATAAGAGCTCGCTGATCAGCCTCGACTGTGCCTTCTAGTT
GCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGA
CAATAGCAGGCATGCTGGGGATGCGGTGGGCTCTATGGAAGCTTGAATTCAGCTGAC
GTGCCTCGGACCGCTAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAGCAACCATA
GTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTTGGGTGATGGTTC
ACGTAGTGGGCCATCGCCCTGATAGACGGTTTTTCGCCCTTTGACGTTGGAGTCCAC
GTTCTTTAATAGTGGACTCTTGTTCCAAACTGGAACAACACTCAACCCTATCTCGGGC
TATTCTTTTGATTTATAAGGGATTTTGCCGATTTCGGCCTATTGGTTAAAAAATGAGC
TGATTTAACAAAAATTTAACGCGAATTTTAACAAAATATTAACGTTTACAATTTTATG
GTGCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTCAT
GAACAATAAAACTGTCTGCTTACATAAACAGTAATACAAGGGGTGTTATGAGCCATA
TTCAACGGGAAACGTCGAGGCCGCGATTAAATTCCAACATGGATGCTGATTTATATG
GGTATAAATGGGCTCGCGATAATGTCGGGCAATCAGGTGCGACAATCTATCGCTTGT
ATGGGAAGCCCGATGCGCCAGAGTTGTTTCTGAAACATGGCAAAGGTAGCGTTGCC
AATGATGTTACAGATGAGATGGTCAGACTAAACTGGCTGACGGAATTTATGCCTCTT
CCGACCATCAAGCATTTTATCCGTACTCCTGATGATGCATGGTTACTCACCACTGCGA
TCCCCGGAAAAACAGCATTCCAGGTATTAGAAGAATATCCTGATTCAGGTGAAAATA
TTGTTGATGCGCTGGCAGTGTTCCTGCGCCGGTTGCATTCGATTCCTGTTTGTAATTG
TCCTTTTAACAGCGATCGCGTATTTCGTCTCGCTCAGGCGCAATCACGAATGAATAA
CGGTTTGGTTGATGCGAGTGATTTTGATGACGAGCGTAATGGCTGGCCTGTTGAACA
AGTCTGGAAAGAAATGCATAAACTTTTGCCATTCTCACCGGATTCAGTCGTCACTCA
TGGTGATTTCTCACTTGATAACCTTATTTTTGACGAGGGGAAATTAATAGGTTGTATT
GATGTTGGACGAGTCGGAATCGCAGACCGATACCAGGATCTTGCCATCCTATGGAAC
TGCCTCGGTGAGTTTTCTCCTTCATTACAGAAACGGCTTTTTCAAAAATATGGTATTG
ATAATCCTGATATGAATAAATTGCAGTTTCATTTGATGCTCGATGAGTTTTTCTAATC
TCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCT
AGTGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGT

TABLE 13
Components of Construct Sequence (SEQ ID NO: 104)

| Components | Position in construct |
|---|---|
| 5′ITR | 12-130 |
| Cloning site | 131-147 |
| MMP15 promoter | 148-1159 |
| hGJB2 minimal promoter | 1160-1287 |
| Cloning site | 1288-1296 |
| Synthetic barcode | 1297-1304 |
| 5′UTR | 1305-1666 |
| GJB2 (exon2) | 1678-2355 |
| 3xFLAG | 2368-2433 |
| 3′UTR (exon2) | 2437-3843 |
| bGHpA | 3865-4089 |
| Cloning site | 4090-4123 |
| 3′ITR | 4124-4253 |

Exemplary Construct sequence
(SEQ ID NO: 105)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCAGGCTACCTCTCAGGCTGACTG
AGTCATGCAGCATAGGCTGCCACGTCTCTGGGCTGGCGGGGCCGTCATTATTCCTGG
CCTCACTGCAGCTAAATTGAAGAAACGTTTGGTTTGTGGGCCACGTCAAGGAATGTG
TAAGAGCTGCCACGTTGTCGGGTCTGGGTTATTGGGCTTTTCCCCTCCTTCAGAGAAG
ATTTCCAGGCGTGTGGGTGGGGTTTCAGAAGAAAATTGATGCCTGCGTGTGAGTGTT
CCCTGGACCTGGACCAGCAGCGGCAATATTACAGACCCGGGGGTTGGGGCAGACTG
AGCCAATCTCTGCACCGTCAAAGTTATGGATACAGAGCCCTGGAAAAAGGCTGAAG
GATAAGATAGCTGACATTTATGAAGTGCTTCATTCATGTAGCAGTGGGCCAAATGCG
TACTTTACACTTGAGGAAGCTGAGGCTGGAGGTTGATAACATGCCTCAAGTCTTCTA
GAGTTAAATAACTTTGACCCAGGACCCAAGCCCAGAGTTCTGACTCAAAAACTAGGC
CTCCTAAACATCCTCTTATATGAGGTTAAATTTCATCTTCCTCTGTTTGGCCTTGGCCT
GGTTGGTGGATGCTCTGCTTCGGGGACCCAGGGCCAGATGACAATGGGTTCTTTGTG
CCCTTCAGACAATGGGAAGGGCTGCCTGGGGAAAGATACAGTAACAAGGCAACAGG
CTGAGTCAGCCTCCAATGTGCTTGAACCTTCTTAGCTTGGCAGCCTTGACATTCAGCC
AGCCACACAAAGGGTATATCAAGGATGATACCACTAGTAGCAGCTTGTCTTGTCTGT
ACCTCTGAACAAGAAAGAGGCTGTTCTGGGTCATCCCTCCAGGCCTGTCCAGCCCTG
GCACTCTGTGAGTCGGTTTAGGCAGCAGCCCCGGAACAGATGAGGCAGGCAGGGTT
GGGACGTTTGGTCAGGACAGCCCACCAGGAGGAAAGAAATGAAAGACAGAGACAG
CTTTGGCTATGGGAGAAGGAGGAGGCCGGGGGAAGGAGGAGACAGGAGGAGGAGG
GACCACGGGGTGGAGGGGAGATAGACCCAGCCCAAAGCTCTGAGGACCCAGAGGCC
GGGCGCGCTCCGCCCGCGGCGCCGCCCCCTCCGTAACTTTCCCAGTCTCCGAGGGAA
GAGGCGGGGTGTGGGGTGCGGTTAAAAGGCGCCACGGCGGGAGACAGGTCTCACCG
GTATACTCTCGTTGCGGCCCCGCAGCGCCCGCGCGCTCCTCTCCCCGACTCGGAGCC
CCTCGGCGGCGCCCGGCCCAGGACCCGCCTAGGAGCGCAGGAGCCCCAGCGCAGAG
ACCCCAACGCCGAGACCCCCGCCCCGGCCCCGCCGCGCTTCCTCCCGACGCAGTTTA
GGACCCTTGTTCGCGAAGAGGTGGTGTGCGGCTGAGACCCGCGTCCTCAGGACGGTT
CCATCAGTGCCTCGATCCTGCCCCACTGGAGGAGGAAGGCAGCCCGAACAGCGCTC
ACCTAACTAACAGCTGCTGAGAGCTGGGTTCCGTGGCCATGCACCTGGGACTGCCTT
GAGAAGCGTGAGCAAACCGCCCAGAGTAGAAGCGCTAGCCACCATGGATTGGGGCA
CGCTGCAGACGATCCTGGGGGGTGTGAACAAACACTCCACCAGCATTGGAAAGATC
TGGCTCACCGTCCTCTTCATTTTTCGCATTATGATCCTCGTTGTGGCTGCAAAGGAGG
TGTGGGGAGATGAGCAGGCCGACTTTGTCTGCAACACCCTGCAGCCAGGCTGCAAG
AACGTGTGCTACGATCACTACTTCCCCATCTCCCACATCCGGCTATGGGCCCTGCAG
CTGATCTTCGTGTCCACGCCAGCGCTCCTAGTGGCCATGCACGTGGCCTACCGGAGA
CATGAGAAGAAGAGGAAGTTCATCAAGGGGGAGATAAAGAGTGAATTTAAGGACAT
CGAGGAGATCAAAACCCAGAAGGTCCGCATCGAAGGCTCCCTGTGGTGGACCTACA
CAAGCAGCATCTTCTTCCGGGTCATCTTCGAAGCCGCCTTCATGTACGTCTTCTATGT
CATGTACGACGGCTTCTCCATGCAGCGGCTGGTGAAGTGCAACGCCTGGCCTTGTCC
CAACACTGTGGACTGCTTTGTGTCCCGGCCCACGGAGAAGACTGTCTTCACAGTGTT
CATGATTGCAGTGTCTGGAATTTGCATCCTGCTGAATGTCACTGAATTGTGTTATTTG
CTAATTAGATATTGTTCTGGGAAGTCAAAAAAGCCAGTTGGATCCCGGGCTGACTAC
AAAGACCATGACGGTGATTATAAAGATCATGACATCGACTACAAGGATGACGATGA
CAAGTAAGAAATAGACAGCATGAGAGGGATGAGGCAACCCGTGCTCAGCTGTCAAG
GCTCAGTCGCTAGCATTTCCCAACACAAAGATTCTGACCTTAAATGCAACCATTTGA
AACCCCTGTAGGCCTCAGGTGAAACTCCAGATGCCACAATGGAGCTCTGCTCCCCTA
AAGCCTCAAAACAAAGGCCTAATTCTATGCCTGTCTTAATTTTCTTTCACTTAAGTTA
GTTCCACTGAGACCCCAGGCTGTTAGGGGTTATTGGTGTAAGGTACTTTCATATTTTA
AACAGAGGATATCGGCATTTGTTTCTTTCTCTGAGGACAAGAGAAAAAAGCCAGGTT
CCACAGAGGACACAGAGAAGGTTTGGGTGTCCTCCTGGGGTTCTTTTTGCCAACTTT
CCCCACGTTAAAGGTGAACATTGGTTCTTTCATTTGCTTTGGAAGTTTTAATCTCTAA
CAGTGGACAAAGTTACCAGTGCCTTAAACTCTGTTACACTTTTTGGAAGTGAAAACT
TTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATT
TAATTTGAAATATGGTCTTTTGGTTATGAATACTTTGCAGCACAGCTGAGAGGCTGTC
TGTTGTATTCATTGTGGTCATAGCACCTAACAACATTGTAGCCTCAATCGAGTGAGA
CAGACTAGAAGTTCCTAGTGATGGCTTATGATAGCAAATGGCCTCATGTCAAATATT
TAGATGTAATTTTGTGTAAGAAATACAGACTGGATGTACCACCAACTACTACCTGTA
ATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAAT
GGGGAGGACAAAATGGGGCAGGGGAGGGAGAAGTTTCTGTCGTTAAAAACAGATTT
GGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACAT
GTGAAAAGAATAGAAGCTAAGGTTTAGATAAATATTGAGCAGATCTATAGGAAGAT
TGAACCTGAATATTGCCATTATGCTTGACATGGTTTCCAAAAAATGGTACTCCACAT
ATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTA
TCAAATACATTTAAAACATTAAAATATAATCTCTATAATAAGAGCTCGCTGATCAGC
CTCGACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCAT
CGCATTGTCTGAGTAGGTGTCATTCTATTCTGGGGGGGGGGTGGGGCAGGACAGCA
AGGGGGAGGATTGGGAAGACAATAGCAGGCATGCTGGGGATGCGGTGGGCTCTATG
GAAGCTTGAATTCAGCTGACGTGCCTCGGACCGCTAGGAACCCCTAGTGATGGAGTT
GGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGCTGCC
TGCAGGGGCGCCTGATGCGGTATTTTCTCCTTACGCATCTGTGCGGTATTTCACACCG
CATACGTCAAAGCAACCATAGTACGCGCCCTGTAGCGGCGCATTAAGCGCGGCGGG
TGTGGTGGTTACGCGCAGCGTGACCGCTACACTTGCCAGCGCCCTAGCGCCCGCTCC
TTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAA
AACTTGATTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAAC
AACACTCAACCCTATCTCGGGCTATTCTTTTGATTTATAAGGGATTTTGCCGATTTCG
GCCTATTGGTTAAAAAATGAGCTGATTTAACAAAAATTTAACGCGAATTTTAACAAA
ATATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTC
AGAGGTTTTCACCGTCATCACCGAAACGCGCGAGACGAAAGGGCCTCGTGATACGC
CTATTTTTATAGGTTAATGTCATGAACAATAAAACTGTCTGCTTACATAAACAGTAAT
ACAAGGGGTGTTATGAGCCATATTCAACGGGAAACGTCGAGGCCGCGATTAAATTC
CAACATGGATGCTGATTTATATGGGTATAAATGGGCTCGCGATAATGTCGGGCAATC
AGGTGCGACAATCTATCGCTTGTATGGGAAGCCCGATGCGCCAGAGTTGTTTCTGAA
ACATGGCAAAGGTAGCGTTGCCAATGATGTTACAGATGAGATGGTCAGACTAAACT
GGCTGACGGAATTTATGCCTCTTCCGACCATCAAGCATTTTATCCGTACTCCTGATGA
TGCATGGTTACTCACCACTGCGATCCCCGGAAAAACAGCATTCCAGGTATTAGAAGA
ATATCCTGATTCAGGTGAAAATATTGTTGATGCGCTGGCAGTGTTCCTGCGCCGGTT
GCATTCGATTCCTGTTTGTAATTGTCCTTTTAACAGCGATCGCGTATTTCGTCTCGCTC
AGGCGCAATCACGAATGAATAACGGTTTGGTTGATGCGAGTGATTTTGATGACGAGC
GTAATGGCTGGCCTGTTGAACAAGTCTGGAAAGAAATGCATAAACTTTTGCCATTCT
CACCGGATTCAGTCGTCACTCATGGTGATTTCTCACTTGATAACCTTATTTTTGACGA
GGGGAAATTAATAGGTTGTATTGATGTTGGACGAGTCGGAATCGCAGACCGATACC
AGGATCTTGCCATCCTATGGAACTGCCTCGGTGAGTTTTCTCCTTCATTACAGAAACG
GCTTTTTCAAAAATATGGTATTGATAATCCTGATATGAATAAATTGCAGTTTCATTTG
ATGCTCGATGAGTTTTTCTAATCTCATGACCAAAATCCCTTAACGTGAGTTTTCGTTC
CACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGAAC
TCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGC
TCACATGT

TABLD 14
Components of Construct Sequence (SEQ ID NO: 105)

| Components | Position in construct |
|---|---|
| 5′ITR | 12-130 |
| Cloning site | 131-147 |
| SPARC promoter | 148-1226 |
| hGJB2 minimal promoter | 1227-1354 |
| Cloning site | 1355-1363 |
| Synthetic barcode | 1364-1371 |
| 5′UTR | 1372-1733 |
| GJB2 (exon2) | 1745-2422 |
| 3xFLAG | 2435-2500 |
| 3′UTR (exon2) | 2504-3910 |
| bGHpA | 3932-4156 |
| Cloning site | 4157-4190 |
| 3′ITR | 4191-4320 |

Exemplary Construct sequence
(SEQ ID NO: 106)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCCAAGGACTCTTTTTTCTAAACTT
CCCTTCATCTTCTAGTTTGACGCCCTTGGTGGGAAAAGTGTCTGAGATAAGGAAAAG
GCATCCTTTCAGTTCTCTGATACTATCTTGAAGCGAGGGATGGAGAAAGGCAAAGAG
AGACACAGGAGAAGCGTATCCCCTGGGAACAGGTGTCTAGTGGAGTCCAGTAACTC
ACAGTCTCTCAGTTCCGTCAGCACTGTCCCTTGGGTCGCAAATTTCTTCCATTAGCCC
TTCCACCAGCTGTATTTCAAATGGGGCTGGACAATAATTGTGGCCAGTGGCCTTGTG
TTGTTTGTACTTGCGGACTAGTAGTTCTCACCTGTCTTTCTCTGACTCCTATTAGCCAC
TGGGATTTCAGCAGCTGGTTCAGCCAATTCTACTCAATTCAACATTAAGTTGCAGTG
GGCTAGAACTCATGGGCCGATTTAACAAGTGAAATTCTACCAAGATACATCAAAAAT
AGCAACAGGACTAGATACTCAGCTCATTTTGTTTTATTTGTAATATACCAGTTGTGGC
TTTAGTGCCAGTCTGATTCATCTCTCTACTACAAAATGAGGCTCTATAAAGGAAAAT
ATTGCAACTGGAGTGAGGAATTTGAATCTTATAGGAAGGAATTTGTCTTCTCATGAA
GACTTCAGTTTACCAGAAGTATCTATTGAGGAAGTGTTTACAAGAAAATGTGCCATT
TAGCTTTATTCTAAATTTGCATAATAACTGAACCAAACAAAAAAATATAGATAGATA
GATTGTTCTATCTATAGATAGATAGGGAACATTGGCAGTAGGTGGCAGTAAGTTCCC
CTGAGCACATGGAGGACACAGTTAAATGCATTTGAGGTATGTGGGAAATGGTTTAA
AGCAGAATTTTATGCCAACTTTTAGTAACGGAAGCCTAACAAATGTTTGTTCTTTCAA
GTGAGAGAAGCAAGCAATCTGGAACTATTCATAAGCTTATTTTCTGTATCCTTAAAC
ATATTTTATAATGAATGTATGATTTAAATAGTAAGTTAAGTGTCTGGGGGTACTGCA
CACCTCCCTTGCATACAGTCAAACTTCTTCAGGGTGATGGGGAAGAGGAGTTATAGG
CTGCCAAGCAAAATTGCCAAACTGGTCTCAGAAATTCACTGCATTGGAGAGCGCGG
GATCCTTGCAACACTGACTTTAGCAGTTAAACTAGAGTGGTTGGGGATGAGTATTCT
AAGCTCTGAGGACCCAGAGGCCGGGCGCGCTCCGCCCGCGGCGCCGCCCCCTCCGT
AACTTTCCCAGTCTCCGAGGGAAGAGGCGGGGTGTGGGGTGCGGTTAAAAGGCGCC
ACGGCGGGAGACAGGTCTCACCGGTGGCACTTCGTTGCGGCCCCGCAGCGCCCGCG
CGCTCCTCTCCCCGACTCGGAGCCCCTCGGCGGCGCCCGGCCCAGGACCCGCCTAGG
AGCGCAGGAGCCCCAGCGCAGAGACCCCAACGCCGAGACCCCCGCCCCGGCCCCGC
CGCGCTTCCTCCCGACGCAGTTTAGGACCCTTGTTCGCGAAGAGGTGGTGTGCGGCT
GAGACCCGCGTCCTCAGGACGGTTCCATCAGTGCCTCGATCCTGCCCCACTGGAGGA
GGAAGGCAGCCCGAACAGCGCTCACCTAACTAACAGCTGCTGAGAGCTGGGTTCCG
TGGCCATGCACCTGGGACTGCCTTGAGAAGCGTGAGCAAACCGCCCAGAGTAGAAG
CGCTAGCCACCATGGATTGGGGCACGCTGCAGACGATCCTGGGGGGTGTGAACAAA
CACTCCACCAGCATTGGAAAGATCTGGCTCACCGTCCTCTTCATTTTTCGCATTATGA
TCCTCGTTGTGGCTGCAAAGGAGGTGTGGGGAGATGAGCAGGCCGACTTTGTCTGCA
ACACCCTGCAGCCAGGCTGCAAGAACGTGTGCTACGATCACTACTTCCCCATCTCCC
ACATCCGGCTATGGGCCCTGCAGCTGATCTTCGTGTCCACGCCAGCGCTCCTAGTGG
CCATGCACGTGGCCTACCGGAGACATGAGAAGAAGAGGAAGTTCATCAAGGGGGAG
ATAAAGAGTGAATTTAAGGACATCGAGGAGATCAAAACCCAGAAGGTCCGCATCGA
AGGCTCCCTGTGGTGGACCTACACAAGCAGCATCTTCTTCCGGGTCATCTTCGAAGC
CGCCTTCATGTACGTCTTCTATGTCATGTACGACGGCTTCTCCATGCAGCGGCTGGTG
AAGTGCAACGCCTGGCCTTGTCCCAACACTGTGGACTGCTTTGTGTCCCGGCCCACG
GAGAAGACTGTCTTCACAGTGTTCATGATTGCAGTGTCTGGAATTTGCATCCTGCTG
AATGTCACTGAATTGTGTTATTTGCTAATTAGATATTGTTCTGGGAAGTCAAAAAAG
CCAGTTGGATCCCGGGCTGACTACAAAGACCATGACGGTGATTATAAAGATCATGAC
ATCGACTACAAGGATGACGATGACAAGTAAGAAATAGACAGCATGAGAGGGATGA
GGCAACCCGTGCTCAGCTGTCAAGGCTCAGTCGCTAGCATTTCCCAACACAAAGATT
CTGACCTTAAATGCAACCATTTGAAACCCCTGTAGGCCTCAGGTGAAACTCCAGATG
CCACAATGGAGCTCTGCTCCCCTAAAGCCTCAAAACAAAGGCCTAATTCTATGCCTG
TCTTAATTTTCTTTCACTTAAGTTAGTTCCACTGAGACCCCAGGCTGTTAGGGGTTAT
TGGTGTAAGGTACTTTCATATTTTAAACAGAGGATATCGGCATTTGTTTCTTTCTCTG
AGGACAAGAGAAAAAAGCCAGGTTCCACAGAGGACACAGAGAAGGTTTGGGTGTCC
TCCTGGGGTTCTTTTTGCCAACTTTCCCCACGTTAAAGGTGAACATTGGTTCTTTCAT
TTGCTTTGGAAGTTTTAATCTCTAACAGTGGACAAAGTTACCAGTGCCTTAAACTCTG
TTACACTTTTTGGAAGTGAAAACTTTGTAGTATGATAGGTTATTTTGATGTAAAGATG
TTCTGGATACCATTATATGTTCCCCCTGTTTCAGAGGCTCAGATTGTAATATGTAAAT
GGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAA
CATTGTAGCCTCAATCGAGTGAGACAGACTAGAAGTTCCTAGTGATGGCTTATGATA
GCAAATGGCCTCATGTCAAATATTTAGATGTAATTTTGTGTAAGAAATACAGACTGG
ATGTACCACCAACTACTACCTGTAATGACAGGCCTGTCCAACACATCTCCCTTTTCCA
TGACTGTGGTAGCCAGCATCGGAAAGAACGCTGATTTAAAGAGGTCGCTTGGGAATT
TTATTGACACAGTACCATTTAATGGGGAGGACAAAATGGGGCAGGGGAGGGAGAAG
TTTCTGTCGTTAAAAACAGATTTGGAAAGACTGGACTCTAAAGTCTGTTGATTAAAG
ATGAGCTTTGTCTACTTCAAAAGTTTGTTTGCTTACCCCTTCAGCCTCCAATTTTTTAA
GTGAAAATATAGCTAATAACATGTGAAAAGAATAGAAGCTAAGGTTTAGATAAATA
TTGAGCAGATCTATAGGAAGATTGAACCTGAATATTGCCATTATGCTTGACATGGTT
TCCAAAAAATGGTACTCCACATATTTCAGTGAGGGTAAGTATTTTCCTGTTGTCAAG
AATAGCATTGTAAAAGCATTTTGTAATAATAAAGAATAGCTTTAATGATATGCTTGT
AACTAAAATAATTTTGTAATGTATCAAATACATTTAAAACATTAAAATATAATCTCT
ATAATAAGAGCTCGCTGATCAGCCTCGACTGTGCCTTCTAGTTGCCAGCCATCTGTTG
TTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCAT
GCTGGGGATGCGGTGGGCTCTATGGAAGCTTGAATTCAGCTGACGTGCCTCGGACCG
CTAGGAACCCCTAGTGATGGAGTTGGCCACTCCCTCTCTGCGCGCTCGCTCGCTCACT
GAGGCCGGGCGACCAAAGGTCGCCCGACGCCCGGGCTTTGCCCGGGCGGCCTCAGT
GAGCGAGCGAGCGCGCAGCTGCCTGCAGGGGCGCCTGATGCGGTATTTTCTCCTTAC
GCATCTGTGCGGTATTTCACACCGCATACGTCAAAGCAACCATAGTACGCGCCCTGT
AGCGGCGCATTAAGCGCGGCGGGTGTGGTGGTTACGCGCAGCGTGACCGCTACACTT
GCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCA
TCGCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTTTTGATTT
ATAAGGGATTTTGCCGATTTCGGCCTATTGGTTAAAAAATGAGCTGATTTAACAAAA
ATTTAACGCGAATTTTAACAAAATATTAACGTTTACAATTTTATGGTGCACTCTCAGT
ACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACC
GTCTCCGGGAGCTGCATGTGTCAGAGGTTTTCACCGTCATCACCGAAACGCGCGAGA
CGAAAGGGCCTCGTGATACGCCTATTTTTATAGGTTAATGTCATGAACAATAAAACT
GTCTGCTTACATAAACAGTAATACAAGGGGTGTTATGAGCCATATTCAACGGGAAAC
GTCGAGGCCGCGATTAAATTCCAACATGGATGCTGATTTATATGGGTATAAATGGGC
TCGCGATAATGTCGGGCAATCAGGTGCGACAATCTATCGCTTGTATGGGAAGCCCGA
TGCGCCAGAGTTGTTTCTGAAACATGGCAAAGGTAGCGTTGCCAATGATGTTACAGA
TGAGATGGTCAGACTAAACTGGCTGACGGAATTTATGCCTCTTCCGACCATCAAGCA
TTTTATCCGTACTCCTGATGATGCATGGTTACTCACCACTGCGATCCCCGGAAAAAC
AGCATTCCAGGTATTAGAAGAATATCCTGATTCAGGTGAAAATATTGTTGATGCGCT
GGCAGTGTTCCTGCGCCGGTTGCATTCGATTCCTGTTTGTAATTGTCCTTTTAACAGC
GATCGCGTATTTCGTCTCGCTCAGGCGCAATCACGAATGAATAACGGTTTGGTTGAT
GCGAGTGATTTTGATGACGAGCGTAATGGCTGGCCTGTTGAACAAGTCTGGAAAGA
AATGCATAAACTTTTGCCATTCTCACCGGATTCAGTCGTCACTCATGGTGATTTCTCA
CTTGATAACCTTATTTTTGACGAGGGGAAATTAATAGGTTGTATTGATGTTGGACGA
GTCGGAATCGCAGACCGATACCAGGATCTTGCCATCCTATGGAACTGCCTCGGTGAG
TTTTCTCCTTCATTACAGAAACGGCTTTTTCAAAAATATGGTATTGATAATCCTGATA
TGAATAAATTGCAGTTTCATTTGATGCTCGATGAGTTTTTCTAATCTCATGACCAAAA
TCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAA
GGTAACTGGCTTCAGCAGAGCGCAGATACCAAATACTGTCCTTCTAGTGTAGCCGTA
GTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAG
CGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGAT
GCTCGTCAGGGGGGCGGAGCCTATGGAAAAACGCCAGCAACGCGGCCTTTTTACGG
TTCCTGGCCTTTTGCTGGCCTTTTGCTCACATGT

TABLD 15
Components of Construct Sequence (SEQ ID NO: 106)

| Components | Position in construct |
|---|---|
| 5′ITR | 12-130 |
| Cloning site | 131-147 |
| TSPAN8 promoter | 148-1370 |
| hGJB2 minimal promoter | 1371-1498 |
| Cloning site | 1499-1507 |
| Synthetic barcode | 1508-1515 |
| 5′UTR | 1516-1877 |
| GJB2 (exon2) | 1889-2566 |
| 3xFLAG | 2579-2644 |
| 3′UTR (exon2) | 2648-4054 |
| bGHpA | 4076-4300 |
| Cloning site | 4301-4334 |
| 3′ITR | 4335-4464 |

Exemplary Construct sequence
(SEQ ID NO: 107)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTATTCACAATGCATTCCCTCTGCC
CACCACATTAATTATCAACTCCTTTTCCTGGCATTTACTCATCCAACGCATGGCCCCA
CGTTAACTTTCAGTTCCCTTTCTCCCCTACAAATACTCCATAATCCAGCAACCCTGGG
ATCCCTGAGATGATGAAGAGGACCAGTGCCCATTCCAGGAGACATCACCGCAGCCC
TGAGGAATCGGCTATGGGCACCAGCAGGGCACAGTGCCACACCTCGCCAATGCCTT
GTCCTCCTTTTCCATAGTGAGTCAGTCAGCAAGCGTGTAGAAGTGAGTTCCACACTC
TCTTCCTCCCATAGGGAGATCACTTTTCTCATTCTAAGGGTTCCAGGCACACTCACAA
TGGTGGCATTTGCTGAGCAGTGGCTTGAATAAAGGGCTCTCAGAAAGCAAGATGTA
ACTCAGAGCATAGGCTAGCCCCAGGAATGCTCTTGGGGAATGACCTGCAGCCTCCCA
GTGAAAGAGAGAATAAAAGAAAGCCCCAGCAGGCGAGCTGGGCAGTAGAGAGTCC
TGTAATTCCACCTTGGCAAGCACCATTTGCAAGAACGAACTGGGATAAGGTAAACA
AAATATTGCCTAAAAGAGGCTTGTCCAAAGAAGTCAGAATACGCTCTTCATTTACCT
CTAAATTTCAGTACACCATAAATCTAAATACTCAAAAAAACCTGTGCCTTTTCAATC
AAGGTCAATTGCACGAATTCTTTTGGAAAACAGGACCTATGGCATTTCCCAGACAAA
TCACCGTGAACCCTGTACTGTGCATTGCTGTCCTAAAATCCAAAGATTCTGTCATTTG
TGTTACATAATTGCCTTTCATTTGAACTCATTAATCAAATTGGGGTTTTTAAGCAACA
CCTAATTAATTCTTTAACTGGCTCATCCACTGATCACTGAGTTCTATTTTGAAACTAC
GGACGTCGAGTTTCCTCTTTCACCCAGAATTTTCAGATCTTGTTTAAAAAGTTGGGTG
TGGTTTCATGGGGGGAGGGGGAAGAGCGAGAGGAGACCAGAGGGACGGGGGGGG
GACTCTGCAAGAAAAACCTTCCCGGTGCAATCGTGATCTAAGCTCTGAGGACCCAGA
GGCCGGGCGCGCTCCGCCCGCGGCGCCGCCCCCTCCGTAACTTTCCCAGTCTCCGAG
GGAAGAGGCGGGGTGTGGGGTGCGGTTAAAAGGCGCCACGGCGGGAGACAGGTCTC
ACCGGTTTTCAGGTGTTGCGGCCCCGCAGCGCCCGCGCGCTCCTCTCCCCGACTCGG
AGCCCCTCGGCGGCGCCCGGCCCAGGACCCGCCTAGGAGCGCAGGAGCCCCAGCGC
AGAGACCCCAACGCCGAGACCCCCGCCCCGGCCCCGCCGCGCTTCCTCCCGACGCAG
TTTAGGACCCTTGTTCGCGAAGAGGTGGTGTGCGGCTGAGACCCGCGTCCTCAGGAC
GGTTCCATCAGTGCCTCGATCCTGCCCCACTGGAGGAGGAAGGCAGCCCGAACAGC
GCTCACCTAACTAACAGCTGCTGAGAGCTGGGTTCCGTGGCCATGCACCTGGGACTG
CCTTGAGAAGCGTGAGCAAACCGCCCAGAGTAGAAGCGCTAGCCACCATGGATTGG
GGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAG
GAGGTGTGGGGAGATGAGCAGGCCGACTTTGTCTGCAACACCCTGCAGCCAGGCTG
CAAGAACGTGTGCTACGATCACTACTTCCCCATCTCCCACATCCGGCTATGGGCCCT
GCAGCTGATCTTCGTGTCCACGCCAGCGCTCCTAGTGGCCATGCACGTGGCCTACCG
GAGACATGAGAAGAAGAGGAAGTTCATCAAGGGGGAGATAAAGAGTGAATTTAAG
GACATCGAGGAGATCAAAACCCAGAAGGTCCGCATCGAAGGCTCCCTGTGGTGGAC
CTACACAAGCAGCATCTTCTTCCGGGTCATCTTCGAAGCCGCCTTCATGTACGTCTTC
TATGTCATGTACGACGGCTTCTCCATGCAGCGGCTGGTGAAGTGCAACGCCTGGCCT
TGTCCCAACACTGTGGACTGCTTTGTGTCCCGGCCCACGGAGAAGACTGTCTTCACA
GTGTTCATGATTGCAGTGTCTGGAATTTGCATCCTGCTGAATGTCACTGAATTGTGTT
ATTTGCTAATTAGATATTGTTCTGGGAAGTCAAAAAAGCCAGTTGGATCCCGGGCTG
ACTACAAAGACCATGACGGTGATTATAAAGATCATGACATCGACTACAAGGATGAC
GATGACAAGTAAGAAATAGACAGCATGAGAGGGATGAGGCAACCCGTGCTCAGCTG
TCAAGGCTCAGTCGCTAGCATTTCCCAACACAAAGATTCTGACCTTAAATGCAACCA
TTTGAAACCCCTGTAGGCCTCAGGTGAAACTCCAGATGCCACAATGGAGCTCTGCTC
CCCTAAAGCCTCAAAACAAAGGCCTAATTCTATGCCTGTCTTAATTTTCTTTCACTTA
AGTTAGTTCCACTGAGACCCCAGGCTGTTAGGGGTTATTGGTGTAAGGTACTTTCAT
ATTTTAAACAGAGGATATCGGCATTTGTTTCTTTCTCTGAGGACAAGAGAAAAAAGC
CAGGTTCCACAGAGGACACAGAGAAGGTTTGGGTGTCCTCCTGGGGTTCTTTTTGCC
AACTTTCCCCACGTTAAAGGTGAACATTGGTTCTTTCATTTGCTTTGGAAGTTTTAAT
CTCTAACAGTGGACAAAGTTACCAGTGCCTTAAACTCTGTTACACTTTTTGGAAGTG
AAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACT
ATGATTTAATTTGAAATATGGTCTTTTGGTTATGAATACTTTGCAGCACAGCTGAGAG
GCTGTCTGTTGTATTCATTGTGGTCATAGCACCTAACAACATTGTAGCCTCAATCGAG
TGAGACAGACTAGAAGTTCCTAGTGATGGCTTATGATAGCAAATGGCCTCATGTCAA
ATATTTAGATGTAATTTTGTGTAAGAAATACAGACTGGATGTACCACCAACTACTAC
CTGTAATGACAGGCCTGTCCAACACATCTCCCTTTTCCATGACTGTGGTAGCCAGCAT
CGGAAAGAACGCTGATTTAAAGAGGTCGCTTGGGAATTTTATTGACACAGTACCATT
TAATGGGGAGGACAAAATGGGGCAGGGGAGGGAGAAGTTTCTGTCGTTAAAAACAG
ATTTGGAAAGACTGGACTCTAAAGTCTGTTGATTAAAGATGAGCTTTGTCTACTTCA
AAAGTTTGTTTGCTTACCCCTTCAGCCTCCAATTTTTTAAGTGAAAATATAGCTAATA
ACATGTGAAAAGAATAGAAGCTAAGGTTTAGATAAATATTGAGCAGATCTATAGGA
AGATTGAACCTGAATATTGCCATTATGCTTGACATGGTTTCCAAAAAATGGTACTCC
ACATATTTCAGTGAGGGTAAGTATTTTCCTGTTGTCAAGAATAGCATTGTAAAAGCA
TTTTGTAATAATAAAGAATAGCTTTAATGATATGCTTGTAACTAAAATAATTTTGTAA
TGTATCAAATACATTTAAAACATTAAAATATAATCTCTATAATAAGAGCTCGCTGAT
CAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTC
TATGGAAGCTTGAATTCAGCTGACGTGCCTCGGACCGCTAGGAACCCCTAGTGATGG
AGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAGC
TGCCTGCAGGGGCGCCTGATGCGGTATTTTCTCCTTACGCATCTGTGCGGTATTTCAC
ACCGCATACGTCAAAGCAACCATAGTACGCGCCCTGTAGCGGCGCATTAAGCGCGG
CGGGTGTGGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCA
AAAAACTTGATTTGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGG
AACAACACTCAACCCTATCTCGGGCTATTCTTTTGATTTATAAGGGATTTTGCCGATT
TCGGCCTATTGGTTAAAAAATGAGCTGATTTAACAAAAATTTAACGCGAATTTTAAC
AAAATATTAACGTTTACAATTTTATGGTGCACTCTCAGTACAATCTGCTCTGATGCCG
CATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGT
GTCAGAGGTTTTCACCGTCATCACCGAAACGCGCGAGACGAAAGGGCCTCGTGATA
CGCCTATTTTTATAGGTTAATGTCATGAACAATAAAACTGTCTGCTTACATAAACAGT
AATACAAGGGGTGTTATGAGCCATATTCAACGGGAAACGTCGAGGCCGCGATTAAA
TTCCAACATGGATGCTGATTTATATGGGTATAAATGGGCTCGCGATAATGTCGGGCA
ATCAGGTGCGACAATCTATCGCTTGTATGGGAAGCCCGATGCGCCAGAGTTGTTTCT
GAAACATGGCAAAGGTAGCGTTGCCAATGATGTTACAGATGAGATGGTCAGACTAA
ACTGGCTGACGGAATTTATGCCTCTTCCGACCATCAAGCATTTTATCCGTACTCCTGA
TGATGCATGGTTACTCACCACTGCGATCCCCGGAAAAACAGCATTCCAGGTATTAGA
AGAATATCCTGATTCAGGTGAAAATATTGTTGATGCGCTGGCAGTGTTCCTGCGCCG
GTTGCATTCGATTCCTGTTTGTAATTGTCCTTTTAACAGCGATCGCGTATTTCGTCTCG
CTCAGGCGCAATCACGAATGAATAACGGTTTGGTTGATGCGAGTGATTTTGATGACG
AGCGTAATGGCTGGCCTGTTGAACAAGTCTGGAAAGAAATGCATAAACTTTTGCCAT
TCTCACCGGATTCAGTCGTCACTCATGGTGATTTCTCACTTGATAACCTTATTTTTGA
CGAGGGGAAATTAATAGGTTGTATTGATGTTGGACGAGTCGGAATCGCAGACCGAT
ACCAGGATCTTGCCATCCTATGGAACTGCCTCGGTGAGTTTTCTCCTTCATTACAGAA
ACGGCTTTTTCAAAAATATGGTATTGATAATCCTGATATGAATAAATTGCAGTTTCAT
TTGATGCTCGATGAGTTTTTCTAATCTCATGACCAAAATCCCTTAACGTGAGTTTTCG
TTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGA
GCGCAGATACCAAATACTGTCCTTCTAGTGTAGCCGTAGTTAGGCCACCACTTCAAG
AACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGG
TTTCGCCACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTGCTGGCCTT
TTGCTCACATGT

TABLE 16
Components of Construct Sequence (SEQ ID NO: 107)

| Components | Position in construct |
|---|---|
| 5′ITR | 12-130 |
| Cloning site | 131-147 |
| VIM promoter | 148-1234 |
| hGJB2 minimal promoter | 1235-1362 |
| Cloning site | 1363-1371 |
| Synthetic barcode | 1372-1379 |
| 5′UTR | 1380-1741 |
| GJB2 (exon2) | 1753-2430 |
| 3xFLAG | 2443-2508 |
| 3′UTR (exon2) | 2512-3918 |
| bGHpA | 3940-4164 |
| Cloning site | 4165-4198 |
| 3′ITR | 4199-4328 |

### Pharmaceutical Compositions

Among other things, the present disclosure provides pharmaceutical compositions. In some aspects, compositions provided herein are suitable for administration to an animal for the amelioration of symptoms associated with syndromic and/or nonsyndromic hearing loss.

In some aspects, pharmaceutical compositions of the present disclosure may comprise, e.g., a polynucleotide, e.g., one or more constructs, as described herein. In some aspects, a pharmaceutical composition may comprise one or more AAV particles, e.g., one or more rAAV construct encapsidated by one or more AAV serotype capsids, as described herein.

In some aspects, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some aspects, formulations are in a dosage forms, such as injectable solutions, injectable gels, drug-release capsules, and the like.

In some aspects, compositions of the present disclosure are formulated for intravenous administration. In some aspects compositions of the present disclosure are formulated for intra-cochlear administration. In some aspects, a composition (e.g., a therapeutic composition) is formulated to comprise a lipid nanoparticle, a polymeric nanoparticle, a mini-circle DNA and/or a CELiD DNA.

In some aspects, a composition disclosed herein is formulated as a sterile suspension for intracochlear administration. In some aspects, a composition comprises constructs in an amount of at least 1E11, at least 5E11, at least 1E12, at least 5E12, at least 1E13, at least 2E13, at least 3E13, at least 4E13, at least 5E13, at least 6E13, at least 7E13, at least 8E13, at least 9E13, or at least 1E14 vector genomes (vg) per milliliter (mL). In some aspects, a composition comprises constructs in an amount of at most 1E15, at most 5E14, at most 1E14, at most 5E13, at most 1E13, at most 9E12, at most 8E12, at most 7E12, at most 6E12, at most 5E12, at most 4E12, at most 3E12, at most 2E12, or at most 1E12 vector genomes (vg) per milliliter (mL). In some aspects, a composition comprises constructs in an amount of 1E12 to 1E13, 5E12 to 5E13, or 1E13 to 2E13 vector genomes (vg) per milliliter (mL).

In some aspects, a therapeutic composition is formulated to comprise a synthetic perilymph solution. For example, in some aspects, a synthetic perilymph solution includes 20-200 mM NaCl; 1-5 mM KCl; 0.1-10 mM CaCl₂); 1-10 mM glucose; and 2-50 mM HEPES, with a pH between about 6 and about 9. In some aspects, a therapeutic composition is formulated to comprise a physiologically suitable solution. For example, in some aspects, a physiologically suitable solution comprises commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some aspects, alternative pluronic acids are utilized. In some aspects, alternative ion concentrations are utilized.

In some aspects, any of the pharmaceutical compositions described herein may further comprise one or more agents that promote the entry of a nucleic acid or any of the constructs described herein into a mammalian cell (e.g., a liposome or cationic lipid). In some aspects, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some aspects, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some aspects, a composition provided herein can be, e.g., formulated to be compatible with their intended route of administration. A non-limiting example of an intended route of administration is local administration (e.g., intra-cochlear administration). In some aspects, a provided composition comprises one nucleic acid construct. In some aspects, a provided composition comprises two or more different constructs. In some aspects, a composition that include a single nucleic acid construct comprising a coding sequence that encodes a polypeptide (e.g., a therapeutic polypeptide, a Connexin 26 polypeptide) and/or a functional characteristic portion thereof. In some aspects, compositions comprise a single nucleic acid construct comprising a coding sequence that encodes a polypeptide (e.g., a therapeutic polypeptide, a Connexin 26 polypeptide) and/or a functional characteristic portion thereof, which, when introduced into a mammalian cell, that coding sequence is integrated into the genome of the mammalian cell.

Also provided are kits including any of the compositions described herein. In some aspects, a kit can include a solid composition (e.g., a lyophilized composition including the at least two different constructs described herein) and a liquid for solubilizing the lyophilized composition. In some aspects, a kit can include a pre-loaded syringe including any of the compositions described herein.

In some aspects, the kit includes a vial comprising any of the compositions described herein (e.g., formulated as an aqueous composition, e.g., an aqueous pharmaceutical composition).

In some aspects, the kits can include instructions for performing any of the methods described herein.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some aspects, an animal cell is a human cell (e.g., a human supporting cell or a human hair cell). In other aspects, an animal cell is a non-human mammal (e.g., Simian cell, Felidae cell, Canidae cell etc.). A person skilled in the art will appreciate that the nucleic acids and constructs described herein can be introduced into any animal cell (e.g., the supporting or hair cells of any animal suitable for veterinary intervention). Non-limiting examples of constructs and methods for introducing constructs into animal cells are described herein.

In some aspects, an animal cell can be any cell of the inner ear, including hair and/or supporting cells. Non-limiting examples such cells include: Hensen's cells, Deiters' cells, cells of the endolymphatic sac and duct, transitional cells in the saccule, utricle, and ampulla, inner and outer hair cells, spiral ligament cells, spiral ganglion cells, spiral prominence cells, external saccule cells, marginal cells, intermediate cells, basal cells, inner pillar cells, outer pillar cells, Claudius cells, inner border cells, inner phalangeal cells, or cells of the stria vascularis.

In some aspects, an animal cell is a specialized cell of the cochlea. In some aspects, an animal cell is a hair cell. In some aspects, an animal cell is a cochlear inner hair cell or a cochlear outer hair cell. In some aspects, an animal cell is a cochlear inner hair cell. In some aspects, an animal cell is a cochlear outer hair cell.

In some aspects, an animal cell is in vitro. In some aspects, an animal cell is of a cell type which is endogenously present in an animal, e.g., in a primate and/or human. In some aspects, an animal cell is an autologous cell obtained from an animal and cultured ex vivo. In some aspects, the ex vivo cell is an inner ear cell. In some aspects, the ex vivo cell is an inner ear supporting cell. In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

### Methods

Among other things, the present disclosure provides methods. In some aspects, a method comprises introducing a composition, construct, or polynucleotide as described herein into the inner ear (e.g., a cochlea) of a subject. For example, provided herein are methods that in some aspects include administering to an inner ear (e.g., cochlea) of a subject (e.g., an animal, e.g., a mammal, e.g., a primate, e.g., a human) a therapeutically effective amount of any composition, construct, or polynucleotide described herein. In some aspects of any of these methods, the subject has been previously identified as having a defective inner ear cell target gene (e.g., a supporting and/or hearing cell target gene having a mutation that results in a decrease in the expression and/or activity of a supporting and/or hearing cell target protein encoded by the gene). Some aspects of any of these methods further include, prior to the introducing or administering step, determining that the subject has a defective inner ear cell target gene. Some aspects of any of these methods can further include detecting a mutation in an inner ear cell target gene in a subject. Some aspects of any of the methods can further include identifying or diagnosing a subject as having nonsyndromic or syndromic sensorineural hearing loss.

In some aspects, provided herein are methods of correcting an inner ear cell target gene defect in an inner ear of a subject, e.g., an animal, e.g., a mammal, e.g., a primate, e.g., a human. In some aspects, methods include administering to the inner ear of a subject a therapeutically effective amount of any of the compositions described herein, where the administering repairs and or ameliorates the inner ear cell target gene defect in any cell subset of the inner ear of a subject. In some aspects, the inner ear target cell may be a sensory cell, e.g., a hair cell, and/or a non-sensory cell, e.g., a supporting cell, and/or all or any subset of inner ear cells.

Also provided herein are methods of promoting expression (e.g., increasing the expression level) of an inner ear cell target protein in any subset of inner ear cells of a subject (e.g., an animal, e.g., a mammal, e.g., a primate, e.g., a human) that include: administering to the inner ear of the subject a therapeutically effective amount of any of the compositions described herein, where the administering results in an increase in the expression level of the inner ear cell target protein (e.g., a polypeptide (e.g., a therapeutic polypeptide, a Connexin 26 polypeptide)) in any cell subset of the inner ear of a subject. In some aspects, the inner ear target cell may be a sensory cell, e.g., a hair cell, and/or a non-sensory cell, e.g., a supporting cell, and/or all or any subset of inner ear cells.

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the compositions described herein.

Also provided herein are methods of restoring synapses and/or preserving spiral ganglion nerves in a subject identified or diagnosed as having an inner ear disorder that include: administering to the inner ear of the subject a therapeutically effective amount of any of the compositions described herein.

Also provided herein are methods of reducing the size of, and/or restoring the vestibular aqueduct to an appropriate size. Also provided herein are methods of restoring endolymphatic pH to an appropriate and/or acceptable level in a subject identified or diagnosed as having an inner ear disorder that include: administering to the inner ear of the subject a therapeutically effective amount of any of the compositions described herein.

Also provided herein are methods of expressing a polypeptide (e.g., a therapeutic polypeptide, a Connexin 26 polypeptide) in an inner ear supporting cell of a subject in need thereof. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the polypeptide (e.g., therapeutic polypeptide, a Connexin 26 polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide (e.g., a Connexin 26 polypeptide) is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods of increasing expression of a therapeutic polypeptide (e.g., a Connexin 26 polypeptide) in an inner ear supporting cell of a subject in need thereof. In some aspects, the expression of the therapeutic polypeptide (e.g., a Connexin 26 polypeptide) is reduced, suppressed, or eliminated in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, expression of the therapeutic polypeptide (e.g., a Connexin 26 polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide (e.g., Connexin 26 polypeptide) is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Also provided herein are methods of promoting expression (e.g., increasing expression) of a polypeptide in an inner ear supporting cell of a subject in need thereof. In some aspects, the expression of the polypeptide is reduced, suppressed, or eliminated in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wallIn some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods of decreasing expression of the therapeutic polypeptide (e.g., Connexin 26 polypeptide) in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Also provided herein are methods of decreasing expression of the polypeptide in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the therapeutic polypeptide (e.g., Connexin 26 polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide (e.g., Connexin 26 polypeptide) is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of reducing toxicity associated with expression of the therapeutic polypeptide (e.g., Connexin 26 polypeptide) in an inner ear cell. In some aspects, the inner ear cells are inner ear hair cells, spiral ganglion cells, lateral supporting cells, basilar membrane cells, medial supporting cells, spiral limbus cells, or any combination thereof. In some aspects, expression of the therapeutic polypeptide (e.g., Connexin 26 polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide (e.g., Connexin 26 polypeptide) is reduced in inner ear hair cells, spiral ganglion cells, lateral supporting cells, basilar membrane cells, medial supporting cells, spiral limbus cells, or any combination thereof. In some aspects, the therapeutic protein (e.g., Connexin 26 polypeptide) is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of reducing toxicity associated with expression of the polypeptide in an inner ear cell. In some aspects, the inner ear cells are inner ear hair cells, spiral ganglion cells, lateral supporting cells, basilar membrane cells, medial supporting cells, spiral limbus cells, or any combination thereof. In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of treating hearing loss in a subject suffering from or at risk of hearing loss. In some aspects, expression of the therapeutic polypeptide (e.g., Connexin 26 polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide (e.g., Connexin 26 polypeptide) is reduced in inner ear hair cells, spiral ganglion cells, lateral supporting cells, basilar membrane cells, medial supporting cells, spiral limbus cells, or any combination thereof. In some aspects, the therapeutic protein (e.g., Connexin 26) is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Also provided herein are methods of treating hearing loss in a subject suffering from or at risk of hearing loss. In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods that include administering to an inner ear of a subject a therapeutically effective amount of any of the compositions described herein.

Also provided herein are surgical methods for treatment of hearing loss (e.g., nonsyndromic sensorineural hearing loss or syndromic sensorineural hearing loss). In some aspects, the methods include the steps of: introducing into a cochlea of a subject a first incision at a first incision point; and administering intra-cochlearly a therapeutically effective amount of any of the compositions provided herein. In some aspects, the composition is administered to the subject at the first incision point. In some aspects, the composition is administered to the subject into or through the first incision.

In some aspects of any of the methods described herein, any composition described herein is administered to the subject into or through the cochlea oval window membrane. In some aspects of any of the methods described herein, any of the compositions described herein is administered to the subject into or through the cochlea round window membrane. In some aspects of any of the methods described herein, the composition is administered using a medical device capable of creating a plurality of incisions in the round window membrane. In some aspects, the medical device includes a plurality of micro-needles. In some aspects, the medical device includes a plurality of micro-needles including a generally circular first aspect, where each micro-needle has a diameter of at least about 10 microns. In some aspects, the medical device includes a base and/or a reservoir capable of holding the composition. In some aspects, the medical device includes a plurality of hollow micro-needles individually including a lumen capable of transferring the composition. In some aspects, the medical device includes a means for generating at least a partial vacuum.

In some aspects, technologies of the present disclosure are used to treat subjects with or at risk of hearing loss. In some such aspects, a pathogenic variant causes or is at risk of causing hearing loss.

In some aspects, a subject experiencing hearing loss will be evaluated to determine if and where one or more mutations may exist that may cause hearing loss. In some aspects of any of the methods described herein, the subject or animal is a mammal, in some aspects the mammal is a domestic animal, a farm animal, a zoo animal, a non-human primate, or a human. In some aspects of any of the methods described herein, the animal, subject, or mammal is an adult, a teenager, a juvenile, a child, a toddler, an infant, or a newborn. In some aspects of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some aspects of any of the methods described herein, the subject or mammal is 1, 2, 3, 4, 5, 6, 7, 8, 9, 10, or 11 months of age.

In some aspects of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some aspects a subject (e.g., an animal, e.g., a mammal, e.g., a human) has or is at risk of developing syndromic or nonsyndromic sensorineural hearing loss.

In some aspects, a subject (e.g., an animal, e.g., a mammal, e.g., a human) has been identified as having syndromic or nonsyndromic sensorineural hearing loss.

In some aspects, a subject (e.g., an animal, e.g., a mammal, e.g., a human) has been identified as being at risk of hearing loss (e.g., at risk of being a carrier of a gene mutation). In some such aspects, a subject (e.g., an animal, e.g., a mammal, e.g., a human) may have certain risk factors of hearing loss or risk of hearing loss (e.g., known parental carrier, afflicted sibling, or symptoms of hearing loss). In some such aspects, a subject (e.g., an animal, e.g., a mammal, e.g., a human) has been identified as being a carrier of a mutation in a gene (e.g., via genetic testing) that has not previously been identified ( ). In some such aspects, identified mutations may be novel (i.e., not previously described in the literature), and methods of treatment for a subject suffering from or susceptible to hearing loss will be personalized to the mutation(s) of the particular patient.

In some aspects, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some aspects of any method provided herein, two or more doses of any composition described herein are introduced or administered into a cochlea of a subject. Some aspects of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some aspects of any method provided herein, the composition can be formulated for intra-cochlear administration. In some aspects of any of the methods described herein, the compositions described herein can be administered via intra-cochlear administration or local administration. In some aspects of any of the methods described herein, the compositions are administered through the use of a medical device (e.g., any of the exemplary medical devices described herein).

In some aspects, intra-cochlear administration can be performed using any of the methods described herein or known in the art. For example, in some aspects, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some aspects of any method provided herein, a subject has or is at risk of developing syndromic or nonsyndromic sensorineural hearing loss. In some aspects of any method provided herein, a subject has been previously identified as having a mutation in an inner ear cell target gene, a gene which may be expressed in supporting cells and/or hair cells.

In some aspects of any method provided herein, a subject has been identified as being a carrier of a mutation in an inner ear cell target gene (e.g., via genetic testing). In some aspects of any method provided herein, a subject has been identified as having a mutation in an inner ear cell target gene and has been diagnosed with hearing loss (e.g., nonsyndromic sensorineural hearing loss or syndromic sensorineural hearing loss, e.g., DFNB1, DFNA3). Bart-Pumphrey syndrome, hystrix-like ichthyosis with deafness (HID), palmoplantar keratoderma with deafness, keratitis-ichthyosis-deafness (KID) syndrome, or Vohwinkel syndrome, respectively). In some aspects of any of the methods described herein, the subject has been identified as having hearing loss (e.g., nonsyndromic sensorineural hearing loss or syndromic sensorineural hearing loss). In some aspects, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some aspects, a subject cell is in vitro. In some aspects, a subject cell is originally obtained from a subject and is cultured ex vivo. In some aspects, the ex vivo cell is an inner ear cell. In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, a subject cell has previously been determined to have a defective inner ear cell target gene. In some aspects, a subject cell has previously been determined to have a defective hair cell target gene. In some aspects, a subject cell has previously been determined to have a defective supporting cell target gene.

In some aspects of these methods, following treatment e.g., one or two or more administrations of compositions described herein, there is an increase in expression of an active inner ear cell target protein (e.g., a polypeptide (e.g., a therapeutic polypeptide, a Connexin 26 polypeptide)). In some aspects, an increase in expression of an active inner ear target protein as described herein (e.g., a polypeptide (e.g., a therapeutic polypeptide, a Connexin 26 polypeptide)) is relative to a control level, e.g., as compared to the level of expression of an inner ear cell target protein prior to introduction of the compositions comprising any construct(s) as described herein.

Methods of detecting expression and/or activity of a target protein (e.g., a polypeptide (e.g., a therapeutic polypeptide, a Connexin 26 polypeptide)) are known in the art. In some aspects, a level of expression of an inner ear cell target protein can be detected directly (e.g., detecting inner ear cell target protein or target mRNA. Non-limiting examples of techniques that can be used to detect expression and/or activity of a target RNA or protein (e.g., a polypeptide (e.g., a therapeutic polypeptide, a Connexin 26 polypeptide)) directly include: real-time PCR, Western blotting, immunoprecipitation, immunohistochemistry, mass spectrometry, or immunofluorescence. In some aspects, expression of an inner ear cell target protein can be detected indirectly (e.g., through functional hearing tests).

### Devices, Administration, and Surgical Methods

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some aspects, a medical device includes a plurality of micro-needles.

Also provided herein are surgical methods for treatment of hearing loss (e.g., nonsyndromic sensorineural hearing loss or syndromic sensorineural hearing loss). In some aspects, a method the steps of: introducing into a cochlea of a subject a first incision at a first incision point; and administering intra-cochlearly a therapeutically effective amount of any of the compositions provided herein. In some aspects, a composition is administered to a subject at the first incision point. In some aspects, a composition is administered to a subject into or through the first incision.

In some aspects of any method provided herein, any of the compositions described herein is administered to the subject into or through the cochlea oval window membrane. In some aspects of any method provided herein, any of the compositions described herein is administered to the subject into or through the cochlea round window membrane. In some aspects of any method provided herein, the composition is administered using a medical device capable of creating a plurality of incisions in the round window membrane. In some aspects, a medical device includes a plurality of micro-needles. In some aspects, a medical device includes a plurality of micro-needles including a generally circular first aspect, where each micro-needle has a diameter of at least about 10 microns. In some aspects, a medical device includes a base and/or a reservoir capable of holding a composition. In some aspects, a medical device includes a plurality of hollow micro-needles individually including a lumen capable of transferring a composition. In some aspects, a medical device includes a means for generating at least a partial vacuum.

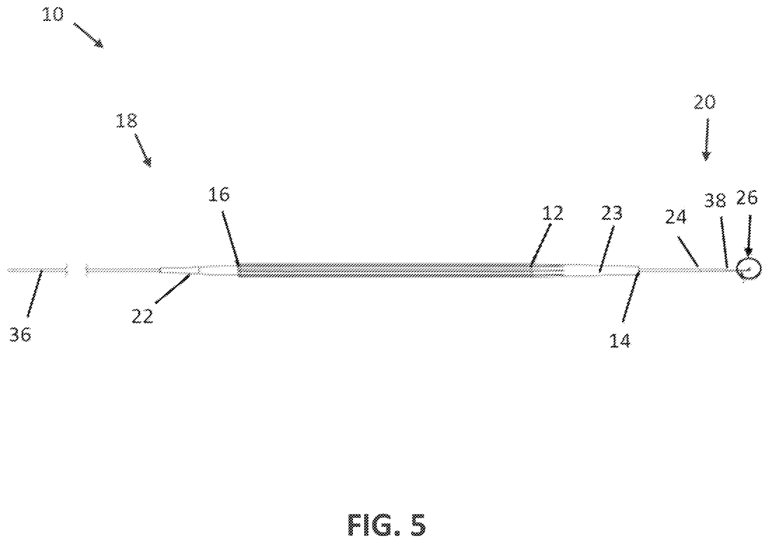

In some aspects, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 5-8 (or microcatheter) to deliver a composition disclosed herein at a controlled flow rate and in a fixed volume, via the round window membrane.

In some aspects, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some aspects, absence of venting during surgery may result in lower AAV vector cochlear cell transduction rates when compared to AAV vector cochlear cell transduction rates following surgeries performed with venting. In some aspects, venting facilitates transduction rates of about 75-100% of IHCs throughout the cochlea. In some aspects, venting permits IHC transduction rates of about 50-70%, about 60-80%, about 70-90%, or about 80-100% at the base of the cochlea. In some aspects, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some aspects, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some aspects, the flow rate is about 20 μL/min, about 30 μL/min, about 40 μL/min, about 50 μL/min, about 60 μL/min, about 70 μL/min, about 80 μL/min, about 90 μL/min or about 100 μL/min. In some aspects, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some aspects, the total volume of a composition disclosed herein that flows to the inner ear may be about 0.09 mL, or from about 0.08 mL to about 0.10 mL, or from about 0.07 mL to about 0.11 mL. In some aspects, the total volume of a composition disclosed herein equates to from about 40% to about 50% of the volume of the inner ear.

Once the delivery has been completed, the device may be removed. In some aspects, a device described herein, may be configured as a single-use disposable product. In other aspects, a device described herein may be configured as a multi-use, sterilizable product, for example, with a replaceable and/or sterilizable needle sub-assembly. Single use devices may be appropriately discarded (for example, in a biohazard sharps container) after administration is complete.

In some aspects, a composition disclosed herein comprises one or a plurality of rAAV constructs. In some aspects, when more than one rAAV construct is included in the composition, the rAAV constructs are each different. In some aspects, an rAAV construct comprises an anti-VEGF coding region, e.g., as described herein. In some aspects, a composition comprises an rAAV particle comprising an AAV construct described herein. In some aspects, the rAAV particle is encapsidated by an Anc80 capsid (e.g., an Anc80L65 capsid). In some aspects, the Anc80 capsid comprises a polypeptide of SEQ ID NO: 44.

In some aspects, a composition disclosed herein can be administered to a subject with a surgical procedure. In some aspects, administration, e.g., via a surgical procedure, comprises injecting a composition disclosed herein via a delivery device as described herein into the inner ear. In some aspects, a surgical procedure disclosed herein comprises performing a transcanal tympanotomy; performing a laser-assisted micro-stapedotomy; and injecting a composition disclosed herein via a delivery device as described herein into the inner ear.

In some aspects, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some aspects, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some aspects, performing a laser-assisted micro-stapedotomy includes using a KTP otologic laser and/or a C02 otologic laser.

As another example, a composition disclosed herein is administered using a device and/or system specifically designed for intracochlear route of administration. In some aspects, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

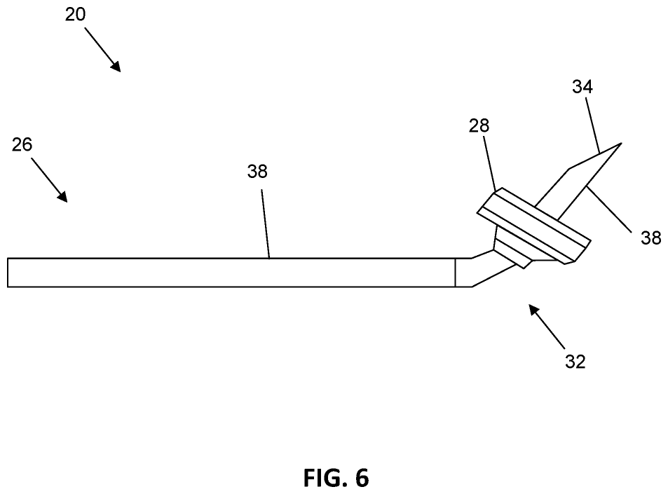

An exemplary device for use in any of the methods disclosed herein is described in FIGS. 5-8. FIG. 5 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 6) with stopper 28 (shown in FIG. 34) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 6) disposed therewithin.

Referring still to FIG. 5, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some aspects, may be coupled to a control system and/or power supply (not shown)). In some aspects, the bent needle 38 (shown in FIG. 6) extends from the distal end 20, through the telescoping hypotube needle support 24, through the tapered portion 23, through the knurled handle 12, and through the strain relief feature 22 and fluidly connects directly to the tubing 36. In other aspects, the bent needle 38 fluidly connects with the hollow interior of the knurled handle (for example, via the telescoping hypotube needle support 24) which in turn fluidly connects at a proximal end 16 with tubing 36. In aspects where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

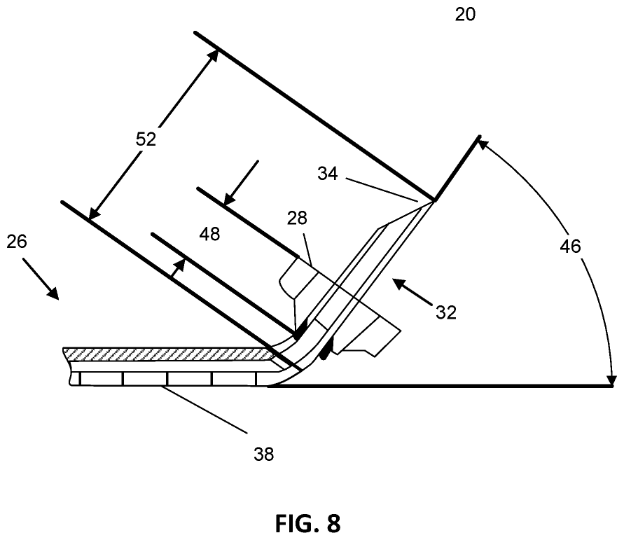

FIG. 6 illustrates a sideview of the bent needle sub-assembly 26, according to aspects of the present disclosed aspects. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 8) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

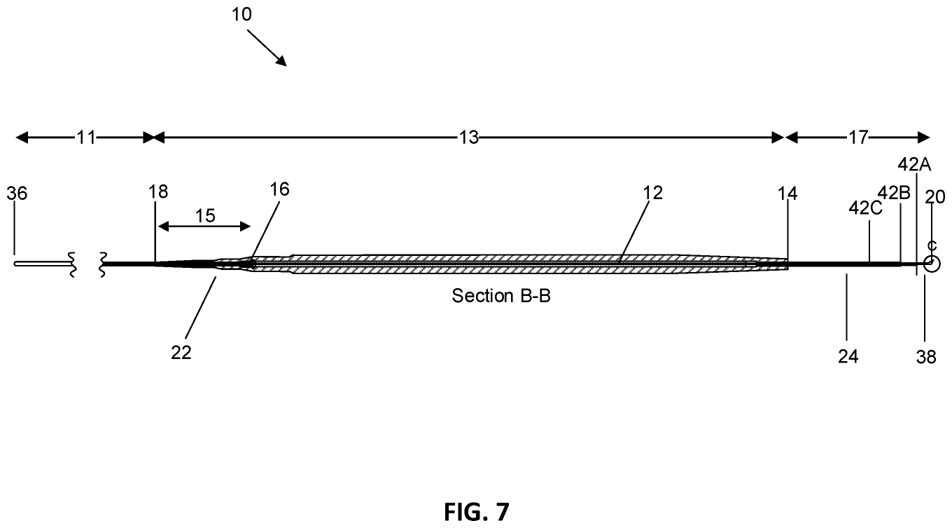

FIG. 7 illustrates a perspective view of exemplary device 10 for delivering fluid to an inner ear. Tubing 36 may be from about 1300 mm in length (dimension 11 in FIG. 7) to about 1600 mm, or from about 1400 mm to about 1500 mm, or from about 1430 mm to about 1450 mm. Strain release feature 22 may be from about 25 mm to about 30 mm in length (dimension 15 in FIG. 7), or from about 20 mm to about 35 mm in length. Handle 12 may be about 155.4 mm in length (dimension 13 in FIG. 7), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 7) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 8 illustrates a perspective view of bent needle sub-assembly 26 coupled to the distal end 20 of device 10, according to aspects of the present disclosed aspects. As shown in FIG. 8, bent needle sub-assembly 26 may include a needle 38 coupled to a bent portion 32. In other aspects, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 8, bent needle sub-assembly 26 may also include a stopper 28 disposed around bent portion 32. FIG. 8 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other aspects, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 8 shows that stopper 28 may have a geometry that is cylindrical, disk-shaped, and/or dome-shaped. A person of ordinary skill will appreciate that other geometries could be used.

### Evaluating Hearing Loss and Recovery

In some aspects, hearing function is determined using auditory brainstem response measurements (ABR). In some aspects, hearing is tested by measuring distortion product optoacoustic emissions (DPOAEs). In some such aspects, measurements are taken from one or both ears of a subject. In some such aspects, recordings are compared to prior recordings for the same subject and/or known thresholds on such response measurements used to define, e.g., hearing loss versus acceptable hearing ranges to be defined as normal hearing. In some aspects, a subject has ABR and/or DPOAE measurements recorded prior to receiving any treatment. In some aspects, a subject treated with one or more technologies described herein will have improvements on ABR and/or DPOAE measurements after treatment as compared to before treatment. In some aspects, ABR and/or DPOAE measurements are taken after treatment is administered and at regular follow-up intervals post-treatment.

In some aspects, hearing function is determined using speech pattern recognition or is determined by a speech therapist. In some aspects, hearing function is determined by pure tone testing. In some aspects, hearing function is determined by bone conduction testing. In some aspects, hearing function is determined by acoustic reflex testing. In some aspects hearing function is determined by tympanometry. In some aspects, hearing function is determined by any combination of hearing analysis known in the art. In some such aspects, measurements are taken holistically, and/or from one or both ears of a subject. In some such aspects, recordings and/or professional analysis are compared to prior recordings and/or analysis for the same subject and/or known thresholds on such response measurements used to define, e.g., hearing loss versus acceptable hearing ranges to be defined as normal hearing. In some aspects, a subject has speech pattern recognition, pure tone testing, bone conduction testing, acoustic reflex testing and/or tympanometry measurements and/or analysis conducted prior to receiving any treatment. In some aspects a subject treated with one or more technologies described herein will have improvements on speech pattern recognition, pure tone testing, bone conduction testing, acoustic reflex testing and/or tympanometry measurements after treatment as compared to before treatment. In some aspects, speech pattern recognition, pure tone testing, bone conduction testing, acoustic reflex testing and/or tympanometry measurements are taken after treatment is administered and at regular follow-up intervals post-treatment.

### Production Methods

AAV systems are generally well known in the art (see, e.g., Kelleher and Vos, Biotechniques, 17(6):1110-17 (1994); Cotton et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some aspects, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some aspects, such a stable host cell contains such component(s) under the control of an inducible promoter. In some aspects, such component(s) may be under the control of a constitutive promoter. In some aspects, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al., J. Virol., 70:520-532 (1993) and U.S. Pat. No. 5,478,745, which are incorporated in their entirety herein by reference).

In some aspects, recombinant AAVs may be produced using a triple transfection method (e.g., as described in U.S. Pat. No. 6,001,650, which is incorporated in its entirety herein by reference). In some aspects, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some aspects, the AAV helper function construct supports efficient AAV construct production without generating any detectable wild-type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the virus particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some aspects, viral construct titers post-purification are determined. In some aspects, titers are determined using quantitative PCR. In certain aspects, a TaqMan probe specific to a construct is utilized to determine construct levels. In certain aspects, the TaqMan probe is represented by SEQ ID NO: 58, while forward and reverse amplifying primers are exemplified by SEQ ID NO: 59 and 60 respectively.

Exemplary Taqman probe for quantification of constructs
(SEQ ID NO: 58)
/56-FAM/TCtGGCTCA/ZEN/CCGTCCTCTTCATTT/3IABkFQ/
Exemplary forward qPCR primer for quantification of constructs
(SEQ ID NO: 59)
CAAACACTCCACCAGCATTG
Exemplary reverse qPCR primer for quantification of constructs
(SEQ ID NO: 60)
CAGCCACAACGAGGATCATA

As described herein, in some aspects, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV 11, and AAV Anc80, as well as variants thereof. In some aspects, an AAV particle is an AAV2/6, AAV2/8, AAV2/9, or AAV2/Anc80 particle (e.g., with AAV6, AAV8, AAV9, or Anc80 capsid (e.g., an Anc80L65 capsid) and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV serotype may be used to deliver a coding sequence described herein. However, the serotypes have different tropisms, e.g., they preferentially infect different tissues. In some aspects, an AAV construct is a self-complementary AAV construct.

The present disclosure provides, among other things, methods of making AAV-based constructs. In some aspects, such methods include use of host cells. In some aspects, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, make and utilize technologies of the present disclosure.

### Example 1: Construction of Viral Constructs Comprising a Polypeptide or Therapeutic Polypeptide

This example provides a description of generating a viral construct as described herein. A recombinant AAV (rAAV) particle was generated by transfection with an adenovirus-free method as used by Xiao et al., J Virol. 73(5):3994-4003, 1999, which is incorporated in its entirety herein by reference. The cis plasmids with AAV ITRs, the trans plasmid with AAV Rep and Cap genes, and a helper plasmid with an essential region from an adenovirus genome were co-transfected in HEK293 cells. The rAAV construct expressed human connexin 26 under a single construct strategy using the constructs described. AAV Anc80 capsid was prepared to encapsulate a unique rAAV connexin 26 protein encoding construct.

Those of ordinary skill in the art will readily understand that similar constructs can be made in accordance with this example. For instance, rAAV constructs that express mammalian, primate, or human connexin 26 under single, dual, or multi construct strategies can be generated. AAV serotypes 1, 2, 3, 4, 5, 6, 7, 8, 9, rh8, rh10, rh39, rh43, and Anc80 can each be prepared to encapsulate four sets of connexin 26 constructs to test (i) a concatemerization-transplicing strategy, (ii) a hybrid intronic-homologous recombination-transplicing strategy, (iii) an exonic homologous recombination strategy, as summarized by Pryadkina et al., Meth. Clin. Devel. 2:15009, 2015, which is incorporated in its entirety herein by reference, and (iv) a single construct strategy. In some aspects, a recombinant AAV (rAAV) particle is generated by transfection with an adenovirus-free method as used by Xiao et al., J Virol. 73(5):3994-4003, 1999, which is incorporated in its entirety herein by reference.

### Example 2: Generating and Purifying Viral Particles

This example provides a description of purification of a viral construct. A recombinant AAV (rAAV) is produced using a triple transfection protocol and purified. The fractions are analyzed by dot blot to determine those containing rAAV genomes. The viral genome number (vg) of each preparation is determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

In some aspects of this example, a recombinant AAV (rAAV) was produced using a standard triple transfection protocol and purified by two sequential cesium chloride (CsCl) density gradients, as described by Pryadkina et al., Mol. Ther. 2:15009, 2015, which is incorporated in its entirety herein by reference. At the end of second centrifugation, 11 fractions of 500 μl were recovered from the CsCl density gradient tube and purified through dialysis in 1×PBS. The fractions were analyzed by dot blot to determine those containing rAAV genomes. The viral genome number (vg) of each preparation was determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

Those of ordinary skill in the art will readily understand that similar production and purifying processes can be conducted in accordance with this example. For instance, rAAV particles may be purified using various column chromatography methods known in the art, and/or viral genomes may be quantified using alternative primer sets.

### Example 3: Formulation of Viral Particles

This example relates to the preparation of compositions comprising rAAV particles, and a physiologically acceptable solution. An rAAV particle was produced and purified to a titer of 1.2×10¹³ vg/mL and was then prepared at dilutions of 6×10⁴, 1.3×10⁵, 1.8×10⁵, 4.5×10⁹, and 1.3×10¹⁰, vg/mL in a physiologically acceptable solution (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68).

In alternative aspects, an rAAV is produced and purified to a known concentration (e.g., a titer of approximately 1×10¹³ vg/mL) and is then prepared at desired concentrations (e.g., dilutions of 6×10⁴, 1.3×10⁵, 1.8×10⁵, 4.5×10⁹, and 1.3×10¹⁰, vg/mL) in a physiologically acceptable buffer (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68; or e.g., artificial perilymph comprising NaCl, 120 mM; KCl, 3.5 mM; CaCl₂), 1.5 mM; glucose, 5.5 mM; HEPES, 20 mM. which is titrated with NaOH to adjust its pH to 7.5 (total Na⁺ concentration of 130 mM) as described in Chen et al., J Controlled Rel. 110:1-19, 2005, which is incorporated in its entirety herein by reference). Those of ordinary skill in the art will readily understand that alternative formulations can be prepared in accordance with this example. For instance, rAAV particles may be purified to an alternative titer, prepared at alternative dilutions, and suspended in alternative suitable solutions.

### Example 4: Device Description

This example relates to a device suitable for the delivery of rAAV particles to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow a construct as described (e.g., an rAAV construct) to enter the cochlear perilymph of the scala tympani at a rate which does not damage the inner ear (e.g., a physiologically acceptable rate, e.g., a rate of approximately 30 μL/min to approximately 90 μL/min), but small enough to heal without surgical repair. The remaining portion of the microcatheter, proximal to the microneedle(s), is loaded with the rAAV/artificial perilymph formulation at a defined titer (e.g., approximately 1×10¹² to 5×10¹³ vg/mL). The proximal end of the microcatheter is connected to a micromanipulator that allows for precise, low volume infusions of approximately 30 μL to approximately 100 μL.

### Example 5: In-Vitro Demonstration of GJB2 mRNA and Connexin 26 Protein Production (Anti-FLAG Antibody)

This example relates to the introduction, regulation, and expression analysis of rAAV constructs expressing a supporting cell protein (e.g., a hGJB2 gene) in mammalian cells grown in vitro or ex vivo.

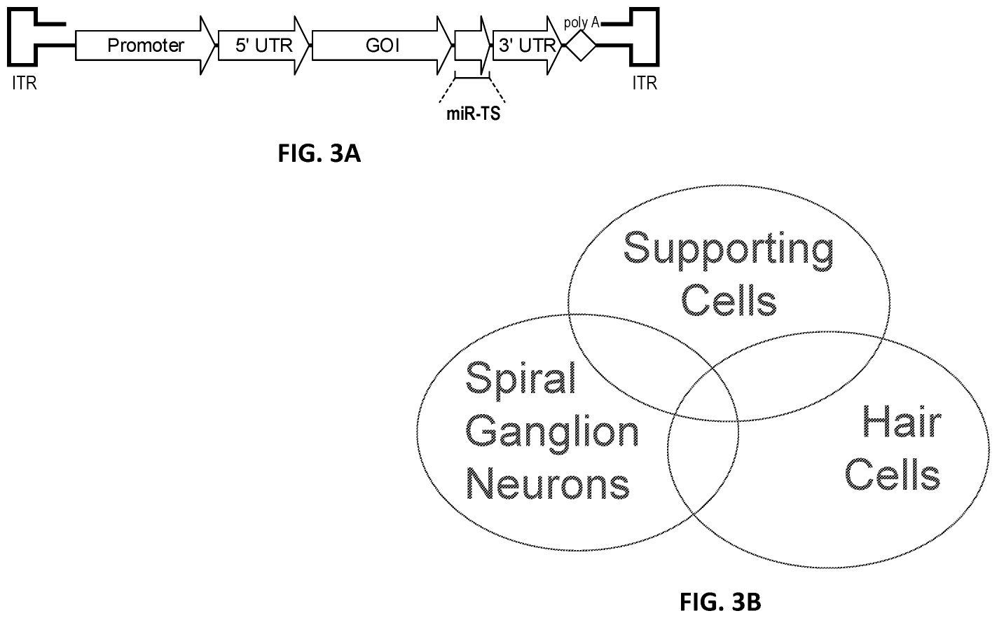

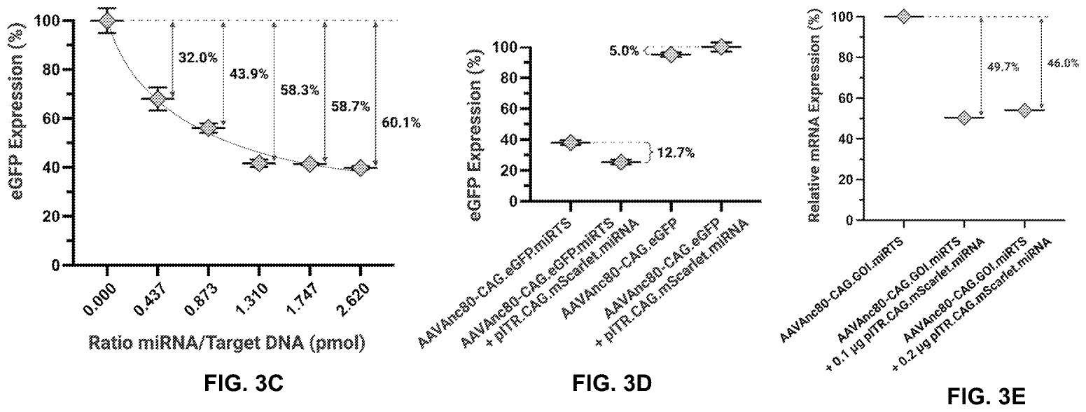

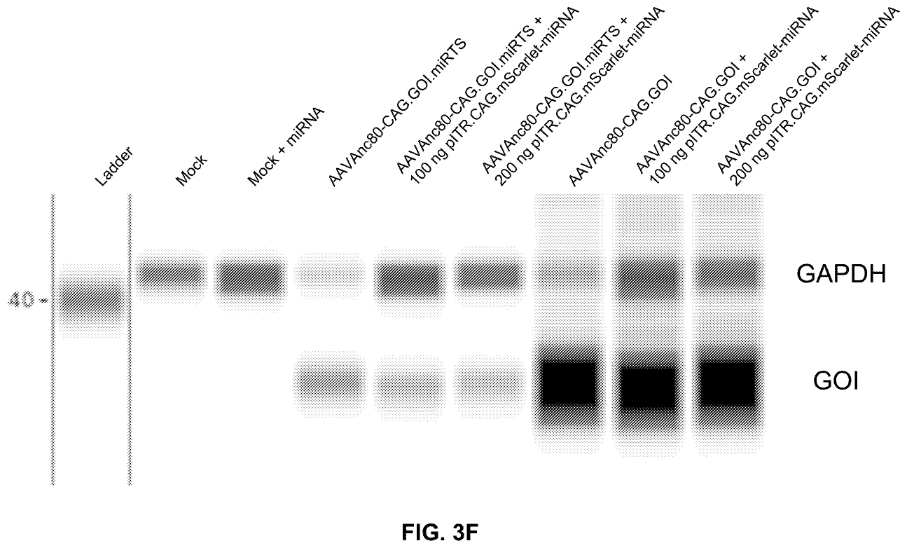

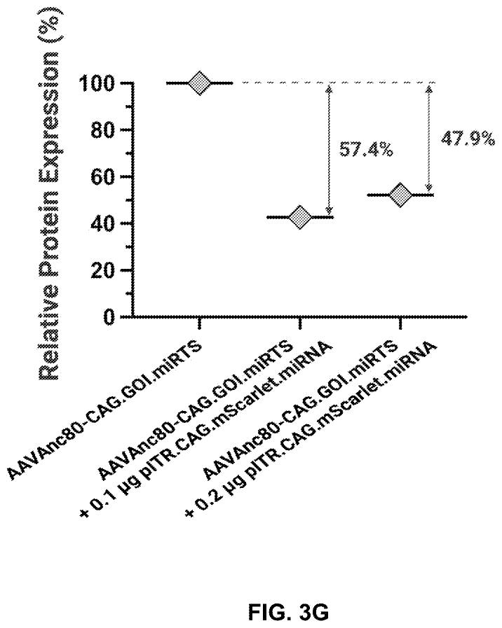

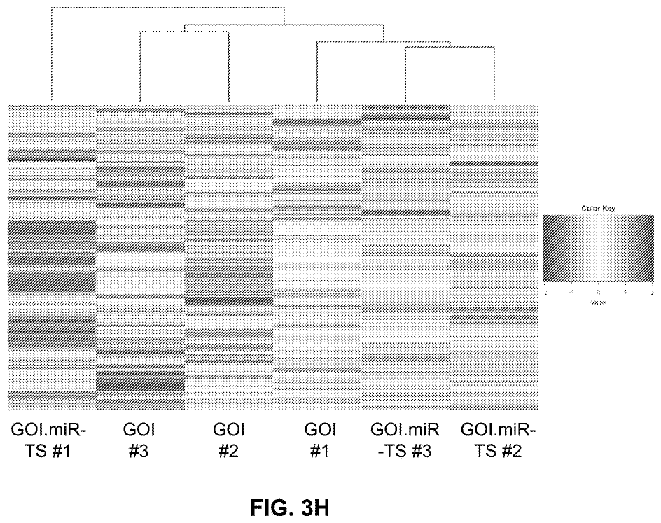

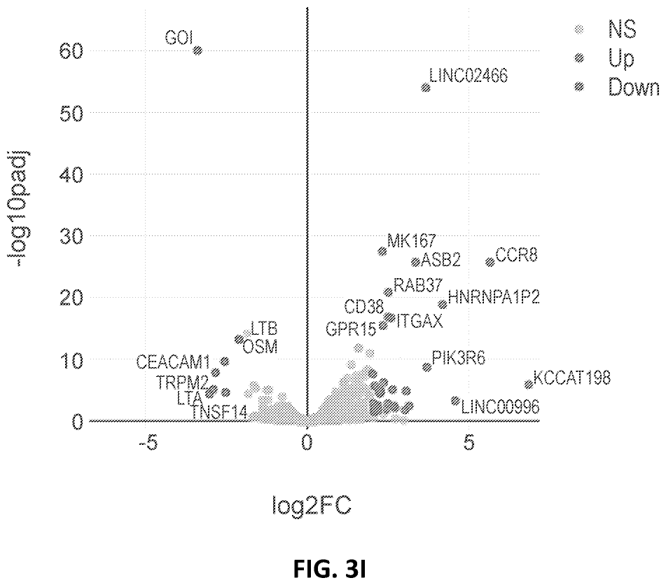

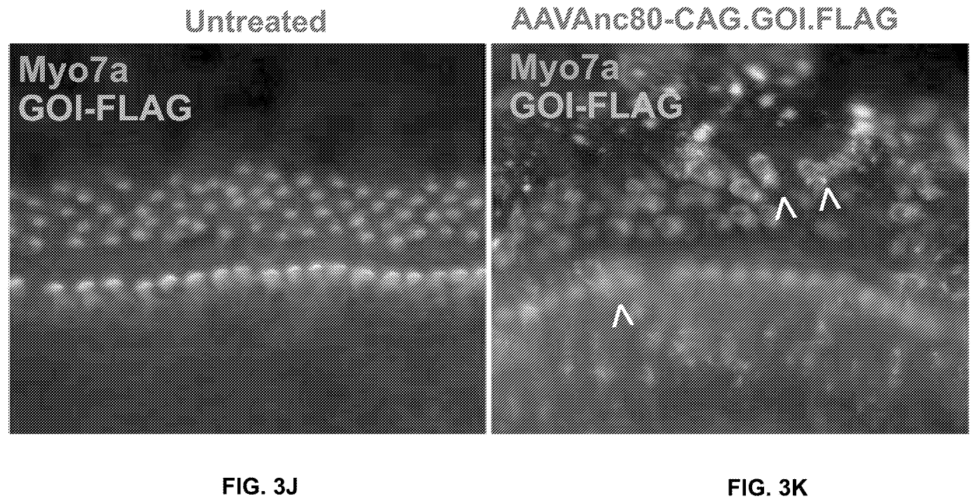

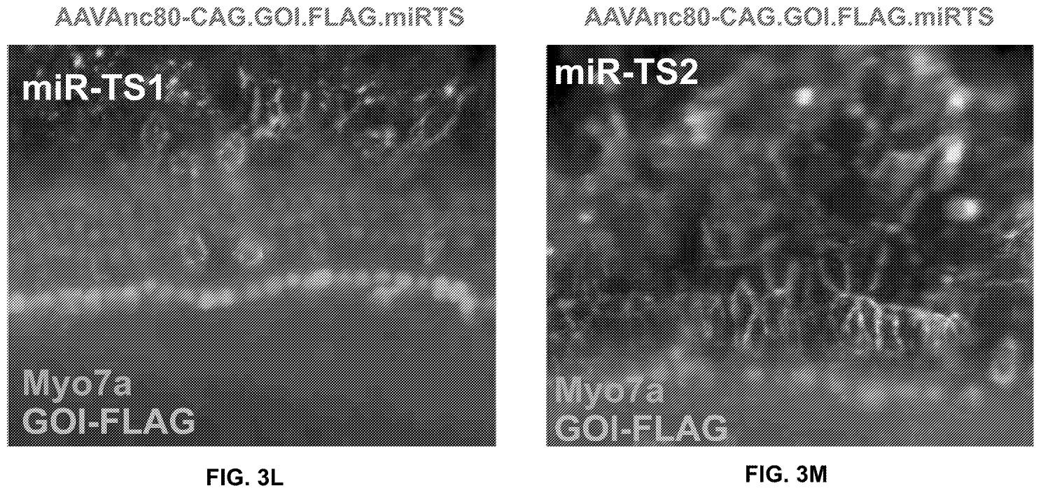

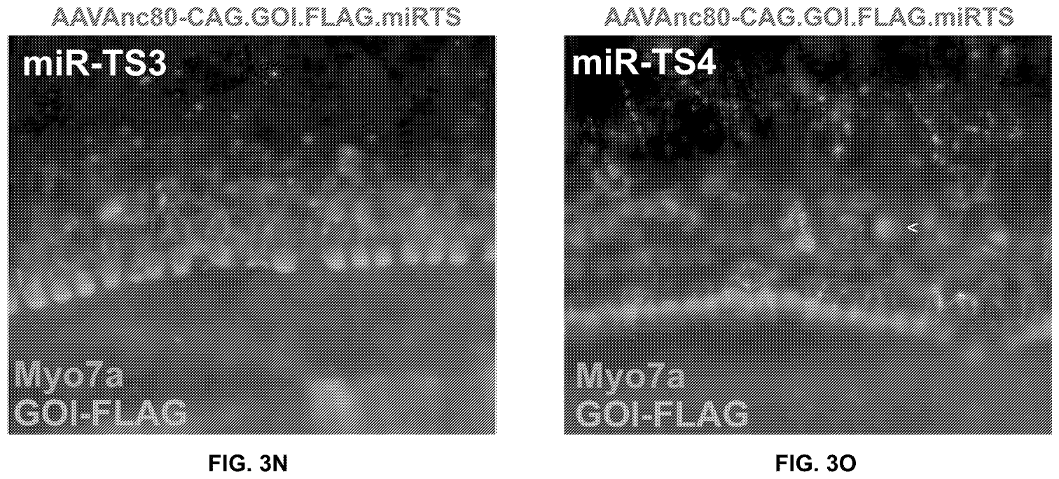

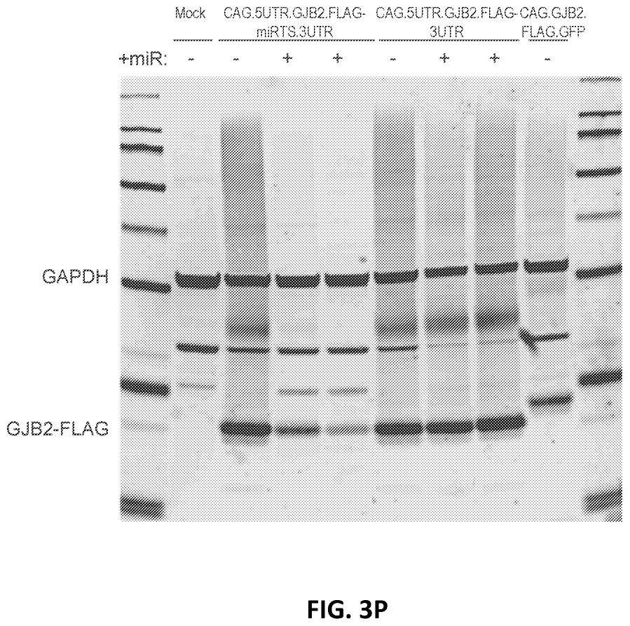

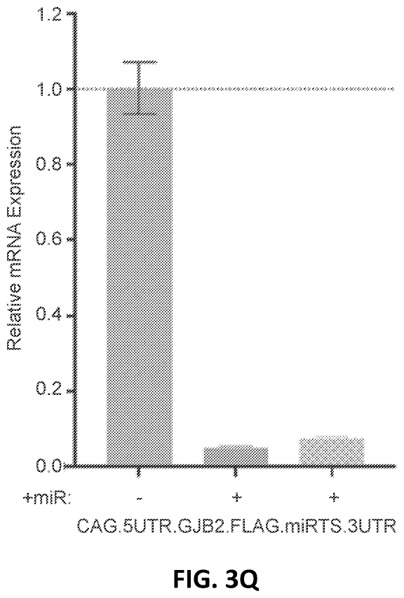

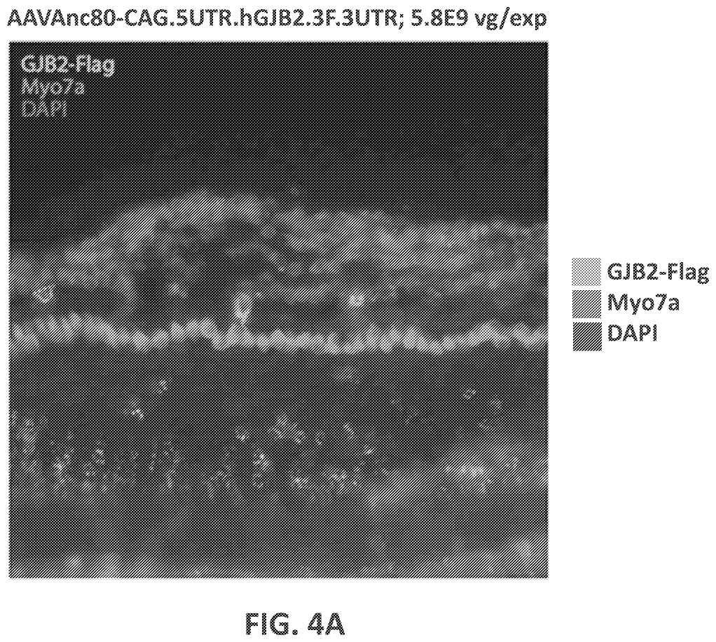

To regulate transgene mRNA expression only in supporting cells, a construct comprising microRNA target site was created. In cell types where the transgene (e.g., a supporting cell gene or a GJB2 gene) may not be well-tolerated (e.g., hair cells), a microRNA recognizes the miRTS and degrades the mRNA, preventing its expression. A schematic of the construct is shown in FIG. 3A. The construct comprises a promoter, 5′ UTR, transgene (GOI), miRTs, 3′ UTR, and polyA, flanked by inverted terminal repeats. To prevent expression of a transgene in hair cells and allow expression in other cochlear cell types, such as supporting cells, microRNAs expressed in hair cells but not in supporting cells can be used (FIG. 3B).

An in vitro experiment was conducted with a plasmid comprising eGFP and a miRTS (eGFP-miRTS) that was co-transfected into HEK293FT cells with a plasmid expressing a microRNA targeting the miRTS (pITR.CMV.mScarlet.miRNA) at range of miRNA to target DNA ratios. eGFP expression was measured by flow cytometry and normalized to eGFP-miRTS expression in the absence of the microRNA. Increasing ratios of pITR.CMV.mScarlet.miRNA to eGFP-miRTS led to a reduction in eGFP expression (FIG. 3C). When cells were transduced with an Anc80 particle comprising a construct expressing eGFP without a miRTS (eGFP) (AAVAnc80-CAG.eGFP) alongside transfection with pITR.CMV.mScarlet.miRNA, a reduction in expression of eGFP was not observed by flow cytometry. Transduction of cells with a contruct comprising eGFP and a miRTS (AAVAnc80-CAG.eGFP.miRTS) alongside transfection of the cells with pITR.CMV.mScarlet.miRNA resulted in decreased eGFP expression (FIG. 3D).

A similar effect on expression of a gene of interest (GOI (GJB2)) was observed when HEK293FT cells were transduced with an AAVAnc80 vector comprising a gene of intrest and miRTS site in its 3′UTR (AAVAnc80-CAG.GOI.miRTS) alongside transfection with pITR.CAG.mScarlet.miRNA, which expresses a microRNA targeting the miRTS. A reduction in the gene of interest mRNA was measured by real-time qPCR and western blotting when cells were transfected with either 100 ng or 200 ng of pITR.CAG.mScarlet.miRNA alongside transduction with AAVAnc80-CAG.GOI.miRTS compared to transduction of AAVAnc80-CAG.GOI.miRTS alone (FIGS. 3E and 3F).

In contrast, transfection of pITR.CAG.mScarlet-miRNA did not reduced the level of the gene of intrest when cells were transduced with a construct comprising the gene of interest without the 3′UTR miRTS (AAVAnc80-CAG.GOI) (FIG. 3G). The reduction in protein level observed by Western blot following AAVAnc80-CAG.GOI.miRTS transduction and pITR.CAG.mScarlet-miRNA was quantified in FIG. 3H.

In order to determine whether there were any substantial off-target effects on gene expression caused by transduction with AAVAnc80-CAG.GOI.miRTS, bulk RNA sequencing was performed. Cluster analysis failed to detect any clear sample clustering based on the transduction with or without the miRTS (FIG. 3I). Differential expression analysis was used to identify genes that were significantly upregulated or downregulated by the expression of AAVAnc80-CAG.GOImiRTS compared to AAVAnc80-CAG.GOI. The only genes significantly altered in expression were associated with T-cell immune response (FIG. 3J), further indicating that there were not significant off-target effects on gene expression caused by the expression of the miRTS-containing construct.

Cochlear explant cultures were transduced with an AAVAnc80 virus expressing a FLAG-tagged gene of interest (GOI (GJB2)) with a miRTS targeted by miRNAs expressed in hair cells (AAVAnc80-CAG.GOI.FLAG.miRTS1-4) or without a miRTS (AAVAnc80-CAG.GOI.FLAG). FIG. 3K (left) shows an untreated cochlear explant with hair cells labeled with MYO7A in red. Following transduction with AAVAnc80-CAG.GOIFLAG the gene of interest is expressed in hair cells (indicated by white arrowheads) and in both lateral and medial supporting cells (FIG. 3K, right). In contrast, following transduction of AAVAnc80-CAG.GOIFLAG.miRTS1, AAVAnc80-CAG.GOI.FLAG.miRTS2, and AAVAnc80-CAG.GOI.FLAG.miRTS3, expression of the gene of interest was limited to supporting cells, as observed by FLAG labeling (green) (FIGS. 3L, 3M, and 3N). Of the constructs expressing the gene of interest with a miRTS in the 3′UTR, only AAVAnc80-CAG.GOI.FLAG.miRTS4 was unsuccessful in limiting expression to supporting cells. FLAG labeling of the gene of interest is colocalized with MYO7A (white arrowhead) in some hair cells (FIG. 3O).Experiments were conducted to demonstrate mRNA expression regulation from rAAV constructs transfected into HEK293FT cells. rAAV constructs comprising hGJB2.FLAG (CAG.5UTR.hGJB2.FLAG.3UTR; SEQ ID NO: 82) and optional miRNA regulatory target sites (miRTS) located in the 3′UTR (CAG.5UTR.hGJB2.FLAG.miRTS.3UTR;

FIG. 2F; SEQ ID NO: 87) were transfected into HEK293FT cells at 300 ng with (+) or without (−) an additional plasmid comprising miRNA coding regions (e.g., miR-182, and miR-183) transfected at 400 ng. At 72 h post transfection the cells were harvested for GJB2 protein and RNA analysis using western blot analysis (see FIG. 3P) and real-time qPCR (see FIG. 3Q). Reduction in GJB2 RNA and protein expression was detected in samples that were co-expressing the target plasmid and miR-182 and miR-183 compared to samples expressing the target plasmid alone. Similar hGJB2.FLAG comprising plasmids that did not include miR-182 and miR-183 target sites were used as control and presented similar hGJB2 protein levels with and without miR-182 and miR-183 co-expression (see FIG. 3P and FIG. 3Q).

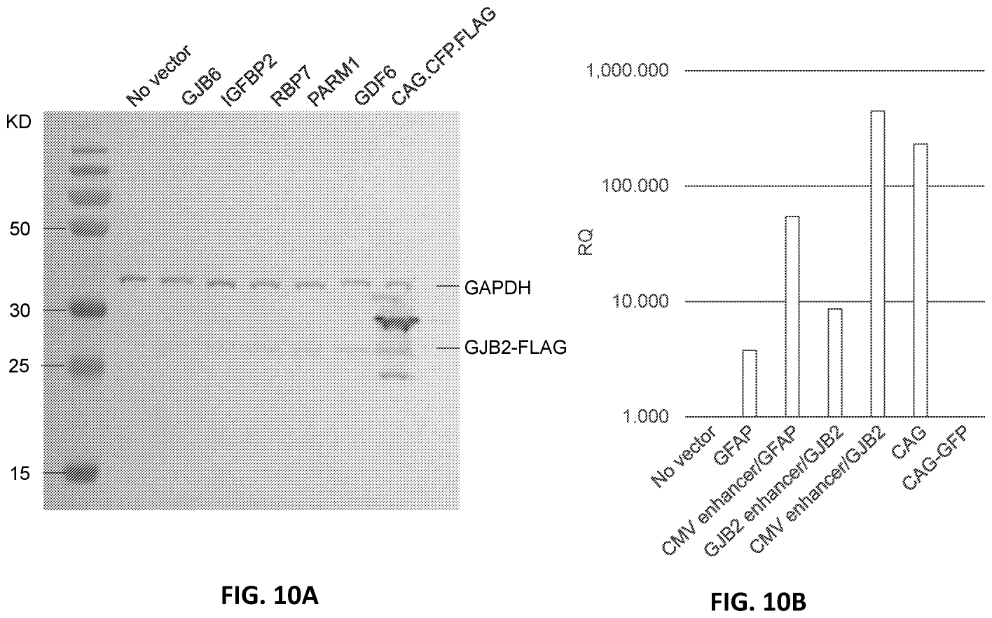

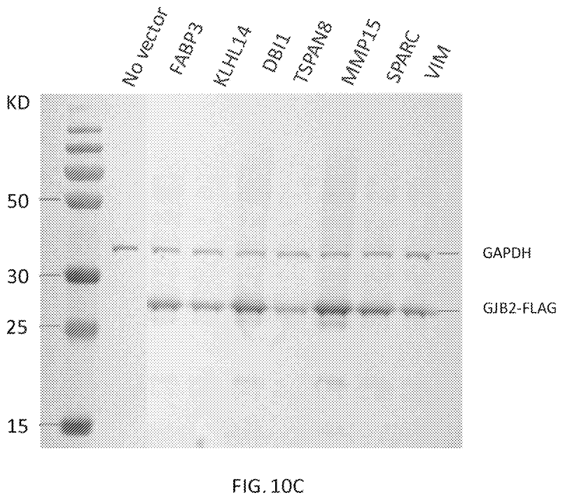

rAAV particles comprising rAAV constructs containing a GJB2 flag tagged polynucleotide operably linked to a supporting cell selective promoter (GFAP, GJB6, IGFBP2, RPB7, PARM1, or GDF6) in combination with a minimal GJB2 promoter were transduced into HEK293FT cells. Expression by a CAG promoter was used as a positive control. Protein and RNA analysis shows that each these constructs were able to express Connexin 26 (FIGS. 10A-10B).

Plasmids comprising a GJB2 flag tagged polynucleotide operably linked to a supporting cell selective promoter (FABP3, KLHL14, DBI2, TSPAN8, MMP15, SPARC, or VIM) in combination with a minimal GJB2 promoter were transfected into HEK293FT cells. Connexin 26 expression was observed from all constructs by western blot (FIG. 10C).

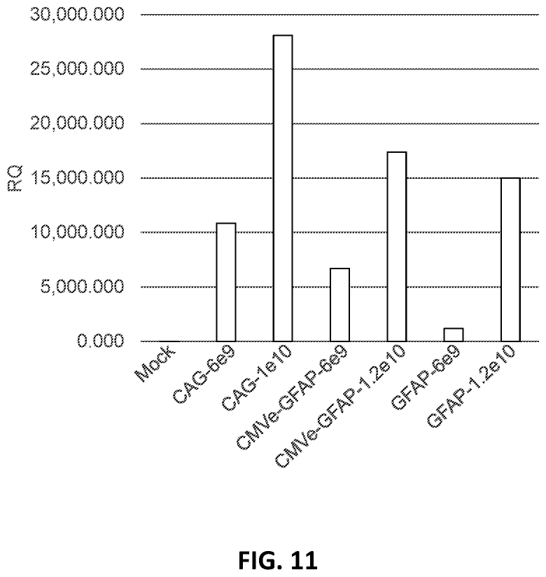

rAAV particles comprising rAAV contructs were encapsidated by Anc80 capisids and transduced into neonate cochlear explants at different doses. RNA analysis shows that GJB2 mRNA expression increased with dosing (FIG. 11).

Those of ordinary skill in the art will readily understand that there are alternative methods of conducting the experiments associated with the current example, for instance, alternative viral titers, MOIs, cell concentrations, time to cellular harvest, reagents utilized for cellular harvesting or mRNA or protein analysis, AAV serotypes, and/or standard modifications to a construct comprising an gene are practical and expected alterations of the current example.

### Example 6: Preliminary Hair Cell Tolerability Assessment of Transgenic GJB2 mRNA Expression and Connexin 26 Protein Production in Neonate Cochlear Explants

This example relates to the introduction, and expression analysis of rAAV constructs overexpressing a GJB2 gene in neonatal cochlear explants. Mock rAAV particles or rAAV particles comprising rAAV constructs (FIG. 2 panels (A)-(H)) encapsidated by Anc80 capsids are prepared and transduced into neonate cochlear explants at a known MOI (e.g., approximately 4.5×10⁹ or 1.3×10¹⁰ vg/per cochlea). Explants are grown to levels appropriate for harvest (e.g., for 72 hours post transduction), and are then prepared for immunofluorescence staining/imaging through fixation using 4% PFA or RNA extraction. RNA samples are prepared and GJB2 gene overexpression is confirmed using quantitative PCR with appropriate reagents in a manner described in a published method (e.g., appropriate according to the RNeasy Micro Kit and quantitative real-time PCR) using construct specific primers and relative to a control. Robust GJB2 mRNA production is observed in explants transduced with test rAAV when compared to mock transduction events. Tolerability and lack of hair cell toxicity is determined using immunofluorescence staining/imaging, antibodies targeting Myo7a (Proteus Biosciences) are utilized to depict inner ear hair cells, while DAPI staining is used to define nuclear positioning. No or low hair cell (Myo7) toxicity is observed after GJB2 overexpression.

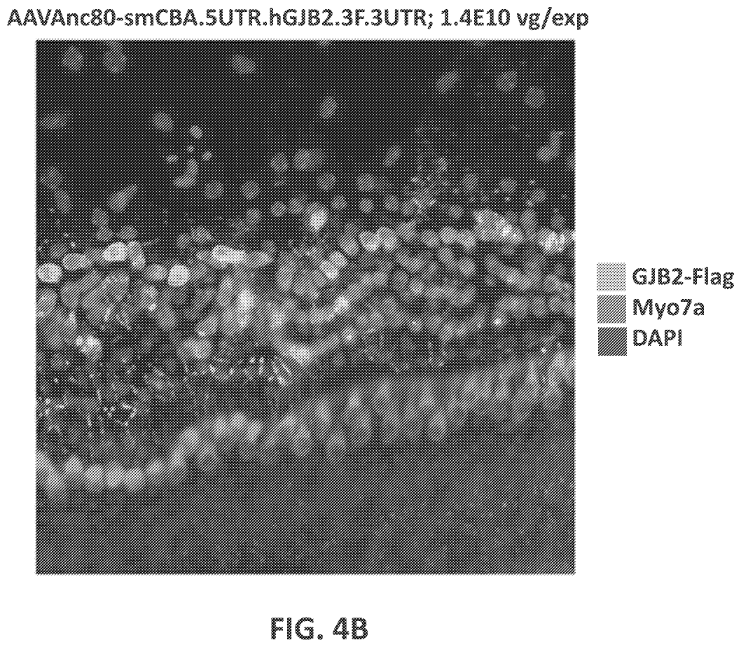

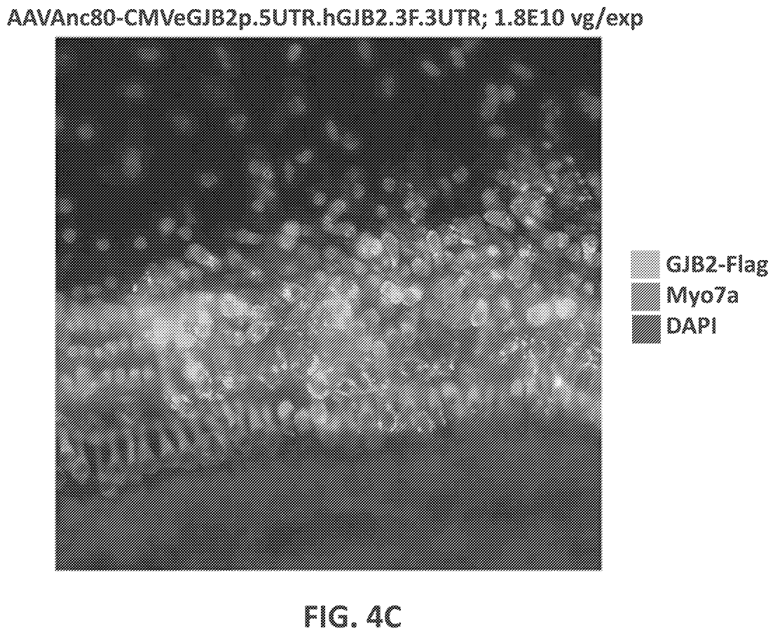

rAAV Anc80 particles comprising rAAV constructs driven by CAG, CMVe-GJB2p, or smCBA promoter/enhancer combinations were prepared and transduced into mouse neonate (P2) cochlear explants at a known MOI (approximately 5.8×10⁹, 1.4×10¹⁰, or 1.8×10¹⁰ vg/per cochlea respectively). Explants were grown to levels appropriate for harvest (e.g., for 72 hours post transduction), and were then prepared for immunofluorescence staining/imaging through fixation using 4% PFA. Explants were then DAPI stained (presented in blue) and immunostained using anti-FLAG antibodies (presented in green), and hair cell specific anti-Myo7a antibodies (presented in red), explants were subsequently imaged (exemplary data presented in FIG. 4A-4C). Robust FLAG signal was observed in the supporting cells of the explants transduced with rAAV particles comprising AAVAnc80-CAG.5UTR.hGJB2.3F.3UTR (as depicted in FIG. 2C, panel (C), SEQ ID NO: 82) at 5.8E9 vg/explant (see FIG. 4A, panel (A)). Robust FLAG signal was observed in the supporting cells in explants transduced with rAAV particles comprising AAVAnc80-smCBA.5UTR.hGJB2.3F.3UTR (as depicted in FIG. 2D panel (D), SEQ ID NO: 83) at 1.4E10 vg/explant (see FIG. 4B, panel (B)). Robust FLAG signal was observed in the supporting cells of the explants transduced with rAAV particles comprising AAVAnc80-CMVeGFAPp.5UTR.hGJB2.3F.3UTR (as depicted in FIG. 2E, panel (E), SEQ ID NO: 84) at 1.8E10 vg/explant (see FIG. 4C, panel (C)). Variation in FLAG expression was detected across samples, likely the results of variability in vector titer.

### Example 7: Surgical Method in Aged Mice

The current example relates to the introduction of constructs described herein to the inner ear of aged mice. rAAV particles comprising an AAV capsid and a construct encoding a connexin 26 protein or characteristic functional portion thereof are prepared in formulation buffer (e.g., artificial perilymph or 1×PBS with pluronic acid F68) and then administered to the scala tympani in mice as described by Shu et al., Human Gene Therapy, 27(9):687-699, 2016, which is incorporated in its entirety herein by reference). Male and female mice older than P15 are anesthetized using an intraperitoneal injection of xylazine (e.g., approximately 5-10 mg/kg) and ketamine (e.g., approximately 90-120 mg/kg). Body temperature is maintained at 37° C. using an electric heating pad. An incision is made from the right post-auricular region and the tympanic bulla and posterior semicircular canal are exposed. The bulla is perforated with a surgical needle and the small hole is expanded to provide access to the cochlea. The bone of the cochlear lateral wall of the scala tympani is thinned with a dental drill so that the membranous lateral wall is left intact. A small hole is then drilled in the posterior semicircular canal (PSCC). Patency of the canalostomy is confirmed by visualization of a slow leak of perilymph. A Nanoliter Microinjection System in conjunction with glass micropipette is used to deliver a total of approximately 1 μL of construct containing buffer (e.g., rAAV constructs described herein at approximately 4.5×10⁹ to 5×10¹⁰ vg/per cochlea in artificial perilymph or 1×PBS with pluronic acid F68) to the scala tympani at a rate of approximately 2 nL/second. The glass micropipette is left in place for 5 minutes post-injection. Following cochleostomy and injection, the opening in the tympanic bulla and the PSCC are sealed with small pieces of fat, and the muscle and skin are sutured. The mice are allowed to awaken from anesthesia and their pain is controlled with 0.15 mg/kg buprenorphine hydrochloride for 3 days.

### Example 8: Transgenic Expression and Imaging of Connexin 26 Protein in Wild-Type Mice

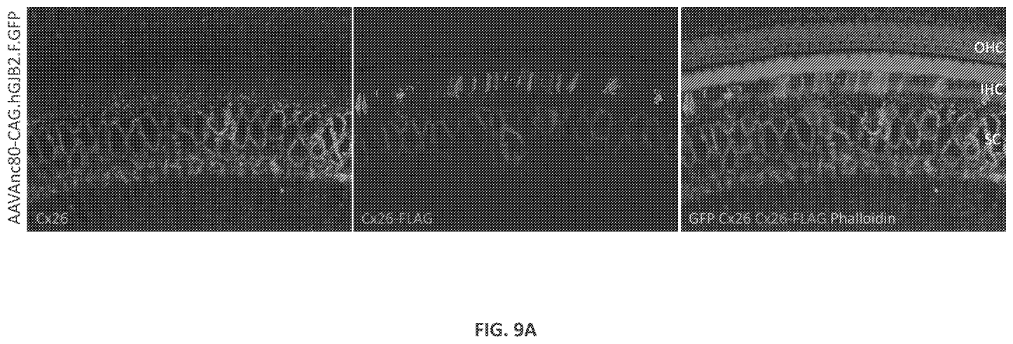

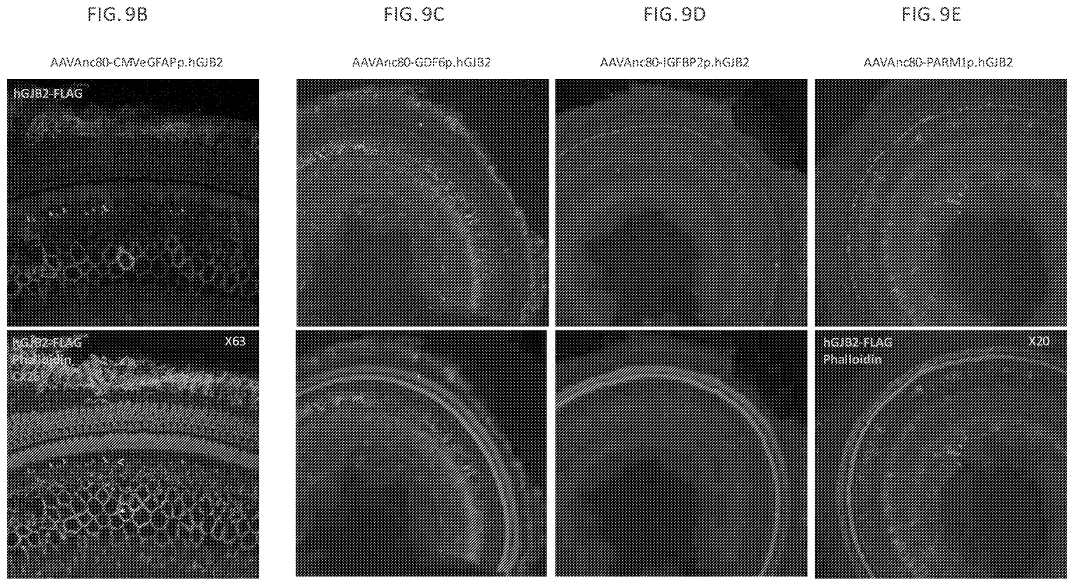

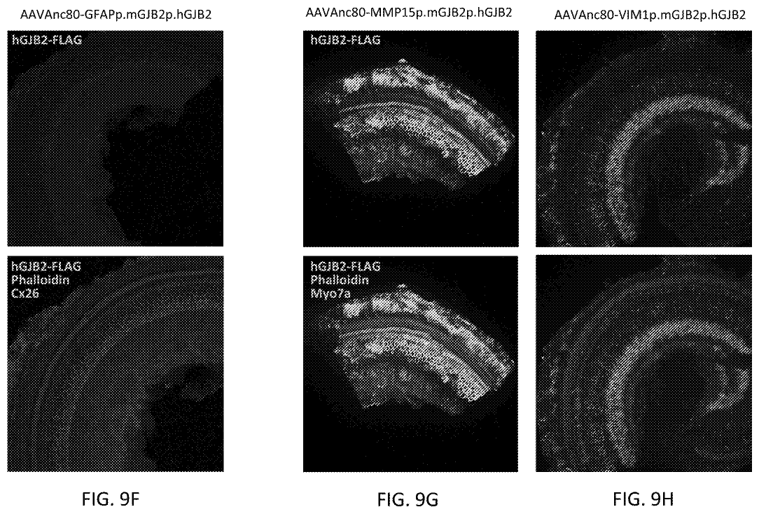

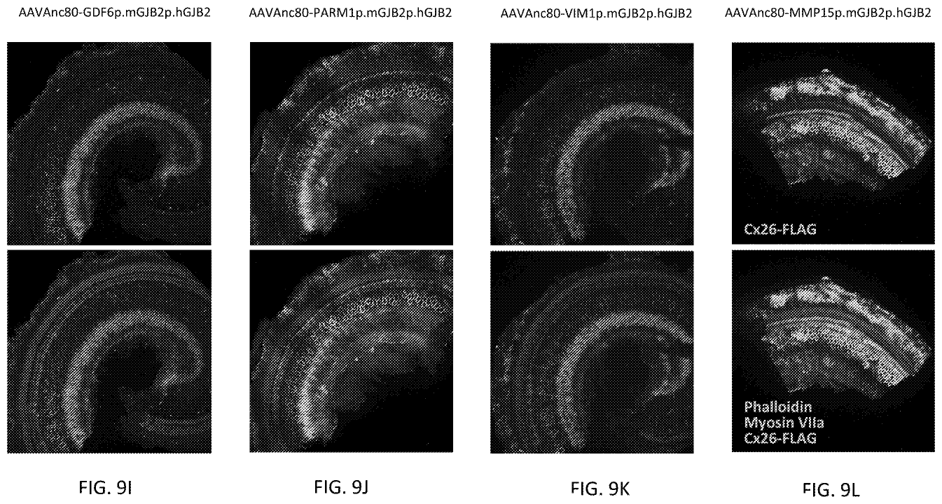

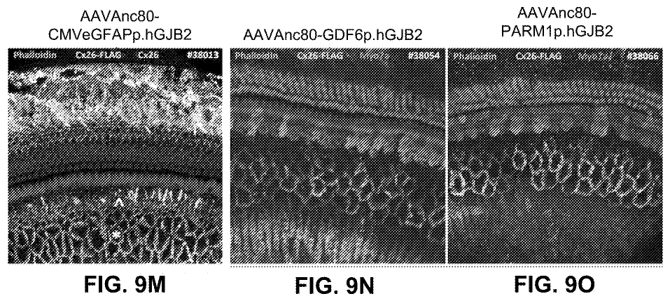

This example relates to the transgenic expression and analysis of transgenic connexin 26 protein in wild-type mice and GJB2 inducible conditional knockout mice. Wild-type mice were administered AAVAnc80 particles (1.2×10¹⁰ vg/cochlea) comprising CAG.hGJB2.FLAG.GFP (schematic provided in FIG. 2H) to the cochlea by the method described in Example 7. 10 days after administration clear and robust of exogenous Connexin 26 (FLAG; purple) was detected in the membrane of the supporting cells of the sensory epithelia (FIG. 9A, middle and right panels). Expression of exogenous Connexin 26 was also detected in the inner hair cells. Endogenous Connexin 26 (red) was detected in all supporting cells (FIG. 9A, left and right panels).

Juvenile wild-type mice were administered 1 μl of AAVAnc80 particles comprising AAVAnc80-CMVeGFAPp.5UTR.hGJB2.FLAG.3UTR (SEQ ID NO: 84), AAVAnc80-GDF6p.mGJB2p.5UTR.hGJB2.FLAG.3UTR (construct comprising SEQ ID NO: 61 and supporting cell selective promoter comprising SEQ ID NO: 90), AAVAnc80-IGFBP2p.mGJB2p.5UTR.hGJB2.FLAG.3UTR (construct comprising SEQ ID NO: 54 and supporting cell selective promoter comrpising SEQ ID NO: 57), AAVAnc80-PARM1p.mGJB2p.5UTR.hGJB2.FLAG.3UTR (construct comprising SEQ ID NO: 7 and supporting cell selective promoter comprising SEQ ID NO: 40), AAVAnc80-GFAPp.mGJB2p.hGJB2, AAVAnc80-MMP15p.mGJB2p.hGJB2, or AAVAnc80-VIMp.mGJB2p.hGJB2. Administration of a hGJB2 construct with a promoter incorporating the CMV-enhancer resulted in supporting cell expression that colocalized with endogenous connexin 26 expression (FIG. 9B; asterisk). However, inner hair cell expression was still detected (arrowhead). In contrast, administration of a hGJB2 construct with promoters derived from supporting cell genes GDF6 (FIG. 9C), IGFBP2 (FIG. 9D), and PARM1 (FIG. 9E) in combination with a minimal GJB2 promoter resulted in supporting cell expression without detection of inner hair cell expression.

Administration of AAVAnc80 particles comprising AAVAnc80-GFAPp.mGJB2p.hGJB2 did not result in supporting cell expression of GJB2 (FIG. 9F). Administration of AAVAnc80 particles comprising AAVAnc80-MMP15p.mGJB2p.hGJB2 or AAVAnc80-VIMp.mGJB2p.hGJB2 resulted in supporting cell expression of flag-tagged hGJB2. Expression of flag-tagged hGJB2 not detected in hair cells as noted by Myo7a staining. Likewise, administration of a hGJB2 construct with promoters derived from supporting cell genes GDF6 (FIG. 9I), PARM1 (FIG. 9J), VIM (FIG. 9K), and MMP15p (FIG. 9L) resulted in supporting cell expression without detection of inner hair cell expression.

Juvenile WT mice were administered with AAVAnc80 particles comprising AAVAnc80.CMVe.GFAP.mGJB2p.hGJB2.FLAG, AAVAnc80.CMVe.GDF6.mGJB2p.hGJB2.FLAG, or AAVAnc80.CMVe.PARM1.mGJB2p.hGJB2.FLAG through the round window membrane with posterior semicircular canal fenestration. 4 weeks post administration, the mice were euthanized, the inner ears were harvested in fixed in PFA, and the injected (left) ear was processed for immunofluorescent staining using phalloidin to label all cells and hair-cells stereocilia bundle, anti-FLAG to label the transgene and the hair cell marker Myo7a. Anti-Cx26 antibody was also used in some of the samples to colocalize the expression of the transgene with endogenous Cx26 expression. Multiple regions from the base, middle and apex of the cochlea were imaged and represented images are presented in FIGS. 9M-90.

Expression of the Cx26-FLAG transgene (green) was detected in supporting cells, overlapping with endogenous Cx26 expression (asterisk), and in some cases, was also observed in inner hair cells (arrowhead) (FIG. 9M). FIGS. 9N and 90 demonstrate robust FLAG expression in supporting cells (green) with no apparent expression in IHCs or hair cell loss. Futher, differential expression patterns were observed between supporting cell subtypes.

1. A polynucleotide comprising a sequence having at least 85%, at least 90%, at least 95%, at least 96%, at least 97%, at least 98%, at least 99%, or 100% identity to any one of SEQ ID NOs: 40, 90, 96, or 99.

2. The polynucleotide of claim 1, which comprises a nucleic acid sequence having at least 85%, at least 90%, at least 95%, at least 96%, at least 97%, at least 98%, at least 99%, or 100% identity to SEQ ID NO: 90.

3. The polynucleotide of claim 1, which comprises a nucleic acid sequence having at least 85%, at least 90%, at least 95%, at least 96%, at least 97%, at least 98%, at least 99%, or 100% identity to SEQ ID NO: 40.

4. The polynucleotide of claim 1, which comprises a nucleic acid sequence having at least 85%, at least 90%, at least 95%, at least 96%, at least 97%, at least 98%, at least 99%, or 100% identity to SEQ ID NO: 96.

5. The polynucleotide of claim 1, which comprises a nucleic acid sequence having at least 85%, at least 90%, at least 95%, at least 96%, at least 97%, at least 98%, at least 99%, or 100% identity to SEQ ID NO: 99.

6. The polynucleotide of claim 1, wherein the polynucleotide is capable of directing transcription of a coding sequence for a polypeptide in an inner ear support cell.

7. A construct comprising the polynucleotide of claim 1 and a nucleic acid sequence comprising the coding sequence for a polypeptide, wherein the polynucleotide is a promoter and is operably linked to the coding sequence, and wherein the polynucleotide is capable of directing transcription of the coding sequence in an inner ear support cell.

8.-18. (canceled)

19. The polynucleotide of claim 6, wherein the polynucleotide is a promoter, and wherein the promoter comprises a nucleic acid sequence having at least 95% identity to any one of SEQ ID NO: 40, 90, 96, or 99.

20. The polynucleotide of claim 6, wherein the inner ear support cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

21. The construct of claim 7, further comprising a minimal GJB2 promoter which is operably linked to the coding sequence for the polypeptide.

22. The construct of claim 7, wherein the coding sequence comprises a GJB2 nucleic acid sequence having at least 85%, at least 90%, at least 95%, at least 96%, at least 97%, at least 98%, at least 99%, or 100% identity to any one of SEQ ID NOs: 117-126.

23.-61. (canceled)

62. The construct of claim 7, wherein the construct further comprises a 5′ UTR and a 3′ UTR.

63.-65. (canceled)

66. The construct of claim 7, further comprising a polyA tail.

67.-68. (canceled)

69. The construct of claim 7, further comprising a 5′ inverted terminal repeat (ITR) and a 3′ ITR, wherein the 5′ ITR and the 3′ ITR flank the promoter and the polynucleotide.

70. (canceled)

71. The construct of claim 69, wherein the 5′ ITR and the 3′ ITR are AAV ITRs derived from a serotype selected from AAV1, AAV2, AAV3, AAV4, AAV5, AAV6, AAV7, AAV8, AAV9, AAV10, AAV 11, and AAV Anc80 ITRs.

72.-80. (canceled)

81. A viral vector comprising the polynucleotide of claim 1.

82.-83. (canceled)

84. An AAV particle comprising the polynucleotide of claim 1.

85. The AAV particle of claim 84, which comprises an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 serotype capsid.

86. The AAV particle of claim 85, wherein the AAV capsid is an AAV Anc80 capsid.

87. A composition comprising the polynucleotide of claim 1.

88. The composition of claim 88, wherein the composition is a pharmaceutical composition further comprising a pharmaceutically acceptable carrier.

89. (canceled)

90. An ex vivo cell comprising the polynucleotide of claim 1.

91.-93. (canceled)

94. A method comprising, transducing an ex vivo cell with:
a. the construct of claim 7; and
b. one or more helper plasmids collectively comprising an AAV Rep gene, AAV Cap gene, AAV VA gene, AAV E2a gene, and AAV E4 gene.

95.-97. (canceled)

98. A method of expressing the polypeptide in an inner ear supporting cell, comprising administering the the construct of claim 7 to the subject.

99. A method of increasing expression of the polypeptide in an inner ear supporting cell, comprising administering the construct of claim 7 to the subject.

100. (canceled)

101. A method of treating hearing loss in a subject suffering from or at risk of hearing loss, comprising administering the construct of claim 7 to the subject.

102.-110. (canceled)

111. A kit comprising the polynucleotide of claim 1.

112.-138. (canceled)

139. An expression construct comprising a polynucleotide encoding a polypeptide operably linked to an inner ear supporting cell selective promoter, wherein the polynucleotide is expressed in an inner ear support cell, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

140.-143. (canceled)

144. The expression construct of claim 139, wherein the inner ear support cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

145.-146. (canceled)

147. The expression construct of claim 139, further comprising a miRNA regulatory target site (miRTS) for a microRNA expressed in an inner ear cell.

148. (canceled)

149. The expression construct of claim 148, wherein the microRNA is one or more of miR-194, miR-140, miR-18a, miR-99a, miR-30b, miR-15a, miR182, or miR-183.

150. A viral vector construct comprising: (i) a 5′ inverted terminal repeat (ITR), (ii) a polynucleotide encoding a polypeptide operably linked to a promoter which is capable of driving transcription of the polynucleotide in an inner ear support cell, and (iii) a 3′ ITR, wherein the promoter is heterologous to the polynucleotide, and wherein the promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

151.-153. (canceled)

154. The viral vector construct of claim 150, comprising: (i) the 5′ inverted terminal repeat (ITR), (ii) the polynucleotide encoding a polypeptide operably linked to a promoter which is capable of driving transcription of the polynucleotide in an inner ear support cell, (iii) a miRNA regulatory target site (miRTS) for a microRNA expressed in an inner ear cell, (iv) a 3′ untranslated region (UTR), and (v) the 3′ ITR.

155.-176. (canceled)

177. The viral vector construct of claim 154, wherein the microRNA is one or more of miR-194, miR-140, miR-18a, miR-99a, miR-30b, miR-15a, miR182, or miR-183.

178.-189. (canceled)

190. The viral vector construct of claim 154, wherein the microRNA regulatory target site comprises a nucleic acid sequence with least 85%, at least 90%, at least 95%, at least 96%, at least 97%, at least 98%, at least 99%, or 100% identity to any one of SEQ ID NOs: 1-6, 78, or 79.

191.-240. (canceled)

241. An ex vivo cell comprising the viral vector construct of claim 154.

242.-248. (canceled)

249. A method of expressing the polypeptide in an inner ear supporting cell of a subject in need thereof, comprising administering the viral vector construct of claim 150 to the subject.

250.-262. (canceled)

263. A method of treating hearing loss in a subject suffering from or at risk of hearing loss, comprising administering the viral vector construct of claim 150 to the subject.

264.-287. (canceled)


SEQUENCE LISTING (129 sequences)

SEQ ID NO: 1 - DNA, 22 bp, Artificial Sequence
  Exemplary mRNA destabilizing domain sequence
tccacatggagttgctgttaca

SEQ ID NO: 2 - DNA, 21 bp, Artificial Sequence
  Exemplary mRNA destabilizing domain sequence
ccgtggttctaccctgtggta

SEQ ID NO: 3 - DNA, 23 bp, Artificial Sequence
  Exemplary mRNA destabilizing domain sequence
ctatctgcactagatgcacctta

SEQ ID NO: 4 - DNA, 22 bp, Artificial Sequence
  Exemplary mRNA destabilizing domain sequence
cacaagatcggatctacgggtt

SEQ ID NO: 5 - DNA, 20 bp, Artificial Sequence
  Exemplary mRNA destabilizing domain sequence
ctgagtgtaggatgtttaca

SEQ ID NO: 6 - DNA, 22 bp, Artificial Sequence
  Exemplary mRNA destabilizing domain sequence
cacaaaccattatgtgctgcta

SEQ ID NO: 7 - DNA, 6915 bp, Artificial Sequence
  Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccgggaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgcaaactggttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 145 bp, Artificial Sequence
  Exemplary 5′ AAV ITR
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 145 bp, Artificial Sequence
  Exemplary 3′ AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 277 bp, Artificial Sequence
  Exemplary CBA promoter
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 11 - DNA, 279 bp, Artificial Sequence
  Exemplary CBA promoter
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 12 - DNA, 658 bp, Artificial Sequence
  Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 13 - DNA, 660 bp, Artificial Sequence
  Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 14 - DNA, 1671 bp, Artificial Sequence
  Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 15 - DNA, 1673 bp, Artificial Sequence
  Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 16 - DNA, 735 bp, Artificial Sequence
  GJB6 promoter
aaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcag

SEQ ID NO: 17 - DNA, 6696 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtcccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcgtgtgttgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 381 bp, Artificial Sequence
  Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 19 - DNA, 1013 bp, Artificial Sequence
  Exemplary SV-40 synthetic intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 20 - DNA, 178 bp, Artificial Sequence
  Exemplary 5′ UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaag

SEQ ID NO: 21 - DNA, 206 bp, Artificial Sequence
  Exemplary 5′ UTR Sequence
tttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 22 - DNA, 1432 bp, Artificial Sequence
  Exemplary 3′ UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 23 - DNA, 82 bp, Artificial Sequence
  Exemplary splice donor intron
gtaagtatcaaggttacaagacaggtttaaggagaccaatagaaactgggcttgtcgagacagagaagactcttgcgtttct

SEQ ID NO: 24 - DNA, 51 bp, Artificial Sequence
  Exemplary splice acceptor intron
gataggcacctattggtcttactgacatccactttgcctttctctccacag

SEQ ID NO: 25 - DNA, 225 bp, Artificial Sequence
  Exemplary bGH poly(A) signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 26 - DNA, 122 bp, Artificial Sequence
  Exemplary SV40 poly(A) signal sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 27 - DNA, 8 bp, Artificial Sequence
  Synthetic barcode C
cgtgtgtt

SEQ ID NO: 28 - DNA, 1097 bp, Artificial Sequence
  RBP7 promoter
cccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatag

SEQ ID NO: 29 - DNA, 22 bp, Artificial Sequence
  Exemplary cloning site A
ttgtcgacgcggccgcacgcgt

SEQ ID NO: 30 - DNA, 39 bp, Artificial Sequence
  Exemplary cloning site B
ctcctgggcaacgtgctggttattgtgaccggtgccacc

SEQ ID NO: 31 - DNA, 24 bp, Artificial Sequence
  Exemplary cloning site C
taagagctcgctgatcagcctcga

SEQ ID NO: 32 - DNA, 38 bp, Artificial Sequence
  Exemplary cloning site D
aagcttgaattcagctgacgtgcctcggaccgcctagg

SEQ ID NO: 33 - DNA, 9 bp, Artificial Sequence
  Exemplary cloning site E
taagagctc

SEQ ID NO: 34 - DNA, 15 bp, Artificial Sequence
  Exemplary cloning site F
gctgatcagcctcga

SEQ ID NO: 35 - DNA, 64 bp, Artificial Sequence
  Exemplary cloning site G
ggcattccggtactgttggtaaagccaccagcaaaccgcccagagtagaagaccggtggccacc

SEQ ID NO: 36 - DNA, 12 bp, Artificial Sequence
  Exemplary cloning site H
aagcttgaattc

SEQ ID NO: 37 - DNA, 26 bp, Artificial Sequence
  Exemplary cloning site I
agctgacgtgcctcggaccgcctagg

SEQ ID NO: 38 - DNA, 6334 bp, Artificial Sequence
  Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggttcgtgggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 8 bp, Artificial Sequence
  Exemplary synthetic barcode E
gcaaactg

SEQ ID NO: 40 - DNA, 1316 bp, Artificial Sequence
  PARM1 promoter
tgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccggg

SEQ ID NO: 41 - DNA, 8 bp, Artificial Sequence
  Exemplary synthetic barcode D
tcgtgggt

SEQ ID NO: 42 - DNA, 78 bp, Artificial Sequence
  Exemplary 3xFLAG tag sequence
ggatcccgggctgactacaaagaccatgacggtgattataaagatcatgacatcgactacaaggatgacgatgacaag

SEQ ID NO: 43 - DNA, 2208 bp, Artificial Sequence
  Exemplary AAV Anc80 Capsid DNA Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 44 - protein, 736 aa, Artificial Sequence
  Exemplary AAV Anc80 Capsid Amino Acid Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 45 - DNA, 2989 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 46 - DNA, 4421 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - DNA, 3938 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 48 - DNA, 3916 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccctgtaaagccaattccaacccacttgtaattaagagaaaatcccacggttcctaattgaaagtcctttgttctatttcttgggtatttgtgttttaggccttatttttagatgcatcattaaagatttttaaagtcctttcaggcatcaggactgatgatgctgaatgatggagggttgtggataagtttttttgttttttttttaaccaggttaaaggctttcctgttatcctactatgcttaattaagagctgtatttcttaatatcattggtgcctgattagatttaacttttagatacagtctgtaagatttttgaaccagaaaaacctaaataacttatgactgttagcagtcatattctagaagaagcaaatgtactgaattcttatgtacctaggattttaagggagtacatacaaatctttcctcagtagcaggtactttatttttataacacacacatttaagctgagttaaatatgcagaactggttgtacttctttggcaggaaaagggaagcttaggatatcttgtgaccaactacctcttccttctcaaataactggcaaataacttcaggaaaatccagttatgttgtgtcatattgcaccccctaggaagtactggattcttagtcttgagtgacttttaaataaagctacctttttctctttcttacatcgcaagatcttcaaatgtaccattcccgcacagagagtccaaggtaaaaggactgaaaccaaactttgtttttgtaagtattttggtcagtgcaatgagttcagagaccaggaggttaatgattgtgaagtcttgtcaacagcaacaccgtgtatgacctgtggtgcttagatgttcagaaaccccaaggttaaaatgtccctgaccacatatcaggcaaaaggaatgtaaggaaaaccaacttaatccttttgtcaagaagtataaatgatgtatctttccaatcgggttgcattgacttttgggtccaaatagcttgtgtccacaggcatcttcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 49 - DNA, 2894 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 50 - DNA, 2987 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 51 - DNA, 4419 bp, Artificial Sequence
  Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 52 - DNA, 119 bp, Artificial Sequence
  Exemplary 5′ AAV ITR
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53 - DNA, 130 bp, Artificial Sequence
  Exemplary 3′ AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 7112 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctccaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcacaacctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 55 - DNA, 8 bp, Artificial Sequence
  Exemplary synthetic barcode B
cacaacct

SEQ ID NO: 56 - skipped

SEQ ID NO: 57 - DNA, 1513 bp, Artificial Sequence
  Exemplary IGFBP2 promoter
aagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctcc

SEQ ID NO: 58 - DNA, 24 bp, Artificial Sequence
  Exemplary Taqman probe for quantification of constructs
tctggctcaccgtcctcttcattt

SEQ ID NO: 59 - DNA, 20 bp, Artificial Sequence
  Exemplary forward qPCR primer for quantification of constructs
caaacactccaccagcattg

SEQ ID NO: 60 - DNA, 20 bp, Artificial Sequence
  Exemplary reverse qPCR primer for quantification of constructs
cagccacaacgaggatcata

SEQ ID NO: 61 - DNA, 6787 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctcaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgtgtcaccgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 62 - DNA, 8 bp, Artificial Sequence
  Synthetic barcode A
gtgtcacc

SEQ ID NO: 63 - DNA, 382 bp, Artificial Sequence
  Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 64 - DNA, 245 bp, Artificial Sequence
  Exemplary chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacag

SEQ ID NO: 65 - DNA, 1044 bp, Artificial Sequence
  Exemplary GJB2 enhancer
cttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcagg

SEQ ID NO: 66 - DNA, 362 bp, Artificial Sequence
  Exemplary 5′ UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 67 - DNA, 1407 bp, Artificial Sequence
  Exemplary 3′ UTR Sequence
gaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 68 - DNA, 1248 bp, Artificial Sequence
  Exemplary 3′ UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 69 - DNA, 1372 bp, Artificial Sequence
  Exemplary 3′ UTR Sequence
gagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 70 - DNA, 14 bp, Artificial Sequence
  Exemplary cloning site J
gcggccgcacgcgt

SEQ ID NO: 71 - DNA, 17 bp, Artificial Sequence
  Exemplary cloning site K
gcggccgcacgcgtggt

SEQ ID NO: 72 - DNA, 33 bp, Artificial Sequence
  Exemplary cloning site L
ctcctgggcaacgtgctggttattgtgaccggt

SEQ ID NO: 73 - DNA, 11 bp, Artificial Sequence
  Exemplary cloning site M
cgctagccacc

SEQ ID NO: 74 - DNA, 17 bp, Artificial Sequence
  Exemplary cloning site N
accggtcgctagccacc

SEQ ID NO: 75 - DNA, 21 bp, Artificial Sequence
  Exemplary cloning site O
gagctcgctgatcagcctcga

SEQ ID NO: 76 - DNA, 34 bp, Artificial Sequence
  Exemplary cloning site P
aagcttgaattcagctgacgtgcctcggaccgct

SEQ ID NO: 77 - DNA, 12 bp, Artificial Sequence
  Exemplary linker sequence
ggatcccgggct

SEQ ID NO: 78 - DNA, 24 bp, Artificial Sequence
  Exemplary mRNA destabilizing domain sequence
agtgtgagttctaccattgccaaa

SEQ ID NO: 79 - DNA, 22 bp, Artificial Sequence
  Exemplary mRNA destabilizing domain sequence
agtgaattctaccagtgccata

SEQ ID NO: 80 - DNA, 124 bp, Artificial Sequence
  Exemplary mRNA destabilizing domain sequence
gagctcagtgtgagttctaccattgccaaactcgagcagtgaattctaccagtgccataggatccagtgtgagttctaccattgccaaaggtacccagtgaattctaccagtgccatagttaac

SEQ ID NO: 81 - DNA, 69 bp, Artificial Sequence
  Exemplary 3xFLAG tag sequence with stop codon
gactacaaagaccatgacggtgattataaagatcatgacatcgactacaaggatgacgatgacaagtaa

SEQ ID NO: 82 - DNA, 4786 bp, Artificial Sequence
  Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 83 - DNA, 4018 bp, Artificial Sequence
  Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 84 - DNA, 4508 bp, Artificial Sequence
  Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - DNA, 9 bp, Artificial Sequence
  Exemplary cloning site Q
ctcaccggt

SEQ ID NO: 86 - DNA, 128 bp, Artificial Sequence
  Exemplary hGJB2 basal promoter
aagctctgaggacccagaggccgggcgcgctccgcccgcggcgccgccccctccgtaactttcccagtctccgagggaagaggcggggtgtggggtgcggttaaaaggcgccacggcgggagacaggt

SEQ ID NO: 87 - DNA, 4745 bp, Artificial Sequence
  Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 88 - DNA, 4712 bp, Artificial Sequence
  Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagaccggtcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataaaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 89 - DNA, 17 bp, Artificial Sequence
  Exemplary sNRP poly(A) signal sequence
aaataaaatacgaaatg

SEQ ID NO: 90 - DNA, 1188 bp, Artificial Sequence
  Exemplary GDF6 promoter
ccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctc

SEQ ID NO: 91 - DNA, 680 bp, Artificial Sequence
  GFAP promoter
gaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 92 - DNA, 1404 bp, Artificial Sequence
  BACE Promoter
tgtgctgcgagggcttcatctcctaagcactaaatgctaaattccccctcccacgcccatcgccactgtcctcacggatcctcgcagcagcttcccaatcggtctccctgtctccagcctcaccacccccaactaagaccattcatgaaaacagagacaaccaaggagacagtcacccaatgctgtcccttcagcttgcattattttctgacaagacagctctgccatccatggaagcctgtgtttgaagatctctgacataaaggtcccttgcagagctagacgtgattctaaaattgggaacacaggaataaaaatcaaatcttgagtagaagtagctgaaaattgcagtgattcggggaagcttggcttctaactccccactgtttgaagatgggcttgtttgttttttaaaacagccaacataattcagctggaggaggtacaaagaattttctattccttgtttctgtagaaatcgatggactttagcttgtctaattgtcccccctgcctttagtatctaaaataaaataaccctcgttgcttgcattactcaacgcatttctgcgtcttggcgtctatggctaaacgagtattaattagacagtccgcagagagctggctggggatagaaggggaggtgggggagaagggcagggatcacagcagggtggactcgtggccctgatttgggatcctgacagcaacttactaggtggcctgagggctgggtgccaggggaggcagcgggttccagtagcatctgacctgcattagggacaggggcgcggcggagggggcgaagggggcgggggtggggggaaggtggctggggtgaagcccagcttcgcagctagctgtgggcaacagagggagtaagggggggcaatgaggctggggccaggcgccagcagcagccacgccccccacctcccccgatttttagggaaaattctccaaagctctcgcatcctcctctgcctccttccaccctccaccctcccagcctccactgagacctctttaaaaccacccaggggccgccgggggatgaggccggggaacgggctggactgagggcgggggctcgggggcagcggacgggaaacgcctcgaaagcagccagacccggcgactgaaatgaggcggaggagcttggcgaggggaggcgcaggctcggaaaggcgcgcgaggctccaggctccttcccgatccaccgctctcctcgctgacctccgagtcacccccggaagctcccgccactgccgggcgaatagacccccgcggacccccaagcgcgcggggccggggccctagttcaggccctcgctgcccctttaagggttctcgaaactttccccccggtatcagatgagcctcgtcacatccgttggccgtggc

SEQ ID NO: 93 - DNA, 1467 bp, Artificial Sequence
  DBI2 promoter
gaagaaacctgcatttcttacacttcagtgtactttccccatatttaactccaagatttttgttaatttgtttggttttcctttctcaaacaaaattatgctcagactgaaaaccctagatttgttccctattgcatcttcatttcttcccaaacattccataaaacgtgacctacattaagttagcaagttaagtctgaaagcgtctaccttccctggggagggggaaggtgtaggcagggcagagatttgtagtccagccctcttgccacaaattatgaattagagaggaatgactttgcttttttaatgatctccagagaattttccatcatttccctctcttcacccagctcctttgcaaccactgccagagaagtcttcctttagcttcttaaacatcgatcctaaaacacttccagacacctgtgctgctcctttcagttcccatggagattaggctgtgtaacaatctcgcaaagacgttcccctccgtctcctcatcctcttttcaaacccttttacgatttcccatctcactcagcatgacagtcaaagtccctgtgatggccaacttctgcatcacctagccagtctgccaccgccaaaactctccagcctcatcttacacttgttctctgcttggaatcttccctcccctccttgaggaactttctcaaatgtcaccttccctcaatactccccctcctccatttaaaactataaacttccaactctctaagcccctaaagtactctatatttaacttattgtataaactactgtccctacttgtaagttccaagattgcagggattcacccgctttgttcactgctgtctgccaaggtctagaacagtgcaagttacccaacaggagttcaataaacagccattcatttaacaaatatttgctgagcacttcgtcccgtccaagtttgttaaatcaagacaaataagacaccgtccctgcctttaacgcaccagatggagaaatgcaccacagacataaatgtgcaatacaggcctgacactacggccacaagcaagtcaaagaacgtgccaaaagttcagaggaagaagcctcggcttcgcctttcgggagaccagtccagctttccaccatcacgctgctcatcagggaccatctccgggggtctcctctagaccccaagggaggagcgggtcccgcccgccattcccaggtctcagagtttacttgtccagagatgcaacttccggcctcttcaggccgggcaagatttaaggaaagaaaagaaacataaggacctccgttcttcggtctccgtcccctccccttcccccgcgtgccccacctgttcccggcgtccccttcggctactcccggcgtttgcgcaagcggtcccacgtgggctcgggcggggctagcgccgcggcgggggctgggcacgcccctagcgcatagctggcttctgattggctttcc

SEQ ID NO: 94 - DNA, 1752 bp, Artificial Sequence
  FABP3 promoter
taccattctgcctttcacctgatgttgctatcctcctccctcttgtttccttccacccatcctttccctcccacattactctcttatcccaccctattttacaaccagtagcctagggaaaagagcatagctcaaatgaggaagaaggcaggacaggcagtcatggcttagctggactgagctgcagtgcttctccttctggggaagggggtgcactgtcatctgctactgacacatccctccaaggcactcagccctgcagggagcaacctgattctatgactgacatctaatcttcacattcaccttgcaggaaggcaagaagtgatcccagcctccagatggaaagatcaaggcccagagaaggtcagtggtggttggaggcctgaggtcacacagcagccaagtctggagtcactagtcaaggtgaccttgactagccaccccacctccccttccctgccccaccatggccctgggagatctgttgtcctgtgagggaaaggggctccaggctgggctgcatctgaagcccctagatccagagacttcatttcttaggctatctataaaatccaccttcctttcttttcccaggacccccataccctgctcccagcatcgtctgcctcagctaagccatggggattgagagaccaggcctggtgcccagataaactgaccctgggtgaggggacaggggcccagaatgggcaggtagagactgaatactgaagaagaatcctctggagtctgttagcagaagcagatgggccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaacctcctaccatagagttgggaacactgaagaaaagagactggcccaaggtcacagagaaggcagggtgaacactgtcacagggagagccagtgtagaataatggttaagccacgcaagctctagaaccactctatctgagtgcaaatcctggctgtcatctggtacttgcttcctggaacacatctggcctcagactcctgaggccaagacacactccctgccctaagacttgctggttctatggcaggcagaggcagaaagagccccaccatcattcccagcaaatgggaaaagttcccagttgcagatattaggggtgggatggggcgggggtagtcagcaaccatagacttagaccctgaagaggcaaaaaaggagggccatgttcttgggtcagcagagcttctactcagcttcttcagcctctagctctttcctggtgctagtagcacattctctagtggaggcatccagatggcagggagggtccaggaaacagctgaacatgctgagcaggcctcccttgtccccgctccccatggccccatggatcatccggtgctgcagctcatctcattggctggcttctggttactcatctctcctcttctccatcttcccagcctgtggttgccgtggaaacatagaacagtgacctcaccataggatgagggctggggagatgctgttcttggcaggcgct

SEQ ID NO: 95 - DNA, 1255 bp, Artificial Sequence
  KLHL14 promoter
gaaacagcagccattgatgtagctcagggttctgtggatctgtcatttggagcatgttggttctcctgtctcagctgggctcattcatgcatctgagttcagctattgggcaatctggggaatgttttgtccatgtgatgtgtcatcttctaccaggctagcctgggcttcatcacatggtatctggcagggctctaagagggagagttgaaacacacaaggcctcttgaagcttagactcagaattggcacaaggtcgcttctggcacattccattggtcaaagcaagttacaaggccagctcacattcaaggattaggtaagtcgattccactcttgatgagaagtctgaaggatttggaacagtgtccaccatgcagtaataaactcaataagtagtagccattattattctgttagaggttgccaggaaaagttttatagtggaaagaaatctgagtttactcttgagaggtaagtggaatttctatttgtagagaatgaaggcctctcaaaaagacacagcctaacaataggtgctgcagtttaacagtggagcgtgtccagaacaggctgcccttttaggcaagggctagtgtctttcaggacagacccaaaccccaaataccaaaacagaataaagtagtgtcttagcatactttgagatcagactgtttctgcatttcacagtgctgggggtgggggggaggtgtggggggaagggaaaagcagcataccaatgtagtgaaatctggaaacaacagccaaaaaaagtttgcatattgcacagagcacttgaagatcataaatctatgcatgagaaagatgtagtggaaattttgggggggattagagtttatttttgtcatctctgtgagacagctactcattcatccagatcacagctaagaaaaaagctggtcacagaaattagcagtttcagctcagcagcgaagtcgccagcctgtgaaggcagagagaaattgactaattagcaatgcgcactaaaacttgacggttctttatagagagagagaagagagagggagagagagggagagggagggagggggggctcgctttttccccttctttcttccaaagatgtttgaaatcgcagtcatttacgctcgacaatttttacaatagccttgagccataattttgcgagtctctccagcatccatccccctgtatggtctctctctactggccaagcacgaccgtttctctccccaaccgtggatttcctatt

SEQ ID NO: 96 - DNA, 1012 bp, Artificial Sequence
  MMP15 promoter
ccttcctcctccagggccctctgcagaccaggctgagatggaggaacctgctaaaatcgatggagatgcttctagcctcccagtaggaggccccagccatgccttcaacctggcaggaggtgtagccactcctcatccttgggttgcaggttgggtgctgctgttgtggtccttcccagaaactgccagtagagggcagcctgggcatcctaatgcttactctggttgttacacaaagaaaatattggggtcactggcgagcccacccacactcaccagaatctccactgtagtccccctaacaaacagcccttcacttcctctcccacttcagcaatttgtattttgatgccattggcctcagatcagagtgttttaaatcatcacgccctggcttatccctggtcgagccaggacacggggtgcttcagtgggtctgtcaccctctctccttgaagcatgttgcttttatttatttacttttactctcaccctgctcctgtaccagcaggggccacttcaaagccaaggtacagggtgataacttgtggtccagcatcagttttctccacttctttctcccactcacccccagcaaggtgcctggggagacttgagcagatgtttcattttggcctggccagtggctgaaagccaggcctccaatgcactgtgacctctggcttccccagcagctttcccagagaggcagagggagtcttcattcttcccaggcggggagaccacgccttccctgcctcctccctccgcggggggtcgcgttggaggtcacccccgccccctaggcgctgggttgggagtgacgcggggtgggctggagaggtttcctgccgtctgggaagcgtaaacggaccgcccacctgtcgggcctcggccgcccgcacctgcttgtgagaagcctgcggctggggcaccgcccccggtccccgcccgggtccgcgcattgggagcacactggccctttaagagcgcggcggccgcggcgcgcggg

SEQ ID NO: 97 - DNA, 1079 bp, Artificial Sequence
  SPARC promoter
caggctacctctcaggctgactgagtcatgcagcataggctgccacgtctctgggctggcggggccgtcattattcctggcctcactgcagctaaattgaagaaacgtttggtttgtgggccacgtcaaggaatgtgtaagagctgccacgttgtcgggtctgggttattgggcttttcccctccttcagagaagatttccaggcgtgtgggtggggtttcagaagaaaattgatgcctgcgtgtgagtgttccctggacctggaccagcagcggcaatattacagacccgggggttggggcagactgagccaatctctgcaccgtcaaagttatggatacagagccctggaaaaaggctgaaggataagatagctgacatttatgaagtgcttcattcatgtagcagtgggccaaatgcgtactttacacttgaggaagctgaggctggaggttgataacatgcctcaagtcttctagagttaaataactttgacccaggacccaagcccagagttctgactcaaaaactaggcctcctaaacatcctcttatatgaggttaaatttcatcttcctctgtttggccttggcctggttggtggatgctctgcttcggggacccagggccagatgacaatgggttctttgtgcccttcagacaatgggaagggctgcctggggaaagatacagtaacaaggcaacaggctgagtcagcctccaatgtgcttgaaccttcttagcttggcagccttgacattcagccagccacacaaagggtatatcaaggatgataccactagtagcagcttgtcttgtctgtacctctgaacaagaaagaggctgttctgggtcatccctccaggcctgtccagccctggcactctgtgagtcggtttaggcagcagccccggaacagatgaggcaggcagggttgggacgtttggtcaggacagcccaccaggaggaaagaaatgaaagacagagacagctttggctatgggagaaggaggaggccgggggaaggaggagacaggaggaggagggaccacggggtggaggggagatagacccagccca

SEQ ID NO: 98 - DNA, 1223 bp, Artificial Sequence
  TSPAN8 promoter
ccaaggactcttttttctaaacttcccttcatcttctagtttgacgcccttggtgggaaaagtgtctgagataaggaaaaggcatcctttcagttctctgatactatcttgaagcgagggatggagaaaggcaaagagagacacaggagaagcgtatcccctgggaacaggtgtctagtggagtccagtaactcacagtctctcagttccgtcagcactgtcccttgggtcgcaaatttcttccattagcccttccaccagctgtatttcaaatggggctggacaataattgtggccagtggccttgtgttgtttgtacttgcggactagtagttctcacctgtctttctctgactcctattagccactgggatttcagcagctggttcagccaattctactcaattcaacattaagttgcagtgggctagaactcatgggccgatttaacaagtgaaattctaccaagatacatcaaaaatagcaacaggactagatactcagctcattttgttttatttgtaatataccagttgtggctttagtgccagtctgattcatctctctactacaaaatgaggctctataaaggaaaatattgcaactggagtgaggaatttgaatcttataggaaggaatttgtcttctcatgaagacttcagtttaccagaagtatctattgaggaagtgtttacaagaaaatgtgccatttagctttattctaaatttgcataataactgaaccaaacaaaaaaatatagatagatagattgttctatctatagatagatagggaacattggcagtaggtggcagtaagttcccctgagcacatggaggacacagttaaatgcatttgaggtatgtgggaaatggtttaaagcagaattttatgccaacttttagtaacggaagcctaacaaatgtttgttctttcaagtgagagaagcaagcaatctggaactattcataagcttattttctgtatccttaaacatattttataatgaatgtatgatttaaatagtaagttaagtgtctgggggtactgcacacctcccttgcatacagtcaaacttcttcagggtgatggggaagaggagttataggctgccaagcaaaattgccaaactggtctcagaaattcactgcattggagagcgcgggatccttgcaacactgactttagcagttaaactagagtggttggggatgagtattct

SEQ ID NO: 99 - DNA, 1087 bp, Artificial Sequence
  VIM promoter
attcacaatgcattccctctgcccaccacattaattatcaactccttttcctggcatttactcatccaacgcatggccccacgttaactttcagttccctttctcccctacaaatactccataatccagcaaccctgggatccctgagatgatgaagaggaccagtgcccattccaggagacatcaccgcagccctgaggaatcggctatgggcaccagcagggcacagtgccacacctcgccaatgccttgtcctccttttccatagtgagtcagtcagcaagcgtgtagaagtgagttccacactctcttcctcccatagggagatcacttttctcattctaagggttccaggcacactcacaatggtggcatttgctgagcagtggcttgaataaagggctctcagaaagcaagatgtaactcagagcataggctagccccaggaatgctcttggggaatgacctgcagcctcccagtgaaagagagaataaaagaaagccccagcaggcgagctgggcagtagagagtcctgtaattccaccttggcaagcaccatttgcaagaacgaactgggataaggtaaacaaaatattgcctaaaagaggcttgtccaaagaagtcagaatacgctcttcatttacctctaaatttcagtacaccataaatctaaatactcaaaaaaacctgtgccttttcaatcaaggtcaattgcacgaattcttttggaaaacaggacctatggcatttcccagacaaatcaccgtgaaccctgtactgtgcattgctgtcctaaaatccaaagattctgtcatttgtgttacataattgcctttcatttgaactcattaatcaaattggggtttttaagcaacacctaattaattctttaactggctcatccactgatcactgagttctattttgaaactacggacgtcgagtttcctctttcacccagaattttcagatcttgtttaaaaagttgggtgtggtttcatggggggagggggaagagcgagaggagaccagagggacgggggcggggactctgcaagaaaaaccttcccggtgcaatcgtgatct

SEQ ID NO: 100 - DNA, 4634 bp, Artificial Sequence
  BACE2 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttgtgctgcgagggcttcatctcctaagcactaaatgctaaattccccctcccacgcccatcgccactgtcctcacggatcctcgcagcagcttcccaatcggtctccctgtctccagcctcaccacccccaactaagaccattcatgaaaacagagacaaccaaggagacagtcacccaatgctgtcccttcagcttgcattattttctgacaagacagctctgccatccatggaagcctgtgtttgaagatctctgacataaaggtcccttgcagagctagacgtgattctaaaattgggaacacaggaataaaaatcaaatcttgagtagaagtagctgaaaattgcagtgattcggggaagcttggcttctaactccccactgtttgaagatgggcttgtttgttttttaaaacagccaacataattcagctggaggaggtacaaagaattttctattccttgtttctgtagaaatcgatggactttagcttgtctaattgtcccccctgcctttagtatctaaaataaaataaccctcgttgcttgcattactcaacgcatttctgcgtcttggcgtctatggctaaacgagtattaattagacagtccgcagagagctggctggggatagaaggggaggtgggggagaagggcagggatcacagcagggtggactcgtggccctgatttgggatcctgacagcaacttactaggtggcctgagggctgggtgccaggggaggcagcgggttccagtagcatctgacctgcattagggacaggggcgcggcggagggggcgaagggggcgggggtggggggaaggtggctggggtgaagcccagcttcgcagctagctgtgggcaacagagggagtaagggggggcaatgaggctggggccaggcgccagcagcagccacgccccccacctcccccgatttttagggaaaattctccaaagctctcgcatcctcctctgcctccttccaccctccaccctcccagcctccactgagacctctttaaaaccacccaggggccgccgggggatgaggccggggaacgggctggactgagggcgggggctcgggggcagcggacgggaaacgcctcgaaagcagccagacccggcgactgaaatgaggcggaggagcttggcgaggggaggcgcaggctcggaaaggcgcgcgaggctccaggctccttcccgatccaccgctctcctcgctgacctccgagtcacccccggaagctcccgccactgccgggcgaatagacccccgcggacccccaagcgcgcggggccggggccctagttcaggccctcgctgcccctttaagggttctcgaaactttccccccggtatcagatgagcctcgtcacatccgttggccgtggcaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcctacgctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 101 - DNA, 4697 bp, Artificial Sequence
  DBI2 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgaagaaacctgcatttcttacacttcagtgtactttccccatatttaactccaagatttttgttaatttgtttggttttcctttctcaaacaaaattatgctcagactgaaaaccctagatttgttccctattgcatcttcatttcttcccaaacattccataaaacgtgacctacattaagttagcaagttaagtctgaaagcgtctaccttccctggggagggggaaggtgtaggcagggcagagatttgtagtccagccctcttgccacaaattatgaattagagaggaatgactttgcttttttaatgatctccagagaattttccatcatttccctctcttcacccagctcctttgcaaccactgccagagaagtcttcctttagcttcttaaacatcgatcctaaaacacttccagacacctgtgctgctcctttcagttcccatggagattaggctgtgtaacaatctcgcaaagacgttcccctccgtctcctcatcctcttttcaaacccttttacgatttcccatctcactcagcatgacagtcaaagtccctgtgatggccaacttctgcatcacctagccagtctgccaccgccaaaactctccagcctcatcttacacttgttctctgcttggaatcttccctcccctccttgaggaactttctcaaatgtcaccttccctcaatactccccctcctccatttaaaactataaacttccaactctctaagcccctaaagtactctatatttaacttattgtataaactactgtccctacttgtaagttccaagattgcagggattcacccgctttgttcactgctgtctgccaaggtctagaacagtgcaagttacccaacaggagttcaataaacagccattcatttaacaaatatttgctgagcacttcgtcccgtccaagtttgttaaatcaagacaaataagacaccgtccctgcctttaacgcaccagatggagaaatgcaccacagacataaatgtgcaatacaggcctgacactacggccacaagcaagtcaaagaacgtgccaaaagttcagaggaagaagcctcggcttcgcctttcgggagaccagtccagctttccaccatcacgctgctcatcagggaccatctccgggggtctcctctagaccccaagggaggagcgggtcccgcccgccattcccaggtctcagagtttacttgtccagagatgcaacttccggcctcttcaggccgggcaagatttaaggaaagaaaagaaacataaggacctccgttcttcggtctccgtcccctccccttcccccgcgtgccccacctgttcccggcgtccccttcggctactcccggcgtttgcgcaagcggtcccacgtgggctcgggcggggctagcgccgcggcgggggctgggcacgcccctagcgcatagctggcttctgattggctttccaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgccaaagcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 102 - DNA, 4982 bp, Artificial Sequence
  FABP3 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttaccattctgcctttcacctgatgttgctatcctcctccctcttgtttccttccacccatcctttccctcccacattactctcttatcccaccctattttacaaccagtagcctagggaaaagagcatagctcaaatgaggaagaaggcaggacaggcagtcatggcttagctggactgagctgcagtgcttctccttctggggaagggggtgcactgtcatctgctactgacacatccctccaaggcactcagccctgcagggagcaacctgattctatgactgacatctaatcttcacattcaccttgcaggaaggcaagaagtgatcccagcctccagatggaaagatcaaggcccagagaaggtcagtggtggttggaggcctgaggtcacacagcagccaagtctggagtcactagtcaaggtgaccttgactagccaccccacctccccttccctgccccaccatggccctgggagatctgttgtcctgtgagggaaaggggctccaggctgggctgcatctgaagcccctagatccagagacttcatttcttaggctatctataaaatccaccttcctttcttttcccaggacccccataccctgctcccagcatcgtctgcctcagctaagccatggggattgagagaccaggcctggtgcccagataaactgaccctgggtgaggggacaggggcccagaatgggcaggtagagactgaatactgaagaagaatcctctggagtctgttagcagaagcagatgggccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaacctcctaccatagagttgggaacactgaagaaaagagactggcccaaggtcacagagaaggcagggtgaacactgtcacagggagagccagtgtagaataatggttaagccacgcaagctctagaaccactctatctgagtgcaaatcctggctgtcatctggtacttgcttcctggaacacatctggcctcagactcctgaggccaagacacactccctgccctaagacttgctggttctatggcaggcagaggcagaaagagccccaccatcattcccagcaaatgggaaaagttcccagttgcagatattaggggtgggatggggcgggggtagtcagcaaccatagacttagaccctgaagaggcaaaaaaggagggccatgttcttgggtcagcagagcttctactcagcttcttcagcctctagctctttcctggtgctagtagcacattctctagtggaggcatccagatggcagggagggtccaggaaacagctgaacatgctgagcaggcctcccttgtccccgctccccatggccccatggatcatccggtgctgcagctcatctcattggctggcttctggttactcatctctcctcttctccatcttcccagcctgtggttgccgtggaaacatagaacagtgacctcaccataggatgagggctggggagatgctgttcttggcaggcgctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtccatccacgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 103 - DNA, 4485 bp, Artificial Sequence
  KLHL14 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgaaacagcagccattgatgtagctcagggttctgtggatctgtcatttggagcatgttggttctcctgtctcagctgggctcattcatgcatctgagttcagctattgggcaatctggggaatgttttgtccatgtgatgtgtcatcttctaccaggctagcctgggcttcatcacatggtatctggcagggctctaagagggagagttgaaacacacaaggcctcttgaagcttagactcagaattggcacaaggtcgcttctggcacattccattggtcaaagcaagttacaaggccagctcacattcaaggattaggtaagtcgattccactcttgatgagaagtctgaaggatttggaacagtgtccaccatgcagtaataaactcaataagtagtagccattattattctgttagaggttgccaggaaaagttttatagtggaaagaaatctgagtttactcttgagaggtaagtggaatttctatttgtagagaatgaaggcctctcaaaaagacacagcctaacaataggtgctgcagtttaacagtggagcgtgtccagaacaggctgcccttttaggcaagggctagtgtctttcaggacagacccaaaccccaaataccaaaacagaataaagtagtgtcttagcatactttgagatcagactgtttctgcatttcacagtgctgggggtgggggggaggtgtggggggaagggaaaagcagcataccaatgtagtgaaatctggaaacaacagccaaaaaaagtttgcatattgcacagagcacttgaagatcataaatctatgcatgagaaagatgtagtggaaattttgggggggattagagtttatttttgtcatctctgtgagacagctactcattcatccagatcacagctaagaaaaaagctggtcacagaaattagcagtttcagctcagcagcgaagtcgccagcctgtgaaggcagagagaaattgactaattagcaatgcgcactaaaacttgacggttctttatagagagagagaagagagagggagagagagggagagggagggagggggggctcgctttttccccttctttcttccaaagatgtttgaaatcgcagtcatttacgctcgacaatttttacaatagccttgagccataattttgcgagtctctccagcatccatccccctgtatggtctctctctactggccaagcacgaccgtttctctccccaaccgtggatttcctattaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcccgttctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 104 - DNA, 4242 bp, Artificial Sequence
  MMP15 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccttcctcctccagggccctctgcagaccaggctgagatggaggaacctgctaaaatcgatggagatgcttctagcctcccagtaggaggccccagccatgccttcaacctggcaggaggtgtagccactcctcatccttgggttgcaggttgggtgctgctgttgtggtccttcccagaaactgccagtagagggcagcctgggcatcctaatgcttactctggttgttacacaaagaaaatattggggtcactggcgagcccacccacactcaccagaatctccactgtagtccccctaacaaacagcccttcacttcctctcccacttcagcaatttgtattttgatgccattggcctcagatcagagtgttttaaatcatcacgccctggcttatccctggtcgagccaggacacggggtgcttcagtgggtctgtcaccctctctccttgaagcatgttgcttttatttatttacttttactctcaccctgctcctgtaccagcaggggccacttcaaagccaaggtacagggtgataacttgtggtccagcatcagttttctccacttctttctcccactcacccccagcaaggtgcctggggagacttgagcagatgtttcattttggcctggccagtggctgaaagccaggcctccaatgcactgtgacctctggcttccccagcagctttcccagagaggcagagggagtcttcattcttcccaggcggggagaccacgccttccctgcctcctccctccgcggggggtcgcgttggaggtcacccccgccccctaggcgctgggttgggagtgacgcggggtgggctggagaggtttcctgccgtctgggaagcgtaaacggaccgcccacctgtcgggcctcggccgcccgcacctgcttgtgagaagcctgcggctggggcaccgcccccggtccccgcccgggtccgcgcattgggagcacactggccctttaagagcgcggcggccgcggcgcgcgggaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtttcactgggttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 105 - DNA, 4309 bp, Artificial Sequence
  SPARC construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtcaggctacctctcaggctgactgagtcatgcagcataggctgccacgtctctgggctggcggggccgtcattattcctggcctcactgcagctaaattgaagaaacgtttggtttgtgggccacgtcaaggaatgtgtaagagctgccacgttgtcgggtctgggttattgggcttttcccctccttcagagaagatttccaggcgtgtgggtggggtttcagaagaaaattgatgcctgcgtgtgagtgttccctggacctggaccagcagcggcaatattacagacccgggggttggggcagactgagccaatctctgcaccgtcaaagttatggatacagagccctggaaaaaggctgaaggataagatagctgacatttatgaagtgcttcattcatgtagcagtgggccaaatgcgtactttacacttgaggaagctgaggctggaggttgataacatgcctcaagtcttctagagttaaataactttgacccaggacccaagcccagagttctgactcaaaaactaggcctcctaaacatcctcttatatgaggttaaatttcatcttcctctgtttggccttggcctggttggtggatgctctgcttcggggacccagggccagatgacaatgggttctttgtgcccttcagacaatgggaagggctgcctggggaaagatacagtaacaaggcaacaggctgagtcagcctccaatgtgcttgaaccttcttagcttggcagccttgacattcagccagccacacaaagggtatatcaaggatgataccactagtagcagcttgtcttgtctgtacctctgaacaagaaagaggctgttctgggtcatccctccaggcctgtccagccctggcactctgtgagtcggtttaggcagcagccccggaacagatgaggcaggcagggttgggacgtttggtcaggacagcccaccaggaggaaagaaatgaaagacagagacagctttggctatgggagaaggaggaggccgggggaaggaggagacaggaggaggagggaccacggggtggaggggagatagacccagcccaaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtatactctcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 106 - DNA, 4453 bp, Artificial Sequence
  TSPAN8 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccaaggactcttttttctaaacttcccttcatcttctagtttgacgcccttggtgggaaaagtgtctgagataaggaaaaggcatcctttcagttctctgatactatcttgaagcgagggatggagaaaggcaaagagagacacaggagaagcgtatcccctgggaacaggtgtctagtggagtccagtaactcacagtctctcagttccgtcagcactgtcccttgggtcgcaaatttcttccattagcccttccaccagctgtatttcaaatggggctggacaataattgtggccagtggccttgtgttgtttgtacttgcggactagtagttctcacctgtctttctctgactcctattagccactgggatttcagcagctggttcagccaattctactcaattcaacattaagttgcagtgggctagaactcatgggccgatttaacaagtgaaattctaccaagatacatcaaaaatagcaacaggactagatactcagctcattttgttttatttgtaatataccagttgtggctttagtgccagtctgattcatctctctactacaaaatgaggctctataaaggaaaatattgcaactggagtgaggaatttgaatcttataggaaggaatttgtcttctcatgaagacttcagtttaccagaagtatctattgaggaagtgtttacaagaaaatgtgccatttagctttattctaaatttgcataataactgaaccaaacaaaaaaatatagatagatagattgttctatctatagatagatagggaacattggcagtaggtggcagtaagttcccctgagcacatggaggacacagttaaatgcatttgaggtatgtgggaaatggtttaaagcagaattttatgccaacttttagtaacggaagcctaacaaatgtttgttctttcaagtgagagaagcaagcaatctggaactattcataagcttattttctgtatccttaaacatattttataatgaatgtatgatttaaatagtaagttaagtgtctgggggtactgcacacctcccttgcatacagtcaaacttcttcagggtgatggggaagaggagttataggctgccaagcaaaattgccaaactggtctcagaaattcactgcattggagagcgcgggatccttgcaacactgactttagcagttaaactagagtggttggggatgagtattctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtggcacttcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 107 - DNA, 4317 bp, Artificial Sequence
  VIM promoter
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtattcacaatgcattccctctgcccaccacattaattatcaactccttttcctggcatttactcatccaacgcatggccccacgttaactttcagttccctttctcccctacaaatactccataatccagcaaccctgggatccctgagatgatgaagaggaccagtgcccattccaggagacatcaccgcagccctgaggaatcggctatgggcaccagcagggcacagtgccacacctcgccaatgccttgtcctccttttccatagtgagtcagtcagcaagcgtgtagaagtgagttccacactctcttcctcccatagggagatcacttttctcattctaagggttccaggcacactcacaatggtggcatttgctgagcagtggcttgaataaagggctctcagaaagcaagatgtaactcagagcataggctagccccaggaatgctcttggggaatgacctgcagcctcccagtgaaagagagaataaaagaaagccccagcaggcgagctgggcagtagagagtcctgtaattccaccttggcaagcaccatttgcaagaacgaactgggataaggtaaacaaaatattgcctaaaagaggcttgtccaaagaagtcagaatacgctcttcatttacctctaaatttcagtacaccataaatctaaatactcaaaaaaacctgtgccttttcaatcaaggtcaattgcacgaattcttttggaaaacaggacctatggcatttcccagacaaatcaccgtgaaccctgtactgtgcattgctgtcctaaaatccaaagattctgtcatttgtgttacataattgcctttcatttgaactcattaatcaaattggggtttttaagcaacacctaattaattctttaactggctcatccactgatcactgagttctattttgaaactacggacgtcgagtttcctctttcacccagaattttcagatcttgtttaaaaagttgggtgtggtttcatggggggagggggaagagcgagaggagaccagagggacgggggcggggactctgcaagaaaaaccttcccggtgcaatcgtgatctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggttttcaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 108 - DNA, 8 bp, Artificial Sequence
  Exemplary barcode
cctacgct

SEQ ID NO: 109 - DNA, 8 bp, Artificial Sequence
  Exemplary barcode
gccaaagc

SEQ ID NO: 110 - DNA, 8 bp, Artificial Sequence
  Exemplary barcode
ccatccac

SEQ ID NO: 111 - DNA, 8 bp, Artificial Sequence
  Exemplary barcode
cccgttct

SEQ ID NO: 112 - DNA, 8 bp, Artificial Sequence
  Exemplary barcode
ttcactgg

SEQ ID NO: 113 - DNA, 8 bp, Artificial Sequence
  Exemplary barcode
atactctc

SEQ ID NO: 114 - DNA, 8 bp, Artificial Sequence
  Exemplary barcode
ggcacttc

SEQ ID NO: 115 - DNA, 8 bp, Artificial Sequence
  Exemplary barcode
tttcaggt

SEQ ID NO: 116 - DNA, 145 bp, Artificial Sequence
  Synthetic sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 117 - DNA, 678 bp, Artificial Sequence
  Exemplary Human GJB2 cDNA coding sequence
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 118 - DNA, 681 bp, Artificial Sequence
  Exemplary Human GJB2 cDNA coding sequence
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 119 - DNA, 3074 bp, Artificial Sequence
  Exemplary spliced Human GJB2 isoform 1 cDNA including untranslated regions
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 120 - DNA, 2318 bp, Artificial Sequence
  Exemplary spliced Human GJB2 isoform X1 cDNA including untranslated regions
tttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 121 - DNA, 5469 bp, Artificial Sequence
  Exemplary Human GJB2 Genomic DNA sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaactttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 122 - DNA, 12513 bp, Artificial Sequence
  Exemplary expanded Human GJB2 Genomic DNA Sequence including certain regulatory regions
gactgtgaacttaaggcacagcagagctggggctgctcttaaggccctgctgtctctcctcttagtaacaacaccatttcacatgaagtgacagtggtatcttttgttgccctggaaatggaatacaacaatggctttccaacttttctgtggcagagacctacagacagaagtacattttacactggatccaggacacacatcagtctgaaaacacacacatgaaccaaacgtttcctaaagcattacttatccttgctaatagcaacacattctcatattcttttatacttcatttaatttcatataaaaaagaaaaggaaaggaaagaaatctatttctcagcccattaataaggtcaggagcagcaacaccagactagaagaaaagcttacctatagatttttctgccacctcttgagtgcgtccagctttccgacaagtctcagtgccatctactgtgcgctctgggtattgcaattgctttttttttttttttttttttttttttagaatgagactaagtcagagaacacaaagaacttctttccccacagtggagatggctctgaaagcgtttaaggaatagcttagatgagtggctaacacattctcccggttctgaattctaagaccacagactccatgtccagtccccaaagagaggctttgcaagctacagaatacccctctgactgggacctcaggagctaaactgaccacgtaattggttctagaaagtgaaacgttttaatttgaaacatccaaatgagcattttgtgaaaagctactgccgtccatcaaatacaacacagccagggagtcatcgctctattgcccttgtcaatcctacatctatagttttttttgctacagcagttcatgagtgttgactctattctaacttgttccagaagcccttcaagatgatagatagcacaatatttttgtagccagagctagaatgtagagctctttttggcttccttgtgaatgatccagaatttccatgttggcaagccaccataatttacagaatttactttttatattcaatagaagtaaaaaaaatttacctatttaaggagttatagctctggattcatttctgaccaaaatgtgctttttgacacaaatacaattggaaatgtctttgtaatttatccacagtctgcctagataatcataaaagaactgcatggatatatttgtgagtaagagcacgtgtccattcagcaaaaccaaggagatcaactaattctaccattgccttgaaacggagacacatctagcagtttgaatttcccccaaaagattgtatgtgtgaaaataagaatagaatgaggaaaatttaaaagcctatataataatttcagtcacaacttggcaattagaattttatgagatgtctttaatttggaagcaaagaacaattaaattattgaaggctggaatttttttttaactctttgaatggaacaacagattttccccaaaagatttgactttaacaattttcagaaaacataagtcagggtgtggttcaattacacagagagaaattgtagtgaaatagtgttccctgtaataattacccacaaaggagcacagtggagccactcctgcattaaaattacagtatcatatgtaagttattattaattaaccagagatgccaggagcttgtcagtttccaactgctattttgaggagagctaaagtttctctttttttgccagttattattattattaatatttcaacagcaaggcaagaaaagggaatgtggtccattaactaatggctcttgaaaagacactcaatgaatccaacttgccctaaaactgccaagtggtaggacagtctcttcgcgtcttgcatcattttctgccatcacctacgtgtgattcgtgagtcggaaattcaaccaagacatgtttaatgtatatttagagcattcttcccggcgggaattcacggtgccattccatcaggcagttggcaagcagtcacttgaaatattaagaaatatgatttgtgtcacactgatttattgcaaaacagcaacttctttctttttggttcatttataaaacaactgtcaaattaaaatgccaaatagctttaaacattagcattttcaccttataaccttacaagtgcatcactttaaacatctgagtaaaagttcagctcgatgacaatcacctgggatttacctgcatggtactaagcatatatgtaaaaatattactgatgggtatctctggcactctgaagtgacaaagtgtagccttcacagatctttgtcagttaatcatcaatagttacctgaaaagtgcccacttgccatcattcaagatcaaccaggcagacaccacagtgagttttccatcaaaaaaccttctctatctggtcagtctctgcacgtcaatgagacaaaggtgtatgctgcacgcagcagtactatcctaagctccctgtgtcctcaccatggggctgggtggctggggtggaggaacacaggattgggcttcagcttctctagggactggtacattaagagatgaagacataaaaggtgagaaaaacatggtttatttccaatgtttccatttctgttaaaagtaatgctttcaacagaaaaaaaatgcagcaatataagtgtgtaatttacaaaataatttcaggatttctttaatcattaatttgtggtgtcatctgttaactggatttacgtctaagctcatttgtaaataacttcaaatatccaagccttccctcacccttttcccacctcacctctcctccttctcctcccctacactggaggacactatgtacatgcatataatgtcctgccctagaggagtcctgagcctacttgggaagaaaacaccaactcacaggaaaacagcagaaatcacacaaaacagaataaaagcaagcgctgatctgtaagtgaagacttaagtgctataggacttccagctacaaatcctgaaaacacggagtggctgtgataatacgactagccaacatcacacagtaattttgcacataaggagaactaaatcaaagaaaacaaggaaaagaaagttgagcctataatcgtgatacaggcactaaaatctcaggtgacatttttcaatgggggaaagtcagtcaacttccgatctccaaaccatctttactagcgagcttcccacaatggttctagaaccttccttcattccaacccaaccaggattccaacagactcataaacaccacagcctttgagaaattaaagggagaacccaccaaccggcgccccactccccaccccaagtcacctctggctcaaccaagatgcgctcaggccaagaaagctgccccaccccacaggctttgcctgtcatttttaacaagccgactcagcacatctctcagatgggccatgcaaggcttttcgcagctcctggggctttgcctcttcatgagcagacactccctcttagactaagacctggagctggaaagtaggtggtaaccgcggtacaaaactcacgctcgtccctgcagaaactgcctaggtcggcccatggccacggggcgccaatttttcaaggaaaagtcaatgctaataatggtggcaatcacgggaaatccattctgaggccagatctgacttgtcaggattaatcatcatttccacttaacttcgaactgacctgggtaaaaacgtgagcgcgaggggaccaggctgcacctctgacctggctcccctctgcaaaaatcgcgaagtgggtgcccgaggtggggcgggggttgggggagacctccccgggagtccccacccagcctgctctgcacatcttagtccctcatccgcttgcgctgtgcaaatctgtcttctgtcatttgtatcgcaagacatcaaaatccccaaccaaatgcaaatactgagacctcataatctgagacaaagtttcacggtatccagaaagcccccagcaggtgtgcagtgcagagccagccccccagcggtcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaactttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcaaattttaatttcatctatcactttttatataaatacattaatgttttatatttcataacaccaatgggtaagttgccagagtgtctgaccccattctgccccagttacagaaaagcttctgtcaccagaaagtttggtggggaaggaagggaggaagatgatttctacctaaccccgtgcccacctctaccaggtttttgaggcatatcagtctatggacaatgtggtgtttggtctggaaacgtaccttggtgaatgctgagttggctggacatgacccgtttagctcctggatgaatcccagaagtggaccttcaaaatgttactcatagcatgaccttggctcactgcaacctctgcctcccaggctcaagcgatcctcccacctcagcgtcccaagtagctgggaccactggagtgtgccaccacactccactaattttttcattttttgtagaaacgaggtcccactatattgcccagtctggtctcgaactcctgggctgaagggatccccctgcctcagtctcctaaagtgcaaggattacaggcatgggccaccgcacctggcctgaaactgctttttattcctcagtgcccacttccatgggaaataagcctgccaggtcagcctgtccccatgggagtgactgcctgctacccccacaggcttgcccggccctcgtgagcctctcccagagacaccaccaacagttctgttctttcatggtacaagatttccatccaaggatttcaaagcatttcacacatcaataattagaagtattttcatagaggaccatacacttttaaaatggatttcaaagaacaaaaaccagtcaactatcacccaggtaatagaaaatgggaaatggtttctacctgacttccaaaatgctctgcacatagactgtgaaaataggattttttaagctgggtgcagaggcttatacctataatcccaacactttgggaggctgagacgagaggatcacttgagcccaggagttcaaaaccagcctgggcaatatagggagacattgtttctataaaaaataaaaatgttagccaggcaggcgtggtaacatgtgcctgtagtctcagctactcaggaggctgaggtgggaagattgcttgaacctgggaggtccatgctgcagtgagctgagattgtgccactgcactccagcctaggcgacagcaagatcctgtcccaaacaacaacaacatcaaaaaacacagaacttttaaaataagtacattcacttctacaagctatgtagattattactctcaagctattaaaagaccaagccaaaataattatgggctactctcgaccacttgtaggaatggatagagaggtctggtcacatgcctggaaattagagcttgagctctgaaaatgataatcctgactatatctcaaagcatcagtctgcactttgtatggagcaagaaaaagccttgtggaagcggcctcccacccagccgagccctcggcgtggacaagctctgctttttatgagcagtgggtgcagcctcgctgctccctcctcctgtcaaaagacagtcacagctggggtgagcagatcgggcccacttgggaggccccaaggaatatgctgcaggggtcgggcctgagccacccccacgggttggtctttgacaactagagagcagctgagaggtgggtaaaagctcactcacttaccctgacctcagtgtcctcatcttaaaatgggtttcctgaatctttccccggcttagtggcaatgaaataagataatttatgtaaacgttctccacatagtaaagcactaagtaacatatgactgtcatctgttttccactagacagatcccaacctggaagagtgacagatggtatttcagatacaagtgactcaagcaaagcttgataaactgggggctggaaaaaaatgcacatttacacaaagcctggagtaactgc

SEQ ID NO: 123 - DNA, 678 bp, Artificial Sequence
  Exemplary codon optimized Human GJB2 DNA sequence
atggactggggcaccctgcagactatcctggggggcgtcaataagcattcaactagcatcggaaagatttggctgactgtcctgtttatctttcggatcatgatcctggtggtggcagcaaaggaagtgtggggcgacgagcaggccgatttcgtgtgcaacacactgcagccaggctgcaagaacgtgtgctacgaccactattttcccatctctcacatcaggctgtgggccctgcagctgatcttcgtgagcacccctgccctgctggtggcaatgcacgtggcctatcggagacacgagaagaagcgcaagtttatcaagggcgagatcaagagcgagttcaaggatatcgaggagatcaagacacagaaggtgaggatcgagggctccctgtggtggacctacacaagctccatcttctttcgcgtgatcttcgaggccgcctttatgtacgtgttctatgtgatgtacgacggcttttctatgcagcggctggtgaagtgcaacgcctggccctgtcctaatacagtggattgtttcgtgtccagacccaccgagaagacagtgttcaccgtgtttatgatcgccgtgtctggcatctgcatcctgctgaacgtgaccgagctgtgctatctgctgatccggtactgtagtggaaagagcaaaaaacccgtg

SEQ ID NO: 124 - DNA, 678 bp, Artificial Sequence
  Exemplary codon optimized Human GJB2 DNA sequence
atggactggggaacattgcaaactattttgggaggagtcaacaagcattcaactagcatcgggaagatctggctgaccgtgctgttcatctttcgcatcatgattctcgtggtggccgctaaggaagtctggggcgatgaacaggccgacttcgtgtgtaacacgctgcagcccggttgcaaaaacgtctgctacgatcactacttccccatctcacacattagactgtgggcgctgcagctgattttcgtgtccaccccggcacttcttgtggcgatgcacgtggcctaccggcggcacgagaagaaaaggaagttcattaagggcgaaatcaagtccgagttcaaggacatcgaagaaatcaagacccagaaggtccgcattgagggctccctctggtggacctacacctcgtccatcttcttccgggtcatattcgaggccgcctttatgtacgtgttttacgtgatgtacgacggtttcagcatgcaaagactcgtcaagtgcaacgcttggccttgccccaataccgtggattgcttcgtgtcccgcccgaccgagaaaactgtgttcactgtgttcatgatcgccgtgtccggcatctgcatcctgctgaacgtgaccgagctgtgctatctcctgatccggtactgtagcggaaagtcgaagaagcctgtg

SEQ ID NO: 125 - DNA, 678 bp, Artificial Sequence
  Exemplary codon optimized Human GJB2 DNA sequence
atggattgggggacgctccagactatacttggcggggtaaacaaacattccacctcaattggcaaaatctggctcacagtcctcttcatcttcagaataatgatactcgtggttgccgctaaagaagtttggggtgacgagcaagccgatttcgtctgtaacaccctccaaccaggttgcaaaaatgtctgttacgatcactactttcctattagccatattagactctgggccctgcaacttatcttcgtttccactcctgctctgctcgtcgctatgcacgttgcctatcgccgccatgaaaaaaaacggaaattcattaagggagagattaagagtgaattcaaggatattgaagagattaaaacgcaaaaagttagaattgagggatcactgtggtggacttataccagtagcatcttttttagggtcattttcgaagctgctttcatgtatgttttctatgtaatgtacgacggtttctccatgcaacgcttggttaaatgtaacgcctggccatgccctaatacggttgattgctttgtctcccgccctactgaaaagacagtgtttaccgttttcatgatcgccgtaagtggaatttgtatccttcttaacgtgaccgagttgtgctatctccttattcgctactgttcaggaaaaagtaaaaaaccagta

SEQ ID NO: 126 - DNA, 681 bp, Artificial Sequence
  Exemplary codon optimized Human GJB2 DNA sequence
atggactggggcacgctgcagactatcctggggggtgtcaacaagcattcaactagcatcggaaagatctggctgaccgtcctgttcatctttcgcatcatgatcctcgtggtggccgctaaggaagtgtggggcgacgagcaggccgatttcgtgtgtaacaccctgcagccaggttgcaaaaacgtctgctacgatcactactttcccatctcccacattagactgtgggccctgcagctgatcttcgtgtccacccctgcgctgctagtggccatgcacgtggcctatcggcgacacgagaagaaacggaagttcattaagggcgagatcaagagcgagttcaaggatatcgaagagatcaagacccagaaggtccgcattgagggctccctgtggtggacctacaccagctccatcttctttcgggtcatcttcgaggccgcctttatgtacgtgttctatgtgatgtacgacggtttctccatgcaacggctggtgaagtgcaacgcctggccttgccctaatactgtggattgcttcgtgtcccgccccaccgagaagacagtgttcaccgtgttcatgatcgccgtgtctggcatctgcatcctgctgaacgtgaccgagctgtgctatctcctgatccggtactgtagtggaaagtcaaaaaaaccagtgtaa

SEQ ID NO: 127 - protein, 226 aa, Artificial Sequence
  Exemplary Human Connexin 26 Protein Sequence
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 128 - DNA, 1044 bp, Artificial Sequence; identical to SEQ ID NO: 65
  Exemplary Regulatory sequence C3

SEQ ID NO: 129 - DNA, 1044 bp, Artificial Sequence; identical to SEQ ID NO: 65
  Exemplary Regulatory sequence D7